(19) United States
(12) Patent Application Publication
(10) Pub. No.: US 2003/0211476 A1
(43) Pub. Date: Nov. 13, 2003

(54) Genetic analysis of peyer's patches and M cells and methods and compositions targeting peyer's patches and M cell receptors

(76) Inventors: Daniel O'Mahony, Blackrock (IE); David Brayden, Glenageary (IE); Daragh Byrne, Baumont (IE); Imelda Lambkin, Sutton (IE); Lia Higgins, Donabate (IE)

Correspondence Address: CAESAR, RIVISE, BERNSTEIN, COHEN & POKOTILOW, LTD., ATTN: ELAN, 12TH FLOOR, SEVEN PENN CENTER, 1635 MARKET STREET, PHILADELPHIA, PA, 19103-2212, US

(21) Appl. No.: 10/116,275

(22) Filed: Apr. 4, 2002

Related U.S. Application Data
(60) Provisional application No. 60/281,387, filed on Apr. 4, 2001.
(60) Provisional application No. 60/302,591, filed on Jul. 2, 2001.

(51) Int. Cl. C12Q 1/68

(52) U.S. Cl. 435/6

(57) ABSTRACT

Methods of increasing of or decreasing the levels of a protein in a PP cell; methods of increasing antigen, vaccine, DNA vaccine delivery to M cells, use of human serum albumin and other transport enhancing proteins to enhance oral drug delivery; use of calreticulin to enhance oral antigen delivery, use of other cell surface proteins, receptors, and transporters to enhance delivery to M cells of antigens or vaccine delivery vehicles, use of other cytoplasmic proteins to regulate intracellular trafficking and delivery to mucosal immune sampling and processing systems.

### CROSS REFERENCE TO RELATED APPLICATIONS

This application claims the benefit of U.S. provisional application No. 60/281,387 filed Apr. 4, 2001, and U.S. provisional application No. 60/302,591 filed Jul. 2, 2001.

### FIELD OF THE INVENTION

This invention relates to the genetic analysis of M cells and methods and compositions targeting M cell receptors.

### BACKGROUND OF THE INVENTION

The Peyer's patch of the intestinal lining is a specialized tissue that allows the immune system to identify foreign antigens that require an immune response. It is also a potential pathway for orally delivered drugs to cross the intestinal barrier into the bloodstream. Central to these properties are M cells, which populate the patch's epithelial sheet. In view of the importance of the Peyer's patch and its M cells for the immune response and drug delivery, it is desirable to identify the cell proteins important for these phenomena. It is also desirable to increase the amounts of such important proteins in order to either facilitate the immune response and drug delivery or promote the conversion of non-M cells to M cells.

Similarly, it is important to identify and further decrease the levels of proteins whose absence or down-regulation in expression facilitates the immune response and drug delivery, or promotes the conversion of non-M cells to M cells.

### BRIEF SUMMARY OF THE INVENTION

Increasing the Levels of a Protein or Antigen-Protein Combination

In a first general aspect, the invention is a method of increasing the levels of a protein in a Peyer's patch cell, said method comprising delivering to said cell a nucleic acid coding for a protein, wherein absent said increase, the levels of said protein or its mRNA is greater than in a non-Peyer's patch cell.

Peyer's patch cells of particular interest are M cells. The levels of a protein or its mRNA in Caco-2 cells co-cultured with Raji B cells are considered herein to be representative of such levels in a human Peyer's patch M cell. Monoculture Caco-2 cells are considered herein to be an appropriate non-Peyer's patch cell for purposes of comparison of such protein or mRNA levels.

The levels of a protein or its mRNA in rat Peyer's patch epithelial cells can be compared to their respective levels in a culture of rat normal gut epithelial cells. Absent evidence to the contrary, results of rat cells are assumed to be predictive of the results in human cells.

The presence of Increased levels of an mRNA, and therefore presumptively its protein, are indicated in the Table 2 and 3 by a ** a *, or an expression Fold Change greater than 1.00. Preferred are those indicated by a ** or an expression Fold Change greater than 2.00. Most highly preferred are those indicated by a **. The presence of decreased levels of an mRNA, and presumptively its protein, are indicated by a minus sign (−) or an expression Fold Change less than 1.00. Preferred targets are those indicated by a minus sign or an expression Fold Change less than 0.50.

In embodiments of particular interest, the protein is a receptor, a transporter, cell surface antigen, or cell adhesion molecule, especially a receptor. In other embodiments of particular interest, the protein is selected from the group consisting of nucleoside diphosphate kinases and member of the 14-3-3 family.

In the methods of greatest interest, the nucleic acid is delivered to a human cell. There are many delivery options, one of which is to deliver it by the oral route with the cell in a human, another to deliver it to a cell outside a human.

In an important variation of the method, a nucleic acid coding for a tumor antigen or foreign peptide is also delivered to the Peyer's patch cell. The purpose of this aspect of the invention is to improve the immune response to a tumor antigen or the foreign peptide. Normally, therefore, the foreign peptide will be that of a virus or infectious microorganism. A tumor antigen is one that is more abundant in a tumor cell than its normal counterpart.

Decreasing the Levels of a Protein

Another general aspect of the invention is a method of decreasing the levels of a protein in a Peyer's patch cell, said method comprising delivering to said cell an anti-sense nucleic acid molecule, a ribozyme nucleic acid molecule, an RNA interference (RNAi) nucleic acid molecule, said anti-sense, ribozyme or RNAi nucleic acid being complementary to a sequence of at least 10 nucleotides of the mRNA for said protein, wherein absent said anti-sense nucleic acid molecule, ribozyme or RNAi nucleic acid, the levels of said protein or its mRNA are less than in a non-Peyer's patch cell. More preferably the anti-sense nucleic acid is complementary to a sequence of at least 15 nucleotides of the mRNA of the protein, and most preferably to a sequence of at least 30 nucleotides of the mRNA of the protein. It is preferred that the protein is coded for by a gene with an expression Fold Change denoted by a minus sign (−) or an expression Fold Change less than 0.50.

In a particular embodiment, the latter method comprises delivering to said cell an anti-sense nucleic acid molecules, a ribozyme or RNAi nucleic acid molecules, said anti-sense, ribozyme or RNAi nucleic acid being complementary to a sequence of at least 10 nucleotides of the mRNA for at least 5 different proteins, wherein absent said anti-sense, ribozyme or RNAi nucleic acid molecule, the levels of each of said proteins or its mRNA are less than in a non-Peyer's patch cell.

Alternatively described, the latter invention is a method of deceasing the levels of a protein in a Peyer's patch cell, said method comprising delivering to said cell an anti-sense nucleic acid molecule, ribozyme or RNAi nucleic acid molecules, said anti-sense, ribozyme or RNAi nucleic acid forming a double-stranded molecule with part or all of the mRNA for said protein, wherein absent said anti-sense, ribozyme or RNAi nucleic acid molecule, the levels of said protein or its mRNA are less than in a non-Peyer's patch cell.

Cells of the Invention

A human or rat cell to which any of the above methods in this Brief Summary of the Invention section has been applied, or the progeny of said cell, is also an aspect of the present invention.

Delivery Enhancement using a Targeting Ligand which Targets a Receptor, a Transporter or a Cell-Surface Molecule Expressed on Surface of M Cells or Pever's Patch Tissue Cells

In another general aspect, the invention is a method of targeting an antigen or a drug delivery vehicle containing an antigen, or a drug delivery vehicle containing an antigen and adjuvant, or a drug delivery vehicle containing a drug, or a viral vector, or a bacterio-phage vector such as, but without limitation M13 or Fd, or a bacterial vector or a gene delivery vector expressing an antigen of interest, or a viral vector, or a bacterio-phage vector such as, but without limitation M13 or Fd, or a bacterial vector or a gene delivery vector expressing a gene product(s) to M cells of Peyer's patch tissue, by targeted delivery to receptors, or to transporters or to other cell surface proteins which are found to be expressed on the cell surface of M cells or other cells found within Peyer's patch tissue, or which are found to be differentially expressed on these cells. Said gene product(s) coded by the viral vector, or a bacterio-phage vector such as, but without limitation M13 or Fd, or a bacterial vector or a gene delivery vector regulate the function of Peyer's patch cells to M cell phenotype or regulate M cell function to increase their immuno-surveillance or antigen presentation to the mucosal immune system.

In one embodiment, a phage display library such as M13 or Fd which express random peptide sequences on the surface of the phage, coded by example gene III or gene VII of M13 or Fd bacteriophage, can be screened by in vivo panning against example Peyer's patch tissue found in vivo in the GIT, in order to discover and identify phage or targeting ligands which specifically target M cells or Peyer's patch tissue in vivo in the GIT; such phage which target M cells and Peyer's patch tissue can subsequently be genetically engineered to encode a gene or genes of interest such as a DNA vaccine gene, a gene coding for an antigen of interest together with gene(s) which modify M cell function and which enhance the immuno-responsiveness of the M cells to the antigen or DNA vaccine product coded by the genetically engineered bacteriophage genome.

Delivery Enhancement using Transport Enhancing Proteins

Another invention disclosed herein is a method for enhancing transport of a drug through the gastrointestinal tract, said method comprising orally administering said drug in a composition that comprises a transport-enhancing protein, said transport-enhancing protein selected from the group consisting of human serum albumin (HSA), clusterin, T-cell surface glycoprotein CD5 precursor, HSP84, and Ca²⁺-dependant phospholipase A₂ (Ca2+ pla2), or a homolog that has at least 80% amino acid identity with said transport-enhancing protein over a length of said transport-enhancing protein identical to the homolog. In a preferred embodiment, the homolog has at least 90% amino acid identity with the transport-enhancing protein over a length of the transport-enhancing protein identical to the homolog. In a more preferred embodiment, the transport-enhancing protein is selected from the group consisting of human serum albumin (HSA), clusterin, T-cell surface glycoprotein CD5 precursor, HSP84, and Ca2+ pla2.

Method of Delivering a Vaccine to a Target Cell

Further invention disclosed herein is a method of delivering a vaccine to a target cell, said method comprising utilizing as the target cell a Peyer's patch cell in which a protein or mRNA is upregulated.

Method of Decreasing the Levels of a Protein

Yet, another invention disclosed herein is a method of decreasing the levels of a protein in a Peyer's patch cell, said method comprising delivering to said cell a DNA molecule coding for an anti-sense nucleic acid molecule, a ribozyme nucleic acid molecule, an RNA interference nucleic acid molecule (RNAi), said anti-sense molecule, ribozyme or RNAi nucleic acid being complementary to a sequence of at least 10 nucleotides of the mRNA for said protein, wherein absent said anti-sense molecule, ribozyme or RNAi nucleic acid, the levels of said protein or its mRNA is less than in a non-Peyer's patch cell.

Method of Increasing the Extent to which the Function of a Protein is Carried Out

Another invention disclosed herein is a method of increasing the extent to which the function of a protein is carried out in a Peyer's patch cell, said method comprising delivering to said cell a nucleic acid coding for said protein, wherein absent said delivery, the level of said protein or its mRNA is greater in said cell than in a non-Peyer's patch cell.

Chimeric Protein that Comprises Two or More Segments, each of said Segments Enhancing a Different Step in the Peptide Transport Process

Another invention disclosed herein is a chimeric protein that comprises two or more segments, each of said segments enhancing a different step in the peptide transport process, said steps selected from the group consisting of binding to a cell such as an M cell, transporting the peptide into the cell such as an M cell, presenting the chimeric protein to a protein processing pathway within a cell such as an M cell in order to maximise processing in a way to optimize presentation of the processed chimeric peptides to epitopes suitable for immune activation, transporting the peptide through the cell such as an M cell, and transporting the peptide out of the cell such as an M cell to an underlying immune cell such as a B-cell or T-cell.

Delivery Enhancement using Calreticulin and other Proteins

Another method disclosed herein is a method to facilitate intracellular trafficking of an antigen that has been orally delivered by itself or as part of a composition or particle, said method comprising administering calreticulin.

Related to the latter invention is a chimeric protein comprising the amino acid sequences for (1) calreticulin, rab family proteins and and/or a ribosomal protein, and (2) a second polypeptide. Also related is a method of administering a polypeptide, where said polypeptide is part of the chimeric protein and wherein said chimeric protein is orally administered.

### DETAILED DESCRIPTION OF THE INVENTION

The present invention and the related research were intended to improve targeted vaccine delivery and targeted gene delivery methods, especially as they relate to Peyer's patch cells. In significant part, this was achieved by identifying proteins whose up-regulation or down-regulation would indicate their possible or probable role in cellular functions important to vaccine and or drug delivery. In some cases, such as receptors, the proteins are important from the point of view of cell specificity during the delivery process. In many cases, the proteins have functions that are important after the vaccine or drug enter the cell.

Closely related to those inventions and research goals, was the concept that in M cells there would be proteins that, as compared to M cell precursors, were up-regulated or down-regulated. The identification of such proteins provides a strategy for altering M cell precursors so as to shift their phenotype toward that of M cells.

As indicated, one aim of the research related to the present invention was to determine if there were detectable differences in protein/gene expression between: (1) Peyer's patch (PP) and non-Peyer's patch (NPP) rat gastrointestinal tract (GIT) tissue and (2) M cell enriched follicle-associated epithelium of Peyer's patch (PP FAE) tissue. This was done with a view to finding novel or highly expressed ligand targeting sites on the Peyer's patch or M cells as well as other protein relevant to the delivery of drugs across the GIT.

This invention is based in part on the discovery of over-expression of a range of genes in Peyer's patch (PP) tissue from rat small intestine in comparison to normal non-Peyer's patch (NPP) small intestine tissue.

This invention is also based on the discovery of over-expressed genes in co-cultures of Caco-2 cells. The idea was to use genetic mapping of the M cell co-culture, e.g. Caco-2 cells co-cultured with Raji cells versus a monolayer of Caco-2 cells, to ascertain the differences in epithelial gene expression between M cells and enterocytes. It became immediately apparent that some of these gene products are going to be unique apical membrane proteins (e.g. receptors, transporters, adhesion proteins) in M cells. By examining the differences between M cells and enterocytes in vitro and in vivo, one could identify key targets that can be used to generate M cell specific ligands. These ligands can then be used for targeting oral vaccines in particles.

The identification of over-expressed ribosomal proteins or homologues/related proteins thereof indicates a generally higher protein turnover or protein synthesis capacity in PPs or a possible role for such ribosomal proteins (or homologues thereof) in other cellular functions such as protein chaperoning, endocytosis, trafficking of proteins/antigens/particulates/viruses uptaken from the lumen of the gastrointestinal tract (GIT) and/or from the M cells to underlying immune cells, antigen presenting cells, dendritic cells, B cells, other cell types.

The identification of a series of transcription factors (TFs) that are over-expressed in PP tissue versus the control enterocyte GIT tissue is considered herein to indicate a role for such TFs in the development of M cell phenotype, in conferring M cell phenotype and/or in programming M cells to prime other downstream cellular events leading to a better or more efficacious immune outcome following antigen presentation. The co-delivery of genes coding for such TFs with either antigens themselves and/or with gene(s) coding for antigen(s) of question to M cells and/or PP tissue following oral administration provides the basis for a more efficacious and pronounced immune outcome when the TF coding genes are key or vital for driving M cells/PP tissue to an effective immune outcome.

The general over-expression of a number of proteins species in PPs versus NPPs, both membrane and cytosolic-associated was also determined by a novel technique of enrichment and M cell selection following enrichment of the follicle-associated epithelium (FAE) of Peyer's patch (PP FAE) by ethylene-diamine tetra-acetic acid (EDTA) extraction and recovery of M cells/PP FAE. Such novel or differentially expressed proteins have significant implications for the use of this protein expression information and methods of selection/enrichment of M cells/PP tissue for the targeting of drug/vaccine uptake to Peyer's patches. Among proteins found to be over expressed in rat PP tissue following this enrichment technique was the human serum albumin homolog which is considered here to have implications for drug/cargo transport from the GIT either into oracross intestinal tissue including PP tissue and systemic delivery of same to the blood.

### Incorporation by Reference

All references cited herein are incorporated herein by reference in their entireties.

All GenBank records specified bytheiraccession numbers are incorporated herein by their entireties.

The GenBank amino acid sequences and nucleotide sequences specified by their GenBank ID number are incorporated by reference herein. All GenBank records corresponding to those ID numbers are incorporated herein in their entirety. Absent a date specifying the date of the record, the date of the record is the filing date of this application.

Many of GenBank sequences specified by their GenBank ID numbers are reproduced herein in the section “Amino acid sequences and nucleotide sequences corresponding to selected GenBank ID numbers.” The CDS line refers to the exon(s).

Any GenBank ID numbers specified herein, absent a decimal point and an integer following that decimal point, is for GenBank version 1 of that sequence. Any GenBank ID number that has a decimal point and an integer following it is the GenBank version number.

The invention will be illustrated in more details with reference to the following Examples, but it should be understood that the present invention is not deemed to be limited thereto.

### EXAMPLES

### Example 1

### Preparation of Cytosolic (S100) and Membrane (P100) Proteins from Rat PP and NPP Tissues

Protein samples were prepared from PP and NPP tissue extracted from freshly sacrificed rats. These protein samples underwent electrophoresis on denatured SDS-PAGE gels and were stained using two different standard proteins Commassie Blue stains. Subsequently, fresh PP and NPP tissue samples were fractionated into cytosolic (S100) and membrane (P100) proteins and these samples were also electrophoresed on SDS-PAGE in order to compare S100 and P100 fractions in both PP and NPP tissues.

### Example 2

### Preparation of GIT Tissue or Co-Culture Cell Membrane (P100) and Cytosolic (S 100) Fractions

The fractions were prepared using the following procedure:

1. Scrape the co-culture cells into PBS and pool cells into a universal.

2. Centrifuge the cells for 5 minutes at 1,500 rpm.

3. Remove the supernatant.

4. Re-suspend the cell pellet in 3 volumes of ice-cold HED buffer, and allow it to swell for 5 minutes on ice.

5. Homogenize the cells for 30 seconds.

6. Centrifuge the homogenate in hard walled tubes at 40,000 rpm for 45 minutes at 4° C. in a Beckmann Ultra Centrifuge (rotor Ti90).

7. Remove the supernatant (S 100) and re-suspend the pellet (P100) in 3 volumes of HEDG buffer, before centrifugation again at 1000 rpm for 2 min. Remove the supernatant and store on ice. Repeat the procedure and add the second supernatant to the first.

8. Determine the protein concentration (using the Bio-Rad protein assay).

9. All fractions were stored at −80° C.

The following reagents were used in the above methods: HED buffer (20 mM HEPES pH 7.67), 1 mM EGTA, 0.5 mM dithiothreitol, 1 mM phenylmethylsulphonyl fluoride (PMSF):

| | | |
|---|---|---|
| HEPES (pH to 7.67) | 0.5206 | g |
| EGTA | 38.04 | mg |
| Dithiothreitol | 7.71 | mg |
| Distilled water | to 100 | ml |

10 μl PMSF stock solution was added to 1 ml of buffer prior to use.

HEDG buffer (the same as HED buffer plus 100 mM NaCl, 10% glycerol)

| | | |
|---|---|---|
| NaCl | 0.584 | g |
| Glycerol | 11.4 | ml |
| HEPES (pH to 7.67) | 0.5206 | g |
| EGTA | 38.04 | mg |
| Dithiothreitol | 7.71 | mg |
| Distilled water | to 100 | ml |

10μl PMSF stock solution was added to 1 ml of buffer prior to use.

PMSF(100 mM) stock solution

| | |
|---|---|
| PMSF | 17.42 mg |
| Isopropanol | |

### Example 3

### Isolation of Epithelial Sheaths from Rat Pever's Patch and Non-Pever's Patch Tissue

The M cell is a very elusive cell type, at least in terms of isolating a purified population. Previous attempts have found that when M cells are separated and purified and put into culture they very quickly lose their characteristic morhphology and probably gene/protein expression profile. In many cases this is due to the length of time taken to isolate and purify the cells from the very homogenous mix of cells in a Peyer's patch. We desired a quick and routine method to enrich for M cells is Peyer's patch samples. M cells are only contained in the epithelium of Peyer's patches, the so-called follicle associated epithelium (FAE), while underneath the epithelial layer lays all the B and T lymphocytes, dendritic cells etc. So by isolating the epithelium away from the rest of the Peyer's patch dome, we are greatly enriching it for the M cell population. Previously, treatment of mouse intestinal tissue with EDTA was shown to cause separation of the epithelium as a sheet from the rest of the tissue, allowing for it's specific isolation (Bjerknes M and Cheng H (1981). Methods for the isolation of intact epithelium from the mouse intestine. Anat. Rec., (199):565). This method was adapted for the isolation of FAE from rat Peyer's patch. Control epithelium from normal gut tissue (no Peyer's patches) was used as a control.

Epithelial sheaths were prepared using EDTA method comprising the following steps:

1. Sacrifice the rats (Wistar) by cervical dislocation.

2. Remove the entire length of the GIT tract from (but not including) the stomach to the caecum and place in a dish of PBS (at room temperature).

3. Excise the Peyer's patches, taking care to remove as much normal non-PP GI tissue as is visible. Rinse briefly in PBS.

4. Also take samples of normal non-Peyer's (N PP) tissue close to the patches, rinse in PBS and treat as for the PPs (steps 5-9,11-12).

5. Pool the PP's from the entire GI section in Hank's Buffered Saline Solution (HBSS, Gibco Life Sciences) with 0.011 M glucose and 25 mM Hepes.

6. When pooling is complete, place PP sections into 15-20 ml of HBSS (with 0.011M glucose and 25 mM Hepes) along with 40 mM of EDTA into a small conical flask.

7. Add a stirrer bar to the flask, place on a stirring plate and spin the PP's for 15 min at RT.

8. After 15 minutes pipette the PP solution vigorously with a wide-bore 3 ml plastic pasteur pipette.

9. Strain the supernatant through a 100 micron nylon cell strainer (from FALCON™, 352360).

10. Move the filter to another 50 ml tube and wash out the residue material on the filter with HBSS (with 0.011M glucose and 25 mM Hepes, no EDTA). This residue contains the majority of the PP dome epithelial sheaths.

11. Centrifuge the PP residue material at 3000 rpm for 5 min. Also centrifuge the NPP tissue supernatant at 3000 rpm for 5 min.

12. Snap freeze the cell pellets and store at −70° C.

### Example 4

### Identification of Over-Expressed Proteins in Enriched M cells/PP FAE Cells

Epithelial cell layers of Wistar rat PP (representing enriched M cells/PP FAE cells) and normal villi were extracted using EDTA as described in Example 3 above. Epithelial layers from numerous Patches and rats were pooled and the protein isolated into either cytosolic and membrane fractions following centrifugal separation. 2D gel electrophoresis (between isoelectric points pH 3.5 to 10) was performed on 50 μg of each fraction, wherein the gels were silver stained. The gels were overlaid, and numerous differentially expressed proteins between the membrane fractions of PP and normal villi epithelia were observed. Further, protein samples underwent a second 2D gel electrophoresis, this time the gel was stained with a “special” silver stain, that did not inhibit the mass spectrometry analysis of individual spots. The differentially expressed proteins were identified and highlighted by gel overlay.

Thirty-seven protein spots were identified that were increased in PP over villi epithelial membrane fractions. Of these, 16 spots (the most highly over-expressed) were chosen for mass spectrometry analysis. The spots were digested with endoproteinase Lys-C/trypsin (8:1 ratio) and analyzed on a MALDI-MS. The spots, however, gave very poor spectra and only 4 of the 16 were identifiable. These were:

serum albumin;

calreticulin;

14-3-3 zeta (tentative ID; mouse); and

nucleoside diphosphate kinase B.

One protein showed homologyto human serum albumin (HSA). Work byA. Fasano at Maryland had suggested that Zonulin, the human homologue of ZOT, showed sequence homology to human serum albumin (85% homology across the limited sequence available from the Fasano's work). Given our finding that a protein differentially expressed in rat PP tissue shows homology to HSA, we propose that HSA (or a homologue or splice variant thereof) is involved in drug transport in the GIT, in particular Peyer's patch tissue of the GIT.

Calreticulin is a 46-kDa Ca (2+)-binding chaperone of the endoplasmic reticulum membranes. This protein binds Ca (2+) with high capacity, affects intracellular Ca (2+) homeostasis, and functions as a lectin-like chaperone. Given the over-abundance of expression of this protein in epithelial layers selected from PP tissue and the role of this protein as a lectin-like chaperone, we propose that this protein is a valuable protein target to aid or facilitate the intracellular trafficking of antigens or antigens in particles following targeted delivery to M cells or PP tissue. Proteins comprising chimerics of calreticulin plus a polypeptide with an antigen of choice would therefore prove valuable in that regard.

Members of the 14-3-3 protein family have been identified as regulatory elements in intracellular signaling pathways and cell cycle control. There had been reports that 14-3-3 protein can be used as a marker for Creutzfeldt-Jacob Disease (CJD) in cerebrospinal fluid (CSF). It is proposed that this protein or the gene coding for it is valuable in the control of the M cell phenotype, and as a result it would be advantageous to co-deliver that protein or gene with a protein, antigen, or DNA vaccine.

Nucleoside diphosphate kinases (NDP kinases) form a family of oligomeric enzymes present in all organisms. Eukaryotic NDP kinases are hexamers composed of identical subunits (approximately 17 kDa). A distinctive property of human NDPK-B is its ability to stimulate gene transcription. This property is independent of its catalytic activity and is possibly related to the role of this protein in cellular events including differentiation and tumor metastasis. Given our discovery of the increased expression of nucleoside diphosphate kinase B in M cell enriched PP FAE cells, we propose the importance of this protein in determining or controlling M cell phenotype, in M cell development, and optimal activation or priming of the mucosal immune system.

### Example 5

### Gene Expression Analysis of Rat PP and NPP Tissue Samples

In addition to the proteomic studies highlighted above, PP and NPP tissue samples were sent for gene expression analysis to CLONTECH Laboratories Inc. (a division of Becton Dickinson (BD) Biosciences) who then extracted RNA from the tissues to probe on ATLAS™1.2 rat arrays. The data containing differential expression levels of 1,200 genes many of which are presented in Table 1 below. The data show over-expression of many proteins. In Table 1, over-expressed genes are shown in bold and italicized.

In Table 1, “N/C” means not calculated due to manually-determined inconsistencies in one or both spots, and “?” means low confidence level (small difference).

Also, over-expressed genes from Table 1 that had a fold change above 0, as well as over-expressed genes are shown in Table 2 below with corresponding GenBank accession numbers for rat and human origin.

Based on the results (ratio PP/Normal epithelial tissue) in Table 1, the following proteins are of the particular interest: clusterin, T-cell surface glycoprotein CD5 precursor, HSP84, Ca2+-dependant phospholipase A2 precursor, ribosomal proteins S12, S11, L12, L11, S29, S19, L21, L19, L13, L44, and L36A.

In addition a series of genes coding for different TFs was noted including the following:

Jun-B; c-jun related TF,

Jun-D; c-jun related TF,

STAT 3-signal transducer and activator of transcription 3,

NF-kappaβ Tf p105 subunit,

CREB active TF,

New england deaconess TF,

C-jun proto-oncogene; TF AP-1; RJG-9,

S-myc proto-oncogene; myc related,

Nm23-M2; nucleoside diphosphate kinase B; metastasis reducing protein,

NDK-B; nucleoside diphosphate kinase B; metastasis reducing protein,

Lim-2; embryonic motor neuron topographic organizer; homeobox protein LIM-2, and

C-est-I proto-oncogene; p54.

TF coding genes such as these are considered here to be important in the development of M cell phenotype and in priming the immune system. Their co-delivery or co-targeting with DNA vaccine genes and/or with vaccines is expected to enhance activation of mucosal immunity to the co-delivered DNA vaccine and/or antigen by virtue of their priming of the cells to give a better mucosal immunity outcome.

TABLE 1
GENE EXPRESSION DATA FROM ATLAS 1.2 RAT ARRAY ANALYSIS

| | | Spot Intensity | | | | |
|---|---|---|---|---|---|---|
| # | coordinate | PP1 | NE1 | RATIO | Difference | GENE |
| 1 | A03c | 38 | 6 | 0.16 | −32 | T-cell surface glycoprotein CD5 precursor; lymphocyte glycoprotein LY1 |
| 2 | A03g | 31 | 57 | 1.84 | 26 | CD4 homologue, W3/25 antigen |
| 3 | A04f | 20 | 72 | 3.60 | 52 | signal transducer CD24 precursor; heat stable antigen (HSA); nectadrin |
| 4 | A04i | 17 | 40 | 2.35 | 23 | CD2, membrane glycoprotein, T-cell marker |
| 5 | A04i | 8 | 34 | 4.25 | 26 | scavenger receptor class B type I |
| 6 | A04m | 16 | 31 | 1.94 | 15 | SR13 myelin protein, peripheral myelin protein 22 (PMP-22), CD25 protein |
| 7 | A05a | 4 | 17 | 4.25 | 13 | glutamyl aminopeptidase A |
| 8 | A05i | 52 | 29 | 0.56 | −23 | I-kB (I-kappa B) alpha chain; RL/IF-1 gene product |
| 9 | A05m | 6 | 18 | 3.00 | 12 | interferon regulatory factor 1 (IRF1) |
| 10 | A06a | 7 | 21 | 3.00 | 14 | LIM domain protein CLP36, homologous to rat RIL |
| 11 | A06c | 152 | 78 | 0.51 | −74 | Gax. growth-arrest-specific protein |
| 12 | A07c | 42 | 24 | 0.57 | −18 | G1/S-specific cyclin D3 (CCND3) |
| 13 | A07l | 17 | 3 | 0.18 | −14 | M-phase inducer phosphatase 2 (MPI2); cell division control protein 25 B (CDC25B) |
| 14 | A07n | 37 | 20 | 0.54 | −17 | p55cdc; cell division control protein 20 |
| 15 | A08e | 56 | 24 | 0.43 | −32 | prothymosin-alpha (PTMA) |
| 16 | A08n | 32 | 105 | 3.28 | 73 | antigen peptide transporter 1 |
| 17 | A09g | 13 | 36 | 2.77 | 23 | proteasome delta subunit precursor; macropain delta; multicatalytic endopeptidase |
| | | | | | | complex delta; |
| 18 | A09i | 15 | 27 | 1.80 | 12 | proteasome component C13 precursor; macropain subunit C13; multicatalytic |
| | | | | | | endopeptidase |
| 19 | A09j | 341 | 893 | 2.62 | 552 | apolipoprotein A-I precursor (APO-AI) |
| 20 | A09k | 72 | 723 | 10.04 | 651 | apolipoprotein A-IV precursor (APO-AIV) |
| 21 | A11l | 1 | 13 | 13.00 | 12 | ErbB3 EGF receptor-related proto-oncogene; HER3 |
| 22 | A12c | 21 | 48 | 2.29 | 27 | A-raf proto-oncogene |
| 23 | A12i | 19 | 45 | 2.37 | 26 | rac-alpha serine/threonine kinase (RAC-PK-alpha); protein kinase B (PKB), AKT1 |
| 24 | A13k | 208 | 60 | 0.29 | −148 | HSP84; HSP90-beta; heat shock 90 kD protein |
| 25 | A14c | 7 | 38 | 5.43 | 31 | glutathione S-transferase Ya subunit (GST YA); liqandin subunit 1 alpha |
| 26 | A14d | 65 | 162 | 2.49 | 97 | microsomal glutathione S-transferase (GST12; MGST1) |
| 27 | A14e | 94 | 160 | 1.70 | 66 | glutathione S-transferase Yb subunit; GST subunit 4 mu (GSTM2) |
| 28 | A14q | 129 | 265 | 2.05 | 136 | glutathione S-transferase P subunit; GST subunit 7 pi (GST7-7) |
| 29 | A14n | 7 | 19 | 2.71 | 12 | NADPH-cytochrome P450 reductase (CPR); POR |
| 30 | B01h | 121 | 207 | 1.71 | 86 | copper-zinc-containing superoxide dismutase 1 (Cu—Zn SOD1) |
| 31 | B02f | 4 | 25 | 6.25 | 21 | fructose (glucose) transporter |
| 32 | B05k | 13 | 36 | 2.77 | 23 | sodium channel SCNB2, beta 2 subunit, brain |
| 33 | B06d | 28 | 12 | 0.43 | −16 | potassium channel, inward rectifier 11 |
| 34 | B07k | 2 | 18 | 9.00 | 16 | proton-coupled dipeptide cotransporter |
| 35 | B08b | 59 | 181 | 3.07 | 122 | fibroblast ADP/ATP carrier protein; ADP/ATP translocase 2, adenine nucleotide |
| | | | | | | translocator 2 |
| 36 | B09i | 7 | 43 | 6.14 | 36 | sodium-glucose cotransporter 1 |
| 37 | B09l | 40 | 115 | 2.88 | 75 | Na+/K+ ATPase alpha 1 subunit |
| 38 | B09n | 43 | 78 | 1.81 | 35 | vacuolar ATP synthase 16-kDa proteolipid subunit, ATP6C; MVP; ATPL |
| 39 | B10b | 10 | 78 | 7.80 | 68 | sodium/potassium-transporting ATPase beta 1 subunit (ATP1B1) |
| 40 | B12e | 10 | 68 | 6.80 | 58 | urate transporter/channel |
| 41 | B12f | 143 | 297 | 2.08 | 154 | ATP synthase lipid-binding protein P1 precursor, ATPase protein 9, ATP5G1 |
| 42 | B12q | 21 | 112 | 5.33 | 91 | ATP synthase, subunit c, P2 gene |
| 43 | B12m | 18 | 38 | 2.11 | 20 | annexin IV(ANX4); lipocortin IV;36-kDa zymogen granule membrane-associated |
| | | | | | | protein (ZAP36) |
| 44 | B13f | 37 | 127 | 3.43 | 90 | lipocortin 2 |
| 45 | B14d | 59 | 326 | 5.53 | 267 | fatty acid-binding protein (liver; L-FABP); Z-protein; squalene - & sterol-carrier |
| | | | | | | protein (SCP); P14 |
| 46 | B14f | 49 | 157 | 3.20 | 108 | fatty acid-binding protein (intestinal; I-FABP; FABPI) |
| 47 | C02f | 19 | 78 | 4.11 | 59 | fructose-bisphosphate aldolase B (ALDOB); liver-type aldolase |
| 48 | C02g | 37 | 76 | 2.05 | 39 | fructose-bisphosphate aldolase A (ALDOA); muscle-type aldolase |
| 49 | C02j | 15 | 30 | 2.00 | 15 | testis fructose-6-phosphate 2-kinase/fructose 2,6-biphosphate |
| | | | | | | (testis 6PF-2-K/fru-2,6-P2ase); 6- |
| 50 | C02n | 604 | 1123 | 1.86 | 519 | cytochrome c oxidase subunit Vb & Vla precursor (COX5B) |
| 51 | C03a | 9 | 20 | 2.22 | 11 | cytochrome B5 (CYB5) |
| 52 | C03e | 65 | 138 | 2.12 | 73 | mitochondrial hydroxymethylglutaryl-CoA synthase precursor (HMG-CoA synthase); |
| | | | | | | 3-hydroxy-3- |
| 53 | C03f | 177 | 760 | 4.29 | 583 | cytochrome oxidase, subunit I, Sertoli cells |
| 54 | C03q | 27 | 47 | 1.74 | 20 | ATPase, subunit F, vacuolar (vatf) |
| 55 | C03i | 641 | 1087 | 1.70 | 446 | cvtochrome c oxidase, subunit IV, mitochondrial |
| 56 | C03l | 46 | 147 | 3.20 | 101 | cytochrome c oxidase, subunit Va, mitochondrial |
| 57 | C03l | 317 | 180 | 0.57 | −137 | cytochrome c oxidase, subunit VIIIh |
| 58 | C04b | 189 | 351 | 1.86 | 162 | mitochondrial ATP synthase beta subunit precursor (ATP5B) |
| 59 | C04q | 23 | 127 | 5.52 | 104 | creatine kinase, ubiquitous, mitochondrial |
| 60 | C06c | 6 | 52 | 8.67 | 46 | fatty acid amide hydrolase |
| 61 | C06j | 73 | 175 | 2.40 | 102 | cytochrome P450 17 (CYP17), P450C17, CYPXVII, steroid 17-alpha-hydroxylase/17,20 |
| | | | | | | lyase |
| 62 | C07i | 9 | 51 | 5.67 | 42 | cytochrome P-450 4F1, hepatic tumour |
| 63 | C07l | 19 | 39 | 2.05 | 20 | cytochrome P-450 4F4 |
| 64 | C08a | 17 | 33 | 1.94 | 16 | cytochrome P-450 4F5 |
| 65 | C08l | 8 | 24 | 3.00 | 16 | adenylate kinase 3 |
| 66 | C08m | 32 | 65 | 2.03 | 33 | cAMP-dependent protein kinase type I-alpha regulatory chain |
| 67 | C09e | 7 | 30 | 4.29 | 23 | glutathione synthetase (GSH synthetase; GSH-S; GSS). glutathione synthase |
| 68 | C10e | 25 | 10 | 0.40 | −15 | carbonic anhydrase 4 |
| 69 | C10j | 19 | 9 | 0.47 | −10 | alkaline phosphatase |
| 70 | C10k | 21 | 5 | 0.24 | −16 | dopamine beta-hydroxylase |
| 71 | C10l | 29 | 11 | 0.38 | −18 | acetylcholinesterase, T subunit, glycolipid-anchored |
| 72 | C10m | 228 | 445 | 1.95 | 217 | NADP+ alcohol dehydrogenase; aldehyde reductase (ALR); 3-dG-reducing enzyme |
| 73 | C11b | 19 | 33 | 1.74 | 14 | calcium bindinq protein 2 (CABP2), endoplasmic reticulum stress protein (ERP72); |
| | | | | | | protein disulfide |
| 74 | C11d | 97 | 55 | 0.57 | −42 | 60S ribosomal protein L44; L36A |
| 75 | C11e | 1728 | 527 | 0.30 | −1201 | 40S ribosomal protein S12 |
| 76 | C11q | 400 | 179 | 0.45 | −221 | ribosomal protein L11 |
| 77 | C11h | 670 | 355 | 0.53 | −315 | ribosomal protein L13 |
| 78 | C11i | 967 | 424 | 0.44 | −543 | ribosomal protein L12 |
| 79 | C11k | 812 | 413 | 0.51 | −399 | S19; 40S ribosomal protein S19 |
| 80 | C11l | 554 | 286 | 0.52 | −268 | 60S ribosomal protein L21 |
| 81 | C11m | 1405 | 737 | 0.52 | −668 | 60S ribosomal protein L19 (RPL19) |
| 82 | C11n | 315 | 109 | 0.35 | −206 | 40S ribosomal protein S11 |
| 83 | C12b | 158 | 48 | 0.30 | −110 | Fte-1; putative v-fos transformation effector protein; yeast mitochondrial protein |
| | | | | | | import homolog; 40S |
| 84 | C12d | 1451 | 787 | 0.54 | −664 | elongation factor 2 (EF2) |
| 85 | C12l | 346 | 10 | 0.03 | −336 | clusterin (CLU); testosterone-repressed prostate message 2 (TRPM2); apolipoprotein |
| | | | | | | J; sulfated |
| 86 | C12m | 14 | 0 | Undefined | −14 | activator of apoptosis harakiri (HRK); neuronal death protein 5 (DP5); BID3 |
| 87 | C13c | 19 | 5 | 0.26 | −14 | SURVIVAL OF MOTOR NEURON(RSMN) |
| 88 | D08i | 6 | 17 | 2.83 | 11 | retinoid X receptor alpha (RXR alpha; RXRA); NR2B1 |
| 89 | D12c | 6 | 18 | 3.00 | 12 | INOSITOL TRIPHOSPHATE RECEPTOR SUBTYPE 3 |
| 90 | E01m | 10 | 50 | 5.00 | 40 | neurotrophin 3 precursor (NTF3); neurotrophic factor, HDNF, nerve growth |
| | | | | | | factor 2 (NGF2) |
| 91 | E02c | 19 | 8 | 0.42 | −11 | transforming growth factor, beta 1 |
| 92 | E03k | 40 | 185 | 4.63 | 145 | C-type natriuretic peptide precursor (CNP; NPPC) |
| 93 | E04b | 17 | 7 | 0.41 | −10 | thyroid stimulating hormone, beta |
| 94 | E07c | 33 | 15 | 0.45 | −18 | c-src-kinase (CSK) & negative regulator; tyrosine-protein kinase |
| 95 | E08a | 105 | 249 | 2.37 | 144 | extracellular signal-regulated kinase 1 (ERK1); mitogen-activated protein kinase 1 |
| | | | | | | (MAP kinase 1: |
| 96 | E08l | 8 | 22 | 2.75 | 14 | protein kinase C delta type (PKC-delta) |
| 97 | E09l | 36 | 68 | 1.89 | 32 | insulin receptor-related receptor-alpha (sIRR-1) |
| 98 | E10f | 3 | 17 | 5.67 | 14 | CamK II; calcium/calmodulin-dependent protein kinase brain type II beta |
| 99 | E10q | 3 | 13 | 4.33 | 10 | CamK I, calcium/calmodulin-dependent protein kinase type I + CaM-like protein kinase |
| 100 | E10l | 18 | 42 | 2.33 | 24 | Casein kinase I delta, CKId; 49-kDa isoform |
| 101 | E11e | 15 | 29 | 1.93 | 14 | cyclin-dependent kinase 4 (CDK4); cell division protein kinase 4, PSK-J3 |
| 102 | E12g | 23 | 45 | 1.96 | 22 | protein phosphatase 2C isoform; Mg2+ dependent protein phosphatase beta isoform |
| 103 | E13i | 40 | 19 | 0.48 | −21 | quanine nucleotide-bindinq protein G(I) alpha 2 subunit (GNAI2); adenylate cyclase- |
| 104 | E14i | 268 | 661 | 2.47 | 393 | rab12, ras related GTPase |
| 105 | F01e | 10 | 29 | 2.90 | 19 | RalGDSB; GTP/GDP dissociation stimulator for a ras-related GTPase |
| 106 | F02a | 189 | 97 | 0.51 | −92 | calcium-dependent phospholipase A2 precursor (PLA2); phosphatidylcholine 2- |
| 107 | F02h | 15 | 79 | 5.27 | 64 | phospholipase C beta 3 (PLC-beta 3) |
| 108 | F04e | 33 | 57 | 1.73 | 24 | 14-3-3 protein zeta/delta, PKC inhibitor protein-1, KCIP-1, mitochondrial import |
| | | | | | | stimulation factor |
| 109 | F04k | 15 | 29 | 1.93 | 14 | 14-3-3 protein epsilon; PKC inhibitor protein-1, KCIP-1; mitochondrial import |
| | | | | | | stimulation factor L |
| 110 | F05e | 8 | 21 | 2.63 | 13 | presenilin 1 (PSNL1; PSEN1; PS1); S182 protein |
| 111 | F05j | 6 | 17 | 2.83 | 11 | PDGF-associated protein |
| 112 | F06a | 59 | 143 | 2.42 | 84 | ADP-ribosylation factor 5 (ARF5) |
| 113 | F06j | 12 | 22 | 1.83 | 10 | dipeptidase (DPEP1) |
| 114 | F07m | 7 | 29 | 4.14 | 22 | granzyme M precursor (GZMM). MET-ASE; natural killer cell granular protease; |
| | | | | | | RNK-MET-1 |
| 115 | F08e | 11 | 100 | 9.09 | 89 | angiotensin converting enzyme (ACE; somatic; dipeptidyl carboxypeptidase I, kininase II |
| 116 | F08h | 18 | 48 | 2.67 | 30 | amonipeptidase B |
| 117 | F08i | 39 | 92 | 2.36 | 53 | kidney aminopeptidase M (APM) |
| 118 | F08k | 19 | 81 | 4.26 | 62 | metalloendopeptidase meprin beta subunit |
| 119 | F08l | 5 | 15 | 3.00 | 10 | endothelin converting enzyme |
| 120 | F09a | 3 | 16 | 5.33 | 13 | gelatinase A |
| 121 | F09g | 13 | 27 | 2.08 | 14 | cathepsin L |
| 122 | F09l | 14 | 28 | 2.00 | 14 | proteasome component C3 |
| 123 | F12a | 6 | 16 | 2.67 | 10 | leukocyte common antigen-related tyrosine phosphatase (LAR) |
| 124 | G31 | 12 | 39 | 3.25 | 27 | ornithine decarboxylase (ODC) |
| 125 | G43 | 287 | 1132 | 3.94 | 845 | cytoplasmic beta-actin (ACTB) |
| 126 | G47 | 7327 | 3614 | 0.49 | −3713 | 40S ribosomal protein S29 (RPS29) |

TABLE 2
RAT GENES (PP VS. NPP)

| | Fold | GenBank ID | GenBank ID |
|---|---|---|---|
| GENE | change | Human | Rat |
| activator of apoptosis harakiri (HRK); neuronal death | ** | U76376.1 | D83697 |
| protein 5 (DP5); BID3 | | | |
| RET ligand 1 (RET1) | ** | — | U97142 |
| P2X purinoceptor 1; ATP receptor P2X1; purinergic | ** | P51575 | U14414 |
| receptor; RP-2 protein | | | |
| leukocyte common antigen precursor (LCA); CD45 | ** | Y00638 | M10072 |
| antigen; T200; PTPRC | | | |
| amphiphysin II (AMPH2) | ** | AF001383.1 | Y13380 |
| Jak3 tyrosine-protein kinase; Janus kinase 3 | ** | XM_038595.3 | D28508 |
| DCC; netrin receptor; immunoglobulin gene superfamily | ** | M32292.1 | AH002168.1 |
| member; former tumor suppressor protein candidate | | | |
| c-fgr proto-oncogene | ** | AAA52762.1 | X57018.1 |
| small inducible cytokine A3 precursor (SCYA3); | ** | P10147 | AF119381.1 |
| macrophage inflammatory protein 1 alpha precursor | | | |
| (MIP1-alpha; MIP1A) | | | |
| protein kinase C beta-If type (PKC-beta I) + protein kinase | ** | X06318 | P04410 |
| C beta-II type (PKC-beta II) | | | |
| E-selectin precursor; endothelial leukocyte adhesion | ** | P16581 | L25527 |
| molecule 1 (ELAM-1); leukocyte-endothelial cell adhesion | | | |
| molecule 2 (LECAM2); CD62E | | | |
| T-cell receptor CD3 zeta subunit | ** | J04132.1 | L08447.1 |
| Protein kinase C-binding protein beta15; RING-domain | ** | — | U48248 |
| containing | | | |
| G1/S-specific cyclin C (CCNC) | ** | AAC50825.1 | D14013 |
| maspin; protease inhibitor 5 (PI5); tumor suppressor | ** | U04313.1 | U58857 |
| peptide/histidine transporter | ** | — | AB000280 |
| acetyl-CoA carboxylase (ACC); biotin carboxylase | ** | X68968.1 | AH002123.1 |
| fibroblast growth factor receptor subtype 4 | ** | L03840.1 | M91599 |
| LCR-1; putative chemokine and HIV coreceptor homolog; | ** | — | U54791 |
| G protein-coupled receptor | | | |
| tumor necrosis factor alpha precursor (TNF-alpha; TNFA); | ** | AF043342.1 | X66539 |
| cachectin | | | |
| CC chemokine MIP3 alpha exodus | ** | — | U90447.1 |
| luteinizing hormone, alpha | ** | NM_000735.2 | V01252 |
| Ctk; non-receptor protein tyrosine kinase (batk) | ** | P42679 | L34542.1 |
| RhoGAP; p122 | ** | — | S54293 |
| Adenylyl cyclase type V | ** | M83533.1 | M96159 |
| cathepsin S precursor (CTSS) | ** | P25774 | L03201.1 |
| O-6-methylguanine-DNA methyltransferase (MGMT); | ** | M31767.1 | NM_012861.1 |
| methylated-DNA-protein-cysteine methyltransferase | | | |
| clusterin (CLU); testosterone-repressed prostate message | 34.60 | X14723 | U02391.1 |
| 2 (TRPM2); apolipoprotein J; sulfated glycoprotein 2 | | | |
| (SGP2); dimeric acid glycoprotein (DAG) | | | |
| T-cell surface glycoprotein CD5 precursor; lymphocyte | 6.33 | X04391.1 | D10728 |
| glycoprotein LY-1 (LYT1) | | | |
| M-phase inducer phosphatase 2 (MPI2); cell division | 5.67 | S78187.1 | D16237 |
| control protein 25 B (CDC25B) | | | |
| dopamine beta-hydroxylase | 4.20 | Y00096.1 | L12407 |
| SURVIVAL OF MOTOR NEURON(RSMN) | 3.80 | AAC50473.1 | U75369 |
| HSP84; HSP90-beta; heat shock 90kD protein | 3.47 | XM_055551.3 | S45392 |
| Fte-1; putative v-fos transformation effector protein; yeast | 3.29 | M84711.1 | M84716.1 |
| mitochondrial protein import homolog; 40S ribosomal | | | |
| protein S3A; RPS3A | | | |
| 40S ribosomal protein S12 | 3.28 | X53505 | M18547 |
| 40S ribosomal protein S11 | 2.89 | X06617 | K03250 |
| acetylcholinesterase, T subunit, glycolipid-anchored | 2.64 | M55040.1 | X71089.1 |
| carbonic anhydrase 4 | 2.50 | NM_000717.2 | I52551 |
| thyroid stimulating hormone, beta | 2.43 | S70587.1 | M13897.1 |
| transforming growth factor, beta 1 | 2.38 | M34057 | NM_021578.1 |
| prothymosin-alpha (PTMA) | 2.33 | AF257099.1 | M20035 |
| potassium channel, inward rectifier 11 | 2.33 | — | D42145 |
| ribosomal protein L12 | 2.28 | L06505.1 | X53504.1 |
| ribosomal protein L11 | 2.23 | X79234.1 | X62146.1 |
| c-src-kinase (CSK) & negative regulator; tyrosine-protein | 2.20 | X59932.1 | X58631 |
| kinase | | | |
| alkaline phosphatase | 2.11 | AAA98616.1 | S18408 |
| guanine nucleotide-binding protein G(I) alpha 2 subunit | 2.11 | XM_041507.1 | NM_031035.1 |
| (GNAI2); adenylate cyclase-inhibiting G alpha protein | | | |
| 40S ribosomal protein S29 (RPS29) | 2.03 | NM_001032.2 | X59051 |
| S19; 40S ribosomal protein S19 | 1.97 | P39019 | P17074 |
| Gax, growth-arrest-specific protein | 1.95 | — | Z17223.1 |
| calcium-dependent phospholipase A2 precursor (PLA2); | 1.95 | M22430.1 | U38376.1 |
| phosphatidylcholine 2-acylhydrolase (PLA2-10; PLA2G5) | | | |
| 60S ribosomal protein L21 | 1.94 | P46778 | M27905 |
| 60S ribosomal protein L19 (RPL19) | 1.91 | X63527 | J02650 |
| ribosomal protein L13 | 1.89 | P26373 | X78327.1 |
| p55cdc; cell division control protein 20 | 1.85 | AF099644.1 | AF052695.1 |
| elongation factor 2 (EF2) | 1.84 | X51466 | Y07504.1 |
| I-κB (I-kappa B) alpha chain; RL/IF-1 gene product | 1.79 | X63594.1 | AF388201.1 |
| 60S ribosomal protein L44; L36A | 1.76 | M15661 | P10661 |
| cytochrome c oxidase, subunit VIIIh | 1.76 | J04823.1 | NM_012786.1 |
| G1/S-specific cyclin D3 (CCND3) | 1.75 | NM_001760.2 | NM_012766.1 |
| cytochrome c oxidase, subunit IV, mitochondrial | 0.59 | AF017115.1 | X14209 |
| glutathione 3-transferase Yb subunit; GST subunit 4 mu | 0.59 | — | X04229.1 |
| (GSTM2) | | | |
| copper-zinc-containing superoxide dismutase 1 (Cu-Zn | 0.58 | — | NM_017050.1 |
| SOD1) | | | |
| 14-3-3 protein zeta/delta; PKC inhibitor protein-1; KCIP-1; | 0.58 | U28964.1 | L07913.1 |
| mitochondrial import stimulation factor S1 subunit | | | |
| calcium binding protein 2 (CABP2); endoplasmic reticulum | 0.58 | XM_012077.4 | M86870 |
| stress protein (ERP72); protein disulfide isomerase- | | | |
| related protein precursor | | | |
| ATPase, subunit F, vacuolar (vatf) | 0.57 | AF047436.1 | U43175.1 |
| proteasome component C13 precursor; macropain subunit | 0.56 | P28062 | NM_080767.1 |
| C13; multicatalytic endopeptidase complex subunit C13 | | | |
| PSMB8 | | | |
| vacuolar ATP synthase 16-kDa proteolipid subunit; | 0.55 | NM_001695.1 | M62762.1 |
| ATP6C; MVP; ATPL | | | |
| dipeptidase (DPEP1) | 0.55 | NM_004413.1 | M94056 |
| CD4 homologue, W3/25 antigen | 0.54 | BC025782. | M15768.1 |
| mitochondrial ATP synthase beta subunit precursor | 0.54 | NM_001686.1 | M19044.1 |
| (ATP5B) | | | |
| cytochrome c oxidase subunit Vb & VIa precursor | 0.54 | M59250.1 | X14208.1 |
| (COX5B) | | | |
| insulin receptor-related receptor-alpha (sIRR-1) | 0.53 | — | M90660.1 |
| cyclin-dependent kinase 4 (CDK4); cell division protein | 0.52 | P11802 | P35426 |
| kinase 4; PSK-J3 | | | |
| 14-3-3 protein epsilon; PKC inhibitor protein-1; KCIP-1; | 0.52 | XM_088041.1 | D30739.1 |
| mitochondrial import stimulation factor L subunit | | | |
| SR13 myelin protein; peripheral myelin protein 22 (PMP- | 0.52 | — | M69139.1 |
| 22); CD25 protein | | | |
| cytochrome P-450 4F5 | 0.52 | — | AF288818.1 |
| NADP+ alcohol dehydrogenase; aldehyde reductase | 0.51 | J04794.1 | D10854.1 |
| (ALR); 3-dG-reducing enzyme | | | |
| protein phosphatase 2C isoform; Mg2+ dependent protein | 0.51 | — | S90449.1 |
| phosphatase beta isoform | | | |
| testis fructose-6-phosphate 2-kinase/fructose 2,6- | 0.50 | NM_002625.1 | X15579.1 |
| biphosphate (testis 6PF-2-K/fru-2,6-P2ase); 6- | | | |
| phosphofructo-2-kinase; fructose-2,6-bisphosphatase | | | |
| proteasome component C3 | 0.50 | D00760 | J02897.1 |
| cAMP-dependent protein kinase type I-alpha regulatory | 0.49 | P10644 | P09456 |
| chain | | | |
| cytochrome P-450 4F4 | 0.49 | — | U39206.1 |
| fructose-bisphosphate aldolase A (ALDOA); muscle-type | 0.49 | XM_043948.2 | NM_012495.1 |
| aldolase | | | |
| glutathione S-transferase P subunit; GST subunit 7 pi | 0.49 | — | X02904.1 |
| (GST7-7) | | | |
| ATP synthase lipid-binding protein P1 precursor; ATPase | 0.48 | NM_005175.1 | NM_017311.1 |
| protein 9; ATP5G1 | | | |
| cathepsin L | 0.48 | M20496.1 | Y00697.1 |
| annexin IV(ANX4); lipocortin IV;36-kDa zymogen granule | 0.47 | XM_031596.3 | NM_024155.1 |
| membrane-associated protein (ZAP36) | | | |
| mitochondrial hydroxymethylglutaryl-CoA synthase | 0.47 | P54868 | P22791 |
| precursor (HMG-CoA synthase); 3-hydroxy-3- | | | |
| methylglutaryl-CoA synthase; HMGCS2 | | | |
| cytochrome B5 (CYB5) | 0.45 | M22865.1 | D13205.1 |
| A-raf proto-oncogene | 0.44 | P10398 | X06942 |
| Casein kinase I delta; CKId; 49-kDa isoform | 0.43 | P48730 | Q06486 |
| CD2, membrane glycoprotein, T-cell marker | 0.43 | M14362.1 | X05111.1 |
| kidney aminopeptidase M (APM) | 0.42 | XM_087746.1 | M26710 |
| rac-alpha serine/threonine kinase (RAC-PK-alpha); protein | 0.42 | P31749 | Y15748.1 |
| kinase B (PKB); AKT1 | | | |
| extracellular signal-regulated kinase 1 (ERK1); mitogen- | 0.42 | P27361 | P21708 |
| activated protein kinase 1 (MAP kinase 1; MAPK1); | | | |
| insulin - stimulated microtubule-associated protein-2 | | | |
| kinase; MNK1; PRKM3; ERT2; p44-MAPK | | | |
| cytochrome P450 17 (CYP17); P450C17; CYPXVII; | 0.42 | NM_000102.2 | X69816.1 |
| steroid 17-alpha-hydroxylase/17,20 lyase | | | |
| ADP-ribosylation factor 5 (ARF5) | 0.41 | NM_001662. | NM_024149.1 |
| rab12, ras related GTPase | 0.41 | — | M83676. |
| microsomal glutathione S-transferase (GST12; MGST1) | 0.40 | XM_048886.3 | J03752 |
| apolipoprotein A-I precursor (APO-AI) | 0.38 | X02162 | M00001 |
| presenilin 1 (PSNL1; PSEN1; PS1); S182 protein | 0.38 | XM_007441.1 | D82363 |
| amonipeptidase B | 0.38 | \|XM_087242.1 | U61696 |
| leukocyte common antigen-related tyrosine phosphatase | 0.38 | — | U00477.1 |
| (LAR) | | | |
| NADPH-cytochrome P450 reductase (CPR); POR | 0.37 | S90469 | NM_031576.1 |
| protein kinase C delta type (PKC-delta) | 0.36 | NM_006254.1 | M18330 |
| proteasome delta subunit precursor; macropain delta; | 0.36 | X61971.1 | NM_057099.1 |
| multicatalytic endopeptidase complex delta; proteasome | | | |
| subunit Y; proteasome subunit 5; PSMB6 | | | |
| sodium channel SCNB2, beta 2 subunit, brain | 0.36 | AAC05208.1 | NM_012877. |
| retinoid X receptor alpha (RXR alpha; RXRA); NR2B1 | 0.35 | XM_088424.1 | NM_012805.1 |
| PDGF-associated protein | 0.35 | U41745.1 | U41744.1 |
| Na+/K+ ATPase alpha 1 subunit | 0.35 | AAA51801.1 | M28647 |
| RalGDSB; GTP/GDP dissociation stimulator for a ras- | 0.34 | — | NM_019250.1 |
| related GTPase | | | |
| interferon regulatory factor 1 (IRF1) | 0.33 | XM_034862.1 | M34253 |
| LIM domain protein CLP36, homologous to rat RIL | 0.33 | AJ310549.1 | U23769.1 |
| adenylate kinase 3 | 0.33 | XM_016642.3 | NM_13218.1 |
| INOSITOL TRIPHOSPHATE RECEPTOR SUBTYPE 3 | 0.33 | — | L06096.1 |
| endothelin converting enzyme | 0.33 | Z35307.1 | D29683 |
| fibroblast ADP/ATP carrier protein; ADP/ATP translocase | 0.33 | J02683 | D12771 |
| 2; adenine nucleotide translocator 2 (ANT2) | | | |
| cytochrome c oxidase, subunit Va, mitochondrial | 0.31 | M22760.1 | X15030 |
| fatty acid-binding protein (intestinal; I-FABP; FABPI) | 0.31 | M18079 | M18080.1 |
| ornithine decarboxylase (ODC) | 0.31 | X16277 | D11372.1\| |
| antigen peptide transporter 1 | 0.30 | X57522 | P36370 |
| lipocortin 2 | 0.29 | D00017.1 | S73557 |
| signal transducer CD24 precursor; heat stable antigen | 0.28 | P25063 | U49062 |
| (HSA); nectadrin | | | |
| cytoplasmic beta-actin (ACTB) | 0.25 | M10277.1 | V01217 |
| fructose-bisphosphate aldolase B (ALDOB); liver-type | 0.24 | XM_042788.1 | M10149 |
| aldolase | | | |
| granzyme M precursor (GZMM); MET-ASE; natural killer | 0.24 | NM_005317.2 | Q03238 |
| cell granular protease; RNK-MET-1 | | | |
| scavenger receptor class B type I | 0.24 | — | AB002151.1 |
| glutamyl aminopeptidase A | 0.24 | XM_003595.2 | S73583 |
| metalloendopeptidase meprin beta subunit | 0.23 | — | M88601.1 |
| glutathione synthetase (GSH synthetase; GSH-S; GSS); | 0.23 | U34683.1 | L38615.1 |
| glutathione synthase | | | |
| cytochrome oxidase, subunit I, Sertoli cells | 0.23 | S79304 | S79304 |
| CamK I; calcium/calmodulin-dependent protein kinase | 0.23 | Q14012 | L24907 |
| type I + CaM-like protein kinase | | | |
| C-type natriuretic peptide precursor (CNP; NPPC) | 0.22 | NM_024409.1 | D90219 |
| neurotrophin 3 precursor (NTF3); neurotrophic factor; | 0.20 | M37763.1 | M34643 |
| HDNF; nerve growth factor 2 (NGF2) | | | |
| phospholipase C beta 3 (PLC-beta 3) | 0.19 | NM_000932.1 | M99567 |
| ATP synthase, subunit c, P2 gene | 0.19 | D13119.1 | D13124 |
| gelatinase A | 0.19 | NM_004530.1 | U65656 |
| glutathione S-transferase Ya subunit (GST YA); ligandin | 0.18 | NM_000852.2 | K01932 |
| subunit 1 alpha | | | |
| creatine kinase, ubiquitous, mitochondrial | 0.18 | XM_016524.4 | X59737 |
| fatty acid-binding protein (liver; L-FABP); Z-protein; | 0.18 | NM_001443.1 | M35991 |
| squalene- & sterol-carrier protein (SCP); P14 | | | |
| cytochrome P-450 4F1, hepatic tumour | 0.18 | — | NM_019623.1 |
| CamK II; calcium/calmodulin-dependent protein kinase | 0.18 | NM_001220.1 | M16112 |
| brain type II beta | | | |
| sodium-glucose cotransporter 1 | 0.16 | P13866 | U03120 |
| fructose (glucose) transporter | 0.16 | AAB60641 | D13871.1 |
| urate transporter/channel | 0.15 | — | U67958 |
| sodium/potassium-transporting ATPase beta 1 subunit | 0.13 | NM_001677.1 | NM_013113.1 |
| (ATP1B1) | | | |
| fatty acid amide hydrolase | 0.12 | U82535.1 | U72497 |
| proton-coupled dipeptide cotransporter | 0.11 | — | D50306.1 |
| angiotensin converting enzyme (ACE; somatic; dipeptidyl | 0.11 | NM_000789.1 | NM_012544.1 |
| carboxypeptidase I; kininase II | | | |
| apolipoprotein A-IV precursor (APO-AIV) | 0.10 | XM_052144.2 | P02651 |
| ErbB3 EGF receptor-related proto-oncogene; HER3 | 0.08 | M29366.1 | NM_017218.2 |
| Jun-B; c-jun related TF, | ** | M29039.1 | X54686 |
| S-myc proto-oncogene; myc related, | ** | | M29069 |
| C-est-I proto-oncogene; p54. | 5 | AF193068.1 | X55787.1 |
| Jun-D; c-jun related TF, | 1.79 | X56681.1 | D26307(mouse) |
| NF-kappaβ Tf p105 subunit, | 1.67 | P19838 | L26267.1 |
| Nm23-M2; nucleoside diphosphate kinase B; | 1.47 | | X68193.1 |
| metastasis reducing protein, | | | (mouse) |
| STAT 3 —signal transducer and activator of | 1.16 | NM_003150.1 | NM_012747.1 |
| transcription 3, | | | |
| CREB active TF, | 1 | | M34356.1 |
| New england deaconess TF, | 1 | | U09229 |
| Lim-2; embryonic motor neuron topographic | 1 | | L35572 |
| organizer; homeobox protein LIM-2, and | | | |
| NDK-B; nucleoside diphosphate kinase B; | 0.81 | | U29200.1 |
| metastasis reducing protein, | | | |
| C-jun proto-oncogene; TF AP-1; RJG-9, | 0.5 | J04111.1 | X17215.1 |

Symbols indicating fold changes in Table 2:
** expressed in PP but not NPP, or in co-culture but not in Caco-2 cells.
* expressed in co-culture but not in Caco-2 cells (only repeated once).
— expressed in Caco-2 but not in co-culture.

### Example 5

### ATLAS Array Data on Co-Culture of Human Caco-2 Cells and Raji B-Cells

In order to facilitate the routine study of M cell biology, there was a desire to establish a suitable and representative in-vitro model. In the work carried out by Kernéis et al. (Kerneis S, Caliot E, Stubbe H, Bogdanova A, Kraehenbuhl J, Pringault E (2000). Molecular studies of the intestinal mucosal barrier physiopathology using co-cultures of epithelial and immune cells: a technical update. Microbes Infect Jul. 2, 2000 (9):1119-24), it was reported that Peyer's patch lymphocytes co-cultured with Caco-2 cells trigger the phenotypic conversion of enterocytes into cells that express morphological and functional M-cell properties. This work was further developed by Gullberg et al. (Gullberg E, Leonard M, Karlsson J, Hopkins A M, Brayden D, Baird A W, Artursson P. Expression of specific markers and particle transport in a new human intestinal M-cell model. Biochem Biophys Res Commun Dec. 29, 2000; 279(3): 808-13) to create a simplified in vitro model of the human M-cell. Co-cultures of physically separated human intestinal epithelial Caco-2 cells and B-cell lymphoma Raji cells were established. The co-cultures were characterized under the criteria of morphology, integrity, expression of M-cell markers and cell adhesion molecules (CAMs), and altered particle transport. Using this construct, the epithelial cells were transformed to cells with an M-cell-like morphology and had altered expression of potential human M-cell markers (alkaline phosphatase down-regulation and Sialyl Lewis A antigen up-regulation). The expression of intercellular adhesion molecule-1 and vascular cell adhesion molecule was altered, and there was an increased binding of lectins wheat germ agglutinin and peanut agglutinin with a 40-fold increase in microparticle transport. The particle transport was size-dependent and could be inhibited at 4° C. or by replacing the Raji B-cells with Jurkat T-cells. Thus the comparison of RNA isolated from co-cultured Caco2 cells to that isolated from normal Caco2 cells was designed to simulate a comparison of M cell RNA to normal gut enterocyte RNA.

### Isolation of Total RNA from Co-Cultured Caco-2 Cells

Caco-2 Cell Culture

Caco-2 cells were cultured in Dulbecco's Modified Eagles Medium (DMEM), 4.5 g/L glucose supplemented with 1% Mem, 10% FCS and 1% penicillin/streptomycin a t 37° C. and 5% CO₂ in 95% relative humidity. Cells were grown and expended in Falcon culture flasks and passaged once they attained 100% confluence. Caco-2 cells were seeded on Transwell Clear filters (Costar, 12 mm diameter, 3.0 μm pore size)) at a density of 5×10⁵ cells/cm2 and incubated in a 12 well culture plate with a medium change every second day. 1.0 ml was added to the basolateral side and apical sides.

Raji Cell Culture

Raji B-lymphoma cells were cultured in RPMI 1640 Medium, with 1% (v/v) non-essential amino-acids, 10% FCS and 1% penicillin/streptomycin, 1% L-glutamine at 37° C. and 5% CO₂ in 95% relative humidity. Cells were grown in suspension in Falcon tissue culture flasks and passaged by dilution every 5-7 days.

Co-Culture: Day 14 (Treating with Raji B-Cells)

After 14 days of culturing Caco-2 monolayers, 15-20 ml of Raji cells were removed from the T75 flask and placed in a 20 ml universal. The cells were centrifuged at 1000 rpm for 3 min. Cells were re-suspended at a concentration of 1×10⁶ cells/ml. 1 ml of fresh complete DMEM was added to the apical and basolateral sides of the Caco-2 monolayer filters. 0.5 ml of 1×10⁶ Raji cells/ml cells was added to the basolateral side of the filters. For control filters (non co-culture) 0.5 ml of Raji medium only was added to the basolateral side.

Isolation of Total RNA from Co-Cultured Caco-2 Cells

After 4 days of co-culture the filters were rinsed in PBS. 0.5 ml of PBS was added to the apical side of each filter and the Caco-2 cells were scraped off the filter surface into suspension in the PBS. The cells from all the co-cultured Caco-2 filters were pooled, centrifuged at 1000 rpm for 3 min, the supernatant PBS was removed and the pellet was used for RNA extraction.

Analysis of mRNA Expression

Total cellular RNA was extracted using an acid guanidinium thiocyanate-phenol-chloroform method. RNA's integrity was confirmed by gel electrophoresis and ethidium bromide staining. mRNA was reverse transcribed in the presence of P³² dATP, and the transcribed cDNA was purified by chromatography before being hybridized over night to the array membrane. Membranes were exposed to x-ray film using an intensifying screen for 3 days and the mRNA expression levels were analyzed by scanning the films with a densitometer. Expression levels were normalized relative to internal standards, and relative increases in mRNA levels in co-cultured cells versus monoculture controls were calculated. Two hybridization experiments were performed using mRNA from two separate cell harvests. Results from the two experiments were pooled, and a summary of the findings was tabulated in Tables 3(a)-3(f). The identified genes are from the following groups: oncogenes, tumor suppressor genes, genes involved in the cell cycle, ion channels and transport, stress response genes, modulators and effectors, genes involved in intracellular transduction, genes linked to apoptosis, DNA synthesis, repair & recombination, transcription factors, DNA binding proteins, receptors, cell surface antigens, genes involved in cell adhesion, growth factors, cytokines, chemokines and hormones.

In Table 3, genes which were found to be exclusively over-expressed in the co-culture and not in the control Caco-2 monolayer are represented by **. A single asterisk represents genes that also were expressed in the co-culture and not in the control Caco-2 monolayer. However, these particular genes have been distinguished from the genes labeled with two asterisks as they were not expressed in both hybridization experiments performed, and will require confirmation in the future by PCR so as to rule out false positives/negatives. Genes not expressed in the co-culture but expressed in the Caco-2 monolayer controls are indicated by a minus symbol, “−”.

TABLE 3 a
Oncogenes, Tumor Suppressors, Cell Cycle Regulators

| Gene | Fold change | GenBank ID |
|---|---|---|
| Myeloid cell nuclear differentiation | * | M81750 |
| antigen (MNDA) | | |
| G1/S-specific cyclin D1 (CCND1); | * | X59798 |
| cyclin parathyroid adenomatosis 1 | | |
| (PRAD1); bcl-1 oncogene | | |
| cyclin-dependent kinase 4 inhibitor 2 | * | L27211.1 |
| (CDK4I; CDKN2); p16-INK4; multiple | | |
| tumor suppressor 1 (MTS1) | | |
| cyclin-dependent kinase inhibitor 1C | * | U22398 |
| (CDKN1C); p57-KIP2 | | |
| ezrin; cytovillin 2; villin 2 (VIL2) | 1.69 | X51521 |
| proto-oncogene tyrosine-protein | 1.55 | L04143.1 |
| kinase kit; c-kit; mast/stem cell | | |
| growth factor receptor | | |
| precursor(SCFR); CD117 antigen | | |
| proliferating cell nucleolar anti- | 1.52 | M32110 |
| gen P120; NOL1 | | |
| jun proto-oncogene; avian sarcoma | 1.47 | J04111 |
| virus 17 oncogene homolog; | | |
| transcription factor AP-1 | | |
| C-src proto-oncogene (SRC1) | 1.35 | X59932 |
| CDC-like kinase 3 (CLK3) | 1.35 | L29220 |
| cell division cycle protein 25 | 1.34 | M91815.1 |
| nucleotide exchange factor (CDC25) | | |
| prothymosin alpha (PROT-alpha; PTMA) | 1.32 | M26708 |
| 40S ribosomal protein S19 (RPS19) | 1.31 | M81757 |
| avian myelocytomatosis viral | 1.30 | V00568 |
| oncogene homolog (MYC) | | |
| CDC-like kinase 1 (CLK1) | 1.27 | L29219.1 |
| cyclin-dependent kinase 4 inhibitor | 0.69 | U49399.1 |
| 2D (CDKN2D); p19-INK4D | | |
| vascular endothelial growth factor | 0.62 | XM_039993.2 |
| receptor 1 (VEGFR1); tyrosine- | | |
| protein kinase receptor flt + | | |
| soluble VEGFR; tyrosine-protein | | |
| kinase receptor SFLT | | |
| neogenin | — | U61262.1 |
| webB2 receptor protein-tyrosine | — | M11730.1 |
| kinase; neu proto- | | |
| oncogene; c-erbB2 + | | |
| HER2 receptor | | |
| N-ras; transforming p21 protein | — | AAA60255 |

TABLE 3 b
Ion Channels, Modulators, Effectors

| Gene | Fold change | GenBank ID |
|---|---|---|
| extracellular signal-regulated | ** | X14798.1 |
| kinase 3 (ERK3); mitogen-activated | | |
| protein kinase 6 (MAP kinase 6; | | |
| MAPK6; PRKM6); p97-MAPK | | |
| 40-kDa heat-shock protein 1 (HSP40); | ** | D49547 |
| DNAJ protein homolog 1 (HDJ1; DNAJ1) | | |
| 70-kDa heat shock protein 1 | ** | M11717 |
| (HSP70.1; HSPA1) | | |
| glutaredoxin | ** | X76648 |
| tyrosine kinase receptor tie-1 | * | AAB84296 |
| precursor | | |
| ras-related protein RAB3B | * | NM_002867.1 |
| macMARCKS; MARCKS-related protein | * | P49006\| |
| (MRP); MLP | | |
| mitogen-activated protein kinase 3 | * | P27361 |
| (MAPK3; PRKM3); MAPK1; extra- | | |
| cellular signal-regulated kinase | | |
| 1 (ERK); microtubule-associated | | |
| protein 2 kinase, insulin- | | |
| stimulated MAP2kinase | | |
| mitgoen-activated protein kinase 9 | * | NM_002752.1 |
| (MAP kinase 9; MAPK9; PRKM9); c-jun | | |
| N-terminal kinase 2 (JNK2); JNK55 | | |
| 60-kDa heat shock protein (HSP60); | * | M22382.1 |
| HSPD1; 60-kDa chaperonin; | | |
| mitochondrial matrix protein | | |
| P1 precursor; p60 lymphocyte | | |
| protein; HUCHA60; GROEL | | |
| serine kinase | 2.24 | U09564.1 |
| transferrin receptor (TFRC); CD71 | 1.80 | M11507.1 |
| antigen | | |
| Neurotrophic tyrosine kinase | 1.63 | U55017.1 |
| receptor-related 3: TKT percursor | | |
| phospholipase C (PLCL) | 1.62 | X14034. |
| cAMP-response element binding | 1.59 | M27691.1\| |
| protein (CREB) | | |
| ephrin type-A receptor 1 precursor; | 1.55 | M18391 |
| tyrosine-protein kinase receptor eph | | |
| 27-kDa heat-shock protein (HSP27); | 1.42 | X54079.1 |
| stress-responsive protein 27 | | |
| (SRP27); estrogen-regulated 24-kDa | | |
| protein; HSPB1 | | |
| tyrosine kinase tnk1 | 1.42 | XM_012654.3 |
| ras-related protein RAB3A | 1.38 | XM_054457.2 |
| janus kinase 3 (JAK3); leukocyte | 1.33 | XM_038595.3 |
| janus kinase (L-JAK) | | |
| dual-specificity mitogen-activated | 1.29 | NM_002755.2 |
| protein kinase kinase 1 (MAP kinase | | |
| kinase 1; MAPKK 1; MKK1); | | |
| extracellular signal-regulated | | |
| kinase 1; ERK activator kinase 1 | | |
| calcium/calmodulin-dependent protein | 1.27 | NM_001744.1 |
| kinase type IV catalytic subunit | | |
| (CAMK IV); CAM kinase-GR | | |
| ras-related protein RAB5A | 0.75 | XM_053461.2 |
| colon carcinoma kinase 4 precursor | 0.68 | U33635.1 |
| (CCK4) + transmembrane receptor | | |
| PTK7 | | |
| epithelial discoidin domain receptor | 0.63 | XM_004559.5 |
| 1 precursor (EDDR1; DDR1); cell | | |
| adhesion kinase (CAK); TRKE; | | |
| RTK6; protein tyrosine kinase 3A | | |
| (PTK3A); neuroepithelial tyrosine | | |
| kinase (NEP) | | |
| ras-related protein RAB6 | 0.27 | M28212.1 |
| cAMP-dependent protein kinase type I | 0.23 | M65066.1 |
| beta regulatory subunit (PRKAR1B) | | |
| tyrosine-protein kinase ack | — | CAC15525 |
| T-lymphocyte maturation-associated | — | P21145 |
| protein MAL | | |
| orphan hormone nuclear receptor | — | U04897.1 |
| LIM domain kinase 1 (LIMK-1) | — | P53667 |
| protein kinase C alpha polypeptide | — | NM_002737.1 |
| (PKC-alpha; PKCA) | | |
| dual specificity mitogen-activated | — | P46734 |
| protein kinase kinase 3 (MAP kinase | | |
| kinase 3; MAPKK3; MKK3); ERK | | |
| activator kinase 3; MAPK/ERK | | |
| kinase 3 (MEK3) | | |
| Yamaguchi sarcoma viral-related | — | M16038.1 |
| oncogene homolog; tyrosine-protein | | |
| kinase lyn | | |
| protein-tyrosine phosphatase 1E | — | U12128.1 |

TABLE 3.c
Apoptosis, DNA Synthesis, Repair & Recombination

| Gene | Fold change | GenBank ID |
|---|---|---|
| ubiquitin-conjugating enzyme E2 17- | ** | NM_003336.1 |
| kDa (UBE2A); ubiquitin-protein | | |
| ligase; ubiquitin carrier protein, | | |
| HR6A | | |
| growth arrest & DNA-damage-inducible | ** | S40706.1 |
| protein 153 (GADD153); DNA-damage- | | |
| inducible transcript 3 (DDIT3); C/EBP | | |
| homologous protein (CHOP) | | |
| growth factor receptor-bound protein | * | M96995.1 |
| 2 (GRB2); ASH protein | | |
| glutathione S-transferase A1 (GTH1; | * | M21758.1 |
| GSTA1); HA subunit 1; GST-epsilon | | |
| cytoplasmic dynein light chain 1 | * | U32944.1 |
| (HDLC1); protein inihibitor of | | |
| neuronal nitric oxide synthase (PIN) | | |
| xeroderma pidmentosum group G | * | NM_021141.2 |
| complementing protein (XPG); X-ray | | |
| repair-complementing defective repair | | |
| in Chinese hamster cells 5 (XRCC5) | | |
| xeroderma pigmentosum group D | * | AF035587.1 |
| complementing protein (XPD); X-ray | | |
| repair-complementing defective repair | | |
| in Chinese hamster cells 2 (XRCC2) | | |
| RAD23 homolog A (RAD23A; hHR23A) | * | NM_005053.1 |
| ataxia telangiectasia (ATM) | * | AAB38309 |
| apoptosis regulator bcl-x | 1.60 | Z23115.1 |
| caspase 9 percursor (CASP9); ICE-like | 1.42 | AB020979.1 |
| apoptotic protease 6 (ICE-LAP6); | | |
| apoptotic protease MCH6; | | |
| apoptotic protease activating factor | | |
| 3 (APAF3) | | |
| CD40 receptor-associated factor 1 | 1.39 | U21092.1 |
| (CRAF1) | | |
| SL cytokine precursor; FMS-related | 1.35 | NM_001459.1 |
| tyrosine kinase 3 ligand (FLT3 | | |
| ligand; FLT3LG) | | |
| cytochrome P450 reductase | 1.33 | AAD45961.1 |
| X-ray repair complementing defective | 1.25 | M36089 |
| repair in | | |
| Chinese hamster cells 1 (XRCC1) | | |
| Ku (p70/p80) subunit; ATP-dependent | 0.74 | X57500.1 |
| DNA helicase II 86-kDa subunit; lupus | | |
| ku autoantigen protein; thyroid-lupus | | |
| autoantigen (TLAA); CTC box | | |
| binding factor 85-kDa subunit (CTCBF; | | |
| CTC85); nuclear factor IV | | |
| caspace 10 precursor (CASP10); ICE- | 0.45 | Q92851 |
| LIKE apoptotic protease 4 (ICE-LAP4); | | |
| apoptotic protease MCH4; fas- | | |
| associated death domain | | |
| protein; interleukin 1 beta- | | |
| converting enzyme 2 (FLICE2); | | |
| inhibitor of apoptosis protein 2 | — | Q13490 |
| (HIAP2; IAP2) + IAP homolog B; | | |
| TNFR2-TRAF signaling complex | | |
| protein 2; MIHB | | |
| recA-like protein HsRad51; DNA repair | — | BAA02962.1 |
| protein RAD51 homolog | | |
| DNA damage repair & recombination | — | B56529 |
| protein 52 (RAD52) | | |
| DNA ligase III (LIG3); polydeoxyribo- | — | CAA59230.1 |
| nucleotide synthase | | |

TABLE 3 d
Transcription Factors, DNA Binding Proteins

| Gene | Fold change | GenBank ID |
|---|---|---|
| transcriptional activator hSNF2-alpha | ** | D26155.1\| |
| early growth response protein 1 | 2.71 | M62829.1 |
| (EGR1); transcription factor ETR103; | | |
| KROX24; zinc finger protein 225 | | |
| (ZNF225); AT225 | | |
| homeobox A1 protein (HOXA1); HOX1F | 2.17 | U10421.1 |
| transcription factor NF-ATc | 1.67 | U08015.1 |
| R kappa B DNA-binding protein | 1.66 | U08191.1 |
| transcription initiation factor IID | 1.57 | M55654 |
| 31-kDa subunit (TFIID); TATA-box- | | |
| binding protein-associated factor RNA | | |
| polymerase II G 32-kDa subunit | | |
| (TAFII32; TAF2G); TAFII31 | | |
| homeobox protein hLim1; LHX1 | 1.51 | NM_005568.1 |
| helix-loop-helix protein HLH 1R21; | 1.49 | X69111.1 |
| DNA-binding protein inhibitor Id-3; | | |
| HEIR-1 | | |
| guanine nucleotide-binding protein | 1.46 | NP_000507.1 |
| G-s alpha subunit (GNAS); adenylate | | |
| cyclase-stimulating G alpha protein | | |
| CCAAT-binding transcription factor | 1.45 | AAA40889.1 |
| subunit B (CBF-B); NF-Y protein sub- | | |
| unit A (NF-YA); Hap2; CAAT-box DNA- | | |
| binding protein subunit A | | |
| transcription factor LSF | 1.37 | B53771 |
| homeobox 2.1 protein (HOX2A); HOXB5; | 1.35 | M92299.1 |
| HU1; HHO.C10 | | |
| endothelial transcription factor | 1.34 | M68891.1 |
| GATA2 | | |
| transcription factor Sp1 (TSFP1) | 1.30 | XM_028606.2 |
| transcription factor ZFM1 | 0.62 | G02919 |
| zinc finger protein 161 (ZNFI61); | 0.26 | NP_009077.1 |
| putative transcription activator DB1 | | |
| stem cell protein (SCL); T-cell | — | AAA36598.1 |
| leukemia/lymphoma-5 protein (TCL5); | | |
| T-cell acute lymphocytic leukemia-1 | | |
| protein (TAL1) | | |
| neural retina-specific leucine zipper | — | NP_006168 |
| protein (NRL) | | |
| MSX-1 homeobox protein; HOX7 | — | P28360 |
| basic transcription factor 62-kDa | — | AAA58399.1 |
| subunit (BTF2) | | |
| paired box homeotic protein (PAX8) | — | BAB59039.1 |
| isoforms 8A/8B + isoforms 8C/8D | | |
| brain-specific homeobox/POU domain | — | AAA65605.1 |
| protein 3A (brn-3A); RDC-1; octamer | | |
| binding transcription factor 1 (OTF1) | | |
| transcription factor E2-alpha (E2A); | — | AAA61146.1 |
| immunoglobulin enhancer binding | | |
| factor E12; transcription factor-3 | | |
| (TCF3) | | |
| transcriptional enhancer factor | — | P28347 |
| (TEFI); protein GT-IIC; transcription | | |
| factor 13 (TCF13) | | |
| thioredoxin perodxidase 2 (TDPX2); | — | Q06830 |
| thioredoxin-dependent peroxide | | |
| reductase 2; proliferation- | | |
| associated gene (PAG); natural killer | | |
| cell enhancing factor A (NKEFA) | | |

TABLE 3 e
Receptors, Cell Surface Antigens, Cell Adhesion

| Gene | Fold chance | GenBank ID |
|---|---|---|
| interleukin-2 receptor gamma subunit | * | AAA59145.1 |
| (IL-2R gamma; IL2RG); cytokine | | |
| receptor common gamma chain | | |
| precursor; p64 | | |
| interferon gamma receptor (IFNGR) | * | NM_000416.1\|[ |
| interleukin-1 receptor type I | * | M27492.1 |
| precursor (IL-1R1); IL-1R-aplha; p80; | | |
| CDW121A antigen | | |
| neural-cadherin precursor (N- | * | L34059 |
| cadherin; NCAD); cadherin 2 (CDH2) | | |
| neural cell adhesion molecule L1 | * | M77640 |
| precursor (N-CAM L1); MIC5 | | |
| integrin alpha 3 (ITGA3); galacto- | * | M59911.1 |
| protein B3 (GAPB3); very late antigen | | |
| 3 alpha subunit (VLA3 alpha); CD49C | | |
| antigen | | |
| leukocyte adhesion glycoprotein p150, | * | M81695.1 |
| 95 alpha subunit precursor; leukocyte | | |
| adhesion receptor p150, 95; CD11C | | |
| antigen; leu-M5; integrin alpha X | | |
| (ITGAX) | | |
| integrin beta 4 (ITGB4); CD104 | * | X51841.1 |
| antigen | | |
| CD44 antigen precursor (CD44); | 1.51 | XP_030326.1 |
| phagocytic glycoprotein I (PGP1); | | |
| HUTCH I; extracellular matrix | | |
| receptor III (ECMR III); gp90 | | |
| lymphocyte homing/adhesion receptor | | |
| (LHR); hermes antigen; hyaluronate | | |
| receptor; heparan sulfate | | |
| proteoglycan; epican | | |
| glutamate receptor subunit epsilon 3 | 1.44 | NP_000826.1 |
| precursor (GRIN2C); N-methyl D- | | |
| aspartate receptor subtype 2C | | |
| (NMDAR2C; NR2C) | | |
| CD27L antigen receptor precursor; | 0.7 | P26842 |
| tumor necrosis factor receptor | | |
| superfamily member 7 (TNFRSF7); T14 | | |
| integrin alpha L (ITGAL); leukocyte | 0.45 | P20701 |
| adhesion glycoprotein alpha subunit | | |
| precursor; leukocyte function- | | |
| associated molecule 1 alpha chain | | |
| (LFAI); CD11A antigen | | |
| interleukin 2 receptor alpha subunit | 0.41 | P01589 |
| precursor (IL-2 receptor alpha | | |
| subunit; IL2RA); TAC antigen; CD25 | | |
| antigen | | |
| CDW40 antigen; CD40L receptor | 0.35 | CAA43045.1 |
| precursor; nerve growth factor | | |
| receptor-related B-lymphocyte | | |
| activation molecule | | |
| granulocyte colony stimulating factor | — | Q99062 |
| receptor precursor (GCSF-R); CD114 | | |
| antigen | | |
| low-affinity nerve growth factor | — | AAB59544.1 |
| receptor (NGF receptor; NGFR); GP80- | | |
| LNGFR | | |
| neuromedin B receptor (NMBR); | — | NP_002502.1 |
| neuromedin-B-preferring bombesin | | |
| receptor | | |
| granulocyte-macrophage colony- | — | Q00941 |
| stimulating factor receptor alpha | | |
| (GM-CSFR-alpha); CSW116 antigen | | |
| platelet membrane glycoprotein IIIA | — | P05106 |
| precursor (GP3A); integrin beta 3 | | |
| (ITGB3); CD61 antigen | | |
| integrin alpha 7B precursor (IGA7B) | — | CAA52348.1 |

TABLE 3 f
Growth Factors, Cytokines, Hormones

| Gene | Fold change | GenBank ID |
|---|---|---|
| Interleukin-10 precursor (IL-10); | ** | M57627 |
| cytokine synthesis inhibitory factor | | |
| (CSIF) | | |
| Granulocyte-macrophage colony | * | AAA52578.1 |
| stimulaitng factor (GM-CSF); CSF2 | | |
| FMLP-related receptor I (FMLPRII); | * | AAA58482.1 |
| RMLP-related receptor I (RMLPRI) | | |
| Glia maturation factor beta | * | P17774 |
| (GMF-beta) | | |
| Hepatoma-derived growth factor (HDGF) | * | P51858 |
| Macrophage inflammatory protein 1 | * | P10147 |
| alpha precursor (MIP1-alpha); | | |
| tonsillar lymphocyte LD78 alpha | | |
| protein; G0S19-1 protein; PAT 464.2; | | |
| SIS-beta; small inducible cytokine | | |
| A3 (SCYA3) | | |
| Monocyte chemotactic protein 1 | * | P13500 |
| precursor (MCP1); monocyte | | |
| chemotactic and activating factor | | |
| (MCAF); monocyte secretory protein | | |
| JE; monocyte chemoattractant protein | | |
| 1; HC11; small inducible cytokine A2 | | |
| (SYCA2). | | |
| Oncostatin M (OSM) | * | NP_065391.1 |
| Renin-binding protein (RENBP; RNBP) | | XP_013053.3 |
| Calgranulin B (CAGB); migration | 1.49 | B31848 |
| inhibitory factor-related protein 14 | | |
| (MRP14); leukocyte L1 complex heavy | | |
| chain; S100 calcium binding protein | | |
| A9 (S100A9) | | |
| Placenta growth factors 1 + 2 | 1.42 | CAA38698.1 |
| (PLGF1 + PLGF2) | | |
| Vascular endothelial growth factor | 1.42 | AAA35789.1 |
| precursor (VEGF); vascular | | |
| permeability factor (VPF) | | |
| Hepatocyte growth factor activator | 1.40 | BAA74450.1 |
| (HGF activator) | | |
| Follistatin-related protein precursor | 1.34 | AAA66062.1 |
| Hepatocyte growth factor-like protein; | 1.29 | AAA59872.1 |
| macrophage stimulating protein (MSP) | | |
| Interferon gamma precursor (IFN- | 1.29 | P01579 |
| gamma, IFNG); immune interferon | | |
| WSL protein + TRAMP + Apo-3 + | 0.69 | AAB41432.1 |
| death domain receptor 3 (DDR3) | | |
| Neurotrophin-4 (NT4) | 0.68 | AAA60154.1 |
| Interleukin-13 precursor (IL-13); | 0.39 | P35225 |
| NC30 | | |
| Small inducible cytokine A5 (SYCA5); | 0.38 | XP_035842.1 |
| regulated on activation normal T- | | |
| cell-expressed & secreted | | |
| protein precursor (RANTES); SIS delta | | |
| Estrogen sulfotransferase (STE; EST1) | — | CAA72079.1 |
| Keratinocyte growth factor (KGF); | — | AAA63210.1 |
| fibroblast growth factor 7 (FGF7) | | |
| Endothelial-monocyte activiting | — | AAA62202.1 |
| polypeptide II (EMAP II) | | |
| Leukemia inhibitory factor precursor | — | B36282 |
| (LIF); differentiation-stimulating | | |
| factor (D factor); melanoma derived | | |
| LPL inhibitor (MLPLI); HILDA | | |
| Acidic fibroblast growth factor | — | AAA51672.1 |
| (AFGF) + heparin-binding growth | | |
| factor 1 precursor (HBGF-1) + | | |
| beta-endothelial cell growth | | |
| factor (ECGF-beta) | | |
| Insulin-like growth factor-binding | — | P17936 |
| protein 3 precursor (IGF-binding | | |
| protein 3; IGFBP3; IBP3) | | |

Symbols (Fold Changes)
**Expressed in PP but not NPP, or in Co-culture but not Caco2.
*Expressed in Co-culture but no Caco2 (only repeated once)
—Expressed in Caco2 but not in co-culture.

### Immunity

The events of the cell cycle occur under normal circumstances in a fixed sequence. Traditionally, the cycle is divided into two stages: cell division and the interphase. Cell division or mitosis is followed by cytokinesis and together they constitute the ‘M phase’ of the cell cycle. The interphase is divided up into the S, G₁ and G₂ phases. Briefly, during the S phase, DNA is replicated in preparation for mitosis, while the intermediate G phases are transitional periods involved in protein synthesis and cell growth. Activation of regulatory genes that control and maintain a cell's proliferative state by intracellular signals (discussed below) stimulates proliferation of the cell and initiates cell growth. A number of genes involved in these processes were differentially expressed in the co-culture model (as estimated by relative mRNA abundance) and discussed below.

The epithelial cells of the gut play an important part of the innate and specific immunity. IEC's are considered to be in a continuous controlled state of “physiological” inflammation and active processes continually take place to ensure that the tone of immunosuppression is maintained (Mayer, 2000). These unique regulators appear to control the mucosal immune system's condition. These distinct factors govern the immune response, whether it's immune suppression/tolerance, inflammation or a systemic immune response. A clearer understanding of the immunoregulatory features involved in mucosal immunity is clearly desirable and may lead to new approaches in disease and drug therapy. Genes detected in the co-culture model that may be related to or are involved in immune function in GALT are discussed below.

The gamma subunit if IL-2 receptor plays a pivotal role in formation of the full-fledged IL-2 receptor (Di Santo et al., 1995). In an interesting study where infant rats were studied from pre- to post weaned life Masjedi et al. (1999) assessed alterations in expression and phenotype of cells in the gut-associated lymphoid tissue. At an age when the immune system is believed to be immature and functionally naive they discovered interleukin-2 receptor (IL-2R) expression peaked approximately four-fold at midweaning in Peyer's patches, compared with adult animals (day 70) suggesting that IL-2R expression is an adaptation to the host's environment. In a similar way, the presence of IL-2R specific for cells in the co-culture could be a direct result of the environment. The common gamma c chain of the interleukin 2 receptor, gamma is also a component of the receptors for IL-4, 1 L-7, and IL-9 and plays a critical role in lymphoid development through its participation in the receptors for IL-2, IL-4, IL-7, IL-9, and IL-15 (Di Santo et al., 1995)

Interferon-γ (IFN-γ) exhibits various properties including antigrowth activity in neoplastic and normal cells, and regulatory roles in immune responses (Tsuji et al., 1998). Kjerrulf et al. (1997) found that in IFN-γ receptor knockout mice (IFN-γR^{-/-}) reduced mucosal antibody responses and decreased Th1 and Th2 activity after oral immunization. The presence of IFN-γ receptor in the M cell co-culture model could possibly augment a cross-regulation between the two Th subsets in the gut mucosa. It is noteworthy that the ligand, IFN-γ, mRNA was increased in the co-culture that was supported further by the significant secretion of IFN-γ from co-culture monolayers.

The C-C chemokines macrophage inflammatory protein 1 (MIPLα) and monocyte chemotactic protein (MCP1) are synthesized and expressed by epithelial cells (Vainer et al., 1998; Kolios et al., 1999). The purpose of these chemokines expression in the co-culture model could be to function not only in leukocyte migration, but also as adhesins in the interaction between leukocytes and colonic epithelium. However, another C-C chemokine, RANTES, mRNA was observed to be reduced in the co-culture. The reasons for this are unclear. Perhaps, the chemoaffractant activities of other chemokines such as IL-8, MIP1α and MCP1 are sufficient for the M cell and in the absence of T cells the need for RANTES is not required.

From a gene delivery perspective, a higher capacity for translation and protein synthesis in PP tissue indicates that PP tissue is a preferred tissue to which to deliver genes coding for DNA vaccines or antigens. Thus the proposed higher translational capacity of PP tissue has implications for gene delivery especially DNA vaccine delivery and correspondingly antigen expression and local presentation to the mucosal immune system in the gastrointestinal tract. The TF coding genes may be important in priming M cells or precursor cells to M cells to adopt M cell phenotype and/or to facilitate priming of M cells to give a better immune cell outcome.

M cell receptors identified in Table 3(e) above are of particular interest in that they can be used for vaccine and delivery.

In view of the foregoing, in the method of the invention for increasing the level of a protein in a PP cell, which comprises delivering a nucleic acid coding for a protein, the protein may be selected from the group consisting of an IL-2 receptor, a gamma c chain of an IL-2 receptor, intereron-γ, and a C—C chemokine.

### Proliferation and Growth

Cyclin DI is a protein involved in regulation of the cell cycle. Over-expression of the protein is associated with abnormal growth or neoplasia. This protein is positively induced by the p42/p44 MAP kinases (Lavoie et al., 1996). It would be interesting if the neoplasia seen in M cells resulted from activation of this protein considering the coincidental induction of the p44 MAP kinase (ERK1) below. The reduction in cyclin-dependent kinase 4 inhibitor 2D (CDKN2D) mRNA that normally inhibits cell cycle progression (Guan et al., 1996) would insinuate a similar function in the proliferation of these ‘M cells.’

In contrast, the induction of cell cycle inhibitors such as cyclin-dependent kinase inhibitor (CDKI) and cyclin-dependent kinase 4 inhibitor (CDK4J) would appear to be working to counterbalance proliferative stimuli present in the M cell.

PLC-L (phospholipase C-deleted in lung carcinoma) is a putative tumor suppressor gene. It is believed that irregular (in fact deletion) expression of the PLC-L gene contributes to the growth of human lung carcinoma (Kohno et al., 1995). It is possible then that its upregulation in the M cell model is acting as a negative regulator of growth in the cells, counterbalancing the many proliferative signals present.

Growth factor receptor-bound protein 2, GRB2, involved in growth factor control of ras signalling (Lowenstein et al., 1992).

The intracellular signaling pathways responsible for cell cycle arrest and establishment of differentiated cells along the gut axis remain largely unknown particularly in the case for the development of M cells and the FAE. ERK3/MAPK6 is expressed solely in the co-culture. Extracellular signal-regulated kinases-1 (ERK1) also known as the p44 mitogen-activated protein (MAP) kinase (p44mapk) is also induced specifically in the co-culture model. ERK1 and ERK3 are proline-directed serine/threonine kinases that are activated in response to a variety of extracellular signals, including growth factors, hormones and, neurotransmitters. These MAP kinases are key molecules involved in intracellular signal transduction, and are key regulators of cell proliferation in mammalian cells (Davis, 1995). Results indicate that elevated p42/p44 MAPK activities stimulate cell proliferation of intestinal cells, whereas low sustained levels of MAPK activities have correlated with cell cycle arrest and an increased expression of sucrase isomaltase (Aliaga et al., 1999). It is tempting to speculate that the presence of ERK3 together with the other MAP kinases apart from their proliferative effects are in part responsible for a reduction in sucrase isomaltase, a characteristic effect in M cells.

Lying upstream in the ERK signal cascade the tyrosine/threonine protein kinase, MAPK kinase (MAPKK1) is implicated in the regulation of cell growth and differentiation through the activation of ERK. In addition it is interesting to note that MAPKK3 was deleted in the co-culture cells. MAPKK3 phosphorylates and activates p38 MAP kinase alpha and gamma isoforms (Enslen et al., 1998). The induction of the MAPKK1 gene along with serine kinase coincides with the induction of ERK1, highlighting the ERK cascade as an important signalling cascade in M cell maintenance. It is interesting to note that ERK activation is responsible for terminal differentiation of components of the crypt-villus. (Taupin and Podolsky, 1999)

However, glia maturation factor-β (GMF-β) is potentially offsetting the ERK cascade effects. It is known to inhibit MAP kinases particularly ERK1 and ERK2 and yet promotes the p38 MAPK (Zaheer and Lim, 1996 and 1998).

Findings suggest that positive and negative regulation of MAPK activity are associated with loss of normal growth control and may be involved in carcinogenesis of colon cancers. Jun kinases such as JNK2 (MAP kinase 9) mediate signal transduction of pro-inflammatory cytokines and cellular stress (Uciechowski et al., 1996).

CD40 is a receptor on the surface of B-lymphocytes, the activation of which plays critical role in B cell proliferation and differentiation. CRAF1, (CD40 receptor-associated factor 1) encodes a protein that interacts directly with CD40 receptor (Cheng et al., 1995). Its upregulation in the co-culture is perhaps a main determinant of lymphoepithelial crosstalk as discussed above.

The c-myc gene is commonly amplified and over-expressed in many human tumors (Ryan and Birme, 1996). A member of the myc family of helix-loop-helix transcription factors, c-myc is integral in controlling cell growth and promotes cell proliferation and transformation by activating growth-promoting genes (Thompson, 1998). Prothymosin-α (PT-α) is a nuclear protein and its expression is associated with alterations in the proliferative state of cells and has been reported to be regulated by the c-myc gene in vitro. (Smith, 1995; Mon et al., 1993). The increased activity of c-myc in this model is likely to result in the increase in RT-α mRNA.

PKC-α protein levels regulate certain pathways that lead to the expression of differentiation-dependent genes. In a series of antisense transfection experiments where PKC-α expression in CaCo-2 cells was almost completely deleted, enhanced proliferation and a marked decrease in differentiation was observed, as well as a more aggressive transformed phenotype (Scaglione-Sewell et al., 1998). In a similar fashion, the lack of PKC-α mRNA detected in the co-culture ‘M cells’ may underlie some of the phenotype changes featured.

Glutathione S-transferase A1 (GSTA1) is a member of a multigene family of detoxification and metabolizing enzymes. Induction of GST enzyme activity has been demonstrated to act as a potent anti-proliferative and differentiating agent in Caco-2 cells (Stein et al., 1996) suggesting a similar role in the ‘M cell.’

Transcription factor GATA-2 is thought to maintain and promote the proliferation of early haematopoietic progenitor cells.

The placenta growth factor (PLGF) is a member of the vascular enciotheial growth factor (VEGF) family of growth factors. In addition to PLGF, VEGF mRNA was enhanced in the co-culture cells. These growth factors play a crucial role in angiogenesis during development and/or repair (Andre et al., 2000). The augmented transcription of their mRNA is consequently not a surprising find. However, hypoxia and energy depletion are known to induce angiogenesis by increasing VEGF, expression and so the possibility that the co-culture conditions are responsible for these genes induction cannot be ruled out ratherthan a deliberate mechanism of neogenesis in M cell formation. VEGF receptor 1 (VEGFR1); the receptor for VEGF and PLGF, mRNA is down-regulated and is possibly a consequence of desensitization of the receptor by VEGF and PLGF binding, initiating a reduction in the receptor's RNA.

Coinciding with the above actions, the absence of growth factors such as insulin-like growth factor-binding protein 3 (IGFBP3) and keratinocyte growth factor (KGF) may be modulating enterocytic cell proliferation and differentiation.

Caco-2 cells have been shown to express the type I IL-1 R. (Varilek et al., 1994) II-1Rα binds IL-1 and mediates cell signalling particularly signalling involved in cell proliferation (French et al., 1996). The expression of IL-1 R can be enhanced by IFN-γ (Varilek et al., 1994). Therefore, the expression of IL-1 R type 1 mRNA in the co-culture is interesting when considering the significant expression of IFN-1 expressed in supernatants of the co-culture model.

In view of the foregoing, in the method of the invention for increasing the level of a protein in a PP cell, which comprises delivering a nucleic acid coding for a protein, the protein may be selected from the group consisting of cyclin D1, PLC-L, GRB2, ERK3/MAPK6, ERK1, ERK3, JNK2, CD40, CRAF1, C-MYC, PT-α, IL-R, PKC-α, GSTA1, GATA-2, and PLGF.

### Differentiation

Development of cells or differentiation is dictated by the expression of a cell's genes specific to that cell. This is a particularly important aspect with regards to M cells.

The cortical cytoskeleton not only provides structural support to the plasma membrane but also contributes to important dynamic processes such as endocytosis, exocytosis, and transmembrane signalling pathways. Ezrin, or villin 2, is an F-actin associated molecule and is concentrated in surface projections such as microvilli and membrane ruffles where they link the microfilaments to the membrane and has been reported to be in abundance during development and differentiation of the intestinal epithelium. It was reported that hepatocyte growth factor (HGF/SF) could stimulate the tyrosine phosphorylation of ezrin in a human colon epithelial cell line, which induced the ezrin associated membrane ruffling. It is interesting to note that both hepatocyte growth factor activator (HGF activator) and hepatocyte growth factor-like protein were both upregulated in the co-culture model and taken with the augmented ezrin mRNA the induction of these genes would appear to underlie the mechanism involved in the morphogenesis observed in M-cells.

These data demonstrate that the expression of the ezrin gene is being regulated at the level of mRNA due to effects incurred by the B-cells. It is particularly relevant considering the observations of villin diffusely displayed in M-cells.

One method of actin cytoskeletal reorganization is controlled by the LIMK-1 serine/threonine kinase, which acts by phosphorylating cofilin and subsequently Rac (as previously reported). However, LIMK-1 was deleted in the co-culture model and would appear to rule out the Rac-mediated mechanism of actin reorganization in the M cell model.

The cadherin family of cell adhesion molecules play important role in cell-cell adhesion during tissue differentiation. They have been reported to be linked to the actin cytoskeleton by catenins located in the cytoplasmic compartment of the cell. The specific expression of NCAD in the co-culture suggests a distinct gene involved in the cytoskeletal structure.

Previous reports have shown that neogenin is closely related to the human tumor suppressor molecule DCC (deleted in colorectal cancer) and together they constitute a subgroup of Ig superfamily proteins that have shown to be essential for terminal differentiation of specific cell types in the adult including the human colon. These parallels suggest that neogenin, like DCC, is functionally involved in the transition from cell proliferation to terminal differentiation of specific cell types. Its absence in the co-culture model might represent a period of continued proliferation for the cells and allow a longer period of proliferation.

The helix-loop-helix (HLH) family of transcription factors has been shown by others to play a central role in the regulation of cell growth, differentiation and tumorigenesis. Of particular interest, when HLH 1 R21 was over-expressed in mouse NIH3T3 cells, it induced a morphologically transformed phenotype.

Other genes associated with differentiation including Myeloid cell nuclear differentiation antigen (MNDA) and the LHX1 gene. The LHX1 gene is a member of the LIM/homeobox (Lhx) gene family. It has been shown that it codes for a transcriptional regulatory protein involved in the control of differentiation and development.

In view of the foregoing, in the method of the invention for increasing the level of a protein in a PP cell, which comprises delivering a nucleic acid coding for a protein, the protein may be selected from the group consisting of HGF activator, ezrin, NCAD, MNDA, and LHX1.

### Adhesion

It is clearly evident that modification of the M cell apical surface is a determining factor in M cell apical membrane adherence, and thus, uptake and transport of macromolecules/microorganisms and targeting epitopes on the surface of M cells has been used to promote further adherence and uptake of particles in vaccinology. The specificity of these markers is not only useful for vaccine strategies but also represents targets for understanding adhesion and uptake of bacteria and viruses. Adhesion is not privy to the apical surface. Adhesion molecules on the basolateral surface of M cells, such as cadherin 2, neural cell adhesion molecule, integrin alpha 3, leukocyte adhesion glycoprotein p150, integrin beta 4 are understood to be involved in leukocyte migration and in the development/organization of lymphoid nodules in Peyer's patches. Genes expressed/induced in the co-culture can provide an insight into the mechanisms involved and are discussed below.

The tyrosine kinase receptor TIE 1 is normally located in vascular endothelial and haematopoietic cells and is largely involved in the proliferation and differentiation of miniature haematopoietic cells and would be an appropriate gene specific for M cells. In the brain, TIE mRNA and protein is significantly elevated in lesions composed of abnormal vasculature called arterioyenous malformations (AVMs) and the surrounding vasculature. Like AVMs, the significant upregulation of TIE in M cells may indicate some ongoing neogenesis, and depending on the receptor's polarity could be of potential use in vaccine targeting.

The neuronal cell adhesion molecule L1 (NCAML1) is a transmembrane glycoprotein belonging to the immunoglobulin superfamily and is generally associated with development of the nervous system. As a potent promoter of neurite growth, it is allied with plastic changes. In nerve growth it interacts with the actin cytoskeleton via an ankyrin linkage and promotes specific distribution of F-actin. Such flexibility is ideal in the M cell scenario.

The integrin family consists of a series of related alpha beta heterodimers involved in a variety of cell-matrix and cell-cell adhesion functions. The α₃β₁ integrin is a multiligand extracellular matrix receptor found on many cell types and can function as a receptor for fibronectin, laminin, and collagen. Phagocytosis of molecules by breast cells has also been reported to involve this adhesion molecule, thus, it would appear a suitable candidate as an adhesion target on M cells.

The leukocyte adhesion glycoprotein p150 (CD11 C antigen), also a member of the integrin family, is involved in leukocyte sequestration via interaction of CD11/CD18 similar to that of ICAM-1.

In stratified epithelia p4 integrin (CD104 antigen) has been shown to be important for proper differential expression and crucial for stable adhesion to die basement membrane through its ability to attach externally to laminin and internally to the keratin cytoskeleton. Interestingly, during human intestinal organogenesis receptors have been shown to occur. This integrin would appear to play an important role in epithelial cell-matrix interactions during development but particularly in M cell development.

CD44 is a major surface adhesion molecule involved in cell-cell and cell-matrix interactions and lymphocyte homing and activation. The observed enhanced expression suggests that this molecule is an important feature in the activities of M cells. A non-receptor tyrosine kinase, C-src protooncogene (SRC1) has been shown to cause overexpression of CD44 in the intestine. As well as its effects on proliferation, the enhanced activity of SRC1 seen in the M cell model would appear to have major effects on cell adhesion properties of the M cell. Hepatocyte growth factor activator (HGF activator) is a serine protease produced and normally secreted by the liver. It has been documented as stimulating reparative processes in intestinal epithelial cells and could be why its activity is enhanced in this model. However, stimulation of CD44 in colonic epithelial cells has been reported to augment c-met, the HGF receptor. This in turn stimulates the “inside-out signalling causing an amplified expression of integrins that leads to an increase in vascular adhesion to the epithelium.

It has been reported that the glutamate receptor (NMDA) is generally associated with learning and memory, highly plastic processes in the brain. The high density of NMDA receptors reflects similar plastic changes seen in the co-culture model but would also act as a target epitope for drug delivery.

TKT is a tyrosine-kinase receptor related to TRK and is a member of cell adhesion kinase receptor family. Ephrin (type A) is a tyrosine kinase receptor that has been reported to be involved in neogenesis and tumor formation. Sp1 is a nuclear protein constitutively expressed and mediates basal promoter activity and is the main Vitamin-D receptor promoter in intestine. These are all potential target sites relevant to M cells.

Many of the receptors/cell surface antigen ‘deleted’ (not detectable) in the co-cultures could be putative negative markers of M cells. A good example is the laminin receptor a₇β₁ integrin. Expression of the a₇β₁ integtin correlates with human intestinal cell differentiation and could be used in a similar fashion that was applied with sucrase isomaltase and alkaline phosphatase.

In view of the foregoing, in the method of the invention for increasing the level of a protein in a PP cell, which comprises delivering a nucleic acid coding for a protein, the protein may be selected from the group consisting of cadherin 2, neural cell adhesion molecule, integrin alpha 3, leukocyte adhesion glycoprotein p150, integrin beta 4, TIE, NCAML1, α3β1 integrin, CD11C antigen, CD104 antigen, CD44, NMDA, TKT, ephrin (type A), and Sp1.

### Transport

The RAB proteins are reported to be regulators of polarized membrane traffic in epithelial cells. The RAB3B is localized to the apical pole very near the tight junctions between adjacent epithelial cells where it is reported to be a possible regulator of apical and/or jupctional protein traffic in epithelial tissues. RAB3B is highly homologous to a brain-specific RAB3 isoform (RAB3A) that targets the presynaptic nerve terminal, where it is reported to regulate exocytosis.

In polarized cells, the small GTPase Rab5a is localized to the plasma membrane, clathrin-coated vesicles, and early endosomes and is a regulator of transport between the plasma membrane and early endosomes. The decreased expression of RAB5a seen in the co-culture may deregulate the rate of endocytosis and/or vesicle fusion and could possibly release ‘the brake’ on vesicle trafficking.

RAB6 is another ras related protein also a regulator of intracellular transport in mammalian cells. It controls intra-Golgi transport, either acting as an inhibitor in anterograde transport or as a positive regulator of retrograde transport. Like RAB5a, the pronounced decrease seen in mRNA transcription could be a means of subverting transport regulation in epithelial cells and so optimize the process as observed in M cells.

Protein kinase C (PKC) and the actin cytoskeleton are critical effectors of membrane trafficking in mammalian cells. The F-actin cross-linking protein myristoylated alanine-rich C kinase substrate (MARCKS), a substrate for PKC, has been reported to be a component of the mechanism of endocytosis.

TIR or p71 plays a key role in the control of cell proliferation through the binding of transferrin, the major iron-carrier protein. Located on both apical and basolateral surfaces, the transferring receptor has the ability to internalize and recycle to the surface. Indeed experiments by Hughson and Hopkins (1990) demonstrate pathways from the apical and basolateral surfaces meet in an endosomal compartment. Furthermore, Shah and Shen (1994) discovered that the fungal metabolite brefeldin A (BFA) could relocate receptor distribution and enhance TfR mediated transcytosis. The increased expression of this mRNA in the M cell model suggests a potential delivery mechanism of protein drugs across the intestinal epithelium present in M cells that could be exploited.

In view of the foregoing, in the method of the invention for increasing the level of a protein in a PP cell, which comprises delivering a nucleic acid coding for a protein, the protein may be selected from the group consisting of a RAB protein, PKC, and TfR.

### Signal Transduction

In order for a cell to respond to extracellular signals, which cause it to alter gene expression or cellular function, it must involve the activation of a signal transduction cascade. There are many different types of signalling cascades, which can be unique to a specific type of stimulus. There are two main mechanisms by which these cascades transmit their signal, either through the regulation of enzymes, which produce second messenger molecules or through the regulation of protein phosphorylation. The activation of these cascades is usually mediated through specific cell surface or intracellular receptor proteins. The receptor protein recognizes the incoming extracellular signal and responds accordingly, initiating a specific series of intracellular signal that direct the cell's behavior. A number of genes involved in intracellular signalling were upregulated or induced in the M cell model and are discussed below.

A member of the Janus family of tyrosine kinases, which are non-receptor protein kinases, Jak3 is involved in intracellular signalling mediated by cytokines and growth factors such as IL-2, IL-4, and IL-7. Jak3 has been reported to play a crucial role in Peyer's patch organogenesis. Mutant mice deficient in Jak3 presented defects in lymphocyte production and the absence of Peyer's patch structures. Its induced expression suggests a greater level of activity and possibly a major requirement underlying the M cell phenotype ‘switch’.

The nuclear zinc-finger transcription factor, early growth response factor-I (EGR-1) is an immediate-early gene product expressed in response to diverse stimuli and is involved in growth, development, and differentiation. EGR-1 has been reported to function in growth regulation and suppression of cell transformation by transactivation of the TGFβ gene. TGFβ is capable of stimulating the synthesis of extracellular matrix proteins that can potentially stabilize epithelial cell contact with the substratum. In addition EGR-1 also plays a role in the immune response, regulating targets such as IL-2, CD44, ICAM-1, and TNF. Taken together the considerable induction of EGR-1 mRNA emphasizes the importance of this protein's involvement in M cell behavior.

CaM kinase IV (CAMK IV) is involved in Ca2+-dependent mechanism for regulating MAP kinase pathways. Many kinases activity has been observed to be enhanced in this model and so it is logical that CAMK IV expression is induced as a requirement to function.

The tyrosine kinase Tnk1 has been reported to be involved in signalling pathways involving development in adult tissues and in cells of the lymphohaematopoietic system.

Epithelial discoidin domain receptor 1 (EDDR1) mRNA was reduced in the co-culture. EDDR1 is a collagen receptor involved in controlling cellular responses to the extracellular matrix (ECM). The decrease in this gene would implicate it in the reorganization of the M cell in relation to the ECM.

cAMP-dependent protein kinase type I beta regulatory subunit (PRKAR1 B) stimulates growth by modulating the signalling of camp via its regulation of cAMP-dependent protein kinase (PKA). PRKAR1 B's reduction in the co-culture model may represent an inhibitory role in the cell's growth counterbalancing the proliferative signals.

In view of the foregoing, in the method of the invention for increasing the level of a protein in a PP cell, which comprises delivering a nucleic acid coding for a protein, the protein may be selected from the group consisting of Jak 3, EGR-1, TNK1 and CAMK IV.

### Protection and Repair

Chaperones such as HSP40 and HSP70 participate in many biological processes in which protein folding is involved. These include protein translocation, protein translation, protein assembly and disassembly, and protein degradation. It is understandable that such genes would be induced considering the evolving processes of a phenotype ‘switch.’ However heat shock protein production has been reported to be induced as a result of harsh changes in their environmental conditions such as stress, ishaemia or hypoxia resulting in protein damage. Therefore it cannot be ruled out that the induction of these genes is in fact a protective measure as a consequence of the adverse conditions in the co-culture.

HSP60 has been observed in highly replicating cells e.g. short-living epithelial cells of the intestine. Involved in the import and refolding of nuclear-encoded proteins destined for the mitochondrial matrix.

The 27-kDa heat shock protein (HSP27) is expressed in a variety of tissues, including gut epithelia and in the absence of stress has been reported to regulate actin filament dynamics. Hsp27 induction in the M cell model like the other heat shock proteins (IISPs) may be active in development of resistance to stressful conditions. Activation of HSP27 can contribute to agonist-induced phosphorylation-modulated reorganization of the actin cytoskeleton and, in the case of stress activation, provides an actin-based adaptive response of cells to the new environmental conditions, and is ideal candidate for the plasticity seen in M cells.

Expression of receptors for fMLP on human phagocyles is well established, but there is conflicting evidence regarding the potential expression of fMLP receptors on other cells within the mucosa, particularly the epithelial cells. The reported observation of the receptor for the chemotactic peptide fMLP supports the notion of the intestinal epithelial cell as an early “sensor” of infection and inflammation. It has been reported that, fMLP, present in abundance in the lumen of the gut and that activation of fMLP receptors induces cytotoxic effects such as lysosomal release and superoxide generation. Thus, it would appear that their presence would be a defensive role in the event of infection of microorganisms.

Glutaredoxin (thioltransferase) is a small, heat-stable protein catalyzing glutathione-dependent disulfide oxdoreduction reactions in a coupled system with NADPH, GSH and glutathione reductase. It is important in regulating cell metabolism through the inactivation of oxidated transcription factors thought to be important in cellular responses to oxidant stress. This modulation of transcription factors' binding activity has been demonstrated for a number of transcription factors, including NF-kB/Rel proteins, Fos and Jun proteins and nuclear factor I (NFI) family of transcription factors. The induction of such a gene would appear to provide a protective role and is particularly influential on a number of key transcription factors.

CREB has been implicated as having prominent role in protection. Over-expression of the gene was reported to reverse hypoxia elicited TNF induction. This infers that the increase in the cAMP responsive element binding protein (CREB) mRNA is possibly a protective response to conditions.

Inactive in cells under normal conditions, gadd153 expression is markedly induced in response to a variety of cellular stresses, including nutrient deprivation, DNA damage, and oxidative stress (e.g. free radicals) which normally leads to growth arrest. The arrest in growth is thought to allow critical repair processes to be carried out before any further cell cycling. It would appear that the gadd153 expression in the co-culture is for reparative purposes.

The excision repair proteins XPG and XPD have been reported to be involved in nucleotide repair. In addition, mRNA for ubiquitin-conjugating enzyme (likely to be involved in post-replication repair and induced mutagenesis, RAD23, and ataxia telangiectasia are also expressed in the co-culture. Their expression, coinciding with gadd153 suggests there is a high degree of impairment to genes in the M cell model.

Interleukin-13 (IL-13) is a potent anti-inflammatory cytokine and has been reported to have the same protective properties in inflammation as IL-4 through its ability to modulate and suppress pro-inflammatory cytokines. It is puzzling that in an environment with a high level of pro-inflammatory cytokines produced that IL-13 mRNA is in fact reduced. One possible explanation might be its anti-adhesion effect. It has been reported that IL-13 (secreted from lymhocytes) down regulated cell adhesion molecules in colonic epithelium and so the role of IL-13 in the co-cultured cells is modulating cell adhesion properties and not inflammation.

In view of the foregoing, in the method of the invention for increasing the level of a protein in a PP cell, which comprises delivering a nucleic acid coding for a protein, the protein may be selected from the group consisting of HSP40, HSP70, HSP60, HSO27, fMLP-related receptor, HSP27, glutaredoxin, CREB, gadd 153, XPG, XPD, ubiquitin, conjugating enzyme, RAD 23, and ataxia telengiectasia.

### Apoptosis and Programmed Cell Death

In programmed cell death, apoptosis is programmed in the sense that a genetically directed ‘clock’ selects a given time for the death of certain cells. It has been reported that it provides an important mechanism for the maintenance and renewal of cells in the gut and in development. However, for the epithelium to maintain its barrier functions, the level of apoptosis needs to be regulated, and this is ‘checked’ by several signal transduction systems. Toxic insult or lack of factors that maintain cell survival can also lead to apoptotic death of the cell.

It has been reported that over-expression of c-fos and c-jun (constituents of the AP-1 transcription factor) in the intestine correlates with programmed cell death and subsequent cellular regeneration. Other studies have demonstrated increases in both proximal jejunum and colon jun mRNA level coincide with a period of major changes in intestinal cell proliferation). The c-jun protein product involved in activation of AP-1, transcription is enhanced when it is phosphorylated by stress-activated protein kinases of which there are many in the M cell model.

As intestinal epithelial cells reach the villus apex they undergo apoptosis and, are shed and, in normal circumstances, caspases, a family of cysteine proteases, play a central role in initiating, amplifying, and executing apoptosis. The pattern of caspase activation in this process is not understood. It is interesting to note that the apoptosis regulator, bcl-x, and caspase 9 are induced in the co-culture. The bcl-x gene plays an important role in the regulation of programmed cell death (PCD), depending on its splice variant the bcl-x protein can accelerate apoptosis or delay/prevent programmed cell death (as previously reported). Bcl-x controls apoptosis mechanisms at points upstream of caspase activation. Perhaps, it is responsible for the marked induction of caspase-9. Caspase-9 is a caspase initiator. Once activated, it can proteolytically activate other caspases (including 3, 6 and 7), which in turn activate caspase-2 and 6 (as previously reported). Inhibitor of apoptosis protein 2 (HIAP2) binds to and inhibits caspase-3. Its expression is a mechanism of regulating cell death depending on the particular cellular or environmental signals. Therefore, its absence in the co-culture cells and the increased activity of caspase-9 allows caspase-3 unchecked pro-apoptotic activity.

The death domain receptor 3 (DDR3) member of the TNFR family can induce apotosis as previously reported. Its mRNA expression is also reduced in the co-culture model.

In view of the foregoing, in the method of the invention for increasing the level of a protein in a PP cell, which comprises delivering a nucleic acid coding for a protein, the protein may be selected from the group selected from: bcl-x and capase-9 and more generally in view of the foregoing may be selected from the group consisting of

cyclin D1, PLC-L, GRB2, ERK3/MAPK6, ERK1, ERK3, JNK2, CD40, CRAF1, C-MYC, PT-α, IL-R, CD40, C-MYC, PKC-α, GSTA1, GATA-2, PLGF, ezrin, HGF activator, hepatocyte growth factor-like protein, NCAD, MNDA, LHX1, TIE-1, NCAML1, CD104, CD44, SRC1, NMDA, TKT, ephrin (type A), Sp1, RAB proteins, PKC, TIR, Jak3, EGR-1, TNK1, CAMK IV, HSP40, HSP70, HSP60, HSO27, fMLP-related receptor, HSP27, glutaredoxin, CREB, gadd153, XPG, XPD, ubiqitin-conjugating enzyme, RAD23, cadherin 2, neural cell adhesion molecule, integrin alpha 3, leukocyte adhesion glycoprotein p150, integrin beta 4, TIE, NCAML1, α3β1 integrin, CD11C antigen, CD104 antigen, CD44, NMDA, TKT, ephrin (type A), and Sp1, a RAB protein, PKC, and TfR, bcl-x and capase-9

### Example 6

### Targeted Gene Delivery

Delivery of genes, gene fragments, oligonucleotides or other nucleotide fragments or analogues of the present invention to a living organism can be accomplished by methods currently available in the prior art. For example, various recombinant viruses have been used for the oral delivery of genes, such as adenovirus, retrovirus, adeno-associated virus, vaccinia virus, lenti-virus and plant-derived viruses, wherein the viral genome is replaced with an expression vector for the gene of interest. See, David T. Page and Sally Cudmore (2001). Innovations in oral gene delivery: challenges and potentials. Drug Discovery Today, Vol. 6, No. 2, pp 92-101. Viral mimetic particles such as virosomes and various types of polymers and liposomes, such as cationic and fusogenic, are also employed for gene delivery. See, U.S. Pat. Nos. 4,885,172, 5,047,245, 5,171,578, 5,059,421, 5,399,331, 5,204,112, 1,252,263, 5,376,452, 5,552,155, 6,120,797, 6,087,325, 6,143,716. Examples of polymers are PLGA, PLA co-polymers, chitosan, and fumaric acid/sebacic acid co-polymers. For these systems, the polymer or liposome is formed from component parts in a solution of the gene expression vector, thus encapsulating the genes when particles are formed. Cationic lipids such as DOTAP and polyethylenimine are commonly used whereby the gene expression vector is complexed with and protected by the lipids. (See, Ogris M. et al. (2001). DNA/polyethylenimine transfection particles: Influence of ligands, polymer size, and pegylation on internalization and gene expression. AAPS PharmSci, 3 (3), article 21). Agents such as protamine are used to condense DNA, which due to the reduction in size of the DNA particles are more easily taken up by cells. Recombinant live bacteria (e.g. Shigella spp, Salmonella spp.) have also been exploited for gene delivery to the gut. Oral bioavailability enhancers, (e.g. sodium caprate, Elan's PROMDAS technology) could be used to increase uptake of a gene or encapsulated gene formulation.

In all cases the delivery systems can be targeted with various ligands on the surface of the particles in order to enhance binding to specific cells type and/or to enhance uptake. These ligands could be peptides, proteins, antibodies, peptidomimetics, and lipids that recognize or are being recognized by specific sites/receptors on the cell surface (Maruyama K. (2000). In vivo Targeting by Liposomes. Biol. Pharm. Bull., 23(7), 791-799).

The targeting ligands may be peptide based, peptidomimetic based, antibody based, single chain antibody based, small organic molecule based. The targeting ligands may also be natural substrates for such receptors, transporters or other cell surface molecules found on the surface of M cells or other cell types found in Peyer's patch. The targeting ligands may be engineered so as to be genetically expressed on the surface of viruses, bacteriophages, virosomes, bacteria or other organisms, which can be utilized for vaccine delivery in the gut. Furthermore the targeting ligands can be presented either as direct conjugates to antigens, or on the surface of drug-loaded particulates such as liposomes, PLGA particles, other particulates and at the same time retain recognition by and interaction with the receptors, transporters or other cell surface molecules found on the surface of M cells and/or other cells of Peyer's patch tissue.

Examples of peptides that target the gastro-intestinal tract, in particular, membrane translocating peptides useful for vaccine delivery to M cells along with M cell specific targeting ligands are described in Table 4.

Further, targeting ligands can be genetically engineered into the surface coats of viruses, bacteriophages or bacteria, conjugated directly to antigens conjugated to the lipids in liposomes by covalent methods or streptavidin-biotin linkages, or coated onto the surface of polymers after particle formation (Torchilin V. P. et al. (2001) Proc. Natl. Acad. Sci. USA, Vol. 98, Issue 15, 8786-8791, July 17.

TAT peptide on the surface of the liposomes affords their efficient delivery even at low temperature and in the presence of metabolic inhibitors; Lestini et al. (2002). Surface modification of liposomes for selective cell targeting in cardiovascular drug delivery. J. Controlled Release, 78, 235247; Dokka S. et al. (1997) Cellular delivery of oligonucleotides by synthetic import peptide carrier. Pharm. Res., vol. 14, No. 12, 1759-1764); Wu Y et al. (2000). Gene transfer facilitated by a cellular targeting molecule, retrovirus protein σ1. Gene Therapy, 7, 61-69).

When the delivery of the gene to M cells in the gut is designed to prime or boost the immune system, the genes can be co-delivered/co-encapsulated with adjuvants (e.g. MF59, alum, saponin, QS21, MPL, bacterial toxins such as Lt, CT or mutants there-of, CPG motif nucleotides). Immune response could be boosted at a later stage by methods such as subcutaneous administration of an adjuvant.

In some cases it may be desired to shut off expression of certain genes, so as to enhance the adoption by enterocytes of an M cell phenotype. This can be achieved by the delivery, by methods outlined above, of antisense oligonucleotides, ribozyme, or RNA-interference molecules specific to the gene of interest.

TABLLE 4

Peptides that Target to and/or Enhance Uptake Across the GIT

| | |
|---|---|
| SEQ. ID NO: | PEPTIDE SEQUENCES |

| | |
|---|---|
| SEQ. ID NO: | ADDFMGCMLTLPTSLGGEGSPYNYYDTHEANGPH |
| SEQ. ID NO: | TPTTTATWGTTGPVDLSSLHLLRHPCREF |
| SEQ. ID NO: | MSPDHQYALQSSPVLPCCRPLLVDSDYIHS |
| SEQ. ID NO: | RGYGRLAESCCVNRCIRTVGGCGNSPASDILSAT |
| SEQ. ID NO: | STPGRGSGRDTGANNPADTPYANPSHRDTILSLDPSLL |
| SEQ. ID NO: | RQHLWRDLHGPRFRDTNTGVAGTFSPPVSVADTHRTPD |
| SEQ. ID NO: | SFSNLTAGDEEDDHFSGGRFNHANLTSRSHNRGQLASSA |
| SEQ. ID NO: | RQSVLDSWGGKTSVTGLSERYYASHSHTSAPTPHYASHS |
| SEQ. ID NO: | RQWVGDRDAGEGNTWVDEKYSRDANVISYRSHNHASQGTL |
| SEQ. ID NO: | RASDCDVECNLRWVEDVGGVWYAKTVSRMLSTT |
| SEQ. ID NO: | RQSAGFLGFAPTNIDDTSFNAGCGDTLAIPCRHRSSLISPARPP |
| SEQ. ID NO: | RSGAYESPDGRGGRSYVGGGGGCGNIGRKHNLWGLRTASPACWD |
| SEQ. ID NO: | SPRSFWPWSRHESFGISNYLGCGYRTCISGTMTKSSPIYPRHS |
| SEQ. ID NO: | SSSSDWGGVPGKWRERFKGRGCGISITSVLTGKPNPCPEPKAA |
| SEQ. ID NO: | RVGQCTDSDVRRPWARSCAHQGCGAGTRNSHGCITRPLRQASAH |
| SEQ. ID NO: | SHSGGMNRAYGDVFRELRDRWNATSHHTRPTPQLPRGPN |
| SEQ. ID NO: | SPCGGSWGRFMQGGLFGGRTDGCGAHRNRTSASLEPPSSDY |
| SEQ. ID NO: | RGAADQRRGWSENLGLPRVGWDAIAHNSYTFTSRRPRPP |
| SEQ. ID NO: | SGGEVSSWGRVNDLCARVSWTGCGTARSARTDNKGFLPKHSSLR |
| SEQ. ID NO: | SDSDGDHYGLRGGVRCSLRDRGCGLALSTVHAGPPSFYPKLSSP |
| SEQ. ID NO: | RSLGNYGVTGTVDVTVLPMPGHANHLGVSSASSSDPPRR |
| SEQ. ID NO: | RTTTAKGCLLGSFGVLSGCSFTPTSPPPHLGYPPHSVN |
| SEQ. ID NO: | SPKLSSVGVMTKVTELPTEGPNAISIPISATLGPRNPLR |
| SEQ. ID NO: | RWCGAELCNSVTKKFRPGWRDHANPSTHHRTPPPSQSSP |
| SEQ. ID NO: | RWCGADDPCGASRWRGGNSLFGCGLRCSAAQSTPSGRIHSTSTS |
| SEQ. ID NO: | SKSGEGGDSSRGETGWARVRSHAMTAGRFRWYNQLPSDR |
| SEQ. ID NO: | RSSANNCEWKSDWMRRA.CIARYANSSGPARAVDTKAAP |
| SEQ. ID NO: | SKWSWSSRWGSPQDKVEKTRAGCGGSPSSTNCHPYTFAPPPQAG |
| SEQ. ID NO: | SGFWEFSRGLWDGENRKSVRSGCGFRGSSAQGPCPVTPATIDKH |
| SEQ. ID NO: | SESGRCRSVSRWMTTWQTQKGGCGSNVSRGSPLDPSHQTGHATT |
| SEQ. ID NO: | REWRFAGPPLDLWAGPSLPSFNASSHPRALRTYWSQRPR |
| SEQ. ID NO: | RMEDIKNSGWRDSCRWGDLRPGCGSRQWYPSNMRSSRDYPAGGH |
| SEQ. ID NO: | SHPWYRHWNHGDFSGSGQSRHTPPESPHPGRPNATI |
| SEQ. ID NO: | RYKHDIGCDAGVDKKSSSVRGGCGAHSSPPRAGRGPRGTMVSRL |
| SEQ. ID NO: | SQGSKQCMQYRTGRLTVGSEYGCGMNPARHATPAYPARLLPRYR |
| SEQ. ID NO: | SGRTTSEISGLWGWGDDRSGYGWGNTLRPNYIPYRQATNRHRYT |
| SEQ. ID NO: | RWNWTVLPATGGHYWTRSTDYHAINNHRPSIPHQHPTPI |
| SEQ. ID NO: | SWSSWNWSSKTTRLGDRATREGCGPSQSDGCPYNGRLTTVKPRT |
| SEQ. ID NO: | SGSLNAWQPRSWVGGAFRSHANNNLNPKPTMVTRHPT |
| SEQ. ID NO: | RYSGLSPRDNGPACSQEATLEGCGAQRLMSTRRKGRNSRPGWTL |
| SEQ. ID NO: | SVGNDKTSRPVSFYGRVSDLWNASLMPKRTPSSKRHDDG |
| SEQ. ID NO: | TNAKHSSHNRRLRTR |
| SEQ. ID NO: | SDNAKEPGDYNCCGNGNSTG |
| SEQ. ID NO: | RTRLRRNHSSHKANT |
| SEQ. ID NO: | PEPTIDE SEQUENCES |
| SEQ. ID NO: | GPHRRGRPNSRRSSKT |
| SEQ. ID NO: | GTSNGNGCCNYDGP |
| | Pevers patch and/ or M cell specific targeting ligands: |
| SEQ. ID NO: | ATPPPWLLRTAP |
| SEQ. ID NO: | DGSIHKRNIMPL |
| SEQ. ID NO: | DYDSLSWRSTLH |
| SEQ. ID NO: | GEPTTDMRWRNP |
| SEQ. ID NO: | GLWPWNPVTVLP |
| SEQ. ID NO: | HMLNDPTPPPYW |
| SEQ. ID NO: | KPAYTHEYRWLA |
| SEQ. ID NO: | LETTCASLCYPS |
| SEQ. ID NO: | LGTDWHSVSYTL |
| SEQ. ID NO: | LGTLNAGVPGFP |
| SEQ. ID NO: | LTHSKNPVFLST |
| SEQ. ID NO: | LVPTTHRHWPVT |
| SEQ. ID NO: | LVSNARGFNNLS |
| SEQ. ID NO: | NTRIPEPIRFYM |
| SEQ. ID NO: | NVYTFHSMSPMP |
| SEQ. ID NO: | QHTTLTSHPRQY |
| SEQ. ID NO: | SDFSDTMPHRPS |
| SEQ. ID NO: | SIDTIQILSLRS |
| SEQ. ID NO: | SISWASQPPYSL |
| SEQ. ID NO: | SMVKFPRPLDSR |
| SEQ. ID NO: | SPTLGASVAQTN |
| SEQ. ID NO: | TMSPNVYYTAFG |
| SEQ. ID NO: | TQIPSRPQTPSQ |
| SEQ. ID NO: | VCSNMYFSCRLS |
| SEQ. ID NO: | VPPHPMTYSCQY |
| SEQ. ID NO: | VPRLEATMVPDI |
| SEQ. ID NO: | VPTKPELPVNFT |
| SEQ. ID NO: | WSSDLPQPASTY |
| SEQ. ID NO: | YITPYAHLRGGN |
| SEQ. ID NO: | NVYTDNTLSPTP |
| SEQ. ID NO: | LETTAASLCYPS |
| SEQ. ID NO: | SPYCLSACTTEL |
| SEQ. ID NO: | LETTCASLCYPS |
| SEQ. ID NO: | VPPHPMTYSCQY |
| SEQ. ID NO: | VPPHPMTYSAQY |
| SEQ. ID NO: | VPPHPMTYSSQY |
| SEQ. ID NO: | YQCSYTMPHPPV |
| SEQ. ID NO: | VCSNMYFSCRLS |
| SEQ. ID NO: | VSSNMYFSSRLS |
| SEQ. ID NO: | DYDSLSWRSTLHGGHESSH |
| SEQ. ID NO: | GNPTSTMRW |
| SEQ. ID NO: | PWNSATVL |
| SEQ. ID NO: | NDPTAPPY |
| | Membrane Translocating Peptides: |
| | (underline denotes cyclization) |
| SEQ. ID NO: | KKAAAVLLPVLLAAPFITC-LC |
| SEQ. ID NO: | KKKAAAVLLPVLLAAP |
| SEQ. ID NO: | KKAAAVLLPVLLAAPREDL |
| SEQ. ID NO: | KKCAAVLLPVLLAAPC |
| SEQ. ID NO: | CAAVLLPVLLAAC |
| SEQ. ID NO: | KKCAAVLLPVLLAC |
| SEQ. ID NO: | CAAVLLPVLLC |
| SEQ. ID NO: | CAAVLLPVLC |
| SEQ. ID NO: | CAVLLPVLLAAPC |
| SEQ. ID NO: | CVLLPVLLAAPC |
| SEQ. ID NO: | CLLPVLLAAPC |
| SEQ. ID NO: | CLPVLLAAPC |
| SEQ. ID NO: | AAVLLPVLLAAP |
| SEQ. ID NO: | AAVLLPVLLAA |
| SEQ. ID NO: | KKAAVLLPVLLA |
| SEQ. ID NO: | AAVLLPVLL |
| SEQ. ID NO: | AAVLLPVL |
| SEQ. ID NO: | AVLLPVLLAAP |
| SEQ. ID NO: | VLLPVLLAAP |
| SEQ. ID NO: | LLPVLLAAP |
| SEQ. ID NO: | LPVLLAAP |
| SEQ. ID NO: | AAVLLPVLLAAKKKRKA |
| SEQ. ID NO: | KKKRKAAAAVLLPVLLA |

### Example 7

Use of Bacterial Coatings to Convert Enterocytes to M Cells

Might be nice to have some type of claim capturing the concept from this section Use of bacterial coatings on PLGA particles, co-administered bacterial particles or pro-biotic yogurts as adjuvants for oral vaccination with PLGA particles. The invention is based on converting enterocytes to M cells by using specific bacteria in advance of, or along with the oral vaccine particle of interest. In doing so the capability of absorbing particles through M cells will be increased. This idea is not based on targeting but on the ability of live bacteria or active bacterial components to stimulate cytokine production in Peyer's patches, thus, enabling enterocyte-M cell conversion. As a result, an invention disclosed herein is a method of promoting enterocyte-M cell conversion, said method comprising orally administering an antigen, antigenic composition, or antigen-carrying particle to a person and either simultaneously with, or prior to, said administration, also orally administering a bacteria, or pro-biotic yogurts, or bacterial component to said person.

All references cited herein are incorporated herein by reference in their entireties.

TABLE 5
Miscellaneous GenBank Accession Numbers

| | |
|---|---|
| Human Serum Albumin | NM_000477.3 |
| Calreticulin | M84739 |

### Dates for GenBank Records

To the extent the date of a GenBank record, ratherthan its version number, is relevant for purposes of incorporation by reference, the date of the record is the filing date of this application with the following exceptions:

TABLE 2 Rat genes

| | |
|---|---|
| Mar. 27, 2002 | D83697 through M10149 |
| Mar. 28, 2002 | Q03238 through NM_017218.2 |

TABLE 3 Human genes with a fold change of 0.5 or less

Apr. 02, 2002

TABLE 2 Human genes with a fold change of 0.5 or less

| | |
|---|---|
| Apr. 02, 2002 | U76376.1 through XM_087242.1 |
| Apr. 03, 2002 | S90469 through M29366.1 |

The records specified for Mar. 27, 2002, Mar. 28, 2002, Apr. 2, 2002, and Apr. 3, 2002, do not include those of GenBank IDs: Q07912, P21145, P46734, Q92851, Q13490, NP_{—}006168, P28360, P28347, Q06830, P20701, P01589, P05106, P35225, P17936, S18408, P17074, P10661, P35426, P09456, P22791, Q06486, P21708, Q03238, P10644, P54868, P10398, P48730, P31749, P27361, P25063, Q14012, P13866

AMINO ACID SEQUENCES AND NUCLEOTIDE SEQUENCES CORRESPONDING TO SELECTED
CENBANK ID NUMBERS

GENBANK ID: M81750
VERSION M81750.1 GI:895928
MVNEYKKILLLKGFELMDDYHFTSIKSLLAYDLGLTTKMQEEYN
RIRITDLMEKKFQGVACLDKLIELAKDMPSLKNLVNNLRKEKSKVAKKIKIQEKAPVK
KINQEEVGLAAPAPTARNKLTSEARGRIPVAQKRKTPNKEKTEAKRNKVSQEQSKPPG
PSGASTSAAVDHPPLPQTSSSTFSNTSFTPNQETQAQRQVDARRNVPQNDFVTVVVLK
ATAPFKYESPENGKSTNFHATVASKTQYFHVKVPDINLKEKFVRKKVITISDYSECKC
VMEIKEASSVSDFNQNFEVPNRIIEIANKTPKISQLYKQASGTMVYGLFMLQKKSVHK
KNTIYEIQDNTGSMDVVGSGKWENIKCEKGDKLRLFCLQLRTVDRKLKLVCGSHSFIK
VIKAKKNKEGPMNVN
GENBANK ID: X59798
VERSION X59798.1 GI:35631
MEHQLLCCEVETIRRAYPDANLLNDRVLRAMLKAEETCAPSVSY
FKCVQKEVLPSMRKIVATWMLEVCEEQKCEEEVFPLAMNYLDRFLSLEPVKKSRLQLL
GATCMFVASKMKETIPLTAEKLCIYTDNSIRPEELLQMELLLVNKLKMNLAAMTPHDF
IEHFLSKMPEAEENKQIIRKHAQTFVALCATDVKFISNPPSMVAAGSVVAAVQGLNLR
SPNNFLSYYRLTRFLSRVIKCDPDCLRACQEQIEALLESSLRQAQQNMDPKAAEEEEE
EEEEVDLACTPTDVRDVDI
GENBANK ID: L27211.1
VERSION L27211.1 GI:558656
MEPAAGSSMEPSADMLATAAARGRVEEVRALLEAGALPNAPNSY
GRRPIQVMMMGSARVAELLLLHGAEPNCADFATLTRPVNDAAREGFLDTLVVLHRAGA
RLDVRDAWGRLPVDLAEELGHRDVARYLRAAAGGTRGSNHARIDAAEGPSDIPD
GENBANK ID: U22398
VERSION U22398.1 GI:790247
MSDASLRSTSTMERLVARGTFPVLVRTSACRSLFGPVDNEELSR
ELQARLAELNAEDQNRWDYDFQQDMPLRGPGRLQWTEVDSDSVPAFYRETVQVGRCRL
LLAPRPVAVAVAVSPPLEPAAESLDGLEEAPEQLPSVPVPAPASTPPPVPVLAPAPAP
APAPVAAPVAAPVAVAVLAPAPAPAPAPAPAPAPVAAPAPAPAPAPAPAPAPAPAPDA
APQESAEQGANQGQRGQEPLADQLESGISGRPAAGTAAASANGAAIKKLSGPLISDFF
AKRKRSAPEKSSGDVPAPCPSPSAAPGVGSVEQTPRKRLR
GENBANK ID: X51521
VERSION X51521.1 GI:31282
MPKPINVRVTTMDAELEFAIQPNTTGKQLFDQVVKTIGLREVWY
FGLNYVDNKGFPTMLKLDKKVSAQEVRKENPLQFKFRAKFYPEDVAEELIQDITQKLF
FLQVKEGILSDEIYCPPETAVLLGSYAVQAKFGDYNKEVEKSGYLSSERLIPQRVMDQ
HKLTRDQWEDRIQVWHAERRGMLKDNAMLEYLKIAQDLEMYGINYFEIKNKKGTDLWL
GVDALGLNIYEKDDKLTPKIGFPWSEIRNISFNDKKFVIKPIDKKAPDFVFYAPRLRI
NKRILQLCMGNHELYMRRRKPDTIEVQQMKAQAREEKHQKQLERQQLETEKKRRETVE
REKEQMMREKEELMLRLQDYEEKTKKAERELSEQIQRALQLEEERKRAQEEAERLEAD
RMAALRAKEELERQAVDQIKSQEQLAAELAEYTAKIALLEEARRRKEDEVEEMQHRAK
EAQDDLVKTKEELHLVMTAPPPPPPPVYEPVSYHVQESLQDEGAEPTGYSAELSSEGI
RDDRNEEKRITEAEKNERVQRQLVTLSSELSQARDENKRTRNDIIHNENMRQGRDKYK
TLRQIRQGNTKQRIDEFEAL
GENBANK ID: L04143.1
VERSION L04143.1 GI:180574
THIS ENTRY IS NOT CONTIGUOUS CENOMIC DNA. IT CONTAINS NUMEROUS PIECES OF
INTRONS.

| | |
|---|---|
| 1 | GAGCTCGGAT CCCATCGCAG CTACCGCGAT GAGAGGCGCT CGCGGCGCCT GGGATTTTCT |
| 61 | CTGCGTTCTG CTCCTACTGC TTCGCGTCCA CACAGGTGGG ACACCGCGGC TGGCTCCCCC |
| 121 | ACCGTGCCAC TACTCGCGAA CCCTGTGCCC TGGGAGGGTG CTACCGCCAT GGCATCCGCA |
| 181 | GACAGGACTG CGGGCCCTCA GTGGGCCTGC GTTCCAGCCT CCGGGGAGAC TCCAGGTGGC |
| 241 | CCTCGGACTC TCCGGCGCCC TGCCTCGCTC ACCTGCGCGA GGAGACCCCA GCTGCTGGTG |
| 301 | GTGGGGGACG CGAATCCGGG GTTCTTCGGG AATGGGGACA GCAAGAGGGG GTTAGGCGTG |
| 361 | AGCGAGGCTG CAGGCTCCGT GCGAGTTTGG GGTGGCTTTT GTGCCGACGT TGCGCGGGGG |
| 421 | CGGAGGCGGG GGCTCAGGGT TTGCACCGAG CGCCTTCTCT CTCGGTGCGA GGCCGGCCGC |
| 481 | AGCTTCCTTT TGTTAAAAGT TGCGTGTGTG TGACGGCGCC CGGGCTGCAG CCTCAACCTC |
| 541 | CTGGGCTTAA GTGATCTCCC ACCTCAGCCT CCCGCCTCAG CCTCCCATGT AGCTGGGAAA |
| 601 | ACAGGTTCCC ACTACCATGC CCAGCTAATG TTTTTTCTAG TTTTTGTAGA TGTGTTGGGC |
| 661 | GGTGGGGGCG TCTCACTGCG TTGCCCAGGC TGGTCTCGAA CTCCTAGGCT CAAGCAATCT |
| 721 | TCCCACCTCA GCCTCCCAGA GTGCTGGGAT TACAGGCGTG ACGACGGCAC CTGGCCAGCA |
| 781 | GTTTGTTCTT TAAACCCTGA AATGTATGTG AGGACCATGT GTCACACTAG CATGGACGTT |
| 841 | TTCCCAGCAT CTAGCATGGT GCTTTGTAGA TGATAATTAA TGAATAGGTA TTTGAATACA |
| 901 | TGGAGGCATG CATGGCTGAA TGAAGTGGCT GTTGTAAAAT TTCTAGGGTT CAGGTTTCAT |
| 961 | ATTCAGAGCC TAAAGTTTGC ATCTTATAAA CTAAATAGTT TCCTATCTAG GAAACCTATT |
| 1021 | TAGGCATTAG GGTGTTAAAA CAGGTGTATC ATTTTCTGCC TTAGTGTTTA GAGATTTGTG |
| 1081 | AATAGTTCTC CTTTTGATGA ACATTGCCAT GTAAAGAGAG TTATACAGAA ATAACTGAAT |
| 1141 | TCACACAGTT TTAAGGAAAA GCTATTCTAT GTCATGGTCA TGTATATTCT GCCAAAGAGA |
| 1201 | GTGACAGGCC AGTAGTTTCT TTTTTCTTTT TCCCCATAGT GTGAGATTTT GTTTCTGTTG |
| 1261 | TTTCTAAGCT GGGTGTCTGC ATGTCCACAC TGCGAAGATG GCCCATATCA GAATGAAAAC |
| 1321 | TTGACCCAAT TGTATGTTTA GCCCAGAGAA GGCTGGGGCA TTCAGCACAT CCTGGCTTGG |
| 1381 | GGACCAAATG TGACCCTCAG GATTAATTGA GGGTTGGAGA AAATAATTTT ATAGATGAAC |
| 1441 | TTAAGACATT TTAGACAGAA CTCTCTTTTC AGCCATAAAT AGCAGGGCAG CTTTGTCCTA |
| 1501 | TTTTTATTGT AGAGTACACA GAAGATGGAA CTCAGTATTG GAAGAAGTGC TTTATTTCGC |
| 1561 | CAAGGAAGAA GATCATACTC AACACGATTC TGTTTTTCTT GGCAGGCTCT TCTCAACCAT |
| 1621 | CTGTGAGTCC AGGGGAACCG TCTCCACCAT CCATCCATCC AGGAAAATCA GACTTAATAG |
| 1681 | TCCGCGTGGG CGACGAGATT AGGCTGTTAT GCACTGATCC GGGCTTTGTC AAATGGACTT |
| 1741 | TTGAGATCCT GGATGAAACG AATGAGAATA AGCAGAATGA ATGGATCACG GAAAAGGCAG |
| 1801 | AAGCCACCAA CACCGGCAAA TACACGTGCA CCAACAAACA CGGCTTAAGC AATTCCATTT |
| 1861 | ATGTGTTTGT TAGAGGTAAA TGCTTGGCTT TCTGCAGCAG GTCATGTCAC TTTAGGAGGG |
| 1921 | TTGCTTTTAT GACACCGCAG TTTCATCTAT GAAATGGCAA TAATGATAGT ACTGATCATG |
| 1981 | GGAGGGGCAA TTTGAAGATT AAATGAGATT AAGTGTAATG GTCCAAGCTT AGTGCGTGAT |
| 2041 | ACATGGAAAG CGTTTAATAA ATGTTAATTC TCAATAGTAC TAGATGGATA AATTTTGCTT |
| 2101 | TTGTTTACAC AGAAAAAAGC AGCCATTTGG GCCACTAGTC ATGAAAGGCA ACATATTAGA |
| 2161 | TCTTTAAAAG TGTTTCAGTG TCTGTGACCA GCCATTCCAA CTACTGATTT GGATATGCTT |
| 2221 | CTTATAGATC CTGCCAAGCT TTTCCTTGTT GACCGCTCCT TGTATGGGAA AGAAGACAAC |
| 2281 | GACACGCTGG TCCGCTGTCC TCTCACAGAC CCAGAAGTGA CCAATTATTC CCTCAAGGGG |
| 2341 | TGCCAGGGGA AGCCTCTTCC CAAGGACTTG AGGTTTATTC CTGACCCCAA GGCGGGCATC |
| 2401 | ATGATCAAAA GTGTGAAACG CGCCTACCAT CGGCTCTGTC TGCATTGTTC TGTGGACCAG |
| 2481 | GAGGGCAAGT CAGTGCTGTC GGAAAAATTC ATCCTGAAAG TGAGGCCAGG TACCTTGCTT |
| 2521 | TCTTATCTGC CTCTGGAGTT GAGAACTCAC TTATCTAAAG AGACTTCTCT TCTCGTTGAT |
| 2581 | CGTAAGCTGT ACACATTTGA GGAGAAATGG TAAATCAAAA TTTCATGCTA TAATACAAAT |
| 2641 | TATTTGAGGG GCCACATTTC TTTTCATTCT AGCCTTCAAA GCTGTGCCTG TTGTGTCTGT |
| 2701 | GTCCAAAGCA AGCTATCTTC TTAGGGAAGG GGAAGAATTC ACAGTGACGT GCACAATAAA |
| 2761 | AGATGTGTCT AGTTCTGTGT ACTCAACGTG GAAAAGAGAA AACAGTCAGG TGAGTGAATC |
| 2821 | GCTTCATTCT TCTCATGTTC TGTCTCTGTG GGAGATGATA AGTTTTCTCT TTCAGAAGAG |
| 2881 | TCTGTCCTGA AACTGCCTCG ACTAGTGCGT CTGTCAGAGG AGAAGTTAAT TGCTGCTATT |
| 2941 | TTTAATTTAT CTAGGAAAGA TTCTGAATAT AAATTATATG GTAATCTTCA TTTTTTTTTC |
| 3001 | TCCTTTTCTG AAACCAGCAG ACTAAACTAC AGGAGAAATA TAATAGCTGG CATCACGGTG |
| 3061 | ACTTCAATTA TGAACGTCAG GCAACGTTGA CTATCAGTTC AGCGAGAGTT AATGATTCTG |
| 3121 | GAGTGTTCAT GTGTTATGCC AATAATACTT TTGGATCAGC AAATGTCACA ACAACCTTGG |
| 3181 | AAGTAGTAGG TAAATACCTC TATGGGAATG TTTAAATTAC TGGCAGTAGT GAAAGAAGAA |
| 3241 | ATTATTAGAC AGTTTCTTTT TTATGTAAAT GGAATGTTGA ACAGATTCTT AGAATTTTGT |
| 3301 | TATCACTGAA TGAATGAAAA TTATCCTTGT AGCCTCTTGC AATGAAAGCA CAATTCTGTT |
| 3361 | TTTTTTGTCC AGTAGTTGTA GATAATGTTT CTTTCTGTCT TATTTCATTC TAATTAGATA |
| 3421 | AAGGATTCAT TAATATCTTC CCCATGATAA ACACTACAGT ATTTGTAAAC GATGGAGAAA |
| 3481 | ATGTAGATTT GATTGTTGAA TATGAAGCAT TCCCCAAACC TGAACACCAG CAGTGGATCT |
| 3541 | CTCTGAACAG AACCTTCACT GATAAATGGG AAGATTATCC CAAGTCTGAG AATGAAAGTA |
| 3601 | ATATCAGGTA AGAAATGGAC CTTGCCCTGG GGATTACACA TTACCCCCTT TTCCAGTGGG |
| 3661 | CTTATCAGAT CTTATTTCTG TAACCCGTAA ATCCACGAGA AGATACCTGG TAAAGAAGAA |
| 3721 | AGTCTATTTT GCTAATACTT TACTGAATTA AATGAGTTAT ATTTTTCCTC AAACAGGCAT |
| 3781 | AGATTTCCAG GTAGAAACTG AAAAAGACAT GCCTTCCAAG GCATGCTATC CACAGGTGAT |
| 3841 | TGACTAGTTG TCTTTTCTTT GTAGATACGT AAGTGAACTT CATCTAACGA GATTAAAAGG |
| 3901 | CACCCAAGGA CCCACTTACA CATTCCTAGT GTCCAATTCT GACGTCAATG CTGCCATAGC |
| 3961 | ATTTAATGTT TATGTGAATA GTAACTAACA TGAAGGGCTC TTTTAATTTT TTATTCTTTT |
| 4021 | AAGTTGTGGC TCGTGTTTGT AACAGCTGCA AGGACTCAAC TTGCTGTACT AAAGGTTGTA |
| 4081 | CGGATTTAGA GAGGGAGTGA AGTGAATGTT GCTGACGTTT TCCAGCACTC TGACATATGC |
| 4141 | CCATTTCTGT TTTCCTGTAG CAAAACCAGA AATCCTGACT TACGACAGGC TCGTGAATCC |
| 4201 | CATGCTCCAA TGTGTGGCAC CAGGATTCCC AGAGGCCACA ATAGATTGGT ATTTTTGTCC |
| 4261 | AGGAACTGAG CAGAGGTGAG ATGATTATTT TTGGCACTGC TTATAATGCA GAGGGGAAGG |
| 4321 | ACTGCAATTC ACTTGAATTT CAAATATGTT TTCTGATTTT TTTTAAAAAA GCTTTAACTT |
| 4381 | TGTTTTAAAA GTATGCCACA TCCCAAGTGT TTTATGTATT TATTTATTTT CCTAGAGTAA |
| 4441 | GCCAGGGCTT TTGTTTTCTT CCCTTTAGAT GCTCTGCTTC TGTACTGCCA GTGGATGTGC |
| 4501 | AGACACTAAA CTCATCTGGG CCACCGTTTG GAAAGCTAGT GGTTCAGAGT TCTATAGATT |
| 4561 | CTAGTGCATT CAAGCACAAT CGCACGGTTG AATGTAAGGC TTACAACGAT GTGGGCAAGA |
| 4621 | CTTCTGCCTA TTTTAACTTT GCATTTAAAG GTAACAACAA AGGTATATTT CTTTTTAATC |
| 4681 | CAATTTAAGG GGATGTTTAG GCTCTGTCTA CCATATCAGT CATGATTTTG AGCTCAATTA |
| 4741 | ACCCTCACTA AAGGGAGTCG ACTCGATCCC ATCCTGCCAA AGTTTGTGAT TCCACATTTC |
| 4801 | TCTTCCATTG TAGAGCAAAT CCATCCCCAC ACCCTGTTCA CTCCTTTGCT GATTGGTTTC |
| 4861 | GTAATCGTAG CTGGCATGAT GTGCATTATT GTGATGATTC TGACCTACAA ATATTTACAG |
| 4921 | GTAACCATTT ATTTGTTCTC TCTCCAGAGT GCTCTAATGA CTGAGACAAT AATTATTAAA |
| 4981 | AGGTGATCTA TTTTTCCCTT TCTCCCCACA GAAACCCATG ATGAAGTACA GTGGAAGGTT |
| 5041 | GTTGAGGAGA TAAATGGAAA CAATTATGTT TACATAGACC CAACACAACT TCCTTATGAT |
| 5101 | CACAAATGGG AGTTTCCCAG AAACAGGCTG AGTTTTGGTC AGTATGAAAC AGGGGCTTTC |
| 5161 | CATGTCACCT TTTTGGGTAC ACATAACAGT GACTTTAAGG AACTCCAGTG GCTTCCTTTG |
| 5221 | TTTTGTTCCA CCTGAAACAA TGAGTTTTCT GTGAAATTGC GCCCCTTTTG ATAGGTTTGC |
| 5281 | CATAGAGAAC ATCGTAGGAA AATGTCTCTG GACAACATTG TTTTTAATTC CTTTATTGAT |
| 5341 | TTTGAAACTG CACAAATGGT CCTTCAATTC CACCACCAGC ACCATCACCA CTTACCTTGT |
| 5401 | TGTCTTCCTT CCTACAGGGA AAACCCTGGG TGCTGGAGCT TTCGGGAAGG TTGTTGAGGC |
| 5461 | AACTGCTTAT GGCTTAATTA AGTCAGATGC GGCCATGACT GTCGCTGTAA AGATGCTCAA |
| 5521 | GCGTAAGTTC CTGTATGGTA CTGCATGCGC TTGACATCAG TTTGCCAGTT GTGCTTTTTG |
| 5581 | CTAAAATGCA TGTTTCCAAT TTTAGCGAGT GCCCATTTGA CAGAACGGGA AGCCCTCATG |
| 5641 | TCTGAACTCA AAGTCCTGAG TTACCTTGGT AATCACATGA ATATTGTGAA TCTACTTGGA |
| 5701 | GCCTGCACCA TTGGAGGTAA AGCCGTGTCC AAGCTGCCTT TTATTGTCTG TCAGGTTATC |
| 5761 | AAAACATGAC ATTTTAATAT GATTTTGGCA ATGCTAGATT ATAAACTGCT TGGAACATTT |
| 5821 | TTTTACCCAG ACTGTTGTTC TCTCTTGCTA GATTTTGTTT TCCTCATTGT TCTTAAGAAT |
| 5881 | ATATGGGATT GTATTGGGAC TAAGTAGTCT GATCCACTGA AGCTGAATAT TAATGGCCAT |
| 5941 | GACCACCCTT GGGTATTTTT ATGGGAGGCA GAATTAATCT ATATATCTCA CCTTCTTTCT |
| 6001 | AACCTTTTCT TATGTGCTTT TACCGCCGAC CCTGGTCATT ACAGAATATT GTTGCTATGG |
| 6061 | TGATCTTTTG AATTTTTTGA GAAGAAAACG TGATTCATTT ATTTGTTCAA AGCAGGAAGA |
| 6121 | TCATGCAGAA GCTGCACTTT ATAAGAATCT TCTGCATTCA AAGGAGTCTT CCTGGTAAGA |
| 6181 | CTGATTTACA TAAATAGTTA GCTGTTGACA GGCAGTTCAT GGGGAACTCT TTATTCAAAC |
| 6241 | TTTACATGAC TTTCCTCAAA TTGGTCCAGT CTATTATGTA GCAAAGGGGA TGAGGAGGTA |
| 6301 | GAGCATGACC CATGAGTGCC CTTCTACATG TCCCACTTGA TTCAGTCATG ACTTGTTTCA |
| 6361 | TCTCTCCCAG CAGCGATAGT ACTAATGAGT ACATGGACAT GAAACCTGGA GTTTCTTATG |
| 6421 | TTGTCCCAAC CAAGGCCGAC AAAAGGAGAT CTGTGAGAAT AGGTCAGTAC CTACCTATCA |
| 6481 | AGCAACCAAG AGTAACTTTA CAGAGAGTAT GTATATCATG CTAATGTGGA ATATAACATC |
| 6541 | ATTCCAGTAG CAATGATGCA GACCAGTTCT GCTTTATGGT AGCAGTGCCA ATGGTCAATG |
| 6601 | GCAGTTAGGG GCAAGTTCAC ATTAGTTCAT TGATTACCAG CCTTTGGTAT GTCATTGCCA |
| 6661 | CTGTCTTTTC CTTTCCTGAC CTTTATGGTT GTAATTGCTA AGAAAAATCC TCTCTTCCTC |
| 6721 | ACAGGCTCAT ACATAGAAAG AGATGTGACT CCCGCCATCA TGGAGGATGA CGAGTTGGCC |
| 6781 | CTAGACTTAG AAGACTTGCT GAGCTTTTCT TACCAGGTGG CAAAGGGCAT GGCTTTCCTC |
| 6841 | GCCTCCAAGA ATGTAAGTGG GAGTGATTCT CTAAAGAGTT TTGTGTTTTG TTTTTTTGAT |
| 6901 | TTTTTTTTTT TTTTTTTTTT TTTTGAGAAC AGAGCATTTT AGAGCCATAG TTAAAAGCAG |
| 6961 | AATGTCATTT AAAACAAAAG TATTGGATTT TTTATAATAT AAGCAACACT ATAGTATTAA |
| 7021 | AAAGTTAGTT TTCACTCTTT ACAAGTTAAA ATGAATTTAA ATGGTTTTCT TTTCTCCTCC |
| 7081 | AACCTAATAG TGTATTCACA GAGACTTGGC AGCCAGAAAT ATCCTCCTTA CTCATGGTCG |
| 7141 | GATCACAAAG ATTTGTGATT TTGGTCTAGC CAGAGACATC AAGAATGATT CTAATTATGT |
| 7201 | GGTTAAAGGA AACGTGAGTA CCCATTCTCT GCTTGACAGT CCTGCAAAGG ATTTTTAGTT |
| 7261 | TCAACTTTCG ATAAAAATTG TTTCCGTGAC TTTCATAATG TAAATCCTGT CTAGGGATAT |
| 7321 | CACACATTTT AGCAGTCAAA TGTATTTCAG AGGTGATTGG GATCATCTGA GTTCATATAG |
| 7381 | GTAAAAGGTT TTTGTGAGAT GGTACTCAAG TTATCACTCC ACATTTCAGC AACAGCAGCA |
| 7441 | TCTATAAGAA TATCTTCTGT TCAATTTTGT TGAGCTTCTG AATTAACATT ATTGACTCTG |
| 7501 | TTGTGCTTCT ATTACAGGCT CGACTACCTG TGAAGTGGAT GGCACCTGAA AGCATTTTCA |
| 7561 | ACGTGTATAC ACGTTTGAAA GTGACGTCTG GTCCTATCGG ATTTTTCTTT GGGAGCTGTT |
| 7621 | CTCTTTAGGT AAAATGATCC TTGCCAAAAG ACAACTTCAT TAGACTCAGA GCATCTTGAA |
| 7681 | GTTTCATTGG TGTCCTGCTT CCTTGTGATT AACACTGCTT TGCAAACTGT GTCTCAGGAA |
| 7741 | GCAGCCCCTA TCCTGGAATG CCGGTCGATT CTAAGTTCTA CAAGATGATC AAGGAAGGCT |
| 7801 | TCCGGATGCT CAGCCTGAAC ACGCACCTGC TGAAATGTAA GAGCCAAAAA ATTTTTCCTT |
| 7861 | TAGGTCACGT TTTCCCTTTT ATTTTTCTTT TTAGAGACAG AAACCCAGAT GTTGAGGGTT |
| 7921 | TTCATAACAC AGTTTGAAAT GTCACTTCCA TTCTTTATGA CACACTGGTC AAATGTCATT |
| 7981 | TCTCTAGTTT ATTTTCATAA TCTCTTGTCA CCAAAAATAC AGAAAGTTTC AGTAATATTT |
| 8041 | CATACATGCA GTGTTTTATG TTATCTATAT GTCAGTCCAT ATGTCCAGTT GCATAGCCCT |
| 8101 | CGAATTATTA CTGAAGTTGC TGGATGCCCA TACATTTGAA AACAACCTCA GGGCATTGAC |
| 8161 | GAGGGATAGT AAATGGCCCT TGTCTTGCAG GTATCACATA ATGAAGACTT GCTGGGATGC |
| 8221 | AGATCCCCTA AAAAGACCAA CATTCAAGCA AATTGTTCAG CTAATTGAGA AGCAGATTTC |
| 8281 | AGAGACCACC AATCATGTGA GTATACCCTG CCCAGCCATA GAATCCCCCT TCTCCCAGTT |
| 8341 | CCAGGTGTGT CCTCCTCCTC AGGCTTTCAG GGTGAGGACT AACCTCCCAA CCCCTTCTCT |
| 8401 | CCTAATCTTA GGTTGCAAAT TGGGCTTCAC GTAGGGGAAG TAAAGCAATG GAAACTAGTT |
| 8461 | CTTTTAAGAG TTCCATCAGT TAGTTGTGAT CTTGACACTG TAAGTATGCC TTTTGTTGCT |
| 8521 | ATGTTCGTTG TAGGGACTGC TGTATTCACT ATGGGCTTGT TTTCTCCACA TTTACTCCAA |
| 8581 | CTTAGCAAAC TGCAGCCCCA ACCGACACAA GCCCGTGGTA GACCATTCTG TGCGGATCAA |
| 8641 | TTCTGTCGGC AGCACCGCTT CCTCCTCCCA GCCTCTGCTT GTGCACGACG ATGTCTGAGC |
| 8701 | ACAATCAGTG TTTGGGTCAC CCCTCCAGGA ATCATCTCTT CTTTTGGCTT CCATGATGGT |
| 8761 | TATTTTCTTT TCTTTCAACT TGCATCCAAC TCCAGGATAG TGGGCACCCC ACTGCAATCC |
| 8821 | TGTCTTTCTG AGCACACTTT AGTGGCCGAT GATTTTTGTC ATCAGCCACC ATCCTATTGC |
| 8881 | AAAGCTTCCA ACTGTATATA TTCCCAATAG CAACGTAGCT TCTACCATCA ACAGAAAACA |
| 8941 | TTCTGATTTG GAAAAAGAGA GGCAGGTATG GACTGGGGGC CAGAGTCCTT TCCAAGGCTT |
| 9001 | CTCCAATTCT GCCCAAAAAT ATGGTTGATA GTTTACCTGA ATAAATGGTA GTAATCACAG |
| 9061 | TTGGCCTTCA GAACCATCCA TAGTAGTATG ATGATACAAG ATTAGAAGCT GAAAACCTAA |
| 9121 | GTCCTTTATG TGGAAAACAG AACATCATTA GAACAAAGGA CAGAGTATGA ACACCTGGGC |
| 9181 | TTAAGAAATC TAGTATTTCA TGCTGGGAAT CAGACATAGG CCATGAAAAA AATGATCCCC |
| 9241 | AAGTCTGAAC AAAAGATCCT CTTCTGTCGA CCACTGCATG AGCTTTTATA CTACCGACCT |
| 9301 | GGTPTTTAAA TAGACTTTGC TATTAGAGCA TTGAATTGGA GAGAAGGCCT CCCTAGCCAG |
| 9361 | CACTTGTATA TACGCATGTA TAAATTGTCC GTGTTCATAC ATTTGAGGGG AAAACACCAT |
| 9421 | AAGGTTTCGT TTCTGTATAC AACCCTGGCA TTATGTCCAC TGTGTATAGA AGTAGATTAA |
| 9481 | GAGCCATATA AGTTTGAAGG AAACACTTAA TACCATTTTT TAAGGAAACA ATATAACCAC |
| 9541 | AAAGCACAGT TTGAACAAAA TCTCCTCTTT TAGCTGATGA ACTTATTCTG TAGATTCTCT |
| 9601 | GGAACAAGCC TATCAGCTTC AGAATGGCAT TGTACTCAAT GGATTTGATG CTGTTTGACA |
| 9661 | AAGTTACTGA TTCACTGCAT GGCTCCCACA GGAGTGGGAA AACACTGCCA TCTTAGTTTG |
| 9721 | GATTCTTATG TAGCAGGAAA TAAAGTATAG GTTTAGCCTC CTTCGCAGGC ATGTCCTGGA |
| 9781 | CACCGGGCCA CTATCTATAT ATGTGTATGT ACGTTTGTAT GTGTGTAGAC AAATATTTGG |
| 9841 | AGGGGTATTT TTGCCCTGAG TCCAAGAGGG TCCTTTAGTA CCTGAAAAGT AACTTGGCTT |
| 9901 | TCATTATTAG TACTGCTCTT GTTTCTTTTC ACATAGCTGT CTAGAGTAGC TTACCAGAAG |
| 9961 | CTTCCATAGT GGTGCAGAGG AAGTGGAAGG CATCAGTCCC TATGTATTTG CAGTTCACCT |
| 10021 | GCACTTAAGG CACTCTGTTA TTTAGACTCA TCTTACTGTA CCTGTTCCTT AGACCTTCCA |
| 10081 | TAATGCTACT GTCTCACTGA AACATTTAAA TTTTACCCTT TAGACTGTAG CCTGGATATT |
| 10141 | ATTCTTGTAG TTTACCTCTT TAAAAACAAA ACAAAACAAA ACAAAAAACT CCCCTTCCTC |
| 10201 | ACTGCCCAAT ATAAAAGGCA AATGTGTACA TGGCAGAGTT TGTGTGTTGT CTTGAAAGAT |
| 10261 | TCAGGTATGT TGCCTTTATG GTTTCCCCCT TCTACATTTC TTAGACTACA TTTAGAGAAC |
| 10321 | TGTGGCCGTT ATCTGGAAGT AACCATTTGC ACTGGAGTTC TATGCTCTCG CACCTTTCCA |
| 10381 | AAGTTAACAG ATTTTGGGGT TGTGTTGTCA CCCAAGAGAT TGTTGTTTGC CATACTTTGT |
| 10441 | CTGAAAAATT CCTTTGTGTT TCTATTGACT TCAATGATAG TAAGAAAAGT GGTTGTTAGT |
| 10501 | TATAGATGTC TAGGTACTTC AGGGGCACTT CATTGAGAGT TTTGTCTTGC CATACTTTGT |
| 10561 | CTGAAAAATT CCTTTGTGTT TCTATTGACT TCAATGATAG TAAGAAAAGT GGTTGTTAGT |
| 10621 | TATAGATGTC TAGGTACTTC AGGGGCACTT CATTGAGAGT TTTGTCTTGG ATATTCTTGA |
| 10681 | AAGTTTATAT TTTTATAATT TTTTCTTACA TCAGATGTTT CTTTGCAGTG GCTTAATGTT |
| 10741 | TGAAATTATT TTGTGGCTTT TTTTGTAAAT ATTGAAATGT AGCAATAATC TCTTTTGAAT |
| 10801 | ATTCCCAAGC CCATGAGTCC TTGAAAATAT TTTTTATATA TACAGTAACT TTATGTGTAA |
| 10861 | ATACATAAGC GGCGTAAGTT TAAAGGATGT TGGTGTTCCA CGTGTTTTAT TCCTGTATGT |
| 10921 | TGTCCAATTG TTGACAGTTC TGAAGAATTC TAATAAAATG TACATATATA AATCAA |

GENBANK ID: M32110
VERSION M32110.1 GI:189421
RATPPSPISACHSTMGRKLDPTKEKRGPGRKARKQKGAETELVR
FLPAVSDENSKRLSSRARKRAAKRRLGSVEAPKTNKSPEAKPSPGKLPKGISAGAVQT
AGKKGPQSLFNAPRGKKRPAPGSDEEEEEEDSEEDGNVNHGDLWGSEDDADTVDDYGA
DSNSEDEEEGEALLPIEPAARKQKAREAAAGIQWSEEETEDEBEEKEVTPESGPPKVE
EADGGLQINVDEEPFVLPPAGEMEQDAQAPDLQRVHKRIQDIVGILRDPGAQREEGRS
RSEYLNRLKKQLAIYYSYGDFLLGKLMQLFPLSELVEFLBANEVPRPVTLRTNTLKTR
RRDLAQALINRGVNLDPLGKBSKTGLVVYDSSVPTGATPEYLAGHYMLQGASSMLPVM
ALAPQEHERILDMCCAFGGKTSYMAQLMKNTGVILANDANAERLKSVVGNLHRLGVTN
TIISHYDGRQFPKVVGGFDRVLLDAPCSGTGVISKDPAVKTNKDEKDILRCAHLQKEL
LLSAIDSVNATSKTGGYLVYCTCSITVEENEWVVDYALKKRNVRLVPTGLDFGQEGFT
RFRERRFHPSLRSTRRFYPHTHNMQGFFIAKFKKFSNSIPQSQTGNSETATPTNVDLP
QVIPKSENSSQPAKKAKGAGKTKQQLQKQQHPKKASFQKLNGISKGAQSELSTVPSVT
KTQASSSFQDSSQPAGKAEGIREPKVTGKLKQRSPKLQSSKKVAFLRQNAPPKGTDTQ
TPAVLSPSKTQATLKPKDHHQPLGRAKCVEKQQFAEQPFEKAAFQKQNDTPKGLSLPL
CLPSVPAAPHQQRGRNLSPGATASCCYLRWLKTRRVAHCHCHQVGTLASVRMPSLLCI
PMKFNTHFKTSGH
GenHank ID: J04111
VERSION J04111.1 GI:186624
MTAKMETTFYDDALNASFLPSESGPYGYSNPKTLKQSNTLNLAD
PVGSLKPHLRAKNSDLLTSPDVGLLKLASPELERLIIQSSNGHITTTPTPTQFLCPKN
VTDEQEGFAEGFVRALAELHSGNTLPSVTSAAQPVNGAGMVAPAVASVAGGSGSGGFS
ASLHSEPPVYANLSNFNFGALSSGGGAPSYGAAGLAFPAQPQQQQQPPHHLPQQNPVQ
HPRLQALKEEPQTVPEMPGETPPLSPIDMESQERIKAERKRNRNRIAASKCRKRKLER
IARLEEKVKTLKAQNSELASTANNLREQVAQLKQKVMNHVNSGCQLMLTQQLQTF
GENBANK ID: X59932
VERSION X59932.1 GI:30255
MSAIQAAWPSGTECIAKYNFHGTAEQDLPFCKGDVLTIVAVTKD
PNWYKAKNKVGREGIIPANYVQKREGVKAGTKLSLMPWFHGKITREQAERLLYPPETG
LFLVRESTNYFGDYTLCVSCDGKVEHYRIMYHASKLSIDEEVYFENLMQLVEHYTSDA
DGLCTRLIKPKVMEGTVAAQDEFYRSGWALNMKELKLLQTIGKGEFGDVMLGDYRGNK
VAVKCIKNDATAQAFLAEASVMTQLRHSNLVQLLGVIVEEKGGLYIVTEYMAKGSLVD
YLRSRGRSVLGGDCLLKFSLDVCEAMEYLEGNNFVHRDLAARNVLVSEDNVAKVSDFG
LTKEASSTQDTGKLPVKWTAPEALREKKFSTKSDVWSFGILLWEIYSFGRVPYPRIPL
KDVVPRVEKGYKMDAPDGCPPAVYEVNKNCWHLDAANRPSFLQLREQLEHIKTHELHL
GENBANK ID: L29220
VERSION L29220.1 GI:632969
MHHCIRYRSPEPDPYLSYRWKRRRSYSREHEGRLRYPSRREPPP
RRSRSRSHDRLFYQRRYRERRDSDTYRCEERSPSFGEDYYGPSRSRHRRRSRERGPYR
TRKHAHECHKRRTRSCSSASSMRLWGTWVKAFLARWWSAWTMPEGSLRLP
GENBANK ID: M91815.1
VERSION M91815.1 GI:180169
HOMO SAPIENS FETAL CDNA TO MRNA.

| | |
|---|---|
| 1 | CAGCTGACCC TGCTGGATCA CCTCGCCTTC AAGAAGATTC CTTATGAGGA GTTCTTCGGA |
| 61 | CAAGGATGGA TGAAACTGGA AAAGAATGAA AGGACCCCTT ATATCATGAA AACCACTAAG |
| 121 | CACTTCAATG ACATCAGTAA CTTGATTGCT TCAGAAATCA TCCGCAATGA GGACATCAAC |
| 181 | GCCAGGGTGA GCGCCATCGG GAAGTGGGTG GCCGTAGCTG ACATATGCCG CTGCCTCCAC |
| 241 | AACTACAATG CCGTACTGGA GATCACCTGC TCCATGAACC GCAGTGCAAT CTTCCGGCTC |
| 301 | AAAAAGACGT GGCTCAAAGT CTCTAAGCAG ACTAAAGCTT TGATTGATAA GCTCCAAAAG |
| 361 | CTTGTGTCAT CAGAGGGCAG ATTTAAGAAT CTCAGAGAAG CTCTGAAAAA TTGTGACCCA |
| 421 | CCCTGTGTCC CTTACCTGGG GATGTACCTC ACCGACCTGG CCTTCATCGA GGAGGGGACG |
| 481 | CCCAATTACA CGGAAGACGG CCTGGTCAAC TTCTCCAAGA TGAGGATGAT ATCCCATATT |
| 541 | ATGCCAGAGA TTCGCCAGTT TCAACAAACT GCCTACAAAA TAGAGCACCA AGCAAAGGTA |
| 601 | ACGCAATATT TACTGGACCA ATCTTTTGTA ATGGATGAAG AAAGCCTCTA CGAGTCTTCT |
| 661 | CTCCGAATAG AACCAAAACT CCCCACCTGA AGCTGTGCCC AGACCCAGAC CAGCTGCTCC |
| 721 | CGGGGACATG TGCTAGAIGA TACTGTACAT ATTCGTTTGG TTTCACTGGA TTTTCTTCTT |
| 781 | CAGTATGTGC TTCTCCAAGA AATACAAATC GTCCTTGTTC TTAGATTCCT GTAG |

GENBANK ID: M26708
VERSION M26708.1 GI:190695
MSDAAVDTSSEITTKDLKEKKEVVEEAENGRDAFANGNANEENG
EQEADNEVDEEEEEGGEEEEEEEEGDGEEEDGDEDEEAESATGKRAAEDDEDDDVDTK
KQKTDEDD
GENBANK ID: M81757
VERSION M81757.1 GI:337732
MPGVTVKDVNQQEFVRALAAFLEKSGKLKVPEWVDTVKLAIHKE
LAPYDENWFYTRAASTARHLYLRGGAGVGSNTKIYGGRQRNGVMPSHFSRGSRSVARR
VLQALEGLKMVEKDQDGGRKLTPQGQRDLDRIAGQVAAANEKH
GENBANK ID: V00568
DEFINITION HUMAN MRNA ENCODING THE C-NYC ONCOGENE.
VERSION V00568.1 GI:34815
MPLNV FTNRNYDLDYDSVQPYFYCDEEENFYQQQQQSELQPPA
PSEDIWKKFELLPTPPLSFSRRSGLCSPSYVAVTPFSLRGDNDGGGGSFSTADQLEMV
TELLGGDMVNQSFICDPDDETFIKNIIIQDCNWSGFSAAAKLVSEKLASYQAARKDSG
SPNPARGHSVCSTSSLYLQDLSAAASECIDPSVVFPYPLNDSSSPRSCASQDSSAFSP
SSDSLLSSTESSPQGSPEPLVLHEETPPTTSSDSEEEQEDEEEIDVVSVEKRQAPGKR
SESGSPSAGGHSEPPHSPLVLKRCHVSTHQHNYAAPPSTRKDYPAAKRVKLDSVRVLR
QISNNRKCTSPRSSDTEENVKRRTHNVLERQRRNELKRSFFALRDQIPELENNEIAPK
VVILKKATAYILSVQAEEQKLISEEDLLRKRREQLKHNLEQLRNSCA
GENBANK ID: L29219.1
VERSION L29219.1 GI:632963
MRHSKRTYCPDWDDKDWDYGKWRSSSSHERRERENSSAQENKRC
KYNHSKMCDSHYLESRSINEEDYHSRRYIDEYRNDYTQGCEPGHRQRDHESRYQNHSS
KSSGRSGRSSYKSKHRIHHSTSHRRSHGKSHRRKRTRSVEDDEEGHLICQSGDVLSAR
YEIVDTLGEGAFGKVVECIDHKAGGREVAVKIVKNVDRYCEAARSEIQVLEHLNTTDP
NSTFRCVQMLEWFEHHGHICIVFELLGLSTYDFIKENGFLPFRLDHIRKMAYQICKSV
NFLHSNKLTHTDLKPENILFVQSDYTEAYNPKIKRDERTLINPDIKVVDFGSATYDDE
HHSTLVSTRHYRAPEVILALGWSQPCDVWSIGCILIEYYIGFTVFPTHDSKEHLAMME
RILGPLPKHMIQKTRKRKYFHHDRLDWDEHSSAGRYVSRACKPLKEFMLSQDVEHERL
FDLIQKMLEYDPAKRITLREALKHPFFDLLKKSI
GENBANK ID: U49399.1
VERSION U49399.1 GI:1418220
MLLEEVRAGDRLSGAAARGDVQEVRRLLHRELVHPDALNRFGKT
ALQVMMFGSTAIALELLKQGASPNVQDTSGTSPVHDAARTGFLDTLKVLVEHGADVNV
PDGTGALPIHLAVQEGHTAVVSFLAAESDLHRRDARGLTPLELALQRGAQDLVDILQG
HMVAPL
GENBANK ID: XM_039993.2
VERSION XM_039993.2 GI:16188964
MVSYWDTGVLLCALLSCLLLTGSSS0SKLKDPELSLKGTQHIMQ
AGQTLHLQCRGEAAHKWSLPEMVSKESERLSITKSACGRNGKQFCSTLTLNTAQANHT
GFYSCKYLAVPTSKKKETESAIYIFISDTGRPFVEMYSEIPEIIHMTEGRELVIPCRV
TSPNITVTLKKFPLDTLIPDGKRIIWDSRKGFIISNATYKEIGLLTCEATVN0HLYKT
NYLTHRQTNTIIDVQISTPRPVKLLRGHTLVLNCTATTPLNTRVQMTNSYPDEKNKRA
SVRRRIDQSNSHANIFYSVLTIDKMQNKDKGLYTCRVRSGPSFKSVNTSVHIYDKAFI
TVKHRKQQVLETVAGKRSYRLSMKVKAFPSPEVVWLKDGLPATEKSARYLTRGYSLII
KDVTEEDAGNYTILLSIKQSNVFKNLTATLIVNVKPQIYEKAVSSFPDPALYPLGSRQ
ILTCTAYGIPQPTIKNFNHPCNHNHSEARCDFCSNNEESFILDADSNMGNRIESITQR
MAIIEGKNKMASTLVVADSRISGIYICIASNKVQTVGRNISFYITDVPNGFHVNLEKN
PTEGEDLKLSCTVNKFLYRDVTWILLRTVNNRTMHYSISKQKMAITKEHSITLNLTIM
NVSLQDSGTYACRARNVYTGEEILQKKEITIRDQEAPYLLRNLSDHTVAIESSTTLDC
HANGVPEPQITWFKNNHKIQQEPGIILGPGSSTLFIERVTEEDEGVYHCKATNQKGSV
ESSAYLTVQGTSDKSNLELITLTCTCVAATLFWLLTLFIRKDAKRSSSEIKTDYLSII
MDPDEVPLDEQCERLPYDASKWEFARERLKLGKSLGRGAFGKVVQASAFGIKKSPTCR
TVAVKMLKEGATASEYKALMTELKILTHIGHHLNVVNLLGACTKQGGPLMVIVEYCKY
GNLSNYLKSKRDLFFLNKDAALHMEFKKEKNEPGLEQGKKPRLDSVTSSESFASSGFQ
EDKSLSDVEEEEDSD0FYKEPITMEDLISYSFQVARQNEFLSSRKCIHRDLAARNILL
SENNVVKICDFGLARDIYKNPDYVRKGDTRLPLKWMAPESIFDKIYSTKSDVWSYGVL
LWEIFSLGGSFYPGVQNDEDFCSRLREGNRMRAPEYSTFEIYQIMLDCWHRDPKERPR
FAELVEKLGDLLQANVQQDGKDYIPINAILTGNSGFTYSTPAFSEDFFKESISAPKFN
SGSSDDVRYVNAFKFMSLERIKTFEELLPNATSMFDDYQGDSSTLLASPMLKRFTWTD
SKPKASLKIDLRVTSKSKESGLSDVSRPSFCHSSCGHVSEGKRRFTYDHAELERKIAC
CSPPPDYNSVVLYSTPPI
GENBANK ID: U61262.1
VERSION U61262.1 GI:1621606
CDS 137..4522
/CODON_START=1

| | |
|---|---|
| 1 | GGGCCGGGCC GGGCTGGGCT GGAGCAGCGG CGCCCGGGAG CCGAGCTTGC AGCGAGGGAC |
| 61 | CGGCTGAGGC GCGCGGGAGG GAAGGAGGCA ACGGCTCCGC GGCGCTGTCG CGCTGCCGCT |
| 121 | CACTCTCGGG GAAGAGATGG CGGCGGAGCG GGGAGCCCGG CGACTCCTCA GCACCCCCTC |
| 181 | CTTCTGGCTC TACTGCCTGC TGCTGCTCGG GCGCCGGGCG CCGGGCGCCG CGGCGGCCAG |
| 241 | GAGCGGCTCC GCGCCGCAGT CCCCAGGAGC CAGCATTCGA ACGTTCACTC CATTTTATTT |
| 301 | TCTGGTGGAG CCGGTGGATA CACTCTCAGT TAGAGGCTCT TGTGTTATAT TAAACTGTTC |
| 361 | AGCATATTCT GAGCCTTCTC CAAAAATTGA ATGGAAAAAA GATGGAACTT TTTTAAACTT |
| 421 | AGTATCAGAT GATCGACGCC AGCTTCTCCC GGATGGATCT TTATTTATCA GCAATGTGGT |
| 481 | GCATTCCAAA CACAATAAAC CTGATGAAGG TTATTATCAG TGTGTGGCCA CTGTTGAGAG |
| 541 | TCTTGGAACT ATTATCAGTA GAACAGCGAA CCTCATAGTA GCAGGTCTTC CAAGATTTAC |
| 601 | CAGCCAACCA GAACCTTCCT CAGTTTATGC TGGGAACGGA GCAATTCTGA ATTGTGAAGT |
| 661 | TAATGCAGAT TTGGTCCCAT TTGTGAGGTG GGAACAGAAC AGACAACCCC TTCTTCTGGA |
| 721 | TGATAGAGTT ATCAAACTTC CAAGTGGAAT GCTGGTTATC AGCAATGCAA CTGAAGGAGA |
| 781 | TGGCGGGCTT TATCGCTGCG TAGTGGAAAG TGGTGGGCCA CCAAAGTATA GTCATGAAGT |
| 841 | TGAATTGAAG GTTCTTCCAG ATCCTGAGGT GATATGAGAC TTGGTATTTT TGAAACAGCC |
| 901 | TTCTCCCTTA GTCAGAGTCA TTGGTCACGA TGTAGTGTTG CCATGTGTTG CTTCAGGACT |
| 961 | TCCTACTCCA ACCATTAAAT GGATGAAAAA TGAGGAGGCA CTTGACACAC AAAGCTCTGA |
| 1021 | AAGATTGGTA TTGCTGGCAG GTGGTAGCCT GGAGATCAGT GATGTTACTG AGGATGATGC |
| 1081 | TGGGACTTAT TTTTGTATAG CTGATAATGG AAATGAGACA ATTGAAGCTC AAGCAGAGCT |
| 1141 | TACAGTGCAA GCTCAACCTG AATTCCTGAA GCAGCCTACT AATATATATG CTCACGAATC |
| 1201 | TATGGATATT GTATTTGAAT GTGAAGTGAC TGGAAAACCA ACTCCAACTG TGAAGTGGGT |
| 1261 | CAAAAATGGG GATATGGTTA TCCCAAGTGA TTATTTTAAG ATTGTAAAGG AACATAATCT |
| 1321 | TCAAGTTTTG GGTCTGGTGA AATCAGATGA AGGGTTCTAT CAGTGCATTG CTGAAAATGA |
| 1381 | TGTTGGAAAT GCACAAGCTG GAGCCCAACT GATAATCCTT GAACATGCAC CAGCCACAAC |
| 1441 | GGGACCACTG CCTTCAGCTC CTCGGGATGT CGTGGCCTCC CTGGTCTCTA CCCGCTTCAT |
| 1501 | CAAATTGACG TGGCGGACAC CTGCATCAGA TCCTCACGGA GACAACCTTA CCTACTCTGT |
| 1561 | GTTCTACACC AAGGAAGGGA TTGCTAGGGA ACGTGTTGAG AATACCAGTC ACCCAGGAGA |
| 1621 | GATGCAAGTA ACCATTCAAA ACCTAATGCC AGCGACCGTG TACATCTTTA GAGTTATGGC |
| 1681 | TCAAAATAAG CATGGCTCAG GAGAGAGTTC AGCTCCACTG CGAGTAGAAA CACAACCTGA |
| 1741 | GGTTCAGCTC CCTGGCCCAG CACCTAACCT TCGTGCATAT GCAGCTTCGC CTACCTCCAT |
| 1801 | CACTGTTACG TGGGAAACAC CAGTGTCTGG CAATGGGGAA ATTCAGAATT ATAAGTTGTA |
| 1861 | CTACATGGAA AAGGGGACTG ATAAAGAACA GGATGTTGAT GTTTCAAGTC ACTCTTACAC |
| 1921 | CATTAATGGG TTGAAAAAAT ATACAGAGTA TAGTTTCCGA GTGGTGGCCT ACAATAAACA |
| 1981 | TGGTCCTGGA GTTTCCACAC CAGATGTTGC TGTTCGAACA TTGTCAGATG TTCCCAGTGC |
| 2041 | TGCTCCTCAG AATCTGTCCT TGGAAGTGAG AAATTCAAAG AGTATTATGA TTCACTGGCA |
| 2101 | GCCACCTGCT CCAGCCACAC AAAATGGGCA GATTACTGGC TACAAGATTC GCTACCGAAA |
| 2161 | GGCCTCCCGA AAGAGTGATG TCACTGAGAC CTTGGTAAGC GGGACACAGC TGTCTCAGCT |
| 2221 | GATTGAAGGT CTTGATCGGG GGACTGAGTA TAATTTCCGA GTGGCTGCTC TAACAATCAA |
| 2281 | TGGTACAGGC CCGGCAACTG ACTGGCTGTC TGCTGAAACT TTTGAAAGTG ACCTAGATGA |
| 2341 | AACTCGTGTT CCTGAAGTGC CTAGCTCTCT TCACGTACGC CCGCTCGTTA CTAGCATCGT |
| 2401 | AGTGAGCTGG ACTCCTCCAG AGAATCAGAA CATTGTGGTC AGAGGTTACG CCATTGGTTA |
| 2461 | TGGCATTGGC AGCCCTCATG CCCAGACCAT CAAAGTGGAC TATAAACAGC GGTATTACAC |
| 2521 | CATTGAAAAT CTGGATCCCA GCTCTCACTA TGTGATTACC CTGAAAGCAT TTAATAACGT |
| 2581 | GGGTGAAGGC ATCCCCCTGT ATGAGAGTGC TGTGACCAGG CCTCACACAG ACACTTCTGA |
| 2641 | AGTTGATTTA TTTGTTATTA ATGCTCCATA CACTCCAGTG CCAGATCCCA CTCCCATGAT |
| 2701 | GCCACCACTG GGAGTTCAGG CTTCCATTCT GAGTCATGAC ACCATCAGGA TTACGTGGGC |
| 2761 | AGACAACTCG CTGCCCAAGC ACCAGAAGAT TACAGACTCC CGATACTACA CCGTCCGATG |
| 2821 | GAAAACCAAC ATCCCAGCAA ACACCAAGTA CAAGAATGCA AATGCAACCA CTTTGAGTTA |
| 2881 | TTTGGTGACT GGTTTAAAGC CGAATACACT CTATGAATTC TCTGTGATGG TGACCAAAGG |
| 2941 | TCGAAGATCA AGTACATGGA GTATGACAGC CCATGGGACC ACCTTTGAAT TAGTTCCGAC |
| 3001 | TTCTCCACCC AAGGATGTGA CTGTTGTGAG TAAAGAGGGG AAACCTAAGA CCATAATTGT |
| 3061 | GAATTGGCAG CCTCCCTCTG AAGCCAATGG CAAAATTACA GGTTACATCA TATATTACAG |
| 3121 | TACAGATGTG AATGCAGAGA TACATGACTG GGTTATTGAG CCTGTTGTGG GAAACAGACT |
| 3181 | GACTGACCAG ATACAAGAGT TAACTCTTGA CACACCATAC TACTTCAAAA TCCAGGCACG |
| 3241 | GAACTCAAAG GGCATGGGAC CCATGTCTGA AGCTGTCCAA TTCAGAACAC CTAAAGCGGA |
| 3301 | CTCCTCTGAT AAAATGCCTA ATGATCAAGC CTCAGGGTCT GGAGGGAAAG GAAGCCGGCT |
| 3361 | GCCAGACCTA GGATCCGACT ACAAACCTCC AATGAGCGGC AGTAACAGCC CTCATGGGAG |
| 3421 | CCCCACCTCT CCTCTGGACA GTAATATGCT GCTGGTCATA ATTGTTTCTG TTGGCGTCAT |
| 3481 | CACCATCGTG GTGGTTGTGA TTATCGCTGT CTTTTGTACC CGTCGTACCA CCTCTCACCA |
| 3541 | GAAAAAGAAA CGAGCTGCCT GCAAATCAGT GAATGGCTCT CATAAGTACA AAGGGAATTC |
| 3601 | CAAAGATGTG AAACCTCCAG ATCTCTGGAT CCATCATGAG AGACTGGAGC TGAAACCCAT |
| 3661 | TGATAAGTCT CCAGACCCAA ACCCCATCAT GACTGATACT CCAATTCCTC GCAACTCTCA |
| 3721 | ACATATCACA CCAGTTGACA ACTCCATCCA CAGCAATATC CATCAAAGGC GAAATTCATA |
| 3781 | CAGAGGGCAT GAGTCAGAGG ACAGCATGTC TACACTGGCT GGAAGGCGAG GAATGAGACC |
| 3841 | AAAAATGATG ATGCCCTTTG ACTCCCAGCC ACCCCAGCCT GTGATTAGTG CCCATCCCAT |
| 3901 | CCATTCCCTC GATAACCCTC ACCATCATTT CCACTCCAGC AGCCTCGCTT CTCCAGCTCG |
| 3961 | CAGTCATCTC TACCACCCGG GCAGCCCATG GCCCATTGGC ACATCCATGT CCCTTTCAGA |
| 4021 | CAGGGCCAAT TCCACAGAAT CCGTTCGAAA TACCCCCAGC ACTGACACCA TGCCAGCCTC |
| 4081 | TTCCTCTCAA ACATGCTGCA CTGATCACCA GGACCCTGAA GGTGCTACCA GCTCCTCTTA |
| 4141 | CTTGGCCAGC TCCCAAGAGG AAGATTCAGG CCAGACTCTT CCCACTGCCC ATGTTCGCCC |
| 4201 | TTCCCACCCA TTGAAGAGCT TCGCCGTGCC AGCAATCCCG CCTCCAGGAC CTCCCACCTA |
| 4261 | TGATCCTGCA TTGCCAAGCA CACCATTACT GTCCCAGCAA GCTCTGAACC ATCACATTCA |
| 4321 | CTCAGTGAAG ACAGCCTCCA TCGGGACTCT AGGAAGCAGC CCCCCTCCTA TGCCAGTGGT |
| 4381 | TGTTCCCAGT GCCCCTGAAC TGCAGGAGAC CACAAGGATG TTGGAAGACT CCGAGAGTAG |
| 4441 | CTATGAACCA GATGAGCTGA CCAAAGAGAT GGCCCACCTG GAAGGACTAA TGAAGGACCT |
| 4501 | AAACGCTATC ACAACAGCAT GACGACCTTC ACCAGGACCT GACTTCAAAC CTGAGTCTGG |
| 4561 | AAGTCTTGGA ACTTAACCCT TGAAAACAAG GAATTGTACA GAGTACGAGA GGACAGCACT |
| 4621 | TGAGAACACA GAATGAGCCA GCAGACTGGC CAGCGCCTCT GTGTAGGGCT GGCTCCAGGC |
| 4681 | ATGGCCACCT GCCTTCCCCT GGTCAGCCTG GAAGAAGCCT GTGTCGAGGC AGCTTCCCTT |
| 4741 | TGCCTGCTGA TATTCTGCAG GACTGGGCAC CATGGGCCAA AATTTTGTGT CCAGGGAAGA |
| 4801 | GGCCAGAAGT GCAACCTGCA TTTCACTTTG TGGTCAGGCC GTGTCTTTGT GCTGTGACTG |
| 4861 | CATCACCTTT ATGGAGTGTA GACATTGGCA TTTATGTACA ATTTTATTTG TGTCTTATTT |
| 4921 | TATTTTACCT TCAAAAACAA AAACGCCATC CAAAACCAAG GAAGTCCTTG GTGTTCTCCA |
| 4981 | CAAGTGGTTG ACATTTGACT GCTTGTTCCA ATTATGTATG GAAAGTCTTT GACACTGTGG |
| 5041 | GTCGTTCCTG GGGTTGGCTT GTTTTTTGGT TTCATTTTTA TTTTTTAATT CTGAGTCATT |
| 5101 | GCATCCTCTA CCAGCTGTTA ATCCATCACT CTGAGGGGGA GGAAATGTTG CATTGCTGTT |
| 5161 | TGTAAGCTTT TTTTATTATT TTTTTATTAT AATTATTAAA GGCCTGACTC TTTCCTCTCA |
| 5221 | TCACTGTGAG ATTACAGATC TATTTGAATT GAATGAAATG TAACATTGAA AAAAAAAAAA |
| 5281 | AAAAAAAAAA AAAAAAA |

GENBANK ID: M11730.1
VERSION M11730.1 GI:183986
PRODUCT=“HER2 MRNA”
CDS 151..3918
/CODON_START=1

| | |
|---|---|
| 1 | AATTCTCGAG CTCGTCGACC GGTCGACGAG CTCGAGGGTC GACGAGCTCG AGGGCGCGCG |
| 61 | CCCGGCCCCC ACCCCTCGCA GCACCCCGCG CCCCGCGCCC TCCCAGCCGG GTCCAGCCGG |
| 121 | AGCCATGGGG CCGGAGCCGC AGTGAGCACC ATGGAGCTGG CGGCCTTGTG CCGCTGGGGG |
| 181 | CTCCTCCTCG CCCTCTTGCC CCCCGGAGCC GCGAGCACCC AAGTGTGCAC CGGCACAGAC |
| 241 | ATGAAGCTGC GGCTCCCTGC CAGTCCCGAG ACCCACCTGG ACATGCTCCG CCACCTCTAC |
| 301 | CAGGGCTGCC AGGTGGTGCA GGGAAACCTG GAACTCACCT ACCTGCCCAC CAATGCCAGC |
| 361 | CTGTCCTTCC TGCAGGATAT CCAGGAGGTG CAGGGCTACG TGCTCATCGC TCACAACCAA |
| 421 | GTGAGGCAGG TCCCACTGCA GAGGCTGCGG ATTGTGCGAG GCACCCAGCT CTTTGAGGAC |
| 481 | AACTATGCCC TGGCCGTGCT AGACAATGGA GACCCGCTGA ACAATACCAC CCCTGTCACA |
| 541 | GGGGCCTCCC CAGGAGGCCT GCGGGAGCTG CAGCTTCGAA GCCTCACAGA GATCTTGAAA |
| 601 | GGAGGGGTCT TGATCCAGCG GAACCCCCAG CTCTGCTACC AGGACACCAT TTTGTGGAAG |
| 661 | GACATCTTCC ACAAGAACAA CCAGCTGGCT CTCACACTGA TAGACACCAA CCGCTCTCGG |
| 721 | GCCTGCCACC CCTGTTCTCC GATGTGTAAG GGCTCCCGCT GCTGGGGAGA GAGTTCTGAG |
| 781 | GATTGTCAGA GCCTGACGCG CACTGTCTGT GCCGGTGGCT GTGCCCGCTG CAAGGGGCCA |
| 841 | CTGCCCACTG ACTGCTGCCA TGAGCAGTGT GCTGCCGGCT GCACGGGCCC CAAGCACTCT |
| 901 | GACTGCCTGG CCTGCCTCCA CTTCAACCAC AGTGGCATCT GTGAGCTGCA CTGCCCAGCC |
| 961 | CTGGTCACCT ACAACACAGA CACGTTTGAG TCCATGCCCA ATCCCGAGGG CCGGTATACA |
| 1021 | TTCGGCGCCA GCTGTGTGAC TGCCTGTCCC TACAACTACC TTTCTACGGA CGTGGGATCC |
| 1081 | TGCACCCTCG TCTGCCCCCT GCACAACCAA GAGGTGACAG CAGAGGATGG AACACAGCGG |
| 1141 | TGTGAGAAGT GCAGCAAGCC CTGTGCCCGA GTGTGCTATG GTCTGGGCAT GGAGCACTTG |
| 1201 | CGAGAGGTGA GGGCAGTTAC CAGTGCCAAT ATCCAGGAGT TTGCTGGCTG CAAGAAGATC |
| 1281 | TTTGGGAGCC TGGCATTTCT GCCGGAGAGC TTTGATGGGG ACCCAGCCTC CAACACTGCC |
| 1321 | CCGCTCCAGC CAGAGCAGCT CCAAGTGTTT GAGACTCTGG AAGAGATCAC AGGTTACCTA |
| 1381 | TACATCTCAG CATGGCCGGA CAGCCTGCCT GACCTCAGCG TCTTCCAGAA CCTGCAAGTA |
| 1441 | ATCCGGGGAC GAATTCTGCA CAATGGCGCC TACTCGCTGA CCCTGCAAGG GCTGGGCATC |
| 1501 | AGCTGGCTGG GGCTGCGCTC ACTGAGGGAA CTGGGCAGTG GACTGGCCCT CATCCACCAT |
| 1561 | AACACCCACC TCTGCTTCGT GCACACGGTG CCCTGGGACC AGCTCTTTCG GAACCCGCAC |
| 1621 | CAAGCTCTGC TCCACACTGC CAACCGGCCA GAGGACGAGT GTGTGGGCGA GGGCCTGGCC |
| 1681 | TGCCACCAGC TGTGCGCCCG AGGGCACTGC TGGGGTCCAG GGCCCACCCA GTGTGTCAAC |
| 1741 | TGCAGCCAGT TCCTTCGGGG CCAGGAGTGC GTGGAGGAAT GCCGACTACT GCAGGGGCTC |
| 1801 | CCCAGGGAGT ATCTGAATGC CAGGCACTGT TTGCCCTGCC ACCCTGACTG TCAGCCCCAG |
| 1861 | AATGGCTCAG TGACCTGTTT TGGACCGGAG GCTGACCAGT GTGTGGCCTC TCCCCACTAT |
| 1921 | AAGGACCCTC CCTTCTGCGT GGCCCGCTGC CCCAGCGGTG TGAAACCTGA CCTCTCCTAC |
| 1981 | ATGCCCATCT GGAAGTTTCC AGATGACGAG GGCGCATGCC AGCCTTGCCC CATCAACTGC |
| 2041 | ACGCACTCCT GTGTGGACCT GGATGACAAG GGCTGCCCCG CCGAGCAGAG AGCCAGCCCT |
| 2101 | CTGACGTCCA TCGTCTCTGC GGTGCTTGGC ATTCTGCTGG TCGTGGTCTT GGGGGTGGTC |
| 2161 | TTTGGGATCC TCATCAAGCG ACGGCAGCAG AAGATCCCCA ACTACACCAT CCGGACACTG |
| 2221 | CTGCAGCAAA CGGAGCTGGT GGAGCCCCTG ACACCTAGCG GAGCGATGCC CAACCAGGCG |
| 2281 | CAGATGCGCA TCCTGAAAGA GACGGAGCTG ACGAAGGTGA AGGTGCTTGG ATCTGGCGCT |
| 2341 | TTTGGCACAG TCTACAAGCG CATCTGGATC CCTGATCGGC ACAATGTGAA AATTCCAGTG |
| 2401 | GCCATCAAAG TGTTGAGGGA AAACACATCC CCCAAAGCCA ACAAAGAAAT CTTACACGAA |
| 2461 | GCATACGTGA TGGCTGGTGT GGGCTCCCCA TATGTCTCCC GCCTTCTGGG CATCTCCCTC |
| 2521 | ACATCCACGG TGCAGCTGGT GACACAGCTT ATGCCCTATG GCTGCCTCTT ACACCATGTC |
| 2581 | CGGGAAAACC GCGGACGCCT CGGCTCCCAG GACCTGCTGA ACTGGTGTAT GCAGATTGCC |
| 2641 | AAGGGGATGA GCTACCTGGA GGATGTGCGC CTCCTACACA CGGACTTAGC CGCTCGGAAC |
| 2701 | GTCCTGGTCA AGAGTCCCAA CCATGTCAAA ATTACAGACT TCGGGCTGGC TCGGCTGCTG |
| 2761 | GACATTGACG AGACAGAGTA CCATGCAGAT GGGGGCAAGG TGCCCATCAA GTGGATGGCG |
| 2821 | CTGGAGTCCA TTCTCCGCCG GCGGTTCACC CACCAGAGTG ATGTGTGGAG TTATGGTGTG |
| 2881 | ACTGTGTGGG AGCTGATGAC TTTTGGGGCC AAACCTTACG ATGGGATCCC AGCCCGGGAG |
| 2941 | ATCCCTGACC TGCTCGAAAA GGGGGAGCGG CTGCCCCAGC CCCCCATCTG CACCATTGAT |
| 3001 | GTCTACATGA TCATGGTCAA ATGTTGGATG ATTGACTCTG AATGTCGGCC AAGATTCCGG |
| 3061 | GAGTTGGTGT CTGAATTCTC CCGCATGGCC AGGGACCCCC AGCGCTTTGT GGTCATCCAG |
| 3121 | AATGAGGACT TGGGCCCAGC CAGTCCCTTG GACAGCACCT TCTACCGCTC ACTGCTGGAG |
| 3181 | GACGATGACA TGGGGGACCT GGTGGATGCT GAGGAGTATC TGGTACCCCA GCAGGGCTTC |
| 3241 | TTCTGTCCAG ACCCTGCCCC GCGCGCTGGG GGCATGGTCC ACCACAGGCA CCGCAGCTCA |
| 3301 | TCTACCAGGA GTGGCGGTGG GGACCTGACA CTAGGGCTGG AGCCCTCTGA AGAGGAGGCC |
| 3361 | CCCAGGTCTC CACTGGCACC CTCCGAAGGG GCTGGCTCCG ATGTATTTGA TGGTGACCTG |
| 3421 | GGAATGGGGG CAGCCAAGGG GCTGCAAAGC CTCCCCACAC ATGACGCCAG CCCTCTACAG |
| 3481 | CGGTACAGTG AGGACCCCAC AGTACCCCTG CCCTCTGAGA CTGATGGCTA CGTTGCCCCC |
| 3541 | CTGACCTGCA GCCCCCAGCC TGAATATGTG AACCAGCCAG ATGTTCGGCC CCAGCCCCCT |
| 3601 | TCGCCCCGAG AGGGCCCTCT GCCTGCTGCC CGACCTGCTG GTGCCACTCT GGAAAGGGCC |
| 3661 | AAGACTCTCT CCCCAGGGAA GAATGGGGTC GTCAAAGACG TTTTTGCCTT TGGGGGTGCC |
| 3721 | GTGGAGAACC CCGAGTACTT GACACCCCAG GGAGGAGCTG CCCCTCAGCC CCACCCTCCT |
| 3781 | CCTGCCTTCA GCCCAGCCTT CGACAACCTC TATTACTCGG ACCAGGACCC ACCAGAGCGG |
| 3841 | GGGGCTCCAC CCAGCACCTT CAAAGGGACA CCTACGGCAG AGAACCCAGA GTACCTGGGT |
| 3901 | CTGGACGTGC CAGTGTGAAC CAGAAGGCCA AGTCCGCAGA AGCCCTGATC TGTCCTCAGG |
| 3961 | GAGCAGGGAA GGCCTGACTT CTGCTGGCAT CAAGAGGTGG GAGGGCCCTC CGACCACTTC |
| 4021 | CAGGGGAACC TGCCATGCCA GGAACCTGTC CTAAGGAACC TTCCTTCCTG CTTGAGTTCC |
| 4081 | CAGATGGCTG GAAGGGGTCC AGCCTCGTTG GAAGAGGAAC AGCACTGGGG AGTCTTTGTG |
| 4141 | GATTCTGAGG CCCTGCCCAA TGAGACTCTA GGGTCCAGTG GATGCCACAG CCCAGCTTGG |
| 4201 | CCCTTTCCTT CCAGATCCTG GCTACTGAAA GCCTTAGGGA AGCTGGCCTG AGAGGGGAAG |
| 4261 | CGGCCCTAAG GGAGTGTCTA AGAACAAAAG CGACCCATTC AGAGACTGTC CCTGAAACCT |
| 4321 | AGTACTGCCC CCCATGAGGA AGGAACAGCA ATGGTGTCAG TATCCAGGCT TTGTACAGAG |
| 4381 | TGCTTTTCTG TTTAGTTTTT ACTTTTTTTG TTTTGTTTTT TTAAACACGA AATAAAGACC |
| 4441 | CAGGGGAGAA TGGGTGTTGT ATGGGGAGGC AAGTGTGGGG GGTCCTTCTC CACACCCACT |
| 4501 | TTGTCCATTT GCAAATATAT TTTGGAAAAC |

GENBANK ID: X14798.1
SEQUENCE 1:
MKAAVDLKPTLTIIKTEKVDLELFPSPDMECADVPLLTPSSKEM
MSQALKATFSGFTKEQQRLGIFKDPRQWTETHVRDWVMWAVNEFSLKGVDFQKFCMNG
AALCALGKDCGLELAPDFVGDILWEHLEILQKEDVKPYQVNGVNPAYFESRYTSDYFI
SYGIEHAQCVPPSEFSEPSFITESYQTLHPISSEELLSLKYENDYPSVILRDPLQTDT
LQNDYFAIKQEVVTFDNNCMGRTSRGKLGGQDSFESIESYDSCDRLTQSWSSQSSFNS
LQRVPSYDSFDSEDYPAALPNHKPKGTFKDYVRDRADLNKDKPVIPAAALAGYTGSGP
IQLWQFLLELLTDKSCQSFISWTGDGWEFKLSDPDBVARRWGKRKNKPKMNYEKLSRG
LRYYYDKNIIHKTAGKRYVYRFVCDLQSLLGYTPEELHANLDVKPDADE
SEQUENCE 2;
MKAAVDLKPTLTIIKTEKVDLELFPSPDMECADVPLLTPSSKEM
MSQALKATFSGFTKEQQRLGIPKDFRQWTETHVRDWVNWAVNEFSLKGVDFQKFCMNG
AALCALGKDGFLELAPDFVGDILWEHLEILQKEDVKPYQVNGVNPAYPESRYTSDYFI
SYGIEHAQCVPPSEFSEPSFITESYQTLHPISSEELLSLKYENDYPSVILRDDLQTDT
LQNDYFAIKQEVVTPDNMCNGRTSRGSGPIQLWQFLLELLTDKSCQSFTSWTGDGWEF
KLSDPDEVARRNGKRRNKPKNNYEKLSRGLRYYYDKNIIHKTAGKRYVYRFVCDLQSL
LGYTPEELHAMLDVKPDADE
GENBANK ID: D49547
VERSION D49547.1 GI:710654
MGKDYYQTLGLARGASDEEIKRAYRRQALRYNFDKNKEFGAEEK
FKEIAEAYDVLSDPRKREIFDRYGEEGLKGSGPSGCSGGGANGTSFSYTFHGDPRAMF
AEFFGGRNPFDTFFGQRNGEEGNDIDDFFSGFPMGMGGFTNVNFGRSRSAQEPARKKQ
DPPVTNDLRVSLEEIYSGCTKKMKISHKRLNFDGKSIRNEDKILTIEVKKGWKEGTKI
TEPKEGDQTSNNIPADIVFVLKDKPHNIFKRDGSDVIYPARISLREALCGCTVNVPTL
DGRTIFVVFKDVIRPGMRRKVPGEGLPLPKTPEKRGDLIIEFEVIFPERIPQTSRTVL
EQVLPI
GENBANK ID: M11717
VERSION D49547.1 GI:710654
MGKDYYQTLGLARGASDEEIKRAYRRQALRYHPDKNKEPGAEEK
FKEIAEAYDVLSDPRKREIFDRYGEEGLKGSGPSCGSGGGANGTSFSYTFHGDPHAMF
AEFFGGRNPFDTFFGQRNGEEGNDIDDPFSGFPNGNGGFTNVNFGRSRSAQEPARKKQ
DPPVTHDLRVSLEEIYSGCTKKMKISHKRLNPDGKSIRNEDKILTIEVKKGWKEGTKI
TFPKEGDQTSNNIFADIVFVLKDKPHNIFKRDGSDVIYFARISLREALCGCTVNVPTL
DGRTIPVVFKDVIRPGNRRKVPGEGLFLFKTFEKRGDLIIEFEVIFPERIPQTSRTVL
EQVLPI
GENBANK ID: X76648
VERSION X76648.1 GI:531404
MAQSFVNCKIQFGKVVVFIKPTCPYCRRAQEILSQLPIKQGLLE
FVDITATNHTNEIQDYLQQLTGARTVFRVFIGKDCIGGCSDLVSLQQSGSLLTRLKQI
GALQ
GENBANK ID: NM_011587.1
VERSION NM_011587.1 GI:6755784
MVWWGSSLLLFTLFLASHVGASVDLTLLANLRITDPQRFFLTCV
SGEAGAGRSSDPPLLLSKDDRIVRTIFPGQPLYLARNGSHQVTLRGFSKPSDLVGVFS
CVGGAGARRTRVLYVHNSPGAHLFPDKVTHTVNKGDTAVLSAHVHKEKQTDVIWKNNG
SYFNTLDWQEADDGRFQLQLQNVQPPSSGIYSATYLSASFLGSAFFRLIVRGCGAGRW
GPGCVKDCFGCLHGGVCMDMDGSCVCPPGFTGTRQSQACRSGRFGQSCQSQCPGTAGC
RGLTFCLPDPYGCSCGSGWRGSQCQSACAPDHFGADCRLQCQCQNGGTCDRFSGCVCP
SGWHGVHCEKSDRIPQILSMATSVSFNIGTMPRINCAAAGNPFPVRGSMKLRKPDGTM
LLSTKVIVEPDRTTASFSVFSLTLGDSGFWECRVSTSGGQDSRRFKVNVKVPPVPLTA
PRLLAKQSRQLVVSPLVSFSGDGPISSVRLEYRPQDSTIAWSAIVVDPSSNVTLMNLK
PKTGYNVRVQLSRPGEGGEGGWGPSALMTTDCPEPLLQFWLSSWNVEGPDRLRVSWSL
PSVPLSGDGFLLRLMDGARGQSRRSNISFPQARTALLTGLTPGTHYQLDVRLYHCTLL
GPASPPAMVNLPPSGPPAPRELHAQALSDSEIQLMWQHPSAPSGPISKYIVSIQVAGG
SGDFQWMDVDRPESTSIIVRGLNASTRYLFRVRASVQGLGDWSNTVSSATLGNGLQSE
DPVRESRAASEGLDQQLVLAVVGSVSATCLTILAALLALVCIRRSCLHRRRTFTYQSG
SGEETILQFSSGTLTLTRRFKFQFSPLSYFVLEWEDITFSDLIGSGNFGQVIRAMIKK
DGLKMNAAIKNLKEYASENDMRDFAGELEVLCKLGBHFNIINLLGACSNRGYLYIAIE
YAPYGNLLDFLRKSRVLSTDPAFARSHGTASTLSSRQLLRFASDAANGMQYLSSKQFI
HRDLAARNVLVGENLASKIADFGLSRGEEVYVKKTNGRLPVRWMAISSLNYSVYTTKS
DVWSFGVLLWEIVSLGGTPYCGMTCAELYEKLPQGYRMEQPRNCDDEVYSLMRQCWRD
RRYERPPFAQIALQLGRMLSARKAYVNMSLFENFTYAGIDATASSA
GENBANK ID: NM_002867.1
VERSION NM_002867.1 GI:4506368
MASVTDGKMGVKDASDQNFDYMFKLLIIGNSSVGKTSFLLRYAD
DTFTPAFVSTVGIDFKVKTVYRMSKRVKLQIWDTAGQSRYRTITTAYYRGANGFILMY
DITNESSFNAVQDWATQIKTYSNDNAQVILVGNKCDMEEERVVPTSKGQLLASQLGFD
FFEASAKENISVRQAFSRLVDAICDKMSDSLDTDPSMLGSSKNTRLSDTPPLLQQNCS
C
GENBANK ID: 027361
NO VERSION DATA
LNENQRLAVERILSGDCRPLPYILEQPPGTQKTVTIIEAVLQVH
FALPDSRILVCAPSNSAADLVCLRLHESKVLQPATMVRVGHFTHVFVDEAGQASEPEC
LIPLGLMSDISGQIVLAQDPMQLGPVIKSRLAMAYGLNVSFLERLMSRPAYQRDENAF
GACGANNPLLVTKLVKNYRSHEALLMLPSRLFYHRELEVCADPTVVTSLLGWEKLPKK
GFPLIFHGVRQSEAREGKSPSWFNPAEAVQVLRYCCLLAHSISSQVSASDIGVITPYR
KQVEKIRILLRNVDLMDIKVGSVEEFQGQEYLVIIISTVRSNEDRFEDDRYFLGFLSN
SKRFNVAITRPKALLIVLGNPHVLVRDPCFGALLEYSITNGVYMGCDLPPALQSLQNC
GEGVADPSYPVVPESTGPEKHQEPS
GENBANK ID: NM_002752.1
VERSION NM_002752.1 GI:4506096
MSDSKCDSQFYSVQVADSTFTVLKRYQQLKPIGSGAQGIVCAAF
DTVLGISVAVIKLSRPFQNQTHAKRAYRELVLLKCVNHKNIISLLNVFTPQKTLEEFQ
DVYLVMELMDANLCQVIHMELDHERMSYLLYQMLCGIKHLHSAGIIHRDLKPSNIVVK
SDCTLKILDFGLARTACTNFMMTPYVVTRYYRAPEVILGMGYKENVDIWSVGCIMGEL
VKGCVIFQGTDHIDQMNKVIEDLGTPSAEFMKKLQPTVRNYVENRPKYPGIKFEELFP
DWIFPSESERDKIKTSQARDLLSKMLVIDPDKRISVDEALRHPYITVWYDPAEAEAPP
PQIYDAQLEEREHAIEEWKELIYKEVMDWEERSKNGVVKDQPSAQMQQ
GENBANK ID: M22262.1
VERSION M22382.1 GI:190126
MLRLPTVFRQMRPVSRVLAPHLTRAYAKDVKFGADARALMLQGV
DLLADAVAVTMGPKGRTVIIEQSWGSPKVTKDGVTVAKSIDLKDKYKNIGAKLVQDVA
NNTNEEAGDGTTTATVLARSIAKEGFEKISKGANPVEIRRGVMLAVDAVIAELKKQSK
PVTTPEEIAQVATISANGDKEIGNIISDAMKKVGRKGVITVKDGKTLNDELEIIEGMK
FDRGYISPYFINTSKGQKCEFQDAYVLLSEKKISSIQSIVPALEIANAHRKPLVIIAE
DVDGEALSTLVLNRLKVGLQVVAVKAPGF0DNRKNQLKDMAIATGGAVFGEEGLTLNL
EDVQPHDLGKVGEVIVTKDDAMLLKQKGDKAQIEKRIQEIIEQLDVTTSEYEKEKLNE
RLAKLSDGVAVLKVCGTSDVEVNEKKDRVTDALNATRAAVEEGIVLGGGCALLRCIPA
LDSLTPANEDQKIGIEIIKRTLKIPAMTIAKNAGVEGSLIVEKIMQSSSEVGYDAMAG
DFVNMVEKGIIDPTKVVRTALLDAAGVASLLTTAEVVVTEIPKEEKDPGMGAMGGMGG
GMGGGMF
GENBANK ID: U09564.1
DEFINITION HUMAN SERINE KINASE MRNA, COMPLETE CDS.
VERSION U09564.1 GI:507212
MERKVLALQARKKRTKAKKDKAQRKSETQHRGSAPHSESDLPEQ
EEEILGSDDDEQEDPNDYCKGGYHLVKIGDLFNGRYHVIRKLGWGHFSTVWLSWDIQG
KKFVAMKVVKSAEHYTETALDEIRLLKSVRNSDPNDPNREMVVQLLDDFKISGVNGTH
ICMVFEVLGHHLLKWIIKSNYQGLPLPCVKKIIQQVLQGLDVLHTKCRIIHTDIKPEN
ILLSVNEQYIRRLAAEATENQRSGAPPPSGSAVSTAPQPKPADKMSKNKKKKLKKKQK
RQAELLEKRMQEIEEMEKESGPGQKRPNKQEESESPVERPLKENPPNKMTQEKLEESS
TIGQDQTLMERDTEGGAAEINCNGVIEVINYTQNSNNETLRHKEDLHNANDCDVQNLN
QESSFLSLPNGDSSTSQETDSCTPITSEVSDTMVCQSSSTVGQSFSEQHISQLQESIR
AEIPCEDEQEQEHNGPLDNKGKSTAGNFLVNPLEPKNAEKLKVKIADLGNACWVHKHF
TEDIQTRQYRSLEVLIGSGYNTPADIWSTACMAFELATGDYLFEPNSGEEYTRDEDHI
ALIIELLGKVPRKLIVAGKYSKEFFTKKGDLKNITKLKPNGLFEVLVEKYEWSQEEAA
GFTDFLLPMLELIPEKRATAAECLRHPWLNS
GENBANK ID: M11507.1
VERSION M11507.1 GI:239515
MMDQARSAFSNLFGGEPLSYTRFSLARQVDGDNSHVEMKLAVDE
EENADNNTKANVTKPKRCSGSICYGTIAVIVFFLIGFMIGYLGYCKGVEPKTECERLA
GTESPVREEPGEDFPAARRLVWDDLKRKLSEKLDSTDFTSTIKLLNENSYVPREAGSQ
KDENLALYVENQFREFKLSKVWRDQHFVKIQVKDSAQNSVIIVDKNGRLVYLVENPGG
YVAYSKAATVTGKLVHANFGTKKDFEDLYTPVNGSIVIVRAGKITFAEKVANAESLNA
IGVLIYMDQTKFPIVNAELSFFGHAHLGTGDPYTPGFPSFNHTQFPPSRSSGLPNIFV
QTISRAAAEKLFGNMEGDCPSDWKTDSTCRMVTSESKNVKLTVSNVLKEIKILNIFGV
IKGFVEPDHYVVVGAQRDAWGPGAAKSGVGTALLLKLAQNFSDMVLKDGFQPSRSIIF
ASWSAGDFGSVGATENLEGYLSSLHLKAFTYINLDKAVLGTSNFKVSASPLLYTLIEK
TMQNVKHPVTGQFLYQDSNNASKVEKLTLDNAAFPFLAYSGIPAVSFCFCEDTDYPYL
GTTMDTYKELIERIPELNKVARAAAEVAGQFVIKLTHDVELNLDYERYNSQLLSFVRD
LNQYRADTKEMGLSLQWLYSARCDFFRATSRLTTDFGNAEKTDRFVMKKLNDRVMRVE
YHFLSFYVSPKESPFRHVFWGSGSHTLPALLENLKLRKQNNGAFNETLFRNQLALATW
TIQGAANALSGDVWDIDNEF
GENBANK ID: 055017.1
VERSION 055017.1 GI:1297296
MESYHKFDQQKLQALKDTANRLRISSIQATTAAGSGHPTSCCSA
AEIMAVLFFHTMRYKSQDPRNPHNDIRFVLSKGHAAPILYAVWAEAGFLEAELLNLRK
ISSDLDGNPVFKQAFTDVATGSLGQGLGAACGMAYTGKYFDKASYRVYCLLGDGELSE
GSVWEAMAFASIYKLDNLVAILDINRLGQSDPAPLQNQNDIYQKRCEAFGWHAIIVDG
HSVEELCKAFCQAKHQFTAIIAKTFKGRGITGVEDKESWHGKPLPKNMAEQIIQEIYS
QIQSKKKILATFPQEDAPSVDIANIRMPSLPSYKVGDKIATRKAYGQALAKLGHASDR
IIALDGDTKNSTFSEIFKKEHPDRFIECYIAEQNNVSIAVGCATRNRTVPFCSTFAAF
FTRAFDQIRMAAISESNINLCGSHCGVSIGEDGPSQMALEDLAMFRSVPTSTVFYPSD
GVATEKAVELAANTKGICFIRTSRPENAIIYNNNEDFQVGQAKVVLKSKDDQVTVIGA
GVTLHEALAAAELLKKEKINIRVLDPFTIKPLDRKLILDSARATKCRILTVEDHYYEG
GIGEAVSSAVVGEFGITVTNLAVNRVPRSGKPAELLKMFGIDRDAIAQAVRGLITKA
GENEANK ID: X14034.
VERSION X14034.1 GI:35513
MSTTVNVDSLAEYEKSQIKRALELGTVNTVFSFRKSTFERRTVQ
VIMETRQVAWSKTADKIEGFLDINEIKEIRPGKNSKDFEPAKAVRQKEDCCFTILYGT
QFVLSTLSLAADSKEDAVNWLSGLKILHQEAMNASTPTIIESWLRKQIYSVDQTRRNS
ISLRELKTILPLINFKVSSAKFLKDKFVEIGAHKDELSFEQFHLFYKKLNFEQQKSIL
DEFKKDSSVFILGNTDRPDASAVYLHDFQRFLIHEQQENWAQDLNKVRERMTKFIDDT
MRETAEPFLFVDEFLTYLFSRENSTWDEKYDAVDNQDMNNPLSHYWISSSNNTYLTGD
QLRSESSPEAYIRCLRNGCRCIELDCWDGPDGKFVIYHGWTRTTKIKFDDVVQAIKDH
AFVTSSFPVILSIEEHCSVEQQRHMAKAFKEVFGDLLLTKPTEASADQLPSPSQLREK
IIIKNKKLGFPQDVDVNMEDKKDEHKQQGELYMWDSIDQKNTRHYCAIADAKLSFSDD
IEQTNEEEVFQDIFFTELNFGEKWFHKKVEKRTSAEKLLQEYCNETGGKDGTFLVRES
ETFPNDYTLSFWRSGRVQNCRIRSTNEGGTLKYYLTDNLRFRRNYALIQHYRETHLPC
AEFELRLTDFVFNPNPHESKPWYYDSLSRGEAEDMLNRIPRDGAFLIRKRECSDSYAI
TFRARGKVKHCRINRDGRHFVLGTSAYFESLVELVSYYEKHSLYRKNRLRYFVTPELL
ERYNTERDINSLYDVSRNYVDPSEINPSMPQRTVKALYDYKAKRSDELSFCRGALIHN
VSKEPGGNWKGDYGTRIQQYFFSNYVEDISTADFEELEKQIIEDNPLGSLCRGILDLN
TYNVVKAFQGKNQKSFVFILEPKEQ0DPPVEFATDRVEELFEWFQSIREITWKIDSKE
NNMKYWEKNQSIAIELSDLVVYCKFTSKTKDNLENPDFREIRSFVETKADSIIRQKPV
DLLKYNQKGLTRVYFKQQRVDSSNYDPFRLNLCGSQNVALNFQTADKYNQNNRALFSL
NGRTGYVLQFESNRTEKYDPNFFESQRKILMTLTVKVLGARHLPKLGRSIACPFVEVE
ICGAEYGNNKFKTTVVNDNGLSPIWAPTQEKVTFEIYDFNLAFLRFVVYEEDNFSDPN
FLAHATYPIKAVKSGPRSVFLKNGYSEDIELASLLVFCEMRPVLESEEELYSSCRQLR
RRQEELNNQLFLYDTHQNLRNANRDALVKEFSVNENHSSCTRRNATRG
GENBANK ID: M27691.1I
VERSION 1627691.1 GI:181038
NTMESGAENQQSCDAAVTEAENQQNTVQAQPQIATLAQVSMPAA
HATSSAPTVTLVQLPNGQTVQVHCVIQAAQPSVIQSPQVQTVQISTIAESEDSQESVD
SVTDSQKRREILSRRFSYRKILNDLSSDAFGVPRIEEEKSEEETSAFAITTVTVFTPI
YQTSSGQYIAITQGGAIQLANNGTDGVQGLQTLTNTNAAATQPGTTILQYAQTTDGQQ
ILVPSNQVVVQAASGDVQTYQIRTAPTSTIAPGVVMASSPALPTQPAEEAARKREVRL
MKNREAARECRRKKKEYVKCLENRVAVLENQNKTLIEELKALKDLYCHKSD
GENBANK ID: 1618391
VERSION 1618391.1 GI:339716
MERRWPLGLGLVLLLCAPLPPGARAKEVTTJIVIDTSKAQGELGWLL
DFPKDGWSEQQQILNGTPLYNYQDCPNQGRRDTDNWLRSNWIYRCEEASRVRVELQFT
VRDCKSFPGGAGFLGCKETFNLLYNESDQDVCIQLRRPLFQKVTTVAADQSFTIRDLA
SGSVKLNVERCSLGRLTRRGLYLAFHNPGACVALVSVRVFYQRCPETLNGLAQFPDTL
PGPAGLVEVACTCLPHARASPRFSCAPRNHCSPDGEWLVPVGRCHCEFGYEEGGSGEA
CVACPSGSYRNDMDTPHCLTCFQQSTAESEGATICTCESGHYRAFGEGPQVACTGPPS
APRNLSFSASGTQLSLRWEPPADTGGRQDVRYSVRCSQCQCTAQDGGPCQPCGVGVHF
SPGARALTTPAVEVNGLEFYANYTFNVEAQNGVSGLGSSGHASTSVSISMGNAESLSG
LSLRLVKKEPRQLELTWAGSRPRSPGANLTYELHVLNQDEERYQNVLEFRVLLTELQP
DTTYIVRVRMLTPLGPGPFSPDNEFRTSPFVSRGLTGGEIVAVIFGLLLGAALLLGIL
VFRSRRAQRQRQQRHVTAPPNWIERTSCAEALCGTSRHTRTLHREPWTLPCGWSNFPS
RELDFAWLNVDTVIGEGEFGEVYRGTLRLPSQDCKTVAIKTLKDTSPGCQWWNFLREA
TIMGQFSNFHILHLECVVTKRKPIMIITEFNENAALDAFLREREDQLVPGQLVAMLQG
IASGMNYLSNHNYVNRDLAARNILVNQNLCCKVSDFCLTRLLDDFDGTYETQGGKIPI
RWTAPEAIANRIFTTASDVWSFGIVNWEVLSFGDKPYGENSNQEVNKSIEDCYRLPPP
VDCPAPLYELMKNCWAYDRARRPNFQKLQAHLEQLLANPNSLRTIANFDPRVTLRLPS
LSGSDGIFVRTVSEWLESIRMKRYILHFHSAGLDTMECVLELTAEDLTQNGITLPGHQ
KRILCSIQGFKD
GENBANK ID: X54079.1
VERSION X54079.1 GI:32477
MTERRVPFSLLRGPSWDPERDWYPHSDLFDQAFGLPRLEEWSQ
WLGGSSWFGYVRPLPFAAIESPAVAAFAYSRALSRQLSSGVSEIRHTADRWRVSLDVN
HFAFDELTVKTKDGVVEITGKHEERQDEHGYISRCFTRKYTLFPGVDPTQVSSSLSFE
GTLTVEAPMPKLATQSNEITTPVTFESRAQLGGPEAAKSDETAAK
GENBANK ID: XM_012654.3
VERSION XM_012654.3 GI:14773503
MFGVTLWEMFSGGEEPWACVPPYLILQRLEDFARLPRPPLCSRA
LYSLALDCWAPHPSDRPSFSHLEGLLQEAGPSEACCVRDVTEFGALRNETGDPITVIE
GSSSFHSPDSTIWKGQNGRTFKVGSWPASAVTLADAGGLPATRPVHRGHPCFGRSTPR
KHRWRQKEGKSLGCAFSTGPEEEHAPGEDERNFQESGVSSVPRSSSHRGXVQAPLKXR
QARAXAPGTSRFASTPTFIL
GENBANK ID: XM_054457.2
VERSION XM_054457.2 GI:18590931
MASATDSRYGQKESSDQNFDYNFKILIIGNSSVGKTSFLFRYAD
DSFTPAFVSTVGIDFKVKTIYRNDKRIKLQIWDTAGQERYRTITTAYYRGANGFILMY
DITNEESFNAVGDWSTQIKTYSWDNAQVLLVGNKCDMEDERVVSSERGRQLADHLGFE
FFEASAKDNINVKQTFERLVDVICEKMSESLDTADPAVTGAKQGPQLSDQQVFPHQDC
AC
GENBANK ID: XM_038595.3
VERSION XM_038595.3 GI:18590923
MAPPSEETPLIPQRSCSLLSTEAGALEVLLPARGPGPPQRLSFS
FGDHLAEDLCVQAAKASGILPVYHSLFALATEDLSCWFPPSHIFSVEDASTQVLLYRI
RFYFPNWFGLEKCHRFGLRKDLASAILDLPVLEHLFADHRSDLVSGRLFVGLSLKEQG
ECLSLAVLDLARMAREQAQRPGELLKTVSYKACLPPSLRDLIQGLSFVTRRRIRRTVR
RALRRVAACQADRHSLMAKYIMDLERLDPAGAAETFHVGLPGALGGHDGLGLLRVAGD
GGIAWTQGEQEVLQPFCDFPEIVDISIKQAPRVGPAGEHRLVTVTRTDNQILEAEFPG
LPEALSFVALVDGYFRLTTDSQHFFCKEVAPPRLLEEVAEQCHGPITLDFAINKLKTG
GSRPGSYVLRRSPQDFDSFLLTVCVQNPLGPDYKGCLIRRSPTGTFLLVGLSRFHSSL
RELLATCWDGGLHVDGVAVTLTSCCIPRPKEKSNLIVVQRGHSPFTSSLVQPQSQYQL
SQMTFHKIPADSLEWHENLGHGSFTKIYRGCRHEVVDGEARKTEVLLKVMDAKHKNCM
ESFLEAASLWSQVSYRHLVLLHGVCMAGDSTWVQEFVHLGAIDMYLRKRGHLVPASWK
LQVVKQLAYALNYLEDKGLFHGNVSARKVLLAREGADGSPPFIKLSDFGVSFAVLSLE
MLTDRIPWVAPECLREAQTLSLEADKWGFGATVWEVFSGVTMPISALDPAKKLQFYED
RQQLFAPKWTELALLIQQCMAYEFVQRPSFRAVIRDLNSLISSDYELLSDFTPGALAP
RDGLWNGAQLYACQDPTIFEERHLKYISGLGKGNFGSVELCRYDPLGDNTGALVAVKQ
LQHSGPDQQRDFQREIQILKALHSDFIVKYRGVSYGPGRQSLRLVMEYLPSGCLRDFL
QRHRARLDASRLLLYSSQICKGMEYLGSRRCVHRDLAARNILVESEAHVKIADFGLAK
LLPLDKDYYVVREPGQSPIFWYAPESLSDNIFSRGSDVWSFGVVLYELFTYCDKSCSP
SAEFLRMMGCERDVPALCRLLELLEEGQRLPAPPACPAEVHELMKLCWAPSPQDRPSF
SALGFQLDMLWSGSRGCETHAFTAHFEGKHHSLSFS
GENBANK ID: NM_002755.2
VERSION NM_002755.2 GI:14589898
MPKKKPTPIQLNPAPDGSAVNGTSSAETNLEALQKKLEELELDE
QQRKRLEAFLTQKQKVGELKDDGFEKISELGAGNGGVVFKVSHKPSGLVMARKLTHLE
IKPAIRNQIIRELQVLHECNSFYIVGFYGAFYSDGEISICMEENDGGSLDQVLKKAGR
IPEQILGKVSIAVIKGLTYLREKNKIMNRDVKPSNILVNSRGEIKLCDFGVSGQLIDS
MANSFVGTRSYMSFERLQGTHYSVQSDIWSMGLSLVEMAVGRYPIPPFDAKELELMFG
CQVEGDAAETPPRFRTPGRPLSSYGMDSRPPMAIFELLDYIVNEPFPKLPSGVFSLEF
QDFVNKCLIKNPAERADLKQLMVRAFIKRSDAEEVDFAGWLCSTIGLNQPSTPTHAAG
V
GENBANK ID: NM_001744.1
VERSION NM_001744.1 GI:4502556
MLKVTVFSCSASSCSSVTASAAPGTASLVPDYWIDGSNRDALSD
FFEVESELGRGATSIVYRCKQKQTQKPYALKVLKKTVDKKIVRTEIGVLLRLSHPNII
KLKEIFETFTEISLVLELVTGGELFDRIVEKGYYSERDAADAVKQILEAVAYLHENGI
VHRDLKFENLLYATPAPDAPLKIADFGLSKIVEHQVLMKTVCGTPGYCAPEILRGCAY
GPEVDMWSVGIITYILLCCFEPFYDERGDQFMFRRILNCEYYFISPWWDEVSLNAKDL
VRKLIVLDPKKRLTTFQALQHPWVTGKAANFVHMDTAQKKLQEFNARRKLKAAVKAVV
ASSRLCSASSSHGSIQESRKASRDPSPIQDGNEDMKAIPEGEKIQGDGAQAAVKGAQA
ELMKVQALEKVKGADINAEEAPKMVPKAVEDGIKVADLELEECLAEEKLKTVEEAAAP
REGQGSSAVGFEVPQQDVILPEY
GENBANK ID: XM_053461.2
VERSION XM_053461.2 GI:13553657
MASRGATRPNGPNTGNKICQFKLVLLGESAVGKSSLVLRFVKGQ
FHEFQESTGAAFLTQTVCLDDTTVKFEIWDTAGQERYEHSLAPMYYRGAQAAIVVYDI
TNEESFARAKNMVKELQRQASPNIVIALSGNKADLANKRAVDFQEAQSYADDNSLLFM
ETSAKTSMNVNEIFMAIAKKLPKNEPQNPGANSARGRGVDLTEPTQPTRNQCCSN
GENBANK ID: U33635.1
VERSION U33635.1 GI:1016701
MGAARGSPARPRRLPLLSVLLLPLLGGTQTAIVFIKQPSSQDAL
QGRRALLRCEVEAPGPVHVYWLLDGAPVQDTERRFAQGSSLSFAAVDPLQDSGTFQCV
ARDDVTGEEARSANASFNIKWIEAGPVVLKHPASEAEIQPQTQVKLRCHIDGMPRFTY
QWFRDGTPLSDGQSNHTVSSKERNLTLRPAGPEHSGLYSCCAHSAFSQACSSQNFTLS
IADESFARVVLAPQDVVVARYEEAMFHCQFSAQPPPSLQWLFEDETPITNRSRPPHLR
RATVFANGSLLLTQVRPRNAGIYRCIGQGQRGPPIILEATLHLAEIEDMPLFEPRVFT
AGSEERVTCLPPKGLFEPSVWWEHAGVRLFTHGRVYQKGHELVLANIAESDAGVYTCH
AANLAGQRRQDVNITVATVPSWLKKPQDSQLEECKPGYLDCLTQATPKFTVVWYRNQM
LISEDSRFEVFKNGTLRINSVEVYDGTWYRCMSSTPAGSIEAQAVLQVLEKLKFTPPP
QPQQCMGFDKEATVFCSATGREKPTIKWERADGSSLPEWVPDNAGTLHFARVTRDDAG
NYTCIASNGPQGQIRAHVQLTVAVFITFKVEPERITVYQGHTALLQCEAQGDPKPLIQ
WKGKDRILDPTKLGPRMHIFQNCSLVIHDVAPEDSGRYTCIAGNSCNIKHTEAPLYVV
DKPVPEESEGPGSPPPYKMIQTIGLSVGAAVAYIIAVLGLMFYCKKRCKAKRLQKQPE
GEEPEMECLNGGPLQNGQPSAEIQEEVALTSLGSGPAATNKRHSTSDKMHFPRSSLQP
ITTLGKSEFGEVFLAKAQGLEEGVAETLVLVKSLQSKDEQQQLDFRRELEMFGKLNHA
NVVRLLGLCREAEPHYMVLEYVDLEDLKQFLRISKSKDEKLKSQPLSIKQKVALCTQV
ALGMEHLSNNRFVNKDLAARNCLVSAQRQVKVSALGLSKDVYNSEYYHFRQANVALRW
MSPEAILEGDFSTKSDVNASGVLMWEVFTHGEMPHGGQADDEVLADLQAGKARLFQPE
GCPSKLYRLMQRCWALSPKDRPSESEIASALGDSTVDSKP
GENBANK ID: XM_004559.5
VERSION XM_004559.5 GI:17464405
MGPEALSSLLLLLLVASGDADMKGHFDPAKCRYALGMQDRTIPD
SDISASSSNSDSTAARHSRLESSSDGDGAWCPAGSVFPKEEEYLQVDLQRLHLVALGT
QGRHAGGLGKEFSRSYRLRYSRDGRRWMGWKDRWGQEVIS0NEDFEGVVLKDLGPPMV
ARLVRFYFRADRVMSVCLRVELYGCLWRDGLLSYTAPVGQTNYLSEAVYLNDSTYDGH
TVGGLQYGGLGQLADGVVGLDDFRKSQELRVWPGYDYVGWSNHSFSSGYVEMEFEFDR
LRAFQAMQVHCNNMHTLGARLPGGVECRFRRGFAMAWEGEPMRHNLGGNLGDPRARAV
SVPLGGRVARFLQCRFLFACPNLLFSEISFISDVVNNSSPALGGTFPPAPNNPPGPPP
TNFSSLELEPRGQQPVAKAEGSPTAILIGCINAIILLLLLIIALMLWRLHWRRLLSKA
ERRVLEEELTVHLSVPGDTILINNRPGPREPPPYQEPRPRGNFPHSAPCVPNGSALLL
SNPAYRLLLATYARPPRGPGFPTPAWAKPTNTQAYSGDYMEPEKPGAPLLFPPFQNSV
PEYAEADIVTLQCVTGGNTYAVPALPPGAVGDGPPRVDFPRSRLRFKEKLGEGQFGEV
HLCEVDSPQDLVSLDFPLNVRKGHPLLVAVKILRPDATKNARNDFLKEVKINSRLKDP
NIIRLLGVCVQDDPLCMITDYMENGDLNQELSAHQLEDKAAEGAPGDGQAAQGPTISY
PMLLHVAAQIASGMRYLATLNFVHRDLATRNCLVGENFTIKIAGFGMSRNLYAGDYYR
VQGRAVLPIRWMAWECILNGKFTTASDVWAFGVTLWEVLMLCRAQPEGQLTDEQVIEN
AGEFFRDQGRQVYLSRPPACPQGLYELMLRCWSRESEQRPPFSQLHRFLAEDALNTV
GENBANK ID: M28212.1
VERSION M28212.1 GI:550071
/GENE=“RAB6”
CDS 71..697
/CODON_START=1

| | |
|---|---|
| 1 | AGCTGGCTGG AGCAGGATCG GTCCGGGACG GTCICTAGGC TGAGGCGGCG GCCGCTCCTC |
| 61 | TAGTTCCACA ATGTCCACGC GGGGAGACTT CGCGAATCCG CTGACGAAAT TCAAGCTGGT |
| 121 | GTTCCTGGGG GAGCAAAGCG TTGGAAAGAC ATCTTTGATC ACCAGATTCA TGTATGACAG |
| 181 | TTTTGACAAC ACCTATCAGG CAACAATTGG CATTGACTTT TTATCAAAAA CTATGTACTT |
| 241 | GGAGGATCGA ACAGTACGAT TGCAATTATG GGACACACCA GGTCAAGAGC GGTTCAGGAG |
| 301 | CTTGATTCCT AGCTACATTC GTGACTCCAC TGTGGCAGTT GTTGTTTATG ATATCACAAA |
| 361 | TGTTAACTCA TTCCAGCAAA CTACAAAGTG GATTGATGAT GTCAGAACAG AAAGAGGAAG |
| 421 | TGATGTTATC ATCATGCTAG TAGGAAATAA AACAGATCTT GCTGACAAGA GGCAAGTGTC |
| 481 | AATTGAGGAG GGAGAGAGGA AAGCCAAAGA GCTGAATGTT ATGTTTATTG AAACTAGTGC |
| 541 | AAAAGCTGGA TACAATGTAA AGCAGCTCTT TCGACGTGTA GCAGCAGCTT TGCCGGGAAT |
| 601 | GGAAAGCACA CAGGACAGAA GCAGAGAAGA TATGATTGAC ATAAAACTGG AAAAGCCTCA |
| 661 | GGAGCAACCA GTCAGTGAAG GAGGCTGTTC CTGCTAATGT CCCTAGTCAT CTTCAACCTT |
| 721 | CTTCAGAAGC TCACTGCTTT |

GENBANK ID: M65066.1
VERSION M65066.1 GI:307376
/GENE=“PRKAR1B”
CDS <1..1144
/CODON_START=2

| | |
|---|---|
| 1 | GGCCTCCCCG CCCGCCTGCC CCTCGGAGGA GGACCAGAGC CTGAAGGGCT GTGAGCTGTA |
| 61 | CGTGCAGCTG CACGGGATCC AGCAGGTCCT CAAAGACTGT ATCGTCCACC TCTGCATCTC |
| 121 | CAAGCCCGAA CGCCCCATGA AGTTCCTCCG GGAGCACTTC GAGAAGCTGG AGAAGGAAGA |
| 181 | AAACAGGCAG ATTTTGGCGC GGCAAAAGTC AAACTCACAG TCGGACTCCC ATGATGAGGA |
| 241 | GGTGTCGCCC ACCCCCCCGA ACCCTGTGGT GAAGGCCCGC CGCCGGCGAG GAGGCGTGAG |
| 301 | TGCCGAGGTG TACACCGAGG AGGACGCCGT GTCCTACGTC AGGAAGGTGA TTCCCAAGGA |
| 361 | CTACAAAACC ATGACTGCGC TGGCCAAGGC CATCTCCAAG AACGTGCTCT TCGCTCACCT |
| 421 | GGATGACAAC GAGAGGAGTG ACATATTCGA TGCCATGTTC CCTGTCACTC ACATCGCTGG |
| 481 | GGAGACTGTT ATACAGCAAG GGAATGAAGG AGACAACTTC TATGTCGTTG ATCAAGGGGA |
| 541 | AGTGGATGTG TACGTGAACG GAGAGTGGGT GACCAACATC AGCGAGGGAG GCAGCTTCGG |
| 601 | GGAGCTGGCG CTCATCTACG GCACCCCCAG GGCTGCGACC GTGAAAGCCA AGACGGACCT |
| 661 | CAAGCTCTGG GGGATCGACC CGGACAGCTA CCGGCGCATC CTTATGGGCA GCACGCTGAG |
| 721 | GAAACGCAAG ATGTACGAGG AGTTCCTCAG CAAGGTCTCC ATCCTAGAGT CCCTGGAGAA |
| 781 | GTGGGAGCGT CTGACCGTGG CGGATCGGCT GGAGCCCGTC CAGTTTGAAG ATGGAGAGAA |
| 841 | AATTGTGGTC CAGGGAGAGC CTCGGGACCA CTTTTACATC ATCACGGAGG GCACCGCGTC |
| 901 | CGTGCTGCAG CGCCGGTCCC CCAATGAGGA CTACCTCGAG GTGGGGCGCC TGGGACCCTC |
| 961 | TGACTACTTC GCGGAGATTC CACTCCTCCT GAACCGGCCC CGGGCGGCCA CTGTCGTGGC |
| 1021 | CCGGGGGCCC CTCAAGTGTG TGAAGCTGGA CCGGCCCCGC TTCGAGCGTG TGCTGGGGCC |
| 1081 | CTGCTCTGAG ATCCTCAAGA GGAACATTCA GCGTTACAAC AGCTTCATCT CCCTCACCGT |
| 1141 | CTGAGCACAC GTCCCGCCCT GCAGCCCCAG CTCCCCAGTG TGGTGGCCGT GCCTGCTCGT |
| 1201 | CTGTGTCGGG GGCCCGGGAG CCGCTGTGTG AGGIGIGGCC CGGGTGGGGC TGGGTCCCGG |
| 1261 | CAGCGTGAGG ACTGCCCCTT CCCCGGACTC ACTTTTTGGA ATAAATGATC ACCTTCTCCA |
| 1321 | TTTCCAAATC AAAGGACAAG CGGACAAAAT GCATCCCAAG ATCAAGGAAG GGACAGCCCA |
| 1381 | GCTTCCTCCC CACACGCCTC CCCGGCTGCC TCTGTGGGCT TCTCCTGGGG GGCCCACCCC |
| 1441 | ACCCCTGCCA GTCTCCTGGA GATGCTTGAG GATCGGTCCT CCCCAGAAGC AGGCCAGGAC |
| 1501 | GTTGCCCCTG GCGGCTGGTG ACCCTGTGAG GTCAGGTCCC CCAGATTGAG GTCTGAGTGT |
| 1561 | GGGCAAGTGT GTCAAAAGGG GCTGCCCCCC AGGAGATGAG GCTGAGAGCA GGGAGTTGAG |
| 1621 | GCCGAAGAAG TCAAGGCCCC TCCCGCAAAT GTGTACCCCT GCCCGCGCCA CTGCACCCCG |
| 1681 | CCGCACCCCC ACCTCCCCGG GGGCCCTGCT GCGGATCGCG GACTGGGAGA GTCTCTGAGC |
| 1741 | TATGAGATTG ATCTTGCCCC TAATTGGAGA GGAAGCCGGG CGCCAAGACA CACGGGGCTC |
| 1801 | CTGCCTTGGG AGCCAGGGCC GCGGCCGCAG GTAGACCCCA GTAGGGGGGG CCGGGCTCGA |
| 1861 | AGTTCCTTTG GGAGGGGCTG GCCGGACTCC AGCAGGCCCT CCTCACCTTT CTTAGAAAGT |
| 1921 | CCACCCAGGG CAAGTTGATG TTGGGGGAAA GCAGAAGTCA AGCCAGCCGC GGCCCCACAC |
| 1981 | GCCCCCACCC GCTCAACCGA CTTGTCCCTT AAATGTGTCT TGGATCCCGC AGTGATGACG |
| 2041 | TGAGCCAGCC AGGCCCGAAA AGGTGAGGCC ACTGCAGAGA AGCTTCCCAG GGGATTCCTG |
| 2101 | GTTCCCCCGA AACACAAGCG AGGTCATTGC AGTTCACCCG ATGTTGCTCC TGTCCCGTGC |
| 2161 | GTCCCCGGGA GGCTGTCCTT GGTCCGCATG GCTCGTTGCA GCCCCTCCCC TGCTGGCGGT |
| 2221 | TACGATGCGT GGGGTCCCCC TCCCCACCCA GCCCCGGCAC CGTCGCCGTG TCCCGCCTGT |
| 2281 | GCCTGTGACG TCCCTGTGGA CCTGTGAGCC ATCCCCCCCT TATCTCTGCT CTGAATACTG |
| 2341 | CATGAAGCAT TAAACGTGCA ATGAAG |

GENBANK ID: U04897.1
VERSION U04897.1 GI:451563
CDS 102..1673
/CODON_START=1

| | |
|---|---|
| 1 | GTTTTTTTTT TTTTTTTGGT ACCATAGAGT TGCTCTGAAA ACAGAAGATA GAGGGAGTCT |
| 61 | CGGAGCTCGC ATCTCCAGCG ATCTCTACAT TGGGAAAAAA CATGGAGTCA GCTCCGGCAG |
| 121 | CCCCCGACCC CGCCGCCAGC GAGCCAGGCA GCAGCGGCGC GGACGCGGCC GCCGGCTCCA |
| 181 | GGGAGACCCC GCTGAACCAG GAATCCGCCC GCAAGAGCGA GCCGCCTGCC CCGGTGCGCA |
| 241 | GACAGAGCTA TTCCAGCACC ACCAGAGGTA TCTCAGTAAC GAAGAAGACA CATACATCTC |
| 301 | AAATTGAAAT TATTCCATGC AAGATCTGTG GAGACAAATC ATCAGGAATC CATTATGGTG |
| 361 | TCATTACATG TGAAGGCTGC AAGGGCTTTT TCAGGAGAAG TCAGCAAAGC AATGCCACCT |
| 421 | ACTCCTGTCC TCGTCAGAAG AACTGTTTGA TTGATCGAAC CAGTAGAAAC CGCTGCCAAC |
| 481 | ACTGTCGATT ACAGAAATGC CTTGCCCTAG GGATGTCTCG AGATGCTGTA AAATTTGGCC |
| 541 | GAATGTCAAA AAAGCAGAGA GACAGCTTGT ATGCAGAAGT ACAGAAACAC CGGATGCAGC |
| 601 | AGCAGCAGCG CGACCACCAG CAGCAGCCTG GAGAGGCTGA GCCGCTGACG CCCACCTACA |
| 661 | ACATCTCGGC CAACGGGCTC ACGGAACTTC ACGACGACCT CAGTAACTAC ATTGACGGGC |
| 721 | ACACCCCTGA GGGGAGTAAG GCACACTCCG CCGTCAGCAG CTTCTACCTG GACATACAGC |
| 781 | CTTCCCCAGA CCAGTCAGGT CTTGATATCA ATGGAATCAA ACCAGAACCA ATATGTGACT |
| 841 | ACACACCAGC ATCAGGCTTC TTTCCCTACT GTTCCTTCAC CAACGGCGAG ACTTCCCCAA |
| 901 | CTGTGTCCAT GGCAGAATTA GAACACCTTG CACAGAATAT ATCTAAATCG CATCTGGAAA |
| 961 | CCTGCCAATA CTTGAGAGAA GAGCTCCAGC AGATAACGTG GCAGACCTTT TTACAGGAAG |
| 1021 | AAATTGAGAA CTATCAAAAC AAGCAGCGGG AGGTGATGTG GCAATTGTGT GCCATCAAAA |
| 1081 | TTACAGAAGC TATACAGTAT GTGGTGGAGT TTGCCAAACG CATTGATGGA TTTATGGAAC |
| 1141 | TGTGTCAAAA TGATCAAATT GTGCTTCTAA AAGCAGGTTC TCTAGAGGTG GTGTTTATCA |
| 1201 | GAATGTGCCG TGCCTTTGAC TCTCAGAACA ACACCGTGTA CTTTGATGGG AAGTATGCCA |
| 1261 | GCCCCGACGT CTTCAAATCC TTAGGTTGTG AAGACTTTAT TAGCTTTCTG TTTGAATTTG |
| 1321 | GAAAGAGTTT ATCTTCTATG CACCTGACTG AAGATGAAAT TGCATTATTT TCTGCATTTG |
| 1381 | TACTGATGTC AGCAGATCGC TCATGGCTGC AAGAAAAGGT AAAAATTGAA AAACTGCAAC |
| 1441 | AGAAAATTCA GCTAGCTCTT CAACACGTCC TACAGAAGAA TCACCGAGAA GATGGAATAC |
| 1501 | TAACAAAGTT AATATGCAAG GTGTCTACAT TAAGAGCCTT ATGTGGACCA CATACAGAAA |
| 1561 | AGCTAATGGC ATTTAAAGCA ATATACCCAG ACATTGTGCG ACTTCATTTT CCTCCATTAT |
| 1621 | ACAAGGAGTT GTTCACTTCA GAATTTGAGC CAGCAATGCA AATTGATGGG TAAATGTTAT |
| 1681 | CACCTAAGCA CTTCTAGAAT GTCTGAAGTA CAAACATGAA AAACAAACAA AAAAATTAAC |
| 1741 | CGAGACACTT TATATGGCCC TGCACAGACC TGGAGCGCCA CACACTGCAC ATCTTTTGGT |
| 1801 | GATCGGGGTC AGGCAAAGGA GGGGAAACAA TGAAAACAAA TAAAGTTGAA CTTGTTTTTC |
| 1861 | TCA |

GENBANK ID: P53667
DBEST ID: 1741245
EST NAME: AN07C08.S1
ACTGGGCTCCCCGGTCTCCCATCGCAAGGACCTGGGTCGCTCTGAGTCCCTCCGCGTAGT
CTGCCGGCCACACCGCATCTTCCGGCCGTCGTATCTCATCCACGGTGAGGTGCTGGGCAA
GGGCTGCTTCGGCTATGCTATCAATGTGACATACTGTGAGACAGGTGATGTGATGGTGAT
GAAGGAGCTGATCCGGTTCGACGAGGAGACCCAGAGGACGTTCCTCAACGAGGTGAATGT
CATT
GENBANK ID: NM_002737.1
VERSION NM_002737.1 GI:4506066
CDS 28..2046

| | |
|---|---|
| 1 | GGAGCAAGAG GTGGTTGGGG GGGGACCATG GCTGACGTTT TCCCGGGCAA CGACTCCACG |
| 61 | GCGTCTCAGC ACGTGGCCAA CCGCTTCGCC CCCAAACGCG CGCTGAGGCA GAAGAACGTG |
| 121 | CACGAGGTGA AGGACCACAA ATTCATCGCG CGCTTCTTCA AGCAGCCCAC CTTCTCCAGC |
| 181 | CACTGCACCG ACTTCATCTG GGGGTTTGGG AAACAAGGCT TCCAGTGCCA AGTTTGCTGT |
| 241 | TTTGTGGTCC ACAAGAGCTG CCATGAATTT CTTACTTTTT CTTGTCCGGC TGCGGATAAG |
| 301 | GGACCCGACA CTGATGACCC CAGGAGCAAC CACAAGTTCA AAATCCACAC TTACGCAAGC |
| 361 | CCCACCTTCT GCGATCACTC TGGGTCACTG CTCTATGGAC TTATCCATCA AGCGATGAAA |
| 421 | TCTGACACCT GCGATATGAA CGTTCACAAC CAATGCGTCA TCAATGTCCC CACCGTGTGC |
| 481 | GCAATCGATC ACACTGAGAA GAGGGCGCGG ATTTACCTAA AGGCTGAGCT TGCTGATCAA |
| 541 | AAGCTCCATG TCACAGTACG ACATGCAAAA AATCTAATCC CTATGGATCC AAACGGCCTT |
| 601 | TCAGATCCTT ATGTGAAGCT GAAACTTATT CCTGATCCCA AGAATGAAAG CAACCAAAAA |
| 661 | ACCAAAACCA TCCGCTCCAC ACTAAATCCG CAGTGCAATG ACTCCTTTAC ATTCAAATTG |
| 721 | AAACCTTCAG ACAAAGACCG ACGACTGTCT GTACAAATCT GCGACTGCGA TCGAACAACA |
| 781 | AGGAATCACT TCATGGGATC CCTTTCCTTT GGAGTTTCGC ACCTGATGAA GATCCCGGCC |
| 841 | AGTGGATGGT ACAAGTTCCT TAACCAACAA GAACGTGAGT ACTACAACGT ACCCATTCCC |
| 901 | GAACGGGACG AGGAAGGAAA CATCGAACTC ACGCAGAAAT TCGAGAAACC CAAACTTCCC |
| 961 | CCTGCTGCCA ACAAAGTCAT CAGTCCCTCT GAAGACAGGA AACAACCTTC CAACAACCTT |
| 1021 | GACCGAGTGA AACTCACGGA CTTCAATTTC CTCATGGTGT TGGGAAACGC CAGTTTTCCA |
| 1081 | AAGGTCATGC TTGCC3ACAG GAAGGGCACA GAACAACTGT ATGCAATCAA AATCCTGAAG |
| 1141 | AACGATGTGG TGATTCAGCA TGATGACGTG GAGTGCACCA TGGTAGAAAA GCGACTCTTC |
| 1201 | GCCCTGCTTG ACAAACCCCC GTTCTTGACC CAGCTGCACT CCTCCTTCCA GACAGTGGAT |
| 2261 | CGGCTCTACT TCGTCATGGA ATATGTCAAC GGTGGGGACC TCATGTACCA CATTCAGCAA |
| 1321 | GTAGGA~AAT TTAAGGAACC ACAAGCAGTA TTCTATGCGC CAGAGATTTC CATCGGATTG |
| 1381 | TTCTTTCTTC ATAAAAGAGG AATCATTTAT AGGGATCTCA ACTTACATAA CGTCATGTTG |
| 1441 | GATTCAGAAG GACATATCAA AATTGCTGAC TTTGGGATGT GCAAGGAACA CATGATGGAT |
| 1501 | GGAGTCACGA CCAGGACCTT CTGTGCGACT CCACATTATA TCGCCCCAGA GATAATCCCT |
| 1561 | TATCAGCCGT ATGGAAAATC TGTGGACTGG TGGGCCTATC CCGTCCTGTT GTATGAAATG |
| 1621 | CTTGCCGCGC AGCCTCCATT TGATGGTCAA GATGAAGACG AGCTATTTCA GTCTATCATG |
| 1681 | GAGCACAACG TTTCCTATCC AAAATCCTTG TCCAAGCAGC CTGTTTCTAT CTGCAAAGGA |
| 1741 | CTGATGACCA AACACCCAGC CAAGCGCCTG GGCTGTGGGC CTGAGGGGGA GAGGGACGTG |
| 1801 | AGAGAGCATG CCTTCTTCCG GAGGATCGAC TGGGAAAAAC TGGAGAACAG GGAGATCGAG |
| 1861 | CCACCATTCA AGCCCAAAGT GTGTGGCAAA GGACCACAGA ACTTTGACAA GTTCTTCACA |
| 1921 | CGAGGACAGC CCGTCTTAAC ACCACCTGAT CAGCTGGTTA TTGCTAACAT AGACCAGTCT |
| 1981 | GATTTTGAAG GGTTCTCGTA TGTCAACCCC CAGTTTGTGC ACCCCATCTT ACAGACTGCA |
| 2041 | GTATGAAACT CACCAGCGAG AACAAACACC TCCCCAGCCC CCAGCCCTCC CCGCAGTGGA |
| 2101 | AGTGAATCCT TAACCCTAAA ATTTTAAGGC CACGGCTTGT GTCTGATTCC ATATGGAGGC |
| 2161 | CTGAAAATTG TAGGGTTATT AGTCCAAATG TGATCAACTG TTCAGGGTCT CTCTCTTACA |
| 2221 | ACCAAGAACA TTATCTTAGT GGAAG |

GENBANK ID: M16038.1
VERSION M16038.1 GI:187268
MGCIKSKGKDSLSDDGVDLKTQPVRNTERTIYVRDPTSNKQQRP
VPESQLLPGQRFQTKDPEEQGDIVVALYPYDGIHPDDLSFKKGEKMKVLEEHGEWWKA
KSLLTKKEGFIPSNYVAKLNTLETEEWFFKDITRKDAERQLLAPGNSAGAFLIRESET
LKGSFSLSVRDFDPVHGDVIKHYKIRSLDNGGYYISPRITFPCISDMIKHYQKQADGL
CRRLEKACISPKPQKPWDRDANEIPRESIKLVKRLGAGQFGEVWMGYYNNSTKVAVKT
LKPGTNSVQAFLEEANLMKTLQHDKLVRLYAVVTREEPIYIITEYMAKGSLLDFLKSD
EGGKVLLPKLIDFSAQIAEGMAYIERKNYIHRDLRAANVLVSESLMCKIADFGLARVI
EDNEYTAREGAKFPIKWTAPEAINFGCFTIKSDVWSFGILLYEIVTYGKIPYPGRTNA
DVMTALSQGYRMPRVENCPDELYDIMKNCWKEKAEERPTFDYLQSVLDDFYTATEGQY
QQQP
GENBANK ID: 012128.1
VERSION 012128.1 GI:557287
CDS 218..7690
/GENE=“PTP1E”
/CODON_START=1

| | |
|---|---|
| 1 | CTGATTATGA AGTGCCTCAG AGCCAACCTA TTAAGCTTGG AGATCATCTC AACAGCATAC |
| 61 | TGCTTGGAAT GTGTGAGGAT GTTAITTACG CTCGAGTTTC TGTICGGACT GTGCTGGATG |
| 121 | CTTGCAGTGC CCACATTAGG AATAGCAATT GTGCACCCTC ATTTTCCTAC GTGAAACACT |
| 181 | TGGTAAAACT GGTICTGGGA AATCTTTCTG GGGTAATATG CACGTGTCAC TAGCTGAGGC |
| 241 | CCTGGAGGTT CGGGGTGGAC CACTTCAGGA GGAAGAAATA TGGGCTGTAT TAAATCAAAG |
| 301 | TGCTGAAAGT CTCCAAGAAT TATTCAGAAA AGTAAGCCTA GCTGATCCTG CTGCCCTTGG |
| 361 | CTTCATCATT TCTCCATGGT CTCTGCTGTT GCTGCCATCT GGTAGTGTGT CATTTACAGA |
| 421 | TGAAAATATT TCCAATCAGG ATCTTCCAGC ATTCACTGCA CCAGAGGTTC TTCAAAATCA |
| 481 | GTCACTAACT TCTCTCTCAG ATGTTGAAAA GATCCACATT TATTCTCTTG GAATGACACT |
| 541 | GTATTGGGGG GGTGATTATG AAGTGCCTCA GAGCCAACCT ATTAAGCTTG GAGATCATCT |
| 603 | CAACAGCATA CTGCTTGGAA TGTGTGAGGA TGTTATTTAC GCTCGAGTTT CTGTTCGGAC |
| 661 | TGTGCTGGAT GCTTGCAGTG CCCACATTAG GAATAGCAAT TGTGCACCCT CATTTTCCTA |
| 721 | CGTGAAACAC TTGGTAAAAC TGGTTCTGGG AAATCTTTCT GCGACAGATG AGCTTTCCTG |
| 781 | TAACAGTGAA CAAAAGCCTG ATCGAAGCCA GGCTATTCGA GATCGATTGC GAGGAAAAGG |
| 841 | ATTACCAACA GGAAGAAGCT CTAGTTCTGA TGTACTAGAC ATACAAAAGC CTCCACTCTC |
| 901 | TCATCAGACC TTTCTTAACA AAGGGCTTAG TAAATCTATG GGATTTCTGT CCATCAAAGA |
| 961 | TACACAAGAT GAGAATTATT TCAAGGACAT TTTATCAGAT AATTCTGGAC GTGAAGATTC |
| 1021 | TGAAAATACA TTCTCCCCTT ACCAGTTCAA AACTAGTGGC CCAGAAAAAA AACCCATCCC |
| 1081 | TGGCATTGAT GTGCTTTCTA AGAAGAAGAT CTGGGCTTCA TCCATGGACT TGCTTTGTAC |
| 1141 | AGCTGACAGA GACTTCTCTT CAGGAGAGAC TGCCACATAT CGTCGTTGTC ACCCTGAGGC |
| 1201 | AGTAACAGTG CGGACTTCAA CTACTCCTAG AAAAAAGGAG GCAAGATACT CAGATGGAAG |
| 1261 | TATAGCCTTG GATATCTTTG GCCCTCAGAA AATGGATCCA ATATATCACA CTCGAGAATT |
| 1321 | GCCCACCTCC TCAGCAATAT CAAGTGCTTT CGACCGAATC CGAGAGAGAC AAAAGAAACT |
| 1381 | TCAGGTTCTG AGGGAAGCCA TGAATGTAGA AGAACCAGTT CGAAGATACA AAACTTATCA |
| 1441 | TGGTGATGTC TTTAGTACCT CCAGTGAAAG TCCATCTATT ATTTCCTCTG AATCAGATTT |
| 1501 | CAGACAAGTG AGAAGAAGTG AAGCCTCAAA GAGGTTTGAA TCCAGCAGTG GTCTCCCAGG |
| 1561 | GGTAGATGAA ACCTTAAGTC AAGGCCAGTC ACAGAGACGG AGCAGACAAT ATGAAACACC |
| 1621 | CTTTGAAGGC AACTTAATTA ATCAAGAGAT CATGCTAAAA CGGCAAGAGG AAGAACTGAT |
| 1681 | GCAGCTACAA GCCAAAATGG CCCTTAGACA GTCTCGGTTG AGCCTATATC CAGGAGACAC |
| 1741 | AATCAAAGCG TCCATGCTTG ACATCACCAG GGATCCGTTA AGAGAAATTG CCCTAGAAAC |
| 1801 | AGCCATGACT CAAAGAAAAC TGAGGAATTT CTTTGGCCCT GAGTTTGTGA AAATGACAAT |
| 1861 | TGAACCATTT ATATCTTTGG ATTTGCCACG GTCTATTCTT ACTAAGAAAG GGAAGAATGA |
| 1921 | GGATAACCGA AGGAAAGTAA ACATAATGCT TCTGAACGGG CAAAGACTGG AACTGACCTG |
| 1981 | TGATACCAAA ACTATATGTA AAGATGTGTT TGATATGGTT GTGGCACATA TTGGCTTAGT |
| 2041 | AGAGCATCAT TTGTTTGCTT TAGCTACCCT CAAAGATAAT GAATATTTCT TTGTTGATCC |
| 2101 | TGACTTAAAA TTAACCAAAG TGGCCCCAGA GGGATGGAAA GAAGAACCAA AGAAAAAGAC |
| 2161 | CAAAGCCACT GTTAATTTTA CTTTGTTTTT CAGAATTAAA TTTTTTATGG ATGATGTTAG |
| 2221 | TCTAATACAA CATACTCTGA CGTGTCATCA GTATTACCTT CAGCTTCGAA AAGATATTTT |
| 2281 | GGAGGAAAGG ATGCACTGTG ATGATGAGAC TTCCTTATTG CTGGCATCCT TGGCTCTCCA |
| 2341 | GGCTGAGTAT GGAGATTATC AACCAGAGGT TCATGGTGTG TCTTACTTTA GAATGGAGCA |
| 2401 | CTATTTGCCC GCCAGAGTGA TGGAGAAACT TGATTTATCC TATATCAAAG AAGAGTTACC |
| 2461 | CAAATTGCAT AATACCTATG TGGGAGCTTC TGAAAAAGAG ACAGAGTTAG AATTTTTAAA |
| 2521 | GGTCTGCCAA AGACTGACAG AATATGGAGT TCATTTTCAC CGAGTGCACC CTGAGAAGAA |
| 2581 | GTCACAAACA GGAATATTGC TTGGAGTCTG TTCTAAAGGT GTCCTTGTGT TTGAAGTTCA |
| 2641 | CAATGGAGTG CGCACATTGG TCCTTCGCTT TCCATGGAGG GAAACCAAGA AAATATCTTT |
| 2701 | TTCTAAAAAG AAAATCACAT TGCAAAATAC ATCAGATGGA ATAAAACATG GCTTCCAGAC |
| 2761 | AGACAACAGT AAGATATGCC AGTACCTGCT GCACCTCTGC TCTTACCAGC ATAAGTTCCA |
| 2821 | GCTACAGATG AGAGCAAGAC AGAGCAACCA AGATGCCCAA GATATTGAGA GAGCTTCGTT |
| 2881 | TAGGAGCCTG AATCTCCAAG CAGAGTCTGT TAGAGGATTT AATATGGGAC GAGCAATCAG |
| 2941 | CACTGGCAGT CTGGCCAGCA GCACCCTCAA CAAACTTGCT GTTCGACCTT TATCAGTTCA |
| 3001 | AGCTGAGATT CTGAAGAGGC TATCCTGCTC AGAGCTGTCG CTTTACCAGC CATTGCAAAA |
| 3061 | CAGTTCAAAA GAGAAGAATG ACAAAGCTTC ATGGGAGGAA AAGCCTAGAG AGATGAGTAA |
| 3121 | ATCATACCAT GATCTCAGTC AGGCCTCTCT CTATCCACAT CGGAAAAATG TCATTGTTAA |
| 3181 | CATGGAACCC CCACCACAAA CCGTTGCAGA GTTGGTGGGA AAACCTTCTC ACCAGATGTC |
| 3241 | AAGATCTGAT GCAGAATCTT TGGCAGGAGT GACAAAACTT AATAATTCAA AGTCTGTTGC |
| 3301 | GAGTTTAAAT AGAAGTCCTG AAAGGAGGAA ACATGAATCA GACTCCTCAT CCATTGAAGA |
| 3361 | CCCTGGGCAA GCATATGTTC TAGGAATGAC TATGCATAGT TCTGGAAACT CTTCATCCCA |
| 3421 | AGTACCCTTA AAAGAAAATG ATGTGCTACA CAAAAGATGG AGCATAGTAT CTTCACCAGA |
| 3481 | AAGGGAGATC ACCTTAGTGA ACCTGAAAAA AGATGCAAAG TATGGCTTGG GATTTCAAAT |
| 3541 | TATTGGTGGG GAGAAGATGG GAAGACTGGA CCTAGGCATA TTTATCAGTT CAGTTGCCCC |
| 3601 | TGGAGGACCA GCTGACTTGG ATGGATGCTT GAAGCCAGGA GACCGTTTGA TATCTGTGAA |
| 3661 | TAGTGTGAGT CTGGAGGGAG TCAGCCACCA TGCTGCAATT GAAATTTTGC AAAATGCACC |
| 3721 | TGAAGATGTG ACACTTGTTA TCTCTCAGCC AAAAGAAAAG ATATCCAAAG TGCCTTCTAC |
| 3781 | TCCTGTGCAT CTCACCAATG AGATGAAAAA CTACATGAAG AAATCTTCCT ACATGCAAGA |
| 3841 | CAGTGCTATA GATTCTTCTT CCAAGGATCA CCACTGGTCA CGTGGTACCC TGAGGCACAT |
| 3901 | CTCGGAGAAC TCCTTTGGGC CGTCTGGGGG CCTGCGGGAA GGAAGCCTGA GTTCTCAAGA |
| 3961 | TTCCAGGACT GAGAGTGCCA GCTTGTCTCA AAGCCAGGTC AATGGTTTCT TTGCCAGCCA |
| 4021 | TTTAGGTGAC CAAACCTGGC AGGAATCACA GCATGGCAGC CCTTCCCCAT CTGTAATATC |
| 4081 | CAAAGCCACC GAGAAAGAGA CTTTCACTGA TAGTAACCAA AGCAAAACTA AAAAGCCAGG |
| 4141 | CATTTCTGAT GTAACTGATT ACTCAGACCG TGGAGATTCA GACATGGATG AAGCCACTTA |
| 4201 | CTCCAGCAGT CAGGATCATC AAACACCAAA ACAGGAATCT TCCTCTTCAG TGAATACATC |
| 4261 | CAACAAGATG AATTTTAAAA CTTTTTCTTC ATCACCTCCT AAGCCTGGAG ATATCTTTGA |
| 4321 | GGTTGAACTG GCTAAAAATG ATAACAGCTT GGGGATAAGT GTCACGGTAC TGTTTGACAA |
| 4281 | GGGAGGTGTG AATACGAGTG TCAGACATGG TGGCATTTAT GTGAAAGCTG TTATTCCCCA |
| 4441 | GGGAGCAGCA GAGTCTGATG GTAGAATTCA CAAAGGTGAT CCCGTCCTAC CTGTCAATGG |
| 4501 | AGTTAGTCTA GAAGGAGCCA CCCATAAGCA AGCTCTGGAA ACACTCACAA ATACAGGACA |
| 4561 | GGTGCTTCAT CTGTTATTAG AAAAGGGACA ATCTCCAACA TCTAAAGAAC ATGTCCCGGT |
| 4621 | AACCCCACAG TGTACCCTTT CAGATCAGAA TGCCCAAGGT CAAGGCCCAG AAAAACTGAA |
| 4681 | GAAAACAACT CAGGTCAAAG ACTACAGCTT TGTCACTGAA GAAAATACAT TTGAGGTAAA |
| 4741 | ATTATTTAAA AATAGCTCAG GTCTAGGATT CAGTTTTTCT CGAGAAGATA ATCTTATACC |
| 4801 | GGAGCAAATT AATGCCAGCA TAGTAAGGGT TAAAAAGCTC TTTCCTGGAC AGCCAGCAGC |
| 4861 | AGAAAGTGGA AAAATTGATC TAGGAGATGT TATCTTGAAA GTGAATGGAG CCTCTTTGAA |
| 4921 | AGGACTATGT CAGCAGGAAG TCATATCTGC TCTCAGGGGA ACTGCTGCAG AAGTATTCTT |
| 4981 | GCTTCTCTGC AGACCTCCAC CTGGTGTGCT ACCGGAAATT GATACTGCGC TTTTGACCCC |
| 5041 | ACTTCAGTCT CCAGCACAAG TACTTCCAAA CAGCAGTAAA GACTCTTCTC AGCCATCATG |
| 5101 | TGTGGAGCAA AGCACCAGCT CAGATGAAAA TGAAATGTCA GACAAAAGCA AAAAACAGTG |
| 5161 | CAAGTCCCCA TCCAGAAGAG ACAGTTACAG TGACAGCAGT GGGAGTGGAG AAGATGACTT |
| 5221 | AGTGACAGCT CCAGCAAACA TATCAAATTC GACCTGGAGT TCAGCTTTGC ATCAGACTCT |
| 5281 | AAGCAACATG GTATCACAGG CACAGAGTCA TCATGAAGCA CCCAAGAGTC AAGAAGATAC |
| 5241 | CATTTGTACC ATGTTTTACT ATCCTCAGAA AATTCCCAAT AAACCAGAGT TTGAGGACAG |
| 5401 | TAATCCTTCC CCTCTACCAC CGGATATGGC TCCTGGGCAG AGTTATCAAC CCCAATCAGA |
| 5461 | ATCTGCTTCC TCTAGTTCGA TGGATAAGTA TCATATACAT CACATTTCTG AACCAACTAG |
| 5521 | ACAAGAAAAC TGGACACCTT TGAAAAATGA CTTGGAAAAT CACCTTGAAG ACTTTGAACT |
| 5581 | GGAAGTACAA CTCCTCATTA CCCTAATTAA ATCAGAAAAA GGAAGCCTGG GTTTTACAGT |
| 5641 | AACCAAAGCC AATCAGAGAA TTGGTTGTTA TGTTCATGAT GTCATACAGG ATCCAGCCAA |
| 5701 | AAGTGATGGA AGGCTAAAAC CTCGGGACCG GCTCATAAAG GTTAATGATA CAGATGTTAC |
| 5761 | TAATATGACT CATACAGATG CAGTTAATCT GCTCCGGGCT GCATCCAAAA CAGTCAGATT |
| 5821 | AGTTATTCGA CGAGTTCTAG AATTACCCAG AATACCAATG TTGCCTCATT TGCTACCGGA |
| 5881 | CATAACACTA ACGTGCAACA AAGAGGAGTT GGGTTTTTCC TTATGTGGAG GTCATGACAG |
| 5941 | CCTTTATCAA GTGGTATATA TTAGTGATAT TAATCCAAGG TCCGTCGCAG CCATTGAGGG |
| 6001 | TAATCTCCAG CTATTAGATG TCATCCATTA TGTGAACGGA GTCAGCACAC AAGGAATGAC |
| 6061 | CTTGGAGGAA GTTAACAGAG CATTAGACAT CTCACTTCCT TCATTGGTAT TGAAACCAAC |
| 6121 | AAGAAATGAT CTTCCAGTGG TCCCCAGCTC AAAGAGGTCT GCTGTTTCAG CTCCAAAGTC |
| 6181 | AACCAAAGGC AATGGTTCCT ACAGTGTGGG GTCTTCCAGC CAGCCTGCCC TCACTCCTAA |
| 6241 | TGATTCATTC TCCACGGTTG CTGGGGAAGA AATAAATGAA ATATCGTACC CCAAAGGAAA |
| 6301 | ATGTTCTACT TATCAGATAA AGGGATCACC AAACTTGACT CTGCCCAAAG AATCTTATAT |
| 6261 | ACAAGAAGAT GACATTTATG ATGATTCCCA AGAAGCTGAA GTTATCCAGT CTCTGCTGGA |
| 6421 | TGTTGTGGAT GAGGAAGCCC AGAATCTTTT AAACGAAAAT AATGCAGCAG GATACTCCTG |
| 6481 | TGGTCCAGGT ACATTAAAGA TGAATGGGAA GTTATCAGAA GAGAGAACAG AAGATACAGA |
| 6541 | CTGCGATGGT TCACCTTTAC CTGAGTATTT TACTGAGGCC ACCAAAATGA ATGGCTGTGA |
| 6601 | AGAATATTGT GAAGAAAAAG TAAAAAGTGA AAGCTTAATT CAGAAGCCAC AAGAAAAGAA |
| 6661 | GACTGATGAT GATGAAATAA CATGGGGAAA TGATGAGTTG CCAATAGAGA GAACAAACCA |
| 6721 | TGAAGATTCT GATAAAGATC ATTCCTTTCT GACAAACGAT GAGCTCGCTG TACTCCCTGT |
| 6781 | CGTCAAAGTG CTTCCCTCTG GTAAATACAC GGGTGCCAAC TTAAAATCAG TCATTCGAGT |
| 6841 | CCTGCGGGGT TTGCTAGATC AAGGAATTCC TTCTAAGGAG CTGGAGAATC TTCAAGAATT |
| 6801 | AAAACCTTTG GATCAGTGTC TAATTGGGCA AACTAAGGAA AACAGAAGGA AGAACAGATA |
| 6961 | TAAAAATATA CTTCCCTATG ATGCTACAAG AGTGCCTCTT GGAGATGAAG GTGGCTATAT |
| 7021 | CAATGCCAGC TTCATTAAGA TACCAGTTGG GAAAGAAGAG TTCGTTTACA TTGCCTGCCA |
| 7081 | AGGACCACTG CCTACAACTG TTGGAGACTT CTGGCAGATG ATTTGGGAGC AAAAATCCAC |
| 7141 | AGTGATAGCC ATGATGACTC AAGAAGTAGA AGGAGAAAAA ATCAAATGCC AGCGCTATTG |
| 7201 | GCCCAACATC CTAGGCAAAA CAACAATGGT CAGCAACAGA CTTCGACTGG CTCTTGTGAG |
| 7261 | AATGCAGCAG CTGAAGGGCT TTGTGGTGAG GGCAATGACC CTTGAAGATA TTCAGACCAG |
| 7221 | AGAGGTGCGC CATATTTCTC ATCTGAATTT CACTGCCTGG CCAGACCATG ATACACCTTC |
| 7381 | TCAACCAGAT GATCTGCTTA CTTTTATCTC CTACATGAGA CACATGCACA GATCAGGCCC |
| 7441 | AATCATTACG CACTGCAGTG CTGGCATTGG ACGTTCAGGG ACCCTGATTT GCATAGATGT |
| 7501 | GGTTCTGGGA TTAATCAGTC AGGATCTTGA TTTTGACATC TCTGATTTGG TGCGCTGCAT |
| 7561 | GAGACTACAA AGACACGGAA TGGTTCAGAC AGAGGATCAA TATATTTTCT GCTATCAAGT |
| 7621 | CATCCTTTAT GTCCTGACAC GTCTTCAAGC AGAAGAAGAG CAAAAACAGC AGCCTGAGGT |
| 7681 | TCTGAAGTGA CATGAAAAGA GCCTCTGGAT GCATTTCCAT TTCTCTCCTT AACCTCCAGC |
| 7741 | AGACTCCTGC TCTCTATCCA AAATAAGATC ACAGAGCAGC AAGTTCATAC AACATGCATG |
| 7801 | TTCTCCTCTA TCTTAGAGGG GTATTCTTCT TGAAAATAAA AAATATTGAA ATGCTGTATT |
| 7861 | TTTACAGCTA CTTTAACCTA TGATAATTAT TTACAAAATT TTAACACTAA CCAAACAATG |
| 7821 | CAGATCTTAG GGATGATTAA AGGCAGCATT TGATGATAGC AGACATTGTT ACAAGGACAT |
| 7981 | GGTGAGTCTA TTTTTAATGC ACCAATCTTG TTTATAGCAA AAATGTTTTC CAATATTTTA |
| 8041 | ATAAAGTAGT TATTTTATAG GGGATACTTG AAACCAGTAT TTAAGCTTTA AATGACAGTA |
| 8101 | ATATTGGCAT AGAAAAAAGT AGCAAATGTT TACTGTATCA ATTTCTAATG TTTACTATAT |
| 8161 | AGAATTTCCT GTAATATATT TATATACTTT TTCATGAAAA TGGAGTTATC AGTTATCTGT |
| 8221 | TTGTTACTGC ATCATCTGTT TGTAATCATT ATCTCACTTT GTAAATAAAA ACACACGTTA |
| 8281 | AAACATG |

GENEANK ID: NM_003326.1
VERSION NM_003336.1 GI:4507768
MSTPARRRLNRDFKRLQEDFPAGVSGAPSENNINVWNAVIFGPE
GTPFGDGTFKLTIEFTEEYPNKPPTVRFVSKMFHPNVYADGSICLDILQNRWSPTYDV
SSILTSIQSLLDEPNPNSPANSQAAQLYQENKREYEKRVSAIVEQSWRDC
GENBANK ID: S40706.1
VERSION S40706.1 GI:252001
MAAESLPFSSDTVSWELEAWYEDLQEVLSSDENCGTYVSPPGNE
SEESKIETTLDPASLAWLTEEEPEPAEVTSTSQSPHSPDSSQSSLAQEEEEEDQGRTR
KRKQSGHSPARAGKQRNKEKEQENERKVAQLAEENERLKQEIERLTREVEATRRALID
RMVNLHQA
GENBANK ID: M96995.1
VERSION M96995.1 GI:181975
MEAIAKYDEKATADDELSFKRGDILKVLNEECDQNNYKAELNGK
DGFIPKNYIEMKPHPWFFGKIPRAKAEENLSKQRHDGAFLIRESESAPGDFSLSVKFG
NDVQWFKVLRDGAGKYFLWVVKFNSLNELVDYHRSTSVSRNQQIFLRDIEQVPQQPTY
VQALFDFDPQEDGELGFRRGDFIHVMDNSDPNWWKGACNGQTGMFPRNYVTPVNRNV
GENBANK ID: M21758.1
VERSION M21758.1 GI:183664
MAEKPKLHYFNARGRNESTRWLLAAAGVEFEEKFIKSAEDLDKL
RNDGYLNEQQVPNVEIDGMKLVQTRAILNYIASKYNLYGKDIKERALIDMYIEGIADL
GENILLLPVCPPEEKDAKLALIKEKIKNRYFPAEEKVLKSHGQDYLVGNKLSRADIHL
VELLYYVEELDSSLISSEPLLKALKTRISNLPTVKKFLQPGSPRKPPNDEKSLEEARK
IFRF
GENBANK ID: U32944.1
VERSION U32944.1 GI:1209060
MCDRKAVIKNADMSEENQQDSVECATQALEKYNIEKDIAARIKK
EFDKKYNPTWHCIVGRNFGSYVPMETKHFIYFYLGQVAILLFKSG
GENBANK ID: NM_021141.2
VERSION NM_021141.2 GI:12406650
MVRSGNKAAVVLCNDVGFTMSNSIPGIESPFEQAKKVITMFVQR
QVFAENKDEIALVLFGTDGTDNPLSGGDQYQNITVHRRLMLPDFDLLEDIESKIQPGS
QQADFLDALIVSMDVIQHETIGKKFEKRHIEIFTDLSSRFSKSQLDIIIHSLKKCDIS
LQFFLPFSLGKEDGSGDRGDGPFRLGGHGPSFPLKGITEQQKEGLEIVKMVMISLEGE
DGLDEIYSFSESLRKLCVFKKIERHSIHWPCRLTIGSNLSIRIAAYKSILQERVKKTW
TVVDAKTLKKEDIQKETVYCLNDDDETEVLKEDIIQGFRYQSDIVPFSKVDEEQMKYK
SEGKCFSVLGFCKSSQVQRRFFMGNQVLKVFAARDDEAAAVALSSLINALDDLDMVAI
VRYAYDKRANPQVGVAFPHIKMNYECLVYVQLPFNEDLRQYNFSSLKNSKKYAPTEAQ
LNAVDALIDSNSLAKKDEKTDTLEDLFPTTKIPNPRFQRLRQCLLMRALHPREPLPPI
QQHIWNNLNPPAEVTTKSQIPLSKIKTLEPLIEAKKKDQVTAQEIFQDNMEDGPTAKK
LKTEQGGAMESVSSLAEQSVTSVGSVNPAENFRVLVKQKKASEEEASNQLINHIEQFL
DTNETPYFNKSIDCIRAFREEAIKFSEEQRFNNFLKALQEKVEIKQLNHFNEIVVQDG
ITLITKEEASGSSVTAEEAKKFLAPKDKPSGDTAAVFEEGGDVDDLLDMI
GENBANK ID: NM_005053.1
VERSION NM_005053.1 GI:4826963
MAVTITLKTLQQQTFKIRNEPDETVKVLKEKIEAEKGRDAFPVA
DQKLIYAGKILSDDVPIRDYRIDEKNFVVVNVTKTKAGQGTSAPPEASPTAAPESSTS
FPPAPTSGMSHPPPAAREDKSPSEESAPTTSPESVSGSVPSSGSSGREEDAASTLVTG
SEYETNLTEIMSMGYERERVVAALRASYNNPHRAVEYLLTGIPGSPEPEMGSVQESQV
SEQPATEAAGENPLEFLRDQPQEQNNRQVIQQNPALLPALLQQLGQENPQLLQQISRH
QEQFIQMLNEPPGELADISDVEGEVGAIGEEAPQNNYIQVTPQEKEAIERLKALGEPE
SLVIQAYFACEKNENLAANFLLSQNFDDE
GENBANK ID: Z23115.1
VERSION Z23115.1 GI:510900
MSQSNEELVVDFLSYKLSQEKGYSWSQFSDVEENETEAPEGTESE
METPSAINGNPSWNLADSPAVNGATAHSSSLDAREVIPMAAVKQALREAGDEFELRYR
RAFSDLTSQLHITPGTAYQSFEQVVNELFRDGVNWGRIVAFWSEGGALCVESVDKEMQ
VLVSRIAAWMATYLNDHLETWIQENGGWDTFVELYGNNAAAESRKGQERFNRWFLTGN
TVAGVVLLGSLFSRK
GENBANK ID: AB020979.1
VERSION AB02D979.1 GI:6518501
MDEADRRLLRRCRLRLVEELQVDQLWDALLSSELFRPHNIEDIQ
RAGSGSRRDQARQLIIDLETRGSQALPLFISCLEDTGQDMLASFLETNRQAAKLSKPT
LENLTPVVLRPEIRKPEVLRPETPRPVDIGSGGFGDVEQKDHGFEVASTSTEDESPGS
NPEPDATPFQEGLRTFDQLDAISSLPTPSDIFVSYSTFPGFVSWRDPKSGSWYVETLD
DIFEQWAHSEDLQSLLLRVANAVSVKGIYKQNPGCFNFLRKKLFFKTS
GENBANK ID: U21092.1
VERSION U21092.1 GI:726087
MESSKKNDSPGALQTNPPLKLHTDRSAGTPVFVPEQGGYKEKFV
KTVEDKYKCEKCHLVLCSPKQTECGHRTCESCMAALLSSSSPKCTACQESIVKDKVFK
DNCCKREILALQIYCRNESRGCAEQLTLGHLLVHLKNDCHFEELPCVRPDCKEKVLRK
DLRDHVEKACKYREATCSNCKSQVPMIALQKHEDTDCPCVVVSCPHKCSVQTLLRSEL
SAHLSECVNAPSTCSFKRYGCVFQGTNQQIKAHEASSAVQHVNLLKEWSNSLEKKVSL
LQNESVEKNKSIQSLHNQICSFEIEIERQKEMLRNNESKILHLQRVIDSQAEKLKELD
KEIRPFRQNMEEADSMKSSVESLQNRVTELESVDKSAGQVARNTGLLESQLSRHDQML
SVHDIRLADMDLRFQVLETASYNGVLIWKIRDYKRRKQEAVMGKTLSLYSQPFYTGYF
GYKMCARVYLNGDGMGKGTHLSLFFVIMRGEYDALLPWPFKQKVTLMLMDQGSSRRHL
GDAFKPDPNSSSFKKPTGEMNIASGCPVFVAQTVLENGTYIKDDTIFIKVIVDTSDLP
DP
GENBANK ID: NM_001459.1
VERSION NM_001459.1 GI:4503750
MTVLAPAWSPTTYLLLLLLLSSGLSGTQDCSFQHSPISSDFAVK
IRELSDYLLQDYPVTVASNLQDEELCGALWRLVLAQRWMERLKTVAGSKMQGLLERVN
TEIHFVTKCAFQPPPSCLRFVQTNISRLLQETSEQLVALKPWITRQNFSRCLELQCQP
DSSTLPPPNSPRPLEATAPTAPQPPLLLLLLLPVGLLLLAAAWCLHWQRTRRRTPRPG
EQVPPYPSPQDLLLVEH
GENBANK ID: X57500.1
VERSION M36089.1 GI:340396
MPEIRLRHVVSCSSQDSTHCAENLLKADTYRKWRAAKAGEKTIS
VVLQLEKEEQIHSVDIGNDGSAFVEVLVGSSAG0AGEQDYEVLLVTSSFMSPSESRSG
SNPNRVRMFGPDKLVRAAAEKRWDRVKIVCSQPYSKDSPFGLSFVRFHSPPDKDEAEA
PSQKVTVTKLGQFRVKEEDESANSLRPGALFFSRINKTSPYTASDPAGPSYAAATLQA
SSAASSASPVSRAIGSTSKPQESPKGKRKLDLNQEEKKTPSKPPAQLSPSVPKRPKLP
APTRTPATAPVPARAQGAVTGKPRGEGTEPRRPRAGPEELGKILQGVVVVLSGFQNPF
RSELRDKALELGAKYRPDWTRDSTHLICAFANTPKYSQVLGLGGRIVRKEWVLDCHRM
RRRLPSRRYLMAGPGSSSEEDEASHSGGSGDEAPKLPQKQPQTKTKPTQAAGPSSPQK
PPTPEETKAASPVLQEDIDIEGVQSEGQDNGAEDSGDTEDELRRVAEQKEHRLPPGQE
ENGEDPYA0STDENTDSEEHQEPPDLPVPELPDFFQGKHFFLYGEFPGDERRKLIRYV
TAFNGELEDYMSDRVQFVITAQEWDPSFEEALMDNPSLAFVRPRWIYSCNEKQKLLPH
QLYGVVPQA
GENBANK ID: BAA02962.1
DEFINITION HUMAN MRNA FOR RECA-LIKE PROTEIN HSRAD51, COMPLETE CDS.
VERSION 013804.1 GI:397826
CDS 212..1231
/CODON_START=1

| | |
|---|---|
| 1 | GAATTCCGGT AAGGAGAGTG CGGCCCTTCC CGAGGCGTGC AGCTGGGAAC TGCAACTCAT |
| 61 | CTGGGTTGTG CGCAGAAGGC TGGGGCAAGC GAGTAGAGAA GTGCACCGTA AGCCAGCGGG |
| 121 | CTTGGGCGCC GTCCGGCCGG GTCGCGTGCA GCCCCGCGGG GTGAAGTCGG AGCGCGGGGC |
| 181 | CTGCTGGAGA GAGGAGCGCT GCGACCGAGT AATGGCAATG CAGATGCAGC TTGAAGCGAA |
| 241 | TGCAGATACT TCAGTCGAAG AAGAAAGCTT TGGCCCACAA CCCATTTGAC GGTTAGAGCA |
| 301 | GTGTGGCATA AATGCCAACG ATGTGAAGAA ATTCGAAGAA GCTGGATTCC ATACTGTGGA |
| 361 | GGCTGTTGCC TATGCGCCAA ACAAGCAGCT AATAAATATT AAGGGAATTA GTGAAGCCAA |
| 421 | AGCTGATAAA ATTCTGGCTG AGGCAGCTAA ATTAGTTCCA ATGGGTTTCA CCACTGCAAC |
| 481 | TGAAPTCCAC CAAACGCGGT CAGAGATCAT ACAGATTACT ACTGGCTCCA AAGAGCTTGA |
| 541 | CAAACTACTT CAAGGTGGAA TTGAGACTGG ATCTATCACA GAAATGTTTG GAGAATTCCG |
| 601 | AACTGGGAAG ACCCAGATCT GTCATACGCT AGCTGTCACC TGCCAGCTTC CCATTGACCG |
| 661 | GGGTGGAGGT GAAGGAAACG CCATGTACAT TGACACTGAG GGTACCPTTA GGCCAGAACG |
| 721 | GCTGCTGGCA GTGGCTGAGA GGTATGCTCT CTCTGGCAGT GATGTCCTGG ATAATGTAGC |
| 781 | ATATGCTCGA GCGTTCAACA CAGACCACCA GACCCAGCTC CTTTATCAAG CATCAGCCAT |
| 841 | GATGGTAGAA TCTAGGTATG CACTGCTTAT TGTAGACAGT GCCACCGCCC TTTACAGAAC |
| 901 | AGACTACTGG GGTCGAGGTG AGCTTTCAGC CAGGCAGATG CACTTGGCCA GGTTTCTGCG |
| 961 | GATCCTTCTG CGACTCGCTG ATGAGTTTGG TGTAGCAGTG GTAATCACTA ATCAGGTGGT |
| 1021 | AGCTCAAGTG GATGGAGCAG CGATGTTTGC TGCTGATCCC AAAAAACCTA TTGGAGGAAA |
| 1081 | TATCATCGCC CATGCATCAA CAACCAGATT GTATCTGAGG AAAGGAAGAG GGGAAACCAG |
| 1141 | AATCTGCCAA ATCIACGACT CTCCCTGTCT TCCTGAAGCT GAAGCTATGT TCGCCATTAA |
| 1201 | ICCAGAIGGA GTGGGAGATG CCAAAGACTG AATCATTGGG TTTTTCCTCT GTTAAAAACC |
| 1261 | TTAAGTGCTG CAGCCTAATG AGAGTGCACT GCTCCCTGGG GTTCTCTACA GGCCTCTTCC |
| 1321 | TGTTGTGACT GCCAGGATAA AGCTTCCGGG AAAACAGCTA TTATATCAGC TTTTCTGATG |
| 1381 | GTATAAACAG GAGACAGGTC AGTAGTCACA AACTGATCTA AAATGGTTTA TTCCTTCTGT |
| 1441 | AGTGTATTAA TCTCTGTGTG TTIICTTTGG TTTTGGAGGA GGGTATGAAG TATCTTTGAC |
| 1501 | ATGGTGCCTT AGGAATGACT TGGGTTTAAC AAGCTGTCTA CTGGACAATC TTATGTTTCC |
| 1561 | AAGAGAACTA AAGCTGGAGA GACCTGACCC TTCTCTCACT TCTAAATTAA TGGTAAAATA |
| 1621 | AAAGTCCTCA GCTATGTAGC AAAGG |

GENBANK ID: B56529
AGGAGGTGCAGGAGAACAGAAGTGTGCCCTGTGCTCTTCTGAGCAGAGAAGCACCATGAG
CTGGGGCAGGCAAACCCCACTGGGGCTGGCATGGCTCGCTGGGGCTGGCACGTGGAGGGA
AGTGCTGCCTCCCCAGGCCTCTGCTTTAATGATCAGCTTAGTCACTGGTGTGACTGTGCC
CTGGGCTATTGCCTGAGGTGAAACCTTTACCTGCTCCCTGGTCTATCTTGGTAGAATTGA
TCTATTTCAAAGGTATACAGCTAAGCAGATTCTTATTTCTGAGAATACCACCTGTGTGGC
ACCTCCTTTCCAGCTCCTCAGGGAATGTGAGACATGTGAGGAGCTGCCACACTCCTTGCC
AGTAGTCACAGGAAAGGGTGGTTAACAAGTTAAAGTAACCAAGAGGAATATGTGTGTTGA
GTCAGCTGATGGCGTTTGCAAGTGGAATGTCCTTCTTACC
GENBANK ID: CAA59230.1
DEFINITION H. SAPIENS MRNA FOR DNA LIGASE III.
VERSION X84740.1 GI:860962
CDS 334..3102
/CODON_START=1

| | |
|---|---|
| 1 | CCACGCGTCC GGCAGCCTGT ATGAGCAAGT GCCGAGGCCT ACGGTGAGCG CCGGAGCCGG |
| 61 | AGAGGCAGCT ATATGTCTTT GGCTTTCAAG ATCTTCTTTC CACAAACCCT CCGTGCACTC |
| 121 | AGCCGAAAAG AACTGTGCCT ATTCCGAAAA CATCACTGGC GTGATGTAAG ACAATTCAGC |
| 181 | CAGTGGTCAG AAACAGATCT GCTTCATGGA CATCCCCTCT TCCTGAGAAG AAAGCCTGTT |
| 241 | CTATCATTCC AGGGAAGCCA TCTAAGATCA CGTGCCACCT ACCTTGTTTT CTTGCCAGGG |
| 301 | TTGCATGTGG GACTCTGCAG TGGCCCCTGT GAGATGGCTG AGCAACGGTT CTGTGTGGAC |
| 361 | TATGCCAAGC GTGGCACAGC TGGCTGCAAA AAATGCAAGG AAAAGATTGT GAAGGGCGTA |
| 421 | TGCCGAATTG GCAAAGTGGT GCCCAATCCC TTCTCAGAGT CTGGGGGTGA TATGAAAGAG |
| 481 | TGGTACCACA TTAAATGCAT GTTTGAGAAA CTAGAGCGGG CCCGGGCCAC CACAAAAAAA |
| 541 | ATCGAGGACC TCACAGAGCT GGAAGGCTGG GAAGAGCTGG AAGATAATGA GAAGGAACAG |
| 601 | ATAACCCAGC ACATTGCAGA TCTGTCTTCT AAGGCAGCAG GTACACCAAA GAAGAAAGCT |
| 661 | GTTGTCCAGG CTAAGTTGAC AACCACTGGC CAGGTGACTT CTCCAGTGAA AGGCGCCTCA |
| 721 | TTTGTCACCA GTACCAATCC CCGGAAATTT TCTGGCTTTT CAGCCAAGCC CAACAACTCT |
| 781 | GGGGAAGCCC CCTCGAGCCC CACCCCTAAG AGAAGTCTGT GTTCAAGCAA ATGTGACCCC |
| 841 | AGGCATAAGG ACTGTCTGCT ACGGGAGTTT CGAAAGTTAT GCGCCATGGT GGCCGATAAT |
| 901 | CCTAGCTACA ACACGAAGAC CCAGATCATC CAGGACTTCC TTCGGAAAGG CTCAGCAGGA |
| 961 | GATGGTTTCC ACGGTGATGT GTACCTAACA GTGAAGCTGC TGCTGCCAGG AGTCATTAAG |
| 1021 | ACTGTTTACA ACTTGAACGA TAAGCAGATT GTGAAGCTTT TCAGTCGCAT TTTTAACTGC |
| 1081 | AACCCAGATG ATATGGCACG GGACCTAGAG CAGGGTGACG TGTCAGAGAC AATCAGAGTC |
| 1141 | TTCTTTGAGC AGAGCAAGTC TTTCCCCCCA GCTGCCAAGA GCCTCCTTAC CATCCAGGAA |
| 1201 | GTGGATGAGT TCCTTCTGCG GCTGTCCAAG CTCACCAAGG AGGATGACCA GCAACAGGCC |
| 1261 | CTACAGGACA TTGCCTCCAG GTGTACAGCC AATGACCTTA AATGCATCAT CAGGTTGATC |
| 1321 | AAACATCATC TGAAGATGAA CTCAGGTGCA AAACATGTGT TAGACGCCGT TGACCCCAAT |
| 1381 | GCCTATGAAG CCTTCAAAGC CTCGCCCAAC CTGCAGGATG TGGTGGAGCG GGTCCTTCAC |
| 1441 | AACGCGCAGG AGGTGGAGAA GGAGCCGGGC CAGAGACCAG CTCTGAGCGT CCAGGCCTCG |
| 1501 | CTGATGACAC CTGTGCAGCC CATGTTGGCG GAGGCCTGCA AGTCCGTTGA GTATGCAATG |
| 1561 | AAGAAATGTC CCAATGGCAT GTTCTCTGAG ATCAAGTACG ATGGAGAGCG AGTCCAGGTG |
| 1621 | CATAAGAATG GAGACCACTT CAGCTACTTC AGCCGCAGTC TCAAGCCCGT CCTTCCTCAC |
| 1681 | AAGGTGGCCC ACTTTAAGGA CTACATTCCC CAGGCTTPTC CTGGGGGCCA CAGCATGATC |
| 1741 | TTGGATTCTG AAGTGCTTCT GATTGACAAC AAGACAGGCA AACCACTGCC CTTTGGGACT |
| 1801 | CTGGGAGTAC ACAAGAAAGC AGCCTTCCAG GATGCTAATG TCTGCCTGTT TGTTTTTGAT |
| 1861 | TGTATCTACT TTAATGATGT CAGCTTGATG GACAGACCTC TGTGTGAGCG GCGGAAGTTT |
| 1921 | CTTCATGACA ACATGGTTGA AATTCCAAAC CGGATCATGT TCTCAGAAAT GAAGCGAGTC |
| 1981 | ACAAAAGCTT TGGACTTGGC TGACATGATA ACCCGGGTGA TCCAGGAGGG ATTGGAGGGG |
| 2041 | CTGGTGCTGA AGGATGTGAA GGGTACATAT GAGGCTGGGA AGCGGCACTG GCTGAAAGTG |
| 2101 | AAGAAAGACT ATTTGAACGA GGGGGCCATG GCCGACACAG CTGACCTGGT GGTCCTTGGA |
| 2161 | GCCTTCTATG GGCAAGGGAG CAAAGGCGGC ATGATGTCAA TCTTCCTCAT GGGCTGCTAC |
| 2221 | GACCCTGGCA GCCAGAAGTG GTGCACAGTC ACCAAGTGTG CAGGAGGCCA TGATGATGCC |
| 2281 | ACGCTTGCCC GCCTGCAGAA TGAACTAGAC ATGGTGAAGA TCAGCAAGGA CCCCAGCAAA |
| 2341 | ATACCCAGCT GGTTGAAGGT CAACAAGATC TACTATGCTG ACTTCATCGT CCCAGACCCA |
| 2401 | AAGAAAGCTG CCGTGTGGGA GATCACAGGG GCTGAATTCT CCAAATCGGA GGCTCATACA |
| 2461 | GCTGACGGGA TCTCCATCCG ATTCCCTCGC TGCACCCGAA TCCGAGATGA TAAGGACTGG |
| 2521 | AAATCTGCCA CTAACCTTCC CCAACTCAAG GAACTGTACC AGTTGTCCAA GGAGAAGGCA |
| 2581 | GACTTCACTG TAGTGGCTGG AGATGAGGGG AGCTCCACTA CAGGGGGTAG CAGTGAAGAG |
| 2641 | AATAAGGGTC CCTCAGGGTC TGCTGTGTCC CGCAAGGCCC CCAGCAAGCC CTCAGCCAGT |
| 2701 | ACCAAGAAAG CAGAAGGGAA GCTGAGTAAC TCCAACAGCA AAGATGGCAA CATGCAGACT |
| 2761 | GCAAAGCCTT CCGCTATGAA GGTGGGGGAG AAGCTGGCCA CAAAGTCTTC TCCAGTGAAA |
| 2821 | GTAGGGGAGA AGCGGAAAGC TGCTGATGAG ACGCTGTGCC AAACAAAGGT ATTGCTGGAC |
| 2881 | ATCTTCACTG GGGTGCGGCT TTACTTGCCA CCCTCCACAC CAGACTTCAG CCGTCTCAGA |
| 2941 | CGCTACTTTG TGGCATTCGA CGGGGACCTG GTACAGGAAT TTGATATGAC TTCAGCCACG |
| 3001 | CACGTGCTGG GTAGCAGGGA CAAGAACCCT GCGGCCCAGC AGGTCTCCCC AGAGTGGATT |
| 3061 | TGGGCATGTA TCCGGAAACG GAGACTGGTA GCTCCCTGCT AGGTTTGCTG TCTTCCCTCT |
| 3121 | CCCTCAGGCC ATACTCTCCT TTACCATACT ATTGGACTGG ACTCAGGCTG GAGGCAGATA |
| 3181 | GACACAGTAT AGGGGGAATG GGCTTGCTTC TCCCAAACCC ACCAGTTCTC CACTGTCTCT |
| 3241 | TCTGGACCAG GAATTAGTTG CTGTGGGTGC CACAGCTGAA GTCAGTTTGT CTTGCTGGTT |
| 3301 | TAAATAGATC TTTCAGAGCT GGGTGCTGGG TTTGCCATCT TTTTGTTTTC TTTGAAAAGC |
| 3361 | AGCTTAGTTA CCCTTTTTAT AAATAAAATA TCTTGCAGTT AAAAAAAAAA AAAAAAAAAA |
| 3421 | AA |

GENBANK ID: D26155.1
VERSION D26155.1 GI:505086
MSTPTDPGAMPHPGPSPGPGPSPGPILGFSPGPGPSPGSVESMM
GESPGPPSVSHPMPTNGSTDFPQEGMHQMHKPIDGIHDKGIVEDTHCGSNKGTGMRPP
HPGNGPPQSPMDQHSQGMSPHPSPLGAPEHVSSPMSGGGPTPPQYSPPSQPGALIPGD
PQAMSQPNRGPSPFSPVQLHQLRAQILAYKMLARGQFLPETLQLAVQGKRTLPGLQQQ
QQQQQGQQQQQQQQQQQQQPQQQPQQQPQTQQQQQPALVNYNRPSGPGPELSGPSTPQ
KLPVPAPGGRPSPAPPAAAQPFAAAVPGPSVPQFAPGQPSFVLQLQQKQSRISPIQKP
QGLDPVEILQEREYRLQARIAHRIQELENLFGSLPPDLRTKATVELKALRLLNFQRQL
REEVVACNRRDTTLETALNSKAYKRSKRQTLREARMTEKLEKQQKIEQERKRRQKHQE
YLNSILQHAKDFKEYNRSVAGKIQKLSKAVATWHANTEREQKKETERIEKERMRRLMA
EDEESYRKLIDQKKDRRLAYLLQQTGEYVANLTNLVWEHKQAQAAKEKKKRRRRKKKA
EENAEGGESALGPDGEPIDSSSQNSDLPVKVTNTETGKVLFGPEAPKASQLDANLEMN
PGYEVAPRSDSSESDSDYEEEDEEEESSRQETEEKILLDPNSEEVSEKDAKQIIETAK
QDVDDEYSMQYSARGSQSYYTVAHAISERVEKQSALLINGTLKHYQLQGLEWMVSLYN
NNLNGILADEMGLGKTIQTIALITYLMEHKRLNGPYLIIVFLSTLSNNTYEFDKWAPS
VVKISYKGTPAMRRSLVPQLRSGKFNVLLTTYEYIIKDKHILAKIRWKYMIVDEGNRM
KNHHCKLTQVLNTEYVAPRRILLTGTPLQNKLPELWALLNELLPTIFKSCSTFEQWFN
APFAMTGERVDLNEEETILIIRRLEKVLRPFLLRRLKKEVESQLPEKVEYVIKCDMSA
LQKILYRHMQAKGILLTDGSEKDKKGKGGAKTLMNTIMQLRKICNHPYNFQHIESSFA
EHLGYSNGVINGAELYRASGKFELLDRILPKLRATNHRVLLFCQMTSLNTIMEDYFAF
RNFLYLRLDGTTKSEDRAALLKKFNEPGSQYFIFLLSTRAGGLGLNLQAAHTVVIFDS
DWNPHQDLQAQDRARRIGQQNEVRVLRLCTVNSVEEKILAAAKYKLNV0QKVIQAGMF
DQKSSSHERRAFLQAILEHEEENEEEDEVPDDETLNQMIARREEEFDLFMRMDMDRRR
EDARNFKRKPRLNESDELPSWIIKDDAEVERLTCEEEEEKIFGRGSRQRRDVDYSDAL
TEKQWLRAIEDGNLEEMEEEVRLKKRKRRRNVDKDFAKEDVEKAKHRRGRPPAEKLSP
NPPKLTKQMNAIIDTVINYKDSSGRQLSEVFIQLPSRKELPEYYELIRKPVDFKKIKE
RIRNHRYRSLGDLEKDVMLLCHNAQTFNLEGSQIYEDSIVLQSVFKSARQKIAKEEES
EDESNEEEEEEDEEESESEAKSVKKIKLNKKDDKCRDKGKGKKRPNRGKAKFVVSDF
DSDEEQDEREQSEGSGTDDE
GENBANK ID: M62829J
VERSION M62829.1 GI:182262
MAAAKAEMQLMSPLQISDPFGSFPHSPTMDNYPKLEEMMLLSNG
APQFLGAAGAFBGSGSNSSSSSSGGGGGGGGGSNSSSSSSTENPQADTGEQPYEHLTA
ESFFDISLNNEKVLVETSYFSQTTRLPFITYTGRFSLEPAPNSGNTLWPEFLFSLVSG
LVSNTNPPASSSSAPSFAASSASASQSPPLSCAVBSNDSSPIYSAAFTFPTPNTDIFP
EPQSQAFPGSAGTALQYPPPAYPAAKGGFQVFNIPDYLFPQQQGDLGLCTPDQKPFQG
LESRTQQPSLTPLSTIKAFATQSGSQDLKALNTSYQSQLIKPSRMRKYFNRPSKTPPH
ERPYACPVESCDRRFSRSDELTRHIRIHTGQKPFQCRICMRNFSRSDHLTTHIRTHTG
EKPFACDICGRKFARSDERKRETKIHLRQKDKKADKSVVASRATSSLSSYFSPVATSY
PSPVTTSYPSPATTSYPSPVPTSFSSPGSSTYPSPVHSGFPSPSVATTYSSVPPAFPA
QVSSFPSSAVTNSFSASTGLSDNTATFSFRTIEIC
GENBANK ID: U10421.1
VERSION U10421.1 GI:500756
MDNARMNSFLEYPILSSGGSGTCSARAYFSDBRITTFQSCAVSA
NSCGGDDRFLVGRGVQIGSPHHHHHHHHHHPQPATYQTSGNLGVSYSHESCQPSYGSQ
NFSAPYSPYALNQEADVSGGYPQCAPAVYSGNLSSPMVQHHHHHQGYAGGAVGSPQYI
HHSYGQEHQSLALATYNNSLSPLHASHQEACRSPASETSSPAQTFDWNKVKRNFPKTG
KVGEYGYLGQFNAVRTNFTTKQLTELEKEFHFNKYLTRARRVEIAASLQLNETQVKIW
FQNRRMKQKKREKEGLLPISPATPPGNDEKAEESSEKSSSSPCVPSPGSSTSDTLTTS
H
GENBANK ID: U08015.1
VERSION U08015.1 GI:500631
MPSTSFPVPSKFPLGPAAAVFGRGETLGPAPRAGGTMKSAEEEH
YGYASSNVSPALPLPTAHSTLPAPCHNLQTSTPGIIPPADHPSGYGAALDGGPAGYFL
SSGHTRPDGAFALESPRIEITSCLGLYHNNNQFFHDVEVEDVLPSSKRSPSTATLSLF
SLEAYRDPSCLSPASSLSSRSCNSEASSYESNYSYPYASPQTSPWQSPCVSPKTTDPE
EGFPRGLGACTLLGSPQHSFSTSPRASVTEESWLGARSSRPASPCNKRKYSLNGRQPP
YSPHHSPTPSPHGSPRVSVTDDSWLGNTTQYTSSAIVAAINALTTDSSLDLGDGVPVK
SRKTTLEQPPSVALKVEPVGEDLGSPPPPADFAPEDYSSFQEIRKGGFCDQYLAVPQN
PYQWAKPKPLSPTSYNSPTLPALDWQLPSHSGPYELRIEVQPKSHHRAHYETEGSRGA
VKASAGGHPIVQLHGYLENEPLMLQLFIGTADDRLLRPHAFYQVHRITGKTVSTTSHE
AILSNTKVLEIPLLPENSMRAVIDCAGILKLRNSDIELRKGETDICRKNTRVRLVFRV
HVPQPSGRTLSLQVASNPIECSQRSAQELPLVEKQSTDSYPVVGGKKMVLSGHNFLQD
SKVIFVEKAPDGHHVWENEAKTDRDLCKPNSLVVEIPPFRNQRITSPVHVSFYVCNGK
RKRSQYQRFTYLPANGNAIFLTVSREHERVGCFF
GENBANK ID: U08191.1
VERSION U08191.1 GI:476273
MTRVNAGRKGSLAALYDLAVLKKKVKEKEEKKKKKIRIIKSEAE
DLAEPLSSTEGVAPLSQAPSPLAIPAIKEEPLEDLKPCLGINEISSSFFSLLLEILLL
ESQASLFNLEERVLDWQSSPASSLNSWFSAAPNWAELVLPALQYLAGESRAVPSSFSP
FVEFKEKTQQWKLLGQSQDNEKELAALFQLNLETKDQAFCKQENEGSSDATTPVPRVR
TDYVVRPSTGEEKRVFQEQERYRYSQPHKAFTFRMHGFESVVGPVKGVFDKETSLNKA
REHSLLRSDRFAYVTILSLVRDAAARLPNGEGTRAEICELLKDSQFLAFDVTSTQVNT
VVSGALDRLHYEKDPCVKYDIGRKLWIYLER0RSEEEFERIHQAQAAAAKARKALQQK
PKPPSKVKSSSKESSIKVLSSGPSEQSQMSLSDSSNPFTPVTPVTPTTPALPAIPISP
PPVSAVNKSGFSTVSEPAKSSSGVLLVSSFTNPHLGTMLSPASSQTAPSSQAAARVVS
HSGSAGLSQVRVVAQPSLPAVPQQSGGPAQTLPQMPAGPQIRVFATATQTKVVPQTVM
ATVPVKAQTTAATVQRPGPGQTGLTVTSLPATASPVSKPATSSPGTSAPSASTAAVIQ
NVTGQNIIKQVAITGQLGVKPQTGNSIPLTATNFRIQGKDVLRLFPSSITTDAKGQTV
LRITPDNMATLAKSQVTTVKLTQDLFGTGGNTTGKGISATLHVTSNFVNAADSPAKAS
SASAPSSTPTGTTVVKVTPDLKPTEASSSAFRLNPALGVSVADQKGKSTVASSEARPA
ATIRIVQGLGVNPPKAGQTITVATHAKQGASVASGSGTVHTSAVSLPSNNAAVSKTVA
VASGAASTPISISTGAPTVRQVPVSTTVVSTSQACKLPTRITVPLSVISQPMKGKSVV
TAPIIKGNLGANLSGLGRNIILTTMPAGTKLIAGNKPVSFLTAQQLQQLQQQGQATQV
RIQTVPASISNREQLLAPPKQSPLLL
GENBANK ID: M55654
VERSION M55654.1 GI:339491
MDQNNSLPPYAQGLASPQGAMTPGIPIFSPMMPYGTGLTPQPIQ
NTNSLSILEEQQRQQQQQQQQQQQQQQQQQQQQQQQQQQQQQQQQQQQQQQAVAAAAV
QQSTSQQATQGTSGQAPQLFHSQILTTAPLPGTTPLYPSPMTPNTPITPATPASESSG
IVPQLQNIVSTVNLGCKLDLKTIALRARNAEYNPKRFAAVINRIREPRTTALIFSSGK
MVCTGAKSEEQSRLAARKYARVVQKLGEPAKELDFKIQNNVGSCDVKFPIRLEGLVLT
HQQFSSYEPELFPGLIYRMIKPRIVLLIFVSQKVVLTGAKVRAEIYEAFENIYPILKG
FRKTT
GENBANK ID: NM_005568.1
VERSION NM_005568.1 GI:5031866
MVHCAGCKRPILDRFLLNVLDRAWHVKCVQCCECKCNLTEKCFS
REGKLYCKNDFFRCFGTKCAGCRQGISPSDLVRRARSKVFHLNCFTCNNCNKQLSTGE
ELYIIDENKFVCKEDYLSNSSVAKENSLRSATTGSDPSLSPDSQDPSQDDAKDSESAN
VSDKEAGSNEN0DQNLGAERRGPGTTIKAKQLETLKAAFAATPKPTREIREQLAQETG
LNMRVIQVNFQNRRSKERRMKQLSALAGHAFERSPRRMRPLVDRLEPGELIPNGFFSF
YGDYQSEYYGPGGNYDFFPQGPPSSQAQTPVDLPFVPSSGPSGTPLGGLEHPLPGHHP
SSEAQRFTDILAHPPGDSPSPEPSLPGPLHSNSAEVFGPSPPFSSLSVNGGASYGNHL
SHPPEMNEAAVW
GENBANK ID: X69111.1
VERSION X69111.1 GI:32294
MKALSPVRGCYEAVCCLSERSLAIARGRGKGPAAEEPLSLLDDM
NHCYSRLRELVPGVPRGTQLSQVEILQRVIDYILDLQVVLAEPAPGPPDGPHLPIQTA
ELAPELVISNDKRSFCH
GENBANK ID: NP_000507.1
VERSION NP_000507.1 GI:4504047

| | |
|---|---|
| 1 | MGCLGNSETE DQRNEEKAQR EANKKIEKQL QKDKQVYRAT HRLLLLGAGE SGKSTIVKQM |
| 61 | RILHVNGFNG EGGEEDPQAA RSNSDGEKAT KVQDIKNNLK EAIETIVAAM SNLVPPVELA |
| 121 | NPENQFRVDY ILSVMNVPDF DWPPEFYEHA KALWEDEGVR ACYERSNEYQ LIDCAQYFLD |
| 181 | KIDVIEQADY VPSDQDLLRC RVLTSGIFET KFQVDKVNFH NFDVGGQRDE RRKWIQCFND |
| 241 | VTAIIFVVAS SSYNMVIRED NQTNRLQEAL NLFKSIWNNR WLRTISVILF LNKQDLLAEK |
| 301 | VLAGKSKIED YFPEFARYTT PEDATPEPGE DPRVTRAKYF IRDEFLEIST ASGDGRHYCY |
| 361 | PHFTCAVDTE NIRRVFNDCR DIIQRMHLRQ YELL |

GENBANK ID: AAA40889.1
VERSION AAA40889.1 GI:203357

| | |
|---|---|
| 1 | MEQYTANSNS STEQIVVQAG QIQQQQQGGV TAVQLQTEAQ VASASGQGVG TLQVVQGQPL |
| 61 | NVQVSGGQLI TSTGQPINVQ AVPGGQGQTI MQVPVSGTQG LQQIQLVPPG QIQIQGGQAV |
| 121 | QVQGQQGQTQ QIIIQQPQTA VTAGQTQTQQ QIAVQGQQVA QTAEGQTIVY QPVNADGTIL |
| 181 | QQGMITIPAA SLAGAQIVQT GANTNTTSSG QGTVTVTLPV AGNVVNSGGM VMMVPGAGSV |
| 241 | PAIQRIPLPG AEMLEEEPLY VNAKQYHRIL KRRQARAKLE AEGKIPKERR KYLHESRHRH |
| 301 | AMARKRGEGG RFFSPKEKDS PHMQDPNQAD EEANTQIIRV S |

GENBANK ID: B53771
VERSION B53771 GI:2136296

| | |
|---|---|
| 1 | MAWALKLPLA DEVIESGLVQ DFDASLSGIG QELGAGAYSM SDVLALPIFK QEESSLPPDN |
| 61 | ENKILPFQYV LCAATSPAVK LHDETLTYLN QGQSYEIRML DNRELGELPE INGKLVKSIF |
| 121 | RVVFHDRRLQ YTEHQQLEGW RWNRPGDRIL DIDIPMSVGI IDPRANPTQL NTVEFLWDPA |
| 181 | KRTSVFIQVH CISTEFTMRK HGGEKGVPFR VQIDTFEENE NGEYTEHLHS ASCQIKVFKP |
| 241 | KGADRKQKTD REKNEKRTPH EKEKYQPSYE TTILTECSPN PEITYVNNSP SPGFNSSHSS |
| 301 | FSLGEGNGSP NHQPEPPPPV TDNLLPTTTP QEAQQWLHRN RFSTFTRLFT NFSGADLLKL |
| 361 | TRDDVIQICG PADGIRLFNA LKGRNVRPRL TIYVCQESLQ LEEQQQQQQQ QQQKHEDGDS |
| 421 | NGTFFVYHAI YLEELTAVEL TEKIAQLFSI SPCQISQIYK QGPTGIHVLI SDEMIQNFQE |
| 481 | EACFILDTMK QETNQSYHII LK |

GENBANK ID: M92299.1
VERSION M92299.2. GI:184292
MSSYFVNSFSGRYPNGPDYQLLNYGSGSSLSGSYRDPAANHTGS
YGYNYNGMDLSVNRSSASSSHEGAVGESSRAFPAPAQEPRFRQAASSCSLSSPESLPC
TNGDSHGAKPSASSPSDQATSASSSANFTEIDEASASSEPEEAASQLSSPSLARAQPE
PMATSTAAPEGQTPQIFPWMRKLHISHDNTCPDGKRARTAYTRYQTLELEKEFHFNRY
LTRRRRIEIAHALCLSERQIKIWFQNRRMKWKKDNKLKSMSLATAGSAEQP
GENBANK ID: M68891.1
VERSION M68891.1 GI:182995
MEVAPEQPGWMAHPAVLNANDPDSERPGLAHNYMEPAHVLPPDE
VDVFFNHLDSQGNPYYANPAEARAAVSYSPAHARLTGSQNCRPHLLHSPGLPWLDGGK
AALSAAAAHHHNPWTVSPFSKTPLHPSAAGGPGGPLSVYPGAGGGSGGGSGSSVASLT
PTAAHSGSHLFGFPPTPPKEVSPDPSTTGAASPASSSAGGSAARGEDKDGVKYQVSLT
ESMKMESGSPLRPGLATMGTQPATHHPIPTYPSYVPAAAHDYSSGLFHPGGFLGGPAS
SFTPKQRSKARSCSEGRECVNCGATATPLWRRDGTGHYLCNACGLYHKNNGQNRPLIK
PKRRLSAARRAGTCCANCQTTTTTLWRRNANGDPVCNACGLYYKLHNVNRPLTMKKEG
IQTRNRKMSNKSKKSKKGAECFEELSKCMQEKSSPFSAAALAGHMAPVGHLPPFSHSG
HILPTPTPIHPSSSLSFGNPEPSSMVTANG
GENBANK ID: XM_028606.2
VERSION XM_028606.2 GI:15304625
MDEMTAVVKIEKGVGGNNGGNGNGGGAFSQARSSSTGSSSSTGG
GGQESQPSHLALLAATCSRIESPNENSNNSQGPSQSGGTGELDLTATQLSQGPMAGRS
SLPPLGLPLPQRNRVAAVPMAAMAVSLPRIAQSLVGSMLCAAPNLQNQQVLTGLPGVM
PNIQYQVIPQFQTVDGQQLQFAATGAQVQQDGSGQIQIIPGANQQIITNRGSGGNIIA
AMPNLLQQAVPLQGLANNVLSGQTQYVTNVPVALNGNITLLPVNSVSAATLTPSSQAV
TISSSGSQESGSQPVTSGTTISSASLVSSQASSSSFFTNANSYSTTTTTSNNGIMNFT
TSGSSGTNSQGQIPQRVSGLQGSDALNIQQNQTSGGSLQAGQQKEGEQNQQTQQQQIL
IQPQLVQGGQALQALQAAPLSGQTFTTQAISQETLQNLQLQAVPNSGPIIIRTPTVGP
NGQVSNQTLQLQNLQVQNPQAQTITLAPMQGVSLGQTSSSNTTLTPIASAASIPAGTV
TVNAAQLSSMPGLQTINLSALGTSGIQVHPIQGLPLAIANAPGDHGAQLGLHGAGGDG
IHDDTAGGEEGENSPDAQPQAGRRTRREACTCPYCKDSEGRGSGDPGKKKQHICHIQG
CGKVYGKTSHLRANLRWHTGERPFNCTWSYCGKRFTRSDELQRHKRTNTGEKKFACPE
CPKRFNRSDHLSKHIKTHQNKKGGPGVALSVGTLPLDSGAGSEGSGTATPSALITTNM
VAMEAICPEGIARLANSGINVMQVADLQSINISGNGF
GENBANK ID: NP_009077.1
DEFINITION HOMO SAPIENS ZINC FINGER PROTEIN 161 (ZNF161), MRNA.
VERSION NB_007146.1 GI:6005967
CDS 42..1592
/CODON_START=4

| | |
|---|---|
| 1 | AGCGGGGGGA GTGGGGAGGA GGGGGGTCGG CCGCCGCAGC CATGGAGGCC AACTGGACCG |
| 61 | CGTTCCTGTT CCAGGCCCAT GAAGCTTCCC ATCACCAACA GCAGGCAGCA CAGAACAGCT |
| 121 | TGCTGCCCCT CCTGAGCTCT GCCGTGGAGC CCCCTGATCA GAAACCATTG CTTCCAATAC |
| 181 | CAATAACTCA GAAACCTCAG GGTGCACCAG AAACATTAAA GGATGCCATT GGGATTAAAA |
| 241 | AAGAAAAACC CAAAACTTCA TTTGTGTGCA CTTACTGCAG TAAAGCTTTC AGGGACAGCT |
| 301 | ATCACCTGAG GCGCCACGAA TCGTGCCACA CAGGGATCAA GTTGGTGTCC CGGCCAAAGA |
| 361 | AAACCCCCAC CACGGTGGTT CCCCTTATCT CTACCATCGC TGGGGACAGC AGCCGAACTT |
| 421 | CGTTGGTCTC GACCATTGCA GGCATCTTGT CAACAGTCAC TACATCTTCC TCGGGCACCA |
| 481 | ACCCCAGTAG CAGTGCCAGC ACCACAGCTA TGCCAGTGAC CCAGTCTGTC AAGAAACCCA |
| 541 | GTAAGCCTGT CAAGAAGAAC CATGCTTGTG AGATGTGTGG GAAGGCCTTC CGAGATGTGT |
| 601 | ACCATCTCAA TCGACACAAG CTCTCCCATT CAGATGAGAA ACCCTTTGAG TGTCCTATTT |
| 661 | GTAATCAGCG CTTCAAGAGG AAGGACCGGA TGACTTACCA TGTGAGGTCT CATGAAGGAG |
| 721 | GCATCACCAA ACCCTATACT TGCAGTGTTT GTGGGAAAGG CTTCTCAAGG CGTGACCACT |
| 781 | TAAGCTGTCA TGTAAAACAT GTCCATTCAA CAGAAAGACC CTTCAAATGC CAAACGTGCA |
| 841 | CTGCTGCCTT TGCCACCAAA GACAGACTGC GGACACACAT GGTGCGCCAT GAAGGCAAGG |
| 901 | TATCATGTAA CATCTGTGGG AAGCTCCTGA GTGCAGCATA CATCACCAGC CACTTAAAGA |
| 961 | CTCATGGGCA GAGCCAAAGT ATCAACTGTA ATACATGTAA ACAAGGCATC AGTAAAACAT |
| 1021 | GCATGAGTGA AGAGACCACT AACCAAAAGC AGCAGCAGCA GCAGCAGCAG CAACAACAAC |
| 1081 | AACAACAACA TGTGACAAGC TGGCCAGGGA AGCAAGTAGA AACACTCAGA CTGTGGGAAG |
| 1141 | AAGCTGTTAA AGCAAGGAAG AAAGAAGCTG CTAACCTGTG CCAAACCTCC ACGGCTGCTA |
| 1201 | CGACACCTGT GACTCTCACT ACTCCATTCA GTATAACATC CTCTGPGTCG TCTGAGACTA |
| 1261 | TGTCAAACCC AGTCACAGTG GCAGCTGCAA TGAGCATGAG AAGTCCACPA AATGTTTCAA |
| 1321 | GTGCAGTTAA CATAACCAGC CCAATGAACA TAGGGCATCC TGTAACTATA ACCAGTCCAT |
| 1381 | TATCCATGAC CTCTCCTTTA ACACTCACTA CCCCAGTCAA CCTCCCCACC CCCGTCACTG |
| 1441 | CCCCAGTGAA TATAGCACAC CCTGTCACCA TCACATCTCC AATGAATCTA CCCACACCTA |
| 1501 | TGACATTAGC CGCCCCTCTC AATATAGCAA TGAGACCTGT AGACAGCATG CCTTTCTTGC |
| 1561 | CCCAAGCTTT GCCTACATCA CCGCCTTGGT AAACAGTATT ATAAAATCAA AATATGGGTA |
| 1621 | AAAGTAAATA TTTACCAGCA ACTTAACTTT TAGTTGATTA AAGCAAAAAG TAAACCATGA |
| 1681 | AATTGGGAGA TTTTATTACA TTAGTTAATA AGAGTGTGGT AGCATTTTTC TCCAATTTGG |
| 1741 | CTGGGATTAT TCAAAGTAGG GTGTGTATGT AACTTATCAC TGGACCACTT TAGTTTAATC |
| 1801 | AGAAATTCCT TTTAGCTGAC AACATTGCTT AAACAGGATA GTAGTTGGCA AGATGAAATG |
| 1861 | CCAGAATTAA AACCAATCAT AAGTAGAACC CACTTCAAAA TAAAAAAACA GCATTACTAT |
| 1921 | TTCTAATCCC AAGGAATCAC TTTATTGTAA ACACTAGCAG AACTCTTCTC CCTATACAAG |
| 1981 | GTGGATGGCT GATTTTAACC TGAAATTTTA AATCCACAGA TTGAGAGCTA GTGTAGAATT |
| 2041 | GTCTGTGTTT ATTGTTTTTA TGAGTAAATA CATGCATTGT CATAATAAAA TGCATTTCAG |
| 2101 | AGAATATGCA TTTTACCTTT GGGAATATGT TAATTTCAGG CAGCATTCCC TATGGGAAAG |
| 2161 | GTGATACCAG CTCTGATATG CAAAGCATAT GATAATTTAT CATTCTAACT TCAACGTATA |
| 2221 | ATAGGGATTG TGACCTGATA TTTGGAGATG TAAATATTGC TCAGCATATT AATCCCGATG |
| 2281 | GAATATAGCA TTGTAGTTGA CTTTTT |

GENBANK ID: AAA36598.1
DEFINITION HUMAN STEM CELL PROTEIN (SCL) MRNA, COMPLETE CDS.
VERSION M29038.1 GI:337958
CDS 81..725
/CODON_START=1

| | |
|---|---|
| 1 | AGTCAGAGTC ACTTTCTGTA AATGGTACTT AGGTAGGCGC GTCCGCCTCG GTTACAGCGG |
| 61 | AGCTGCCCGG CGACGGCCGC ATGGTGCAGC TGAGTCCTCC CGCGCTGGCT GCCCCCGCCG |
| 121 | CCCCCGGCCG CGCGCTGCTC TACAGCCTCA GCCAGCCGCT GGCCTCTCTC GGCAGCGGGT |
| 181 | TCTTTGGGGA GCCGGATGCC TTCCCTATGT TCACCACCAA CAATCGAGTG AAGAGGAGAC |
| 241 | CTTCCCCCTA TGAGATGGAG ATTACTGATG GTCCCCACAC CAAAGTTGTG CGGCGTATCT |
| 301 | TCACCAACAG CCGGGAGCGA TGGCGGCAGC AGAATGTGAA CGGGGCCTTT GCCGAGCTCC |
| 361 | GCAAGCTGAT CCCCACACAT CCCCCGGACA AGAAGCTCAG CAAGAATGAG ATCCTCCGCC |
| 421 | TGGCCATGAA GTATATCAAC TTCTTGGCCA AGCTGCTCAA TGACCAGGAG GAGGAGGGCA |
| 481 | CCCAGCGGGC CAAGACTGGC AAGGACCCTG TGGTGGGGGC TGGTGGGGGT GGAGGTGGGG |
| 541 | GAGGGGGCGG CGCGCCCCCA GATGACCTCC TGCAAGACGT GCTTTCCCCC AACTCCAGCT |
| 601 | GCGGCAGCTC CCTGGATGGG GCAGCCAGCC CGGACAGCTA CACGGAGGAG CCCGCGCCCA |
| 661 | AGCACACGGC CCGCAGCCTC CATCCTGCCA TGCTGCCTGC CGCCGATGGA GCCGGCCCTC |
| 721 | GGTGATGGGT CTGGGCCACC AGGATCAGCC AGGAGGGCGT TCTTAGGCTG CTGGGATGGT |
| 781 | GGGCTTCAGG GCAGGTGGGG TGAGAATTGG GCGGCTCTGA AGCAAGGCGG TGGACTTGAA |
| 841 | CTTTCCTGGA TGTCTGAACT TTGGGAAGCC TTTACTGACC CTGGGGCTGG CTTTTCTGTT |
| 901 | TCCTGTACCA GTAGGAGAIC AGAAAAATGG AGCAAAGTGG TAGGTACTTT TTGTGAAGAC |
| 961 | GGCACGGTCT TCCCTCTTCC CTCAGTCCCA AATCCTTCCC AAGTAAGAGG CTGGAGTTGT |
| 1021 | CACTGCTTTT GGCCTGGAGT TTGGGATCCC TGTCTTTCCT AAGACCTGGG GTTGTCAGCT |
| 1081 | CTCATCTGAG GCATCCAGCA GTCTCTGCCT TGCCTTTAGC CCCTCCCAAG CTGGCTGGGG |
| 1141 | TGGCCTGTGT GGCCACTTCT GTCCATATTT ATAGGTACCC AATAGCTGCC CATTTCGTGA |
| 1201 | GCCCCATCTT CACCCAGGCC TATGTTGATC CATCCAGCTT GCCAGATGCT GCAGAGTCAC |
| 1261 | AAGCCTCGAG GTGCCTTCTT CAGGGCCTGG TTGAAGAAGA TGATCAGTGG ACAGTCTGCT |
| 1321 | CTAGATGAGC TGGGCCGGAG GGTCAGGAAA CCCAGTCGCC CTTACTTCTT GCCCTGGGGA |
| 1381 | TCAAAGTTCT GCTTTCTCCC CAATGAGACT TGCCTTCCTA AGCCTGTGGC TGTGGAGACC |
| 1441 | ATGTCTGCAG CCCTGAGAAA GCCCTGTCGG GCTTTGTGTG AAGGCAGAGA AAGGGACAAT |
| 1501 | GATACTAGAG TGATATGGAG CAAGAGATAT TTTGGGCATG TGGGCTTCAA CTCCTCGACA |
| 1561 | TCACTGTTCA TGCTGGCGAG TGAATGCCAG TGTGCTGATG GGCGTACGCT GGTGCTGAGT |
| 1621 | AGATGCGCAG CCCCATCTGT GCATTCTCCT GGATGCTTAG AGGGATTTCT TTGCTGTAAG |
| 1681 | ATGTCTGTTT GCTGATGGTC TGGTCTATGT TCCGAATTGA GCACAAAACC TGTCCTATGA |
| 1741 | ATGCTTTGCA TTTGGAATTT TTGCTTGACT TCAGTTATTG GTGGAATCTT TAGCGCTCAA |
| 1801 | TAGGACCAGG ATCCAGCCTC ACTTCTAGGG TATGGGAAAT CCAATCAGAG ACCAGGCCCT |
| 1861 | GGCTAAGACC CAAACATATG CACATTCACT TAGCAGAACC TTAAACACCC CTCAGTTGTG |
| 1921 | CAGCTTTTGG TCATCAAGGG TGCGTCTGGG AGGTTGGTTT AATGCAATAG AAGTGCTCCC |
| 1981 | CTCTGAAAGT TGTACATGAA ATTTTTGTAA ATCACATCCT TATCCTTCAT CTTTTAAAGA |
| 2041 | AATAACCACT GCAAGTCCTT TTGTAAAGTG AAGAATCCTT TTGTAGAATG AACCACTGCC |
| 2101 | CCTTCATTGA TTTCCTGTGT CAATCCAGAT GGTGGGATGT GGTTTTCTTA AGGTGAGGCC |
| 2161 | TGTCTGTGAC CTGCATCTAA GCCCATGGGA CAAATTGCAC AGAAGTCCTG TATGTCTGTC |
| 2221 | ATTGTACCCT TAAGTCACCC TAGCCCTCTC CCTCTAGGCT CTGCCTTCGA GGTCAGAGGA |
| 2281 | GAGATAGCCT GTGGCCCTGT CCTGCCATGC AAGAACTCAT CACTGTGGCT GTCTGGAAAG |
| 2341 | CCCCCCCTTA TAGTTTGGGC TTCAGCCTAG TGGCTTGTCC TGACCATGAT GGGGCCCTAA |
| 2401 | TTCAGCCATG TACAGACAGA GAATATGTCT GCTCCTTTCC CCTTCCTTTT AAGTAAGGTC |
| 2461 | CAATTCTCGA GCTTGGGGCA ACATTGTTCA CCTTTGTAGC ACTCAGGCTC TCCATTCAAT |
| 2521 | TTCAGGCTCC CCAGATCATG TTTTGGTGAA AATTAGGGTT GGTTCCTTTC CAACGTTTGG |
| 2581 | AAGATCCTGT GAGGAGCCCC ATCTGTCTAA AGATAGAGTC ATTGCTGTAG GATCTAAGGC |
| 2641 | TGTTTGCTTC ACCGTGGATT CGCTTGAGTT AGGAATGAGA ACTACCGACA GTATGGATGG |
| 2701 | GTGGATGGGT TTTATGAGAT GGATCACATA TTTTATTAAG AACTCAAACT TCTGGCTCCC |
| 2761 | TCTTCTTTCA GACTTGCCAT GTGACTCTGG CTTGGCCTAT CTCCTAGGGC TATGGTGTGG |
| 2821 | ACTGAATGGG ATCATGAAAG TAGACAGTTT TGAGAACGTA AAGAACTTTT TCTTTTCCCT |
| 2881 | CAATCTCAAT CCTGCAGTGG GGTTTCGCAG CCTGAGTCCA CGACCTAGGC AGTAGGCCGG |
| 2941 | TGTGCCTGAC TGCCCAGCAT TTGGGTAATT TAGATTGTAA ACCGCTTTGG CCTGAGTTAT |
| 3001 | TGAGATTGTC CTCATTTCTC CAGATTATCT ATTTGTGTGT GTGTGTGTGT GTGTGTGAGA |
| 3061 | GACGGTGTCT TGTTCTGTCA CTCAGGCTGG AGTACAGTGG TGCCATCATT GCTGTCTGCA |
| 3121 | GCCTTGAACT CTGGGCTCAA GCAATCCTCT CACCTCAGCC TCCCGAGTAG GGAGGACCAC |
| 3181 | AGGTGTGAGC CACCACACCT GGCTAATTTT TACTTTTTTT TTTTTTTGGT AGAGATGGAG |
| 3241 | TCTTGCTATA TTGCCCAGGC TGGTCTTGAA GTCCTGGCTT CAGGCAATTC TCCTGCCTTT |
| 3301 | GCCTCCAGAA GCACTGGGAT CACAGGTGTC AGCCATTGCA CCCAGCCCAG ATTGTCTTAA |
| 3361 | TTTCTATCTT GTTCCAAGGC CAGGGACAGT AATAAGAATG GAAAAGAGAT ATGGGAACAC |
| 3421 | TGGCAGACTG TGTAAAATGT AATGCAACTA CCCAAAACAA GCCTGGTAGG AAAGGGCAAG |
| 3481 | TCTTTAGGTC TTTGTAAGAA CTAAAGAAGA TCTGTAATTT TTATTTTCAC CCTCTGTACC |
| 3541 | CCATGACCTT ATCCTTCCTC TCCTTCCTTG TTACCCATGA AAAACTGGCA ACATTCCAAG |
| 3601 | AATAGCATCT GTACAAAGGG GAAAGAACAT AAAGGTAAAA CAAAACAAAA CAACATTTTG |
| 3661 | AGAACRAAGA TGACCATAAC CACTGAAGGG AATCACATCT TTTAAGACAA ATTCATATTC |
| 3721 | TTTTATTTGT TATGGCAGAT GACAAGATGG TACAACCTTT ATTCTTTTCC AAAATAAAAC |
| 3781 | AAAGGGCACA GCATCTGTAG TCAGCCGACA ACTCTTTCGG CCTTTTGGGG GTGGGTCTGG |
| 3641 | CCGTACTTGT GATTTCGATG GTACGTGACC CTCTGCTGAA GACTTGCCCC CTGCCCGTGT |
| 3901 | ACATAGTGCA TTGTTTCTGT GGGCGGGCCC AGCACTTTCC GTCAACGTTG TACTGTATGT |
| 3961 | GATGAATTGC GTTGGTCTCT GCATTTTTCT GCAGAAGAGG AGTAACCGCT CCAGGTACCT |
| 4021 | TGACCTTTGT ACAGGCCAGA GGCCAACACT GTGGGTGTGT GACTCTTTAG CAAAAAAAAC |
| 4081 | CCATGTGGTG ATGATGTGTC TATATATGTG AGGATGTATC GGGAAGATTT CTAAATAAAA |
| 4141 | GTTTTACAAA GGG |

GENBANK ID: NP_006168
DEFINITION HOMO SAPIENS NEURAL RETINA LEUCINE ZIPPER (NRL), MRNA.
VERSION NM_006177.1 GI:5453801
CDS 118..831
/CODON_START=1

| | |
|---|---|
| 1 | CCAGGCCCTG CTCCATGGAG CCTTCAGTCT CCTGGGAAGC TGTGCCTGTC TGGCTCTGGC |
| 61 | ACTGACCACA TCCTCTCGGC CATTTCTGAA GTGCACTCCT CCCAGCCCAG CTCCAGAATG |
| 121 | GCCCTGCCCC CCAGCCCCCT GGCCATGGAA TATGTCAATG ACTTTGACTT GATGAAGTTT |
| 181 | GAGGTAAAGC GGGAACCCTC TGAGGGCCGA CCTGGCCCCC CTACAGCCTC ACTGGGCTCC |
| 241 | ACACCTTACA GCTCAGTGCC TCCTTCACCC ACCTTCAGTG AACCAGGCAT GGTGGGGGCA |
| 301 | ACCGAGCGCA CCCGGCCAGG CCTGGAGGAG CTGTACTGGC TGGCTACCCT GGAGGAGGAG |
| 361 | CTGGGGGCTG GGGAGGCATT GGGGCTGAGT CCTGAAGAGG CCATGGAGCT GCTGCAGGGT |
| 421 | CAGGGCCCAG TCCCTGTTGA TGGGGCCGAT GGCTACTACC CAGGGAGCCC AGAGGAGACA |
| 481 | GGAGCCCAGC ACGTCCAGCT GGCAGAGCGG TTTTCCGACG CGGCGCTGGT CTCGATGTCT |
| 541 | GTGCGGGAGC TAAACCGGCA GCTGCGGGGC TGCGGGCGCG ACGAGGCGCT GCGGCTGAAG |
| 601 | CAGAGGGGGC GCACGCTGAA GAACCGCGGC TACGCGCAGG CCTGTCGCTC CAAGCGGCTG |
| 661 | CAGGAGGGGG GCGGGCTGGA GGCCGAGCGC GCCCGCCTGG CCGCCCAGCT GGACGCGCTG |
| 721 | CGGGCCGAGG TGGCCCGCCT GGCCCGGGAG CGCGATCTCT ACAAGGCTCG CTGTGACCGG |
| 781 | CTAACCTCGA GCGGCCCCGG GTCCGGGGAC CGCTGCCACG TCTTCCTCTG AGCCGTTCAG |
| 841 | AGCACCTTGT GGTGTAGTGG GGGCTGGGTG GGGTGGCTCC GCCCAGGAGG CGGCTGCACG |
| 901 | GTTCTCTGCA TCGTTACCAG AGCGCCTTCT GGTCCTAGCC ACGCCCTGTA TGACCGCGCA |
| 961 | AATATCCCCA AAGCTTTTGG GTCCTCAAGT CATGCCCGAA TTTAGATGCT GGTCATTTTC |
| 1021 | TGGAGAGGGG TCCCCTCCCC TTACGAACAC AAAAACCCAG CCCACATGAC TAGCACGCTG |
| 1081 | AGCTCTGCAG GGACCAGTGC CAGGCACTGG GGGGTGGAAG TGTGGTGACA CAGTGAATGG |
| 1141 | GAGGTGGAGG AGGGTTGCAG CTCCCACCTC AGTTTAGTTT TTAATTCAGG GTTTTCAACC |
| 1201 | TGTAACACAT TAAAGCTGTA ATTAGCAATG AGGCTGTATT TTCATTCTGA AGCTTGTAAC |
| 1261 | CTCCCCATTT TAGCACTACA GAATTTTCAA GATTTCAATA TCCAACAACT AGATAGATTA |
| 1321 | GGACCTCTAT CGGAGATGCT TTTTCCCTGC CCAACCCTGT GGCCTTCAGG GCTCAGAGCA |
| 1381 | GCAAAGGCCT GAAGAGTGAG CTCTGGGGGT TGTTGGTGTG GGTTGGGAGA GAGCTGTGTG |
| 1441 | CAGAAGTCTG GAAACCTGGG TCCTAGTCCC AGCTCTTCCA TGGGATCCCC CTGTCACCCT |
| 1501 | GAGCAAATCA GTTGCTTCCT GGACTTGTGT TACTTCATCT AATTCTCATG TGGATTGGAC |
| 1561 | GACTTCTCCT CCCTTTCCAG TTCTGGCATC TCCCCAGTAT GGAAGTCCCG GTGGTCTCCC |
| 1621 | CAAGAAGTCC CCAAGACAAT CTCGCCAAAG GCACCTCCTA TCCTCCTGCA GTTTCCCACC |
| 1681 | TGCAGCCTAG GCAGGGCATG CACAGCCCAG GCGAGGAAGC CTGCCTTCTC TGTGAGCACA |
| 1741 | TACGTGGGTC CTCCGCAGCT CCCTCCAGCC TGTCTGGGCC TCCAGACCTG CACAGGGTGC |
| 1801 | TCCTGCCACC TCCCACCTCT CTGAGGGCTG AGGTGAGACT TCTCCTGGGA TGACAATTTG |
| 1861 | CTGAGAGAGT GCAGCTTTTC TGAATTAAAC TTGAAGTCCA CGCAGAATTC TAATGCAATA |
| 1921 | AGCTAAATGT T |

GENBANK ID: AAA58399.1
VERSION M95809.1 GI:179568
/GENE=“BTF2”
CDS 55..1701
/CODON_START=1

| | |
|---|---|
| 1 | AGTTAGTTAC TTCCTGTCTA GAGTTGTAGC TTCCACCTGC ACCTTCTAGC CACCATGCCA |
| 61 | ACCTCATCTG AAGAAGTTTT GCTGATTGTA AAGAAAGTGC GTCAAAAGAA GCAGGATGCA |
| 121 | GCTCTGTACC TCATGGCAGA AAGAATTGCT TGGGCACCTG AAGGCAAAGA TAGATTTACA |
| 181 | ATCAGCCATA TGTATGCAGA TATTAAATGC CAGAAAATTA GTCCAGAAGG AAAAGCTAAA |
| 241 | ATTCAGCTTC AGCTGGTCCT ACATGCAGGG GACACAACTA ACTTCCATTT TTCCAATGAA |
| 301 | ACCACACCAG TGAAAGAGCG AGATGCAGTA AAAGACCTTC TTCAGCAGCT GCTGCCCAAA |
| 361 | TTCAAGAGGA AAGCAAATAA AGAACTGGAA GAGAAGAACA GAATGCTGCA AGAAGATCCT |
| 421 | GTTTTGTTTC AGCTTTATAA AGACCTTGTT GTGAGTCAAG TGATCAGTGC TGAGGAATTC |
| 481 | TGGGCCAATC GTTTAAATGT GAATGCAACA GATAGTTCTT CCACATCCAA TCATAAGCAG |
| 541 | GATGTTGGCA TTTCTGCTGC ATTTCTGGCT GATGTCCGGC CCCAAACTGA TGGCTGTAAC |
| 601 | GGTCTAAGAT ATAATTTAAC TTCTGATATC ATTGAGTCCA TATTTAGGAC CTATCCAGCA |
| 661 | GTAAAAATGA AATATGCAGA AAATGTTCCC CACAACATGA CAGAGAAGGA ATTCTGGACA |
| 721 | CGTTTTTTCC AGTCCCATTA TTTTCACAGG GATCGGCTGA ATACAGGGTC AAAGGATCTC |
| 781 | TTTGCAGAAT GTGCCAAAAT AGATGAAAAA GGCCTAAAAA CAATGGTTTC ATTAGGAGTG |
| 841 | AAAAACCCAC TACTAGATTT AACAGCTTTG GAAGATAAAC CATTAGATGA GGGCTATGGC |
| 901 | ATTTCCTCTG TGCCATCTGC TTCCAATTCT AAATCCATAA AAGAGAATAG TAATGCTGCC |
| 961 | ATCATCAAGA GATTTAACCA TCACAGTGCC ATGGTCCTGG CAGCTGGACT CAGAAAACAA |
| 1021 | GAAGCACAAA ATGAACAAAC TAGTGAGCCC AGCAACATGG ATGGAAATTC CGGAGATGCA |
| 1081 | GACTGCTTTC AGCCAGCAGT CAAAAGGGCG AAATTACAAG AGTCCATTGA ATATGAAGAC |
| 1141 | TTGGGGAAAA ATAATTCTGT AAAAACGATT GCACTAAACC TCAAGAAGTC AGATAGGTAT |
| 1201 | TATCATGGTC CAACTCCAAT CCAGTCACTA CAGTATGCAA CAAGTCAGGA CATTATTAAT |
| 1261 | TCTTTTCAAA GTATTAGACA AGAAATGGAA GGTTATACAC CCAAGTTAAC TCAGGTTCTC |
| 1321 | TCAAGTAGTG CTGCCAGTAG TACCATCACA GCACTGTCAC CTGGAGGGGC ACTTATGCAG |
| 1381 | GGAGGAACAC AGCAAGCCAT AAACCAGATG GTGCCAAATG ATATTCAATC TGAATTGAAA |
| 1441 | CACTTATATG TAGCTGTTGG AGAACTTCTA CGACATTTCT GGTCCTGCTT TCCTGTTAAT |
| 1501 | ACGCCATTCC TAGAAGAAAA GGTAGTGAAA ATGAAAAGTA ATTTGGAACG ATTCCAAGTT |
| 1561 | ACGAAGCTCT GTCCATTCCA AGAAAAGATT CGGAGACACT ATTTAAGCAC AAATTTGGTA |
| 1621 | AGTCACATAG AAGAGATGCT CCAGACAGCC TACAACAAGC TCCACACATG GCAGTCACGG |
| 1681 | CGTCTGATGA AGAAAACGTG AGGTGGCCAT GATGCTTACA GGTTTTGTGA GATTGAGAGA |
| 1741 | ACTATGACCT GCAGCAACTC TGGAAACCTG GCCTGACAGA CAAGCAGATG ACCTCACAGG |
| 1801 | AGTGATAAGA AACATCTGCT CCACGCCAAC TCCCAGAGCT GATGCTATTG TACTTGCACA |
| 1861 | TTGGAGACTG AAAGGAAAGA AGGGACTAAA TGC |

GENBANK ID: AAA65605.1
DEFINITION HUMAN OCTANER BINDING TRANSCRIPTION FACTOR 1 (OTF1) MRNA,
VERSION L20433.1 GI:418015
CDS 235..1497
/CODON_START=1

| | |
|---|---|
| 1 | GCGGGGCTAG AGCTGTCGGA GAAGCGGGAC CGCGAGGCCG GCGCGCGGCG CTCTGCGCGG |
| 61 | TCAGAGGGAG CGCCTGGCAG CAGCAGGAGC AGCAGCAGCA GCCCGCGGCG GGGCCGCCGC |
| 121 | CAGCCGCCGC GACCGCCGCG GCTGCAGCCT CCGAAGGGAG GCCGGGTGAG CCGGCGTACG |
| 181 | CACTTTCCCG CGGACTTTCG GAGTGTTTGT GGATATACAT GCCAAGCCGC CACGATGATG |
| 241 | TCCATGAACA GCAAGCAGCC TCACTTTGCC ATGCATCCCA CCCTCCCTGA GCACAAGTAC |
| 301 | CCGTCGCTGC ACTCCAGCTC CGAGGCCATC CGGCGGGCCT GCCTGCCCAC GCCGCCGCTG |
| 361 | CAGAGCAACC TCTTCGCCAG CCTGGACGAG ACGCTGCTGG CGCGGGCCGA GGCGCTGGCG |
| 421 | GCCGTGGACA TCGCCGTGTC CCAGGGCAAG AGCCATCCTT TCAAGCCGGA CGCCACGTAC |
| 481 | CACACGATGA ACAGCGTGCC GTGCACGTCC ACTTCCACGG TGCCTCTGGC GCACCACCAC |
| 541 | CACCACCACC ACCACCACCA GGCGCTCGAA CCCGGCGATC TGCTGGACCA CATCTCCTCG |
| 601 | CCGTCGCTCG CGCTCATGGC CGGCGCGGGC GGCGCGGGCG CGGCGGCCGG CGGCGGCGGC |
| 661 | GCCCACGACG GCCCGGGGGG CGGTGGCGGC CCGGGCGGCG GCGGCGGCCC GGGCGGCGGC |
| 721 | GGCCCCGGGG GAGGCCGCGC TGGCGGCCCG GGGGGCGGCG GCGGCGGCCC GGGCGGCGGG |
| 781 | CTCCTGGGCG GCTCCGCGCA CCCTCACCCG CATATGCACA GCCTGGGCCA CCTGTCGCAC |
| 841 | CCCGCGGCCG CCGCCGCCAT GAACATGCCG TCCGGGCTGC CGCACCCCGG GCTGGTGGCG |
| 901 | GCGGCGGCGC ACCACGGCGC GGCAGCGGCA GCGGCGGCGG CGTCGGCCGG GCAGGTGGCA |
| 961 | GCGGCATCGG CGGCGGCGGC CGTCGTGGCC GCAGCGGGCC TGGCGTCCAT CTGCGACTCG |
| 1021 | GACACGGACC CGCGCCAGCT CCAGGCCTTC GCGGAGCGCT TCAAGCAGCG GCGCATCAAG |
| 1081 | CTGGGCGTGA CGCAGGCCGA CGTGGGCTCG GCGCTGGCCA ACCTCAAGAT CCCGGGCGTG |
| 1141 | CGCTCACTCA GCCAGAGCAC CATCTGCAGG TTCGAGTCGC TCACGCTCTC GCACAACAAC |
| 1201 | ATGATCGCGC TCAAGCCCAT CCTGCAGGCG TGGCTCCAGG AGGCCGAGGG CCCCCAGCGC |
| 1261 | GAGAAAATGA ACAAGCCTGA GCTCTTCAAC GGCGGCGAGA AGAAGCGCAA GCGGACTTCC |
| 1321 | ATCGCCGCGC CCGAGAAGCG CTCCCTCGAG GCCTACTTCG CCGTGCAGCC CCGGCCCTCG |
| 1381 | TCCGAGAAGA TCGCCGCCAT CGCCGAGAAA CTGGACCTCA AAAAGAACGT GGTGCGGGTG |
| 1441 | TGGTTTTGCA ACCAGAGACA GAAGCAGAAG CGGATGAAAT TCTCTGCCAC TTACTGAGGG |
| 1501 | GGCTGGGAGG TGTCGGGCGG GACAGAATGG GGAGCTGAGG AGGCATTTTT GGGGGGCTTT |
| 1561 | CCTCTGCTTG CCTCCCCTCG GATTTGGAGT GTCCGTTATC CTGCCTGCAT TTGGGGAGTC |
| 1621 | CCTTCTCGCT CTCTTTCCTC CACCCATTCT CTGATTTTCC TGCCTTTGCT GTCCCCTAGC |
| 1681 | CTTGAGGACT GGGGTGCTGG GTGTGGGGAT TGGAGTATAG GGTAGGGGAG AAGGGGGGGA |
| 1741 | GCATTCGGGG GAGTGGGGAG TGGGGGGAAG GAAAGCGGAG ACCGGACCAG GGGTTTTAAG |
| 1801 | GAGCAGGATG GTTCTGGGGT TTGGGTGGGG GGAGACGCGG GAAGGGTAGG AAAATGGACT |
| 1861 | GTTTCTGACC AGAGACACTT ACCTAAATAT CCTGGGGACC AAGGAACTAT GTACAAAAAC |
| 1921 | AAACCTACCA ACCACCAAAA ACTAGACAAA TAAAGACAAA CTAAAACAAA ACAGAACAAA |
| 1981 | AGCAAAGGAA AATGCTTTAG AAATTTTAAC TCCGGGGAGC CATAATCTGC AACTTCATTT |
| 2041 | TCCCCCATAG AAGAGAAAAA AGACCACCAC CATTATTACG ACCTCCCCAA CCCTACACGC |
| 2101 | ACGAACTGAG TCGAAAAACG AAAACCAAAC GAGCGAGAAG TTGAAGTTCT GGGTATCAAA |
| 2161 | GCTAGTTGTT CTGTCTGCGT GTTTAATTTT TCCCTCTCTC ACCTCCACCC CATCCATATC |
| 2221 | CTCTTTATTT CCTCCGTTCC AATGAGAGGC CTATGGCTGC TCTCCAATCC CGGGAAGTGA |
| 2281 | GTGGGAGCAC AGCTGAAAAG AGAGGGTCAG GGGGAGGCTG GCTGCTTGCT TAGGTGGAAT |
| 2341 | CCAACTTTTC CCGTGGCCCT GCCTATACTC TGGTGGCCTG GTCCTGTTGG GGTGGGGGTC |
| 2401 | TTTGGAGAGA AGGGCATAGT CTTTGAGCTA CTAAAAAGCA GAATTCCGGA GCTTCGAGAT |
| 2461 | ATCTTATTCT AGGAAAATGA AACAATTTTA ACAACAGTTT TTTTTCCTCT TATGTCGAAG |
| 2521 | ATCTAGTTTT AGACAATTTC AAAATAAGCT TTTCCCACTC ATAGAACTTT AACTTGCCCT |
| 2581 | TTCAGTTTTA TCTTTTTTTT AGAGAGAGGT TTAAACTACT GATTTTTCCT GTTGATTCAA |
| 2641 | ATAGACTAAT GGGGTGAAAG TTATTAGGAG AGATACTGTC TCCTGTTTTC TCCACTGAAC |
| 2701 | GAGACTCATC TTGCTCTTCT AGGTCCCGTT TCTTCCTCTC TTGGAGGACA TGAAATTATA |
| 2761 | GAAATGTTGA GAAGTTCCTG CTTTCTTTTG CGGTAGGACT TGGCTGTGAG AAAATCACCT |
| 2821 | AAATCCCAGA AAAGAGGAAG ACAGATTTAA AGTGCCCCCA CCCCCATTTG TTTCAAAGAG |
| 2881 | GTCTGCATGT TGGGCGAAAA CAGAACAACT GTGTTTCCTT TTACTTGTTC TTATTATTCA |
| 2941 | AGAGTCATTT ATTACAGGGG ATAAATGTTG GGTAGCAAGA ACTTTAATTT GCACTACCAG |
| 3001 | TCTCCCAAAT AGAAAATCAT GTATAGTATT TCATAGTAAT AATCAGGTAC CTTACAAGCT |
| 3061 | GCTGGTGGAT TTTAAAAAAT TAAGATAGTT GAAGGTGGTT AGGTAAAATG CCTGCTTTGT |
| 3121 | GTACAAGATA CTCTTTGGAT CTCTCGTAGA GATGGTTTGT TACCATCCTT TAATCATAAC |
| 3181 | TAAAACATTG AAAACAGAAC AAATGAGAAA AGAAAAAAAA CCTGCCGATT AACAAGACTG |
| 3241 | AAATCATGCA TGATCTGAAA GGTGTGGAAA GAAACACAAT TAGGTCTCAC TCTGGTTAGG |
| 3301 | CATTATTTAT TTAATTATGT TGTATATCAT TGTTTGCAGG GCAAACATTC TATGCATTTG |
| 3361 | AAACTGAGCA CTAAACTGGG CTAGCTTTCT GGTAGACCGT TTTGTGGCTA GTGCGATTTC |
| 3421 | ACAGTCTACT GCCTGTTTCC ACTGAAAACA TTTTTGTCAT ATTCTTGTAT TCAAAGAAAA |
| 3481 | CAGGAAAAAA GTTATTGTAA ATATTTTATT TAATGCACAC ATTCACACAG TGGTAACAGA |
| 3541 | CTGCCAGTGT TCATCCTGAA ATGTCTCACG GATTGATCTA CCTGTCTATG TATGTCTGCT |
| 3601 | GAGCTTTCTC CTTGGTTATG TTTTTTCTCT TTTACCTTTC TCCTCCCTTA CTTCTATCAG |
| 3661 | AACCAATTCT ATGCGCCAAA TACAACAGGG GGATGTGTCC CAGTACACTT ACAAAATAAA |
| 3721 | ACATAACTGA AAGAAGAGCA GTTTTATGAT TTGGGTGCGT TTTTGTGTTT ATACTGGGCC |
| 3781 | AGGTCCTGGT AGAACCTTTC AACAAACAAC CAAACAAAAA AAAA |

GENBANK ID: AAA61146.1
DEFINITION HUMAN TRANSCRIPTION FACTOR (E2A) MRNA, COMPLETE CDS.
VERSION M31523.1 GI:339477
CDS 31..1995
/CODON_START=1

| | |
|---|---|
| 1 | GCCTGAGGTG CCCGCCCTGG CCCCAGGAGA ATGAACCAGC CGCAGAGGAT GGCGCCTGTG |
| 61 | GGCACAGACA AGGAGCTCAG TGACCTCCTG GACTTCAGCA TGATGTTCCC GCTGCCTGTC |
| 121 | ACCAACGGGA AGGGCCGGCC CGCCTCCCTG GCCGGGGCGC AGTTCGGAGG TTCAGGTCTT |
| 181 | GAGGACCGGC CCAGCTCAGG CTCCTGGGGC AGCGGCGACC AGAGCAGCTC CTCCTTTGAC |
| 241 | CCCAGCCGGA CCTTCAGCGA GGGCACCCAC TTCACTGAGT CGCACAGCAG CCTCTCTTCA |
| 301 | TCCACATTCC TGGGACCGGG ACTCGGAGGC AAGAGCGGTG AGCGGGGCGC CTATGCCTCC |
| 361 | TTCGGGAGAG ACGCAGGCGT GGGCGGCCTG ACTCAGGCTG GCTTCCTGTC AGGCGAGCTG |
| 421 | GCCCTCAACA GCCCCGGGCC CCTGTCCCCT TCGGGCATGA AGGGGACCTC CCAGTACTAC |
| 481 | CCCTCCTACT CCGGCAGCTC CCGGCGGAGA GCGGCAGACG GCACCCTAGA CACCCAGCCC |
| 541 | AAGAAGGTCC GGAAGGTCCC GCCGGGTCTT CCATCCTCGG TGTACCCACC CAGCTCAGGT |
| 601 | GAGGACTACG GCAGGGATGC CACCGCCTAC CCGTCCGCCA AGACCGCCAG CAGCACCTAT |
| 661 | CCCGCCCCCT TCTACGTGGC AGATGGCAGC CTGCACCCCT CAGCCGAGCT CTGGAGTCCC |
| 721 | CCGGGCCAGG CGGGCTTCGG GCCCATGCTG GGTGGGGGCT CATCCCCGCT GCCCCTCCCG |
| 781 | CCCGGTAGCG GCCCGGTGGG CAGCAGTGGA AGCAGCAGCA CGTTTGGTGG CCTGCACCAG |
| 841 | CACGAGCGTA TGGGCTACCA GCTGCATGGA GCAGAGGTGA ACGGTGGGCT CCCATCTGCA |
| 901 | TCCTCCTTCT CCTCAGCCCC CGGAGGCACG TACGGCGGCG TCTCCAGCCA CACGCCGCCT |
| 961 | GTCAGCGGGG CCGACAGCCT CCTGGGCTCC CGAGGGACCA CAGCTGGCAG CTCCGGGGAT |
| 1021 | GCCCTCGGCA AAGCACTGGC CTCGATCTAC TCCCCGGATC ACTCAAGCAA TAACTTCTCG |
| 1081 | TCCAGCCCTT CTACCCCCGT GGGCTCCCCC CAGGGCCTGG CAGGAACGTC ACAGTGGCCT |
| 1141 | CGAGCAGGAG CCCCCGGTGC CTTATCGCCC AGCTACGACG GGGGTCTCCA CGGCCTGCAG |
| 1201 | AGTAAGATAG AAGACCACCT GGACGAGGCC ATCCACGTGC TCCGCAGCCA CGCCGTGGGC |
| 1261 | ACAGCCGGCG ACATGCACAC GCTGCTGCCT GGCCACGGGG CGCTGGCCTC AGGTTTCACC |
| 1321 | GGCCCCATGT CGCTGGGTGG GCGGCACGCA GGCCTGGTTG GAGGCAGCCA CCCCGAGGAC |
| 1381 | GGCCTCGCAG GCAGCACCAG CCTCATGCAC AACCACGCGG CCCTCCCCAG CCAGCCAGGC |
| 1441 | ACCCTCCCTG ACCTGTCTCG GCCTCCCGAC TCCTACAGTG GGCTAGGGCG AGCAGGTGCC |
| 1501 | ACGGCGGCCG CCAGCGAGAT CAAGCGGGAG GAGAAGGAGG ACGAGGAGAA CACGTCAGCG |
| 1561 | GCTGACCACT CGGAGGAGGA GAAGAAGGAG CTGAAGGCCC CCCGGGCCCG GACCAGCCCA |
| 1621 | GACGAGGACG AGGACGACCT TCTCCCCCCA GAGCAGAAGG CCGAGCGGGA GAAGGAGCGC |
| 1681 | CGGGTGGCCA ATAACGCCCG GGAGCGGCTG CGGGTCCGTG ACATCAACGA GGCCTTTAAG |
| 1741 | GAGCTGGGGC GCATGTGCCA ACTGCACCTC AACAGCGAGA AGCCCCAGAC CAAACTGCTC |
| 1801 | ATCCTGCACC AGGCTGTCTC GGTCATCCTG AACTTGGAGC AGCAAGTGCG AGAGCGGAAC |
| 1861 | CTGAATCCCA AAGCAGCCTG TTTGAAACGG CGAGAAGAGG AAAAGGTGTC AGGTGTGGTT |
| 1921 | GGAGACCCCC AGATGGTGCT TTCAGCTCCC CACCCAGGCC TGAGCGAAGC CCACAACCCC |
| 1981 | GCCGGCCACA TGTGAAAGGT ATGCCTCCGT GGGACGAGCC ACCCGCTTTC AGCCCTGTGC |
| 2041 | TCTGGCCCCA GAAGCCGGAC TCGAGACCCC GGGCTTCATC CACATCCACA CCTCACACAC |
| 2101 | CTGTTGTCAG CATCGAGCCA ACACCAACCT GACAAGGTTC GGAGTGATGG GGGCGGCCAA |
| 2161 | GGTGACACTG GGTCCAGGAG CTCCCTGGGG CCCTGGCCTA CCACTCACTG GCCTCGCTCC |
| 2221 | CCCTGTCCCC GAATCTCAGC CACCGTGTCA CTCTGTGACC TGTCCCATGG ATCCTGAAAC |
| 2281 | TGCATCTTGG CCCTGTTGCC TGGGCTGACA GGAGCATTTT TTTTTTTTCC AGTAAACAAA |
| 2341 | ACCTGAAAGC AAGCAACAAA ACATACACTT TGTCAGAGAA GAAAAAAATG CCTTAACTAT |
| 2401 | AAAAAGCGGA GAAATGGAAA CATATCACTC AAGGGGGATG CTGTGGAAAC CTGGCTTATT |
| 2461 | CTTCTAAAGC CACCAGCAAA TTGTGCCTAA GCGAAATATT TTTTTTAAGG AAAATAAAAA |
| 2521 | CATTAGTTAC AAGATTTTTT TTTTCTTAAG GTAGATGAAA ATTAGCAAGG ATGCTGCCTT |
| 2581 | TGGTCTCTGG TTTTTTTAAG CTTTTTTTGC ATATGTTTTG TAAGCAACAA ATTTTTTTGT |
| 2641 | ATAAAAGTCC CGTGTCTCTC GCTATTTCTG CTGCTGTTCC TAGACTGAGC ATTGCATTTC |
| 2701 | TTGATCAACC AGATGATTAA ACGTTGTATT AAAAAGACCC CGTGTAAACC TGAGCCCCCC |
| 2761 | CCGTCCCCCC CCCCGGAAGC CACTGCACAC AGACAGACGG GGACAGGCGG CGGGTCTTTT |
| 2821 | GTTTTTTTGA TGTTGGGGGT TCTCTTGGTT TTGTCATGTG GAAAGTGATG CGTGGGCGTT |
| 2881 | CCCTGATGAA GGCACCTTGG GGCTTCCCTG CCGCATCCTC TCCCCTCAGG AAGGGGACTG |
| 2941 | ACCTGGGCTT GGGGGAAGGG ACGTCAGCAA GGTGGCTCTG ACCCTCCCAG GTGACTCTGC |
| 3001 | CAAGCAGCTG TGGCCCCAGC GGTACCCTAC ACAACGCCCT CCCCAGGCCC CCCTAAGCTG |
| 3061 | CTCTCCCTTG GAACCTGCAC AGCTCTCTGA AATGGGGCAT TTTGTTGGGA CCAGTGACCC |
| 3121 | CTGGCATGGG GACCACACCC TGGAGCCCGG TGCTGGGGAC CTCCTGGACA CCCTGTCCTT |
| 3181 | CACTCCTTGC CCCAGGGACC CAGGCTCATG CTCTGAACTC TGGCTGAGAG GAGTCTGCTC |
| 3241 | AGGAGCCAGC ACAGGACACC CCCCACCCCA CCCCACCATG TCCCCATTAC ACCAGAGGGC |
| 3301 | CATCGTGACG TAGACAGGAT GCCAGGGGCC TGACCAGCCT CCCCAATGCT GGGGAGCATC |
| 3361 | CCTGGCCTGG GGCCACACCT GCTGCCCTCC CTCTGTGTGG TCCAAGGGCA AGAGTGGCTG |
| 3421 | GAGCCGGGGG ACTGTGCTGG TCTGAGCCCC ACGAAGGCCT TGGGCTGTGG CTCCGACCCT |
| 3481 | GCTGCAGAAC CAGCAGGGTG TCCCCTCGGG CCCATCTGTG TCCCATGTCC CAGCACCCAG |
| 3541 | GCCTCTCTCC AGGTCTCCTT TTCTGGTCTT TTGCCATGAG GGTAACCAGC TCTTCCCAGC |
| 3601 | TGGCTGGGAC TGTCTTGGGT TTAAAACTGC AAGTCTCCTA CCCTGGGATC CCATCCAGTT |
| 3661 | CCACACGAAC TAGGGCAGTG GTCACTGTGG CACCCAGGTG TGGGCCTGGC TAGCTGGGGG |
| 3721 | CCTTCATGTG CCCTTCATGC CCCTCCCTGC ATTGAGGCCT TGTGGACCCC TGGGCTGGCT |
| 3781 | GTGTTCATCC CCGCTGCAGG TCGGGCGTCT CCCCCCGTGC CACTCCTGAG ACTCCACCGT |
| 3841 | TACCCCCAGG AGATCCTGGA CTGCCTGACT CCCCTCCCCA GACTGGCTTG GGAGCCTGGG |
| 3901 | CCCCATGGTA GATGCAAGGG AAACCTCAAG GCCAGCTCAA TGCCTGGTAT CTGCCCCCAG |
| 3961 | TCCAGGCCAG GCGGAGGGGA GGGGCTGTCC GGCTGCCTCT CCCTTCTCGG TGGCTTCCCC |
| 4021 | TGCGCCCTGG GAGTTTGATC TCTTAAGGGA ACTTGCCTCT CCCTCTTGTT TTGCTCCTGC |
| 4081 | CCTGCCCCTA GGTCTGGGTG GCAGTGGCCC CATAGCCTCT GGAACTGTGC GTTCTGCATA |
| 4141 | GAATTCAAAC GAGATTCACC CAGCGCGAGG AGGAAGAAAC AGCAGTTCCT GGGAACCACA |
| 4201 | ATTATGGGGG GTGGGGGGTG TGATCTGAGT GCCTCAAGAT GGTTTTCAAA AAATTTTTTT |
| 4261 | TAAAGAAAAT AATTGTATAC GTGTCAACAC AGCTGGCTGG ATGATTGGGA CTTTAAAACG |
| 4321 | ACCCTCTTTC AGGTGGATTC AGACACCTGT CCTGTATATA ACACCACTGT AGCAATAAAC |
| 4381 | GTGACATTTT ATAAAG |

GENBANK ID: NM_000416.1
VERSION NM_000416.1 GI:4557879
MALLFLLPLVMQGVSRAENGTADLGPSSVPTPTNVTIESYNMNP
IVYWEYQIMPQVFVFTVEVKNYCVKNSENIDACTNISHHYCNISDNVCDPSNSLWVRV
KARVCQKESAYAKSEEFAVCRDCKIGPPKLDIRKEEKQINIDIFHFSVFVNCDEQEVD
YDPETTCYIRVYNVYVRMNGSEIQYKILTQKEGDCDEIQCQLAIPVSSLNSQYCVSAE
GVLEVWCVTTEKSKEVCITIFNSSIKCSLWIPVVAALLLWLVLSLVFICFYIKKINPL
KEKSIILPKSLISVVRSATLETKPESKYVSLITSYQPFSLEKEVVCEEFLSPATVPGM
HTEDNPCKVEHTEELSSITEVVTTEENIPDVVPGSELTPIERESSSPLSSNQSEPGSI
ALNSYESRNCSESDHSRNGFDTDSSCLESHSSLSDSEFPPNNKCEIKTEGQELITVIK
APTSFGYDKFHVLVDLLVDDSGKESLIGYRPTEDSKEFS
GENBANK ID: M27492.1
VERSION M27492.1 GI:186289
MKVLLRLICFIALLISSLEADKCKEREEKIILVSSANEIDVRPC
PLNPNEHKGTITWYKDDSKTPVSTEQASRIMQHKEKLWFVFAKVEDSGHYYCVVRNSS
YCLRIKISAKEVENEPNLCYNAQAIFKQKLPVAGDGCLVCPYMEFFKNENNELPKLQW
YKDCKPLLLDNIHFSGVKDRLIVMNVAEKHRGNYTCHASYTYLGKQYPITRVIEFITL
EENKFTRPVIVSPANETNEVDLGSQIQLICNVTGQLSDIAYNKWNGSVIDEDDPVLGE
DYYSVENPANKRRSTLITVLNISEIESRFYKNPFTCFAKNTHGIDAAYIQLIYPVTNF
QKHMIGICVTLTVIIVCSVFIYKIFKIDIVLWYRDSCYDFLPIKASDGKTYDAYILYP
KTVCEGSTSDCDIWVFKVLPEVLEKQCGYKLFIYCRDDYVGEDIVEVINENVKKSRRL
IIILVRETSGFSWLGGSSEEQIANYNALVQDGIKVVLLELEKIQDYEKMPESIKFIKQ
KHGAIRWSCDFTQGFQSAKTRFWKNVRYHMFVQRRSPSSKHQLLSPATKEKLQREAHV
PLG
GENBANK ID: L34059
VERSION L34059.1 GI:506409
MTACAGVLLLLLSLSCALRANNEDLTTRETCKAGFSEDDYTALI
SQNILEGEKLLQVKFSSCVGTKGTQYETNSMDFKVGADGTVFATRELQVPSEQVAFTV
TAWDSQTAEKWDAVVRLLVAQTSSPHSGHKPDKGKKVVALDPSPPPKDTLLPWPQHQN
ANGLRRRKRDWVIFPINVFENSRGPFFQQLVRIRSDKDNDIPIRYSITGVGADQPPME
VFSINSMSGRNYVTRPMDREEHASYHLRAHAVDNNGNKVENPIDLYIYVIDMNDNHPE
FINQVYNCSVDEGSKFGTYVMTITANDADDSTTANGMVRYRTVTQTPQSPSQNNFTIN
SETGDIVTVAAGWDREKVQQYTVIVQATDNEGNLNYGLSNTATAIITVTDVNDNPSEF
TASTFAGEVPENSVETVVANLTVMDRDQPHSBNWNAVYRIISGDPSGHFSVRTDFVTN
EGMVTVVKAVDYELNRAFNLTVNVSNQAPLASGIQNSFQSTAGVTISINDINEAPYFP
SNHKLILEEGVPPGTVLTTFSIAVDRDRFMQQAVRYSKLSDPASWLHINATNGQITTV
AVLDRESLYTKNNVYEATFLAADNCIFPAS0TGTLQIYLIDINDNAFELLPKEAQICE
RPNLNAINITAADADVHPNIGPYVFELPFVFAAVRKNWTITRLNCDYAQLSLRILYLE
AGMYDVFIIVTDSGNPFLSNTSIIKVKVCPCDDNGDCTTIGAVAAAGLGTGAIVAILI
CILILLTMVLLFVMWMKRREKERHTKQLLIDPEDDVREKILKYDEEGGGEEDQDYDLS
QLQQPEAMGHVPSKAPCVRRVDERPVGFEFQYPIRPMVPHPGDIGDFINEGLRAADND
PTAPPYDSLLVFDYEGSGSTAGSVSSLNSSSSGDQDYDYLNDWGPRFKKLADMYGGGE
ED
GENSANK ID: M77640
VERSION M77640.1 GI:186053
MVVALRYVWPLLLCSPCLLIQIPEEYEGHHVNEPPVITEQSPRR
LVVFPTDDISLKCEASGKPEVQFRWTRDGVHFKPKEELGVTVYQSFHSQSFTITGNNS
NFAQRFQGIYRCFASNKLGTAMSHEIRLMAEGAFKWPKETVKPVEVEEGESVVLPCNP
PPSAEFLRIYWMNSKILIHKQDERVTMGQNGNLYFANVLTSDNHSDYICHAEFFGTRT
IIQKEPIDLRVKATNSNIDRKPRLLFPTNSSSHLVALQGQPLVLECIAEGFPTPTIKW
LRPSGPMFADRVTYQNENKTLQLLKVGEEDDGEYRCLAENSLGSAREAYYVTVEAAPY
WLHKPQSHLY0PGETARLDCQVQGRPQPEVTWRINGIFVEELAKDQKYRIQRGALILS
NVQPSDTNVTQCEARNRNGLLLANAYIYVVQLPAKILTADNQTYMAVQGSTAYLLCKA
FGAPVPSVQWLDEDGTTVLQDERFFPYANGTLGIRDLQANDTCRYFCLAANDQNNVTI
MANLKVKDATQITQCFRSTIEKKGSRVTFTCQASFDPSLGPSITWRGDGRDLQELGDS
DKYFIEDGRLVIHSLDYSDQGNYSCVASTELDVVESRAQLLVVGSFGPVFRLVLSDLH
LLTQSQVRVSWSFAEDHNAFIEKYDIEFEDKENAFEKWYSLGKVFGNQTSTTLKLSPY
VHYTFRVTAINKYGPGEPSPVSETVVTPEAAFEKNPVDVKGEGNETTNMVTTWKPLRW
MDNNAFQVQYRVQWRPQGTRGPWQEQIVSDPFLVVSNTSTFVPYEIKVQAVNSQGKGP
EPQVTIGYSGEDYFQAIPELEGIETLNSSAVLVKWRPVDLAQVKGELRGYNVTYWREG
SQRKHSKRHIHKDHVVVFANTTSVILSGLRPYSSYHLEVQAFNGRGSGPASEFTESTP
EGVFGHPEALHLECQSNTSLLLRWQPPLSHNGVLTGYVLSYHPLDEGGKGQLSFNLRD
PELRTHNLTDLSPELRYRFQLQATTKEGPGEAIVREGGTMALSGISDFGNISATAGEN
YSVVSWVFKEGQCNFRFEILFKALGEEKGGASLSPQYVSYNQSSYTQWDLQPDTDYEI
HLFKERIAFRHQNAVKTNGTGRVRLFFAGFATEGWFIGFVSAIILLLLVLLILCIKRS
KGGKYSVKDKEDTQVDSEARPNKDETFGEYRSLESDNEEKAFGSSQPSLNGDIKPLGS
DDSLADYGGSVQVQFNEDGSFIGQYSGKKEKEAAGGNDSSGATSPINPAVALE
GENBANK ID: M59911.1
VERSION M59911.1 GI:186496
MGPGPSRAPRAPRLMLCALALMVAAGGCWSAFNLDTRFLVVKE
AGNPGSLFGYSVALERQTERQQRYLLLAGAPRELAVPDGYTNRTGAVYLCFLTAHKDD
CERMNITVKNDPGEHIIEDNWLGVTVASQGPAGRVLVCAHRYTQVLWSGSEDQRRMVG
KCYVRGNDLELDSSDDWQTYHNENCNSNTDYLETGMCQLGTSGGFTQNTVYFGASGAY
NWKGNSYNIQRKEWDLSEYSYKDPEDQGNLYIGYTMQVGSFILHPKNITIVTGAPRHR
HMGAVFLLSQEAGGDLRRRQVLEGSQVGAYFGSAIALADLNNDGWQDLLVGAPYYFER
KEEVGGAIYVFMNQAGTSFPAHPSLLLHCPSGSAFGLSVASIGDINQDGFQDIAVGAP
FEGLGKVYIYHSSSKGLLRQPQQVIHGERLGLPGLATFGYSLSGQMDVDENFYPDLLV
GSLSDHIVLLRARPVINIVHKTLVPRPAVLDPALCTATSCVQVELCFAYNQSAGNPNY
RRNITLAYTLEADRDRRPPRLRFAGSESAVFHGFFSNPENRCQKLELLLMDNLRDKLR
PIIISMNYSLPLRNPDRPRLGLRSLDAYPILNQAQALENHTEVQFQKECGPDNKCESN
LQMRAAFVSEQQQKLSRLQYSRDVRKLLLSINVTNTRTSERSGEDAHEALLTLVVPPA
LLLSSVRPPGACQANETIFCELGNPFKRNQRNELLIAFEVTGVTLHTRDLQVQLQLST
SSHQDNLWPMTLTLLVDYTLQTSLSMVNHRLQSFFGGTVMGESGMKTVEDVGSPLKYE
FQVGPNGEGLVGLGTLVLGLEWPYEVSNGKWLLYPTETTVHGNGSWPCRPPGDLINPL
NLTLSDPGDRPSSPQRRRRQLDPGGGQGPPFVTLAAAKKAKSETVLTCATGRAHCVWL
ECPIPDAPVVTNVTVKARVWNSTFTEDYRDFDRVRVNGWATLFLRTSIPTINMENKTT
WFSVDIDSELVEELPAETELWLVLVAVGAGLLLLGLIILLLWKCGFFKRARTRALYEA
KRQKAEMKSQPSETERLTDDY
GENBANK ID: M81695.1
VERSION M81695.1 GI:487829
MTRTRAALLLFTALATSLGFNLDTEELTAFRVGSAGFGDSVVQY
ANSWVVVGAPQKITAANQTGGLYQCGYSTGACEPIGLQVPPEAVNMSLGLSLASTTSP
SQLLACGPTVHHECGRNNYLTGLCFLLGPTQLTQRLPVSRQECPRQEQDIVFLIDGSG
SISSRNFATNMNFVRAVISQFQRPSTQFSLNQFSNKFQTHFTFEEFRRTSNPLSLLAS
VHQLQGFTYTATAIQNVVHRLFHASYGARRDATKTLIVITDGKKEGDSLDYKDVTPMA
DAAGIIRYATGVGLAFQNRNSNKELNDTASKPSQEHIFKVEDFDALKDIQNQLKEKIF
AIEGTETTSSSSFELENAQEGFSAVFTPDGPVLGAVGSFTWSGGAFLYPPNNSPTFIN
MSQENVDNRDSYLGYSTELALWKGVQSLVLGAPRYQHTGKAVIFTQVSRQWRNKAEVT
GTQTGSYFGASLCSVDVDTDGSTDLVLTGAPHYYEQTRGGQVSVCPLPRGWRRWWCDA
VLYGEQGHPWGRFGAALTVLGDVNGDKLTDVVIGAPGEEENRGAVYLFHGVLGPSISP
SNSQRTAGSQLSSRLQYFGQALSGGQDLTQDGLVDLAVGARGQVLLLRTRPVLWVGVS
MQFIPAETPRSAFECREQVVSEQTLVQSNTCLYIDKRSKNLLGSRDLQSSVTLDLALD
PGELSPRATFQETKNRSLSRVRVLGLKAHCENFNLLLPSCVEGSVTPTTLRLNFTLVG
KPLLAFRNLRPMLAALAQRYFTASLPFEKNCGADHTCQDNLGISFSFPGLKSLLVGSN
LELNAEVNVWNDGEDSYGTTITFSHPAGLSYRYVAEGGKQGQLRSLHLTCDSAPVGSQ
GTWSTSCRTNHLTFRGGAQTTFLATFDVSPKAVLGDRLLLTANVSSENNTPRTSKTTF
QLELPVKYAVYTVVSSHEQFTKYLNFSESEEKESHVAMHRYQVNNLGQRDLPVSTNFW
VPVELNQEAVWMDVEVSHPQNPSLRCSSEKIAPPASDFLAHIQKNPVLDCSTAGCLRF
RCDVPSFSVQEELDFTLKGNLSFGWVRQTLQKKVSVVSVAETTFDTSVYSQLPGQEAF
MRAQTTTVLEKYKVHNPTPLTVGSSTGGLLLLALITAVLYKVGFFKRQYKEMMEEANG
QTAPENGTQTPSPPSEK
GENBANK ID: X51841.1
VERSION X51841.1 GI:33910
MAGPEPSPWARLLLAALTSVSLSGTLANRCKKAPVKSCTECVRV
DKDCAYCTDEMFRDRRCNTQAELLAAGCQRESIVVMESSFQTTEETQTDTTLRRSQNS
PQGLRVRLRPGFERHFELEVFEPLESPVDLYTLNDFSNSMSDDLDNLKKMGQNLARVL
SQLTSDYTIGFGKFVDKVSVPQTDMRPEKLKEFWPNSDPPFSFKNVISLTEDVDEFRN
KLQGERISGNLDAPEGGFDAILQTAVCTRDIGWRFDSTHLLVESTESAFHYEADGANV
LAGIMSRNDERCHLDTTGTYTQYRTQDYPSVPTLVRLLAKHNIIPIFAVTNYSYSYYE
KLHTYFPVSSLGVLQEDSSNIVELLEEAFNRIRSNLDIRALDSPRGLRTEVTSKMFQK
TRTGSPHIRRGEVGIYQVQLRALEHVDGTHVCQLPEDQKGNIHLKPSFSDGLKMDAGI
ICDVCTCELQKEVRSARCSFNGDFVCGQCVCSEGWSGQTCNCSTGSLSDIQFCLREGE
DKPCSGRGECQCCHCVCYGEGRYEGQFCEYDNFQCPRTSGFLCNDRGRCSMGQCVCEP
GWTGPSCDCPLSNATCIDSNGGICNGRGHCECGRCHCHQQSLYTSTICEINYSATHPG
LCEDLRSCVQCQAWGTCEKKGRTCEECNFKVKMVDELKRAEEVVVRCSFRDEDDDCTY
SYTMEGDGAPGPNSTVLVHKKKDCPPGSFNWLIPLLLLLLPLLALLLLLCWKYCACCK
ACLALLPCCNRGHMVGFKEDHYMLRENLMASDHLDTPMLRSGNLKGRDVVRWKVTNNM
QRPGFATHAASTNPTELVPYGLSLRLARLCTENLLKPDTRECAQLRQEVEENLNEVYR
QISGVHKLQQTKFRQQPNAGKKQDHTTVDTVLMAPRSAKPALLKLTEKQVEQRAFHDL
KVAPGYYTLTADQDARGNVEFQEGVELVDVRVFLFIRPEDDDEKQLLVEAIDVPAGTA
TLGRRLVNITIIKEQARDVVSFEQFEFSVSRGDQVARIPVTRRVLDCGKSQVSYRTQD
GTAQENRDYTPVEGELLFQPGEAWKELQVKLLELQEVDSLLRGRQVRRFHVQLSNPKF
GAHLGQPHSTTIIIRDEDELDRSFTSQMLSSQPPFHGDLGAPQNPNAKAAGSRKIHFN
WLPPSGKPMCYRVKYNIQGDSESEAHLLDSKVFSVELTNLYPYCDYEMKVCAYGAQGE
GEYSSLVSCRTHQEVPSEPGRLAFNVVSSTVTQLSWAEPAETNGEITAYEVCYGLVND
DNREIGPMKKVLVDNPKNRMLLIENLRESQFYRYTVKARNGAGWGPEREAIINLATQP
KRPMSIFIIPDIPIVDAQSGEDYDSFLMYSDDVLRSPSGSQRPSVSDDTEHLVNGRMD
FAFPGSTNSLHRNTTTSAAAYGTHLSPHVPHRVLSTSSTLTRDYNSLTRSEHSHSTTL
PRDYSTLTSVSSHDSRLTAGVFDTPTRLVFSALGPTSLRVSWQEPRCERPLQGYSVEY
QLLNGGELHRLNIFNPAQTSVVVEDLLPNHSYVFRVRAQSQEGWGREREGVITIESQV
HPQSPLCPLPGSAFTLSTPSAPGPLVFTALSPDSLQLSWERPRRFNGDIVGYLVTCEM
AQGGGPATAFRVDGDSPESRLTVPGLSENVPYKFKVQARTTEGFGPEREGIITIESQV
GGPFPQLGSRAGLFQHFLQSEYSSITTTHTSATEPFLVDGPTLGAQHLEACGSLTRHV
TQEFVSRTLTTSGTLSTHMDQQFFQT
GENBANK ID: XP_030326.1
VERSION XP_030326.1 GI:14763626

| | |
|---|---|
| 1 | MDKFWWHAAW GLCLVPLSLA QIDLNITCRF AGVFHVEKNG RYSISRTEAA DLCKAFNSTL |
| 61 | PTMAQMEKAL SIGPETCRYG FIEGHVVIPR IHPNSICAAN NTGVYILTSN TSQYDTYCFN |
| 121 | ASAPFEEDCT SVTDLPNAFD GPITITIVNR DGTRYVQKGE YRTNPEDIYP SNPTDDDVSS |
| 181 | GSSSERSSTS GGYIFYTFST VHPIPDEDSE WITDSTDRIP ATTLNSTSAT ATETATKRQE |
| 241 | TWDWPSWLPL PSESKNHLHT TTQMAGTSSN TISAGWEPNE ENEDERDRHL SFSGSGIDDD |
| 301 | EDFISSTIST TPRAFDHTKQ NQDWTQWNPS HSNPEVLLQT TTRMTDVDRN GTTAYEGNWN |
| 361 | PEAHPPLIHH EHHEEEETPH STSTIQATPS STTEETATQK EQWFGNRWHE GYRQTEKEDS |
| 421 | HSTTGTAAAS AHTSHPMQGR TTPSFEDSSW TDFFNPISHP MGRGHQAGRR MDMDSSHSIT |
| 481 | LQPTANPNTG LVEDLDRTGP LSMTTQQSNS QSFSTSHEGL EEDKDHPTTS TLTSSNRNDV |
| 541 | TGGRRDPNHS EGSTTLLEGY TSHYPHTKES RTFIPVTSAK TGSPGVTAVT VGDSNSNVNR |
| 601 | SLSGDQDTFH PSGGSHTTEG SESDGESHGS QEGGANTTSG PIRTEQIPEW LIILASLLAL |
| 661 | ALILAVCIAV NSRRRCGQKK KLVINSGNGA VEDRKPSGLN GEASKSQEMV HLVNKESSET |
| 721 | PDQFMTADET RNLQNVDMKI GV |

GENBANK ID: NP_000826.1
VERSION NP_000826.1 GI:4504129

| | |
|---|---|
| 1 | MGGALGPALL LTSLPGAWAG LGPGQGEQGM TVAVVFSSSG PPQAQFRARL TPQSFLDLPL |
| 61 | EIQPLTVGVN TTNPSSLLTQ ICGLLGAAHV HGIVPEDNVD TEAVAQILDF ISSQTHVPIL |
| 121 | SISGGSAVVL TPKEPGSAFL QLGVSLEQQL QVLFKVLEEY DWSAFAVITS LHPGHALFLE |
| 181 | GVRAVADASH VSWRLLDVVT LELGEGGPRA RTQRLLRQLD APVPVAYCSR EEAEVLFAEA |
| 241 | AQAGLVGPGH VWLVPNLALG STDAPPATFP VGLISVVTES WRLSLRQKVR DGVAILALGA |
| 301 | HSYWRQHGTL PAPAGDCRVH PGPVSPAREA FYRHLLNVTW EGRDPSFSPG GYLVQPTMVV |
| 361 | IALNRHRLWE MVGRWEHGVL YMKYPVWPRY SASLQPVVDS RHLTVATLEE RPPVIVESPD |
| 421 | PGTGGCVPNT VPCRRQSNHT FSSGDVAPYT KLCCKGFCID ILKKLARVVK FSYDLYLVTN |
| 481 | GKHGKRVRGV WNGMIGEVYY KRADMAIGSL TINEERSEIV DPSVPFVETG ISVMVARSNG |
| 541 | TVSPSAFLEP YSPAVWVMMF VMCLTVVAIT VFMFEYPSPV SYNQNLTRGK KSGGPAFTIG |
| 601 | KSVWLLWALV FNNSVPIENP RGTTSKIMVL VWAFFAVIFL ASYTANLAAF MIQEQYIDTV |
| 661 | SGLSDKKPQR PQDQYPPFRF GIVPNGSTER NIRSNYRDMH THMVKFNQRS VEDALTSLKM |
| 721 | GKLDAFIYDA AVLNYMAGKD EGCKLVTIGS GKVPATTGYG IANQKDSHWK RAIDLALLQF |
| 781 | LGDGETQKLE TVWLSGICQN EKNEVMSSKL DIDNMAGVFY MLLVAMGLAL LVFAWEHLVY |
| 841 | WKLRHSVPNS SQLDPLLAFS RGIYSCPSGV QSLASPPRQA SPDLTASSAQ ASVLKNLQAA |
| 901 | RDMVTTAGVS SSLDRATRTI ENWGGGRRAP PPSPCPTPRS GPSPCLPTPD PPPEPSETGW |
| 961 | GPPDGGRAAL VRRAPQPPGR PPTPGPPLSD VSRVSRRPAW EARWPVRTGH CGRELSASER |
| 1021 | PLSPARCHYS SFPRADRSGR PFLPLFPEPP ELEDLPLLGP EQLARREALL HAAWARGSRP |
| 1081 | RHASLPSSVA EAFARPSSLP AGCTGPACAR PDGHSACRRL AQAQSMCLPI YREACQEGEQ |
| 1141 | AGAPAWQHRQ HVCLHAHAHL PFCWGAVCPH LPPCASHGSW LSGAWGPLGH RGRTLGLGTG |
| 1201 | YRDSGGLDEI SSVARGTQGF PGPCTWRRIS SLESEV |

GENBANK ID: CAA43045.1
DEFINITION HUMAN CDW40 MRNA FOR NERVE GROWTH FACTOR RECEPTOR-RELATED
B-LYMPHOCYTE ACTIVATION MOLECULE.
VERSION X60592.1 GI:29850
CDS 48..881
/CODON_START=1

| | |
|---|---|
| 1 | GCCTCGCTCG GGCGCCCAGT GGTCCTGCCG CCTGGTCTCA CCTCGCCATG GTTCGTCTGC |
| 61 | CTCTGCAGTG CGTCCTCTGG GGCTGCTTGC TGACCGCTGT CCATCCAGAA CCACCCACTG |
| 121 | CATGCAGAGA AAAACAGTAC CTAATAAACA GTCAGTGCTG TTCTTTGTGC CAGCCAGGAC |
| 181 | AGAAACTGGT GAGTGACTGC ACAGAGTTCA CTGAAACGGA ATGCCTTCCT TGCGGTGAAA |
| 241 | GCGAATTCCT AGACACCTGG AACAGAGAGA CACACTGCCA CCAGCACAAA TACTGCGACC |
| 301 | CCAACCTAGG GCTTCGGGTC CAGCAGAAGG GCACCTCAGA AACAGACACC ATCTGCACCT |
| 361 | GTGAAGAAGG CTGGCACTGT ACGAGTGAGG CCTGTGAGAG CTGTGTCCTG CACCGCTCAT |
| 421 | GCTCGCCCGG CTTTGGGGTC AAGCAGATTG CTACAGGGGT TTCTGATACC ATCTGCGAGC |
| 481 | CCTGCCCAGT CGGCTTCTTC TCCAATGTGT CATCTGCTTT CGAAAAATGT CACCCTTGGA |
| 541 | CAAGCTGTGA GACCAAAGAC CTGGTTGTGC AACAGGCAGG CACAAACAAG ACTGATGTTG |
| 601 | TCTGTGGTCC CCAGGATCGG CTGAGAGCCC TGGTGGTGAT CCCCATCATC TTCGGGATCC |
| 661 | TGTTTGCCAT CCTCTTGGTG CTGGTCTTTA TCAAAAAGGT GGCCAAGAAG CCAACCAATA |
| 721 | AGGCCCCCCA CCCCAAGCAG GAACCCCAGG AGATCAATTT TCCCGACGAT CTTCCTGGCT |
| 781 | CCAACACTGC TGCTCCAGTG CAGGAGACTT TACATGGATG CCAACCGGTC ACCCAGGAGG |
| 841 | ATGGCAAAGA GAGTCGCATC TCAGTGCAGG AGAGACAGTG AGGCTGCACC CACCCAGGAG |
| 901 | TGTGGCCACG TGGGCAAACA GGCAGTTGGC CAGAGAGCCT GGTGCTGCTG CTGCAGGGGT |
| 961 | GCAGGCAGAA GCGGGGAGCT ATGCCCAGTC AGTGCCAGCC CCTC |

GENBANK ID: AAB59544.1
DEFINITION HUMAN NERVE GROWTH FACTOR RECEPTOR MRNA, COMPLETE CDS.
VERSION M14764.1 GI:189204
CDS 114..1397
/CODON_START=1

| | |
|---|---|
| 1 | GCCGCGGCCA GCTCCGGCGG GCAGGGGGGG CGCTGGAGCG CAGCGCAGCG CAGCCCCATC |
| 61 | AGTCCGCAAA GCGGACCGAG CTGGAAGTCG AGCGCTGCCG CGGGAGGCGG GCGATGGGGG |
| 121 | CAGGTGCCAC CGGCCGCGCC ATGGACGGGC CGCGCCTGCT GCTGTTGCTG CTTCTGGGGG |
| 181 | TGTCCCTTGG AGGTGCCAAG GAGGCATGCC CCACAGGCCT GTACACACAC AGCGGTGAGT |
| 241 | GCTGCAAAGC CTGCAACCTG GGCGAGGGTG TGGCCCAGCC TTGTGGAGCC AACCAGACCG |
| 301 | TGTGTGAGCC CTGCCTGGAC AGCGTGACGT TCTCCGACGT GGTGAGCGCG ACCGAGCCGT |
| 361 | GCAAGCCGTG CACCGAGTGC GTGGGGCTCC AGAGCATGTC GGCGCCGTGC GTGGAGGCCG |
| 421 | ACGACGCCGT GTGCCGCTGC GCCTACGGCT ACTACCAGGA TGAGACGACT GGGCGCTGCG |
| 481 | AGGCGTGCCG CGTGTGCGAG GCGGGCTCGG GCCTCGTGTT CTCCTGCCAG GACAAGCAGA |
| 541 | ACACCGTGTG CGAGGAGTGC CCCGACGGCA CGTATTCCGA CGAGGCCAAC CACGTGGACC |
| 601 | CGTGCCTGCC CTGCACCGTG TGCGAGGACA CCGAGCGCCA GCTCCGCGAG TGCACACGCT |
| 661 | GGGCCGACGC CGAGTGCGAG GAGATCCCTG GCCGTTGGAT TACACGGTCC ACACCCCCAG |
| 721 | AGGGCTCGGA CAGCACAGCC CCCAGCACCC AGGAGCCTGA GGCACCTCCA GAACAAGACC |
| 781 | TCATAGCCAG CACGGTGGCA GGTGTGGTGA CCACAGTGAT GGGCAGCTCC CAGCCCGTGG |
| 841 | TGACCCGAGG CACCACCGAC AACCTCATCC CTGTCTATTG CTCCATCCTG GCTGCTGTGG |
| 901 | TTGTGGGCCT TGTGGCCTAC ATAGCCTTCA AGAGGTGGAA CAGCTGCAAG CAGAACAAGC |
| 961 | AAGGAGCCAA CAGCCGGCCA GTGAACCAGA CGCCCCCACC AGAGGGAGAA AAACTCCACA |
| 1021 | GCGACAGTGG CATCTCCGTG GACAGCCAGA GCCTGCATGA CCAGCAGCCC CACACGCAGA |
| 1081 | CAGCCTCGGG CCAGGCCCTC AAGGGTGACG GAGGCCTCTA CAGCAGCCTG CCCCCAGCCA |
| 1141 | AGCGGGAGGA GGTGGAGAAG CTTCTCAACG GCTCTGCGGG GGACACCTGG CGGCACCTGG |
| 1201 | CGGGCGAGCT GGGCTACCAG CCCGAGCACA TAGACTCCTT TACCCATGAG GCCTGCCCCG |
| 1261 | TTCGCGCCCT GCTTGCAAGC TGGGCCACCC AGGACAGCGC CACACTGGAC GCCCTCCTGG |
| 1321 | CCGCCCTGCG CCGCATCCAG CGAGCCGACC TCGTGGAGAG TCTGTGCAGT GAGTCCACTG |
| 1381 | CCACATCCCC GGTGTGAGCC CAACCGGGGA GCCCCCGCCC CGCCCCACAT TCCGACAACC |
| 1441 | GATGCTCCAG CCAACCCCTG TGGAGCCCGC ACCCCCACCC TTTGGGGGGG GCCCGCCTGG |
| 1501 | CAGAACTGAG CTCCTCTGGG CAGGACCTCA GAGTCCAGGC CCCAAAACCA CAGCCCTGTC |
| 1561 | AGTGCAGCCC GTGTGGCCCC TTCACTTCTG ACCACACTTC CTGTCCAGAG AGAGAAGTGC |
| 1621 | CCCTGCTGCC TCCCCAACCC TGCCCCTGCC CCGTCACCAT CTCAGGCCAC CTGCCCCCTT |
| 1681 | CTCCCACACT GCTAGGTGGG CCAGCCCCTC CCACCACAGC AGGTGTCATA TATGGCGGGC |
| 1741 | CAACACCAGC GATGGTACTA GGGGCAAGTG ACAAGGCCCC AGAGACTGAG AGGGAGGAAT |
| 1801 | CGAGGAACCA GAGCCATGGA CTCTACACTG TGAACTTGGG GAACAAGGGT GGCATCCCAG |
| 1861 | TGGCCTCAAC CCTCCCTCAG CCCCTCTTGC CCCCCACCCC AGCCTAAGAT GAAGAGGATC |
| 1921 | GGAGGCTTGT CAGAGCTGGG AGGGGTTTTC GAAGCTCAGC CCACCCCCCT CATTTTGGAT |
| 1981 | ATAGGACAGT GAGGCCCAGG GAGAGGGCAT GATTCGCCCA AAGCCAGACA GCAACGGGGA |
| 2041 | GGCCAAGTGC AGGCTGGCAC CGCCTTCTCT AAATGAGGGG CCTCAGGTTT GCCTGAGGGC |
| 2101 | GAGGGGAGGG TGGCAGGTGA CCTTCTGGGA AATGGCTTGA AGCCAAGTCA GCTTTGCCTT |
| 2181 | CCACGCTGTC TCCAGACCCC CACCCCTTCC CCACTGCCTG CCCACCCGTG GAGATGGGAT |
| 2221 | GCTTGCCTAG GGCCTGGTCC ATGATGGAGT CAGGTTTGGG GTTCGTGGAA AGGGTGCTGC |
| 2281 | TTCCCTCTGC CTGTCCCTCT CAGGCATGCC TCTGTGACAT CAGTGGCATG GCTCCAGTCT |
| 2341 | GCTGCCCTCC ATCCCGACAT GGACCCGGAG CTAACACTGG CCCCTAGAAT CAGCCTAGGG |
| 2401 | GTCAGGGACC AAGGACCCCT CACCTTGCAA CACACAGACA CACGCACACA CACACACAGG |
| 2461 | AGGAGAAATC TCACTTTTCT CCATGAGTTT TTTCTCTTGG GCTGAGACTG GATACTGCCC |
| 2521 | GGGGCAGCTG CCAGAGAAGC ATCGGAGGGA ATTGAGGTCT GCTCGGCCGT CTTCACTCGC |
| 2581 | CCCCGGGTTT GGCGGGCCAA GGACTGCCGA CCGAGGCTGG AGCTGGCGTC TGTCTTCAAG |
| 2841 | GGCTTACACG TGGAGGAATG CTCCCCCATC CTCCCCTTCC CTGCAAACAT GGGGTTGGCT |
| 2701 | GGGCCCAGAA GGTTGCGATG AAGAAAAGCG GGCCAGTGTG GGAATGCGGC AAGAAGGAAT |
| 2761 | TGACTTCGAC TGTGACCTGT GGGGATTTCT CCCAGCTCTA GACAACCCTG CAAAGGACTG |
| 2821 | TTTTTTCCTG AGCTTGGCCA GAAGGGGGCC ATGAGGCCTC AGTGGACTTT CCACCCCCTC |
| 2881 | CCTGGCCTGT TCTGTTTTGC CTGAAGTTGG AGTGAGTGTG GCTCCCCTCT ATTTAGCATG |
| 2841 | ACAAGCCCCA GGCAGGCTGT GCGCTGACAA CCACCGCTCC CCAGCCCAGG GTTCCCCCAG |
| 3001 | CCCTGTGGAA GGGACTAGGA GCACTGTAGT AAATGGCAAT TCTTTGACCT CAACCTGTGA |
| 3061 | TGAGGGGAGG AAACTCACCT GCTGGCCCCT CACCTGGGCA CCTGGGGAGT GGGACAGAGT |
| 3121 | CTGGGTGTAT TTATTTTCCT CCCCAGCAGG TGGGGAGGGG GTTTGGTGGC TTGCAAGTAT |
| 3181 | GTTTTAGCAT GTGTTTGGTT CTGGGGCCCC TTTTTACTCC CCTTGAGCTG AGATGGAACC |
| 3241 | CTTTTGGCCC CCAGCTGGGG GCCATGAGCT CCAGACCCCC AGCAACCCTC CTATCACCTC |
| 3301 | CCCTCCTTGC CTCCTGTGTA ATCATTTCTT GGGCCCTCCT GAAACTTACA CACAAAACGT |
| 3361 | TAAGTGATGA ACATTAAATA GCAAAG |

GENEANK ID: NP_002502.1
VERSION NM_002511.1 GI:4505406
CDS 140..1312
/CODON_START=1

| | |
|---|---|
| 1 | GTGCTGTGAG GCTTGCCCGC GGACAGTAAA CTTGCAGGGG CGAGAGGGAG GGACATCGAT |
| 61 | TAAACCTAAA TCGTGGGCGT TCAGTCCTCA GGGCACCGAG CGCGTGAAAA CTCCAGCGGA |
| 121 | CTCTGCTGGA AAGGAGATCA TGCCCTCTAA GTCTCTTTCC AACCTCTCGG TGACCACCGG |
| 181 | CGCGAATGAG AGCGGTTCCG TTCCCGAGGG GTGGGAAAGG GATTTCCTGC CGGCCTCGGA |
| 241 | CGGGACCACC ACGGAGTTGG TGATCCGCTG TGTGATCCCG TCCCTCTACC TGCTCATCAT |
| 301 | CACCGTGGGC TTGCTGGGCA ACATCATGCT GGTGAAGATC TTCATCACCA ACAGCGCCAT |
| 361 | GAGGAGCGTC CCCAACATCT TCATCTCTAA CCTGGCGGCC GGGGACTTGC TGCTGCTGCT |
| 421 | CACCTGCGTC CCGGTGGACG CCTCGCGCTA CTTCTTCGAC GAGTGGATGT TTGGCAAGGT |
| 481 | GGGCTGCAAA CTGATCCCTG TCATCCAGCT CACTTCCGTG GGGGTTTCCG TGTTCACTCT |
| 541 | CACTGCCCTC AGCGCCGACA GGTACAGAGC CATCGTTAAC CCCATCGACA TGCAGACGTC |
| 601 | AGGGCCATTG CTGCGGACCT GTGTGAAGGC CATGGGTATC TGGGTGGTCT CCGTGTTGCT |
| 661 | GGCAGTTCCC GAAGCGGTGT TTTCAGAAGT GGCTCGCATC AGTAGCTTGG ATAATAGCAG |
| 721 | CTTCACAGCA TGTATCCCAT ACCCTCAAAC AGATGAATTA CATCCAAAGA TTCATTCAGT |
| 781 | GCTCATTTTC TTGGTCTATT TCCTCATACC ACTTGCTATT ATTAGCATTT ATTATTATCA |
| 841 | TATTGCAAAG ACCTTAATTA AAAGCGCACA CAATCTTCCT GGAGAATACA ATGAACATAC |
| 901 | CAAAAAACAG ATGGAAACAC GGAAACGCCT GGCTAAAATT GTGCTTGTCT TTCTGGGCTG |
| 981 | TTTCATCTTC TGTTGGTTTC CAAACCACAT CCTTTACATG TATCGGTCTT TCAACTATAA |
| 1021 | TGAGATTGAT CCATCTCTAG GCCACATGAT TGTCACCTTA GTTGCCCGGG TTCTCAGTTT |
| 1081 | TGGCAATTCT TGTGTCAACC CATTTGCTCT TTACCTACTC AGTGAAAGCT TCAGGAGGCA |
| 1141 | TTTCAACAGC CAACTCTGCT GTGGGAGGAA GTCCTATCAA GAGAGAGGAA CCAGCTACCT |
| 1201 | ACTCAGCTCT TCAGCGGTGC GTATGACATC TCTGAAAAGC AATGCTAAGA ACATGGTGAC |
| 1261 | CAATTCTGTT TTACTAAATG GGCACAGCAT GAAGCAGGAA ATGGCAATCT GATTTTGGCC |
| 1321 | ATTCAACTCA CTACCTGGAG AGAACTTAGT AA |

GENBANK ID: Q00941
EST
DNA TYPE: CDNA
CGAAGGCGCGGCGGGCTCGGGGGCGGAGAACCTGACCTGCGAGATCCGAGCCGAGCGCTT
TCTTTTCTGCGCGTGGCGGGAGGGGCCAGCGGCGCCCGCGGACGTCCGGTACTCGCTGCG
AGTCCTTAACTCCACGGGTCACGACGTGGCGCGATGCATGGCCGACCCTGGGGATGACGT
CATGACACAGTGCATTGCGAACGACTTGTCACTGATGGGGAGTGAGGCCTACTTGGTCGT
GACCGGTCGCACCGGAGCGGGGCCAGTGCGGATCCTGGACGACTTGGTGGCTACGAAGGC
GCTCGACCGACTCGATCCCTCACCTGACGTCACCGAGTCCTGTAACTATTCCCACTGCAC
CGAGTCGTGGGCGCCGCCCTAGACCTGTGCATCCTATGAGGTGCGGGACTTGCAGTGTGA
GGTCCAGTGGCAGAGCACATATCCAGGAAGCTCACTCCAGAATGTGCTCATCCGCGAGGA
GAGGCGGTTTGCCTTTCGCACGCTGTTCCGCTCGAGGTTACATTGCTAAAGTGCGCACAG
GGTACACGAGGATGAGCACTGTGGCGAATGGTATAAGCGGCATCCTGTTATGGCTGAG
GENBANK ID: P05106
EST
DNA TYPE: CDNA
CGGCCGCTGCACAGCAGCCCATTGCTGGACATGCAGGTGTCAGTACGCGTGGTACAGTTG
CAGTAGTAGCCGGTCCAGTCGGAGTCACACAGGCAGTCCCCACAGCTGCACTGGCCATGG
CCTGAGCACATCTCCCCCTTGTAGCGGACACAGGAGAAGTCGTCACACTCGCAGTACTTG
CCCGTGATCTTGCCAAAGTCACTGCTGTGGCAGACACATTGACCACAGAGGCACTCGCCC
CGCTGGCTGCAGACGGGCTGACCCTCTCGGGGGCTGCACTCGTCCTGCTGGGAAGGGCGA
TAGTCCTCCTCTGAGCACTCACACTGGGATCCCAGCCAGCCAGGCCCACAACGGCATACC
CCACACTCAAAGGTCCCATTGCCATTGTTGCAGCGATGGCTATTAGGTTCAGCTTGGGCC
TGGCAGGCACAGTCACAATCAAAGGTGACCTGGACGATCAGGCTGTCCTTGAAGCCCACG
GGCTTTATGGTAAAGGACTTCTCCTTCTCCTGGGGACAGCCTCGCACCTTGGCCTCAATG
CTGAAGCTCACCGTGTCTCCAATCTTGAGTCCCATACAAGACTTGAGGCCAGGGATGACC
TCATTGGTGAGGCAGGT
EST GENBANK ACC: BF115658
DNA TYPE: CDNA
GCGGCCGCTGCACAGCAGCCCATTGCTGGACATGCAGGTGTCAGTACGCGTGGTACAGTT
GCAGTAGTAGCCGGTCCAGTCGGAGTCACACAGGCAGTCCCCACAGCTGCACTGGCCATG
GCCTGAGCACATCTCCCCCTTGTAGCGGACACAGGAGAAGTCGTCACACTCGCAGTACTT
GCCCGTGATCTTGCCAAAGTCACTGCTGTGGCAGACACATTGACCACAGAGGCACTCGCC
CCGCTGGCTGCAGACGGGCTGACCCTCCCGGGGGCTGCATTCGTCCTGCTGGGAAGGGCG
ATAGTCCTCCTCTGAGCACTCACACTGGGATCCCAGCCAGCCAGGCCCACAACGGCATAC
CCCACACTCAAAGGTCCCATTGCCATTGTTGCAGCGATGGCTATTAGGTTCAGCTTGGGC
CTGGCAGGCACAGTCACAATCAAAGGTGACCTGNACGATCAGGCTGTCCTTGAAGCCCAC
GGGCTNTATGGTAAAGGACTTCTCCTTCTCCTGGGGACAGCCTCGCACCTTGGCCTCAAT
GCTGAAGCTCACCGTGTCTCCAATCTTGAGTCCCATACAAGACTTGAGGCCAGGGATGAC
CTCATTGTTGAGGCATGTGGCATTGAAGGATAGAGANCACTCTTCAGGACGTCACGCACT
TTCAGCTCGACTTTAGAACGGAATTTCCATAAGCATCAACAATGAGCCTGAGGACATTGC
CTGAATCCATGGACAGAACCCCCACTGTGGTCCC
EST
GENBANK ACC: BFD62996
CGGCCGCTGCACAGCAGCCCATTGCTGGACATGCAGGTGTCAGTACGCGTGGTACAGTTG
CAGTAGTAGCCGGTCCAGTCGGAGTCACACAGGCAGTCCCCACAGCTGCACTGGCCATGG
CCTGAGCACATCTCCCCCTTGTAGCGGACACAGGAGAAGTCGTCACACTCGCAGTACTTG
CCCGTGATCTTGCCAAAGTCACTGCTGTGGCAGACACATTGACCACAGAGGCACTCGCCC
CGCTGGCTGCAGACGGGCTGACCCTCCCGGGGGCTGCATTCGTCCTGCTGGGAAGGGCGA
TAGTCCTCCTCTGAGCACTCACACTGGGATCCCAGCCAGCCAGGCCCACAACGGCATACC
CCACACTCAAAGGTCCCATTGCCATTGTTGCAGCGATGGCTATTATGTTCAGCTTGGGCC
TGGCAGGCACAGTCACAATCAAAGGTGACCTGGACGATCAGGCTGTCCTTGAAGCCCACG
GGCTTTATGGTAAAGGAATTCTCCTTTTCCTTGGGACAGACTCGCACCTTGGCCCTAATG
CTGAAGCTCACCGAGATCTTCAT
GENBANK ID: CAA52348.1
VERSION X74295.1 GI:437781
CDS <1..234
/CODON_START=1

| | |
|---|---|
| 1 | AAGATGGGAT TCTTCAAACG GGCGAAGCAC CCCGAGGCCA CCGTGCCCCA GTACCATGCG |
| 61 | GTGAAGATTC CTCGGGAAGA CCGACAGCAG TTCAAGGAGG AGAAGACGGG CACCATCCTG |
| 121 | AGGAACAACT GGGGCAGCCC CCCGCCGGAC GGCCCGGATG CACACGCCAT CCTCGCTGCT |
| 181 | GACGGGCATC CCCAGCTGGG CCCCAATGGG CATCCAGGGC CAGGCACCGC CTAGGTTCCC |
| 241 | ATGTCCCAGC CTGCGCTGTG GCTGCCCTCC ATCCCTTCCC CAGAGATGGC TCCTTGGGAT |
| 301 | GAAGAGGGTA GAGTCGGCTG CTGGTGTCAC ATCAAGAATT TGGCAGGATC GGCTTCCTCA |
| 361 | GGGGCACAGA CCTCTCCCAC CCACAAGAAC TGCTGCCACG CAACTTCCCC TTAGAGTGCT |
| 421 | GTGAGATGAG AGTGGGTAAA TCAGGGACAG GGCCATGGGG TAGGGTGAGA AGGGCAGGGG |
| 481 | TGTCCTGATG CAAAGGTGGG GAGAAGGATC CTAATCCCTT CCTCTCCCAT TCACCCTGTG |
| 541 | TAACAGGACC CCAAGGACCT GCCTCCCCGG AAGTGCCTTA ACCTAGAGGG TCGGGGAGGA |
| 601 | GGTTGTGTCA CTGACTCAAG GCTGCTCCTT CTCTAGTTTC CCCTCTCATC TGACCTTAGT |
| 661 | TTGCTGCCAT CAGTCTAGTG GTTTCGTGGT TTCGTCTATT TATTAAAAAA TCGGAACCC |

GENBANK ID: M57627
VERSION CAA51942.1 GI:580177

| | |
|---|---|
| 1 | MHSSAL |

GENBANK ID: AAA52578.1
VERSION AAA52578.1 GI:183364

| | |
|---|---|
| 1 | MWLQSLLLLG TVACSISAPA RSPSPSTQPW EHVNAIQEAR RLLNLSRDTA AEMNETVEVI |
| 61 | SEMFDLQEPT CLQTRLELYK QGLRGSLTKL KGPLTMMASH YKQRCPPTPE TSCATQIITF |
| 121 | ESFKENLKDF LLVIPFDCWE PVQE |

GENBANK ID: AAA58482.1
VERSION AAA58482.1 GI:182669

| | |
|---|---|
| 1 | METNFSIPLN ETEEVLPEPA GHTVLWIFSL LVHGVTFVFG VLGNGLVIMV AGFRMTRTVN |
| 61 | TICYLNLALA DFSFSAILPF RNVSVANREK WPFASFLCKL VHVNIDINLF VSVYLITIIA |
| 121 | LDRCICVLHP AWAQNHRTMS LAKRVMTGLW IFTIVLTLPN FIFNTTISTT NGDTYCIFNF |
| 181 | AFWGDTAVER LNVFITNAKV FLILHFIIGF TVPMSIITVC YGIIAAKIHR NHMIKSSRPL |
| 241 | RVFAAVVASF FICWFPYELI GILMAVWLKE NLLNGKYKII LVLINPTSSL AFFNSCLNPI |
| 301 | LYVFMGRNFQ ERLIRSLPTS LERALTEVPD SAQTSNTHTT SASPPEETEL QAM |

GENBANK ID: P17774
VERSION P17774 GI:121324

| | |
|---|---|
| 1 | MSESLVVCDV AEDLVEKLRK FRFRKETNNA ATINKIDMDK RLVVLDEELE GISPDELKDE |
| 61 | LPERQPRFIV YSYKYQHDDG RVSYPLCFIF SSPVGCKPEQ QMMYAGSKNK LVQTAELTKV |
| 121 | FEIRNTEDLT EEWLREKLGF FH |

GENBANK ID: P51858
VERSION P51858 GI:1708157

| | |
|---|---|
| 1 | MSRSNRQKEY KCGDLVFAKM KGYPHWPARI DEMPEAAVKS TANKYQVFFF GTHETAFLGP |
| 61 | KDLFPYEESK EKFGKPNKRK GFSEGLWEIE NNPTVKASGY QSSQKKSCVE EPEPEPEAAE |
| 121 | GDGDKKGNAE GSSDEEGKLV IDEPAKEKNE KGALKRRAGD LLEDSPKRPK EAENPEGEEK |
| 181 | EAATLEVERP LPNEVEKNST PSEPGSGRGP PQEEEEEEDE EEEATKEDAE APGIRDHESL |
| 241 | |

GENBANK ID: P10147
VERSION P10147 GI:127078

| | |
|---|---|
| 1 | MQVSTAALAV LLCTMALCNQ FSASLAADTP TACCFSYTSR QIPQNFIADY FETSSQCSKP |
| 61 | GVIFLTKRSR QVCADPSEEW VQKYVSDLEL SA |

GENBANK ID: P13500
VERSION P13500 GI:126842

| | |
|---|---|
| 1 | MKVSAALLCL LLIAATFIPQ GLAQPDAINA PVTCCYNFTN RKISVQRLAS YRRITSSKCP |
| 61 | KEAVIFKTIV AKEICADPKQ KNVQDSMDHL DKQTQTPKT |

GENBANK ID: NP_065391.1
VERSION NP_065391.1 GI:10092621

| | |
|---|---|
| 1 | MGVLLTQRTL LSLVLALLFP SMASMAAIGS CSKEYRVLLG QLQKQTDLMQ DTSRLLQPYI |
| 61 | RIQGLDVFKL REHCRERPGA FPSEETLRGL GRRGFLQTLN ATLGCVLHRL ADLEQRLFKA |
| 121 | QDLERSGLNI EDLEKLQMAR FNILGLRNNI YCMAQLLDNS DTAEPTKAGR DASQPPTPTP |
| 181 | ASDAFQRKLE GCRFLHGYHR FMESVGRVFS KWGESFNRSR RHSPHQALRK GVRRTRPSRK |
| 241 | GKRLMTRGQL PR |

GENBANK ID: XP_013053.3
VERSION XP_013053.3 GI:14768277

| | |
|---|---|
| 1 | MERERETLQA WEERVGQELD RVVAFWNEHS HDQEEGGFFT CLGREGRVYD DLKYVWLQGR |
| 61 | QVWMYCRLYR TFERFRHAQL LDAAKAGGEF LLRYARVAPP GKKCAFVLTR DGRPVKVQRT |
| 121 | IFSECFYTMA MNELWRATGE VRYQTEAVEM MDQIVHWVQE DASGLGRPQL QGAPAAEPMA |
| 181 | VPMNLLNLVE QLGEADEELA GKYAELGDWC ARRILQHVQR DGQAVLENVS EGGKELPGCL |
| 241 | GRQQNPGHTL EAGWFLLRHC IRIGDPELRA HVIDKWLLLP FHSGWDPDHG GLFYFQDADN |
| 301 | FCPTQLEWAN KLWWPHSEAM IAFLMGYSDS GDPVLLRLFY QVAEYTFRQF RDPEYGENFG |
| 361 | YLSREGKVAL SIKGGPFKGC FHVPRCLAMC EEMLGALLSR PAPAPSPAPT PACRGAE |

GENBANK ID: B31848
VERSION B31848 GI:87005

| | |
|---|---|
| 1 | MTCKNSQLER NIETIINTFH QYSVKLGEPD TLNQGEFKEL VRKDLQNFLK KENKNEKVIE |
| 61 | HIMEDLDTNA DKQLSFEEFI MLMARLTWAS HENNEEGDEG PGHHHKPGLG ECTP |

GENBANK ID: CAA38698.1
VERSION CAA38698.1 GI:35522

| | |
|---|---|
| 1 | MPVNRLFPCF LQLLAGLALP AVFFQQWALS AGNGSSEVEV VFFQEVWGRS YCRALERLVD |
| 61 | VVSEYPSEVE HMFSFSCVSL LRCTGCCGDE NLHCVPVETA NVTMQLLKIR SGDRPSYVEL |
| 121 | TFSQHVRCEC RPLREKMKPE RCGDAVPRR |

GENBANK ID: AAA35789.1
VERSION AAA35789.1 GI:181971

| | |
|---|---|
| 1 | MNFLLSWVHW SLALLLYLHH AKWSQAAPNA EGGGQNHHEV VKFMDVYQRS YCHFIETLVD |
| 61 | IFQEYPDEIE YIFKPSCVFL NRCGGCCNDE GLECVPTEES NITMQINRIK PHQGQHIGEM |
| 121 | SFLQHNKCEC RPKKDRARQE NPCGPCSERR KHLFVQDPQT CKCSCKNTDS RCKARQLELN |
| 181 | ERTCRCDKPR R |

GENBANK ID: AAA66062.1
VERSION AAA66062.1 GI:536898

| | |
|---|---|
| 1 | MWKRWLALAL ALVAVAWVRA EEELRSKSKI CANVFCGAGR ECAVTEKGEF TCLCIEQCKP |
| 61 | HKRFVCGSNG KTYLNHCELH RDACLTGSKI QVDYDGHCKE KKSVSPSASP VVCYQSNRDE |
| 121 | LRRRIIQWLE AEIIFDGWFS KGSNYSEILD KYFKNFDNGD SRLDSSEFLK FVEQNETAIN |
| 181 | ITTYPDQENN KLLRGLCVDA LIELSDENAD WKLSFQEFLK CLNPSFNPPE KKCALEDETY |
| 241 | ADGAETEVDC NRCVCACGNW VCTANTCDGK NQKGAQTQTE EEMTRYVQEL QKHQETAEKT |
| 301 | KRVSTKEI |

GENBANK ID: AAA59872.1
VERSION AAA59872.1 GI:398038

| | |
|---|---|
| 1 | MGWLPLLLLL TQCLGVPGQR SPLNDFQVLR GTELQHLLHA VVPGPWDEDV ADAEECAGRC |
| 61 | GPLMDCRAFH YNVSSHGCQL LFWTQRSPHT RLRRSGRCDL FQKKDYVRTC IMNNGVGYRG |
| 121 | TMATTVGGLP CQAWSHKFPN DHKYTFTLRN GLEENFCRNP DGDPGGFWCY TTDPAVRFQS |
| 181 | CGIKSCREAA CVWCNGEEYR GAVDRTESGR ECQRWQLQHP HQHPFEPGKF LDQGLDDNYC |
| 241 | RNPDGSERPW CYTTDPQIER EFCDLFRCGS EAQFRQEATT VSCFRGKGEG YRGTANTTTA |
| 301 | GVPCQRWDAQ IFHQHRFTPE KYACKDLREN FCRNPDGSEA PWCFTLRPGM RAAFCYQIRR |
| 361 | CTDDVRPQDC YHGAGEQYRG TVSKTRKGVQ CQRWSAETPH KPQFTFTSEP HAQLEENFCR |
| 421 | NPDGDSHGPW CYTMDPRTPF DYCALRRCAD DQPPSILDPP DQVQFENCGK RVDRLDQRRS |
| 481 | KLRVVGGHPG NSFWTVSLRN RQGQHFCGGS LVKEQWILTA RQCFSSCHMP LTGYEVWLGT |
| 541 | LFQNPQHGEP SLQRVPVAKM VCGPSGSQLV LLKLERSVTL NQRVALICLP PEWYVVFFGT |
| 601 | KCEIAGWGET KGTGNDTVLN VAFLNVTSNQ ECNIKHRGRV RESENCTEGL LADVGACEGD |
| 661 | YGGPLACFTH NCWVLEGIII PNRVCARSRW PAVFTRVSVF VDWIHKVMRL G |

GENBANK ID: P01579
VERSION P01579 GI:124479

| | |
|---|---|
| 1 | MKYTSYILAF QLCIVLGSLG CYCQDPYVKE AENLKKYFNA GHSDVADNGT LFLGILKNWK |
| 61 | EESDRKIMQS QIVSFYFKLF KNFKDDQSIQ KSVETIKEDM NVKFFNSNKK KRDDFEKLTN |
| 121 | YSVTDLNVQR KAIHELIQVM AELSPAAKTC KRKRSQMLFR GRRASQ |

GENBANK ID: XP_035842.1
DEFINITION HOMO SAPIENS SMALL INDUCIBLE CYTOKINE A5 (RANTES) (SCYAS),
MRNA.
CDS 22..297
/CODON_START=1

| | |
|---|---|
| 1 | ACAGCCTCTC CCACAGGTAC CATGAAGGTC TCCGCGGCAG CCCTCGCTGT CATCCTCATT |
| 61 | GCTACTGCCC TCTGCGCTCC TGCATCTGCC TCCCCATATT CCTCGGACAC CACACCCTGC |
| 121 | TGCTTTGCCT ACATTGCCCG CCCACTCCCC CGTGCCCACA TCAAGGAGTA TTTCTACACC |
| 181 | AGTGGCAAGT GCTCCAACCC AGCAGTCGTC TTTGTCACCC GAAAGAACCG CCAAGTGTGT |
| 241 | GCCAACCCAG AGAAGAAATG GGTTCGGGAG TACATCAACT CTTTGGAGAT GAGCTAGGAT |
| 301 | GGAGAGTCCT TGAACCTGAA CTTACACAAA TTTGCCTGTT TCTGCTTGCT CTTCTCCTAG |
| 361 | CTTGGGAGGC TTCCCCTCAC TATCCTACCC CACCCGCTCC TTGAAGGGCC CAGATTCTAC |
| 421 | CACACAGCAG CAGTTACAAA AACCTTCCCC AGCCTGGACC TGGTGGCTCA CGCCTGTAAT |
| 481 | CCCAGCACTT TGGGAGGGCA AGGTGGGTGG ATCACTTGAG GTCAGGAGTT CGAGACCAGC |
| 541 | CTGGCCAACA TGATGAAACC CCATCTCTAC TAAAAATACA AAAAATTAGC CGGGCGTGGT |
| 601 | AGCGGGCGCC TGTAGTCCCA GCTACTCGGG AGGCTGAGGC AGGAGAATGG CGTGAACCCG |
| 661 | GGAGGCGGAG CTTGCAGTGA GCCGAGATCG CGCCACTGCA CTCCAGCCTG GGCGACAGAG |
| 721 | CGAGACTCCG TCTCAAAAAA AAAAAAAAAA AAAAAAATAC AAAAATTAGC CGGGCGTGGT |
| 781 | GGCCCACGCC TGTAATCCCA GCTACTCGGG AGGCTAAGGC AGGAAAATTG TTTGAACCCA |
| 841 | GGAGGTGGAG GCTGCAGTGA GCTGAGATTG TGCCACTTCA CTCCAGCCTG GGTGACAAAG |
| 901 | TGAGACTCCG TCACAACAAC AACAACAAAA AGCTTCCCCA ACTAAAGCCT AGAAGAGCTT |
| 961 | CTGAGGCGCT GCTTTGTCAA AAGGAAGTCT CTAGGTTCTG AGCTCTGGCT TTGCCTTGGC |
| 1021 | TTTGCCAGGG CTCTGTGACC AGGAAGGAAG TCAGCATGCC TGTAGAGGCA AGGAGGGGAG |
| 1081 | GAACACTGCA CTCTTAAGCT TCCGCCGTCT CAACCCCTCA CAGGAGCTTA CTGGCAAACA |
| 1141 | TGAAAAATCG G |

GENBANK ID: CAA72079.1
DEFINITION H. SAPIENS MRNA FOR ESTROGEN SULFOTRANSFERASE.
CDS 63..947
/CODON_START=1

| | |
|---|---|
| 1 | GCCTTGAAGA GATATAAACT AGATCAATTT CTTTCACAGG ATCAACTAAA CAGTGTACCA |
| 61 | CAATGAATTC TGAACTTGAC TATTATGAAA AGTTTGAAGA AGTCCATGGG ATTCTAATGT |
| 121 | ATAAAGATTT TGTCAAATAT TGGGATAATG TGGAAGCGTT CCAGGCAAGA CCAGATGATC |
| 181 | TTGTCATTGC CACCTACCCT AAATCTGGTA CAACCTGGGT TAGTGAAATT GTGTATATGA |
| 241 | TCTATAAAGA GGGTGATGTG GAAAAGTGCA AAGAAGATGT AATTTTTAAT CGAATACCTT |
| 301 | TCCTGGAATG CAGAAAAGAA AACCTCATGA ATGGAGTAAA ACAATTAGAT GAGATGAATT |
| 361 | CTCCTAGAAT TGTGAAGACT CATTTGCCAC CTGAACTTCT TCCTGCCTCA TTTTGGGAAA |
| 421 | AGGATTGTAA GATAATCTAT CTTTGCCGGA ATGCAAAGGA TGTGGCTGTT TCCTTTTATT |
| 481 | ATTTCTTTCT AATGGTGGCT GGTCATCCAA ATCCTGGATC CTTTCCAGAG TTTGTGGAGA |
| 541 | AATTCATGCA AGGACAGGTT CCTTATGGTT CCTGGTATAA ACATGTAAAA TCTTGGTGGG |
| 601 | AAAAGGGAAA GAGTCCACGT GTACTATTTC TTTTCTACGA AGACCTGAAA GAGGATATCA |
| 661 | GAAAAGAGGT GATAAAATTG ATACATTTCC TGGAAAGGAA GCCATCAGAG GAGCTTGTGG |
| 721 | ACAGGATTAT ACATCATACT TCGTTCCAAG AGATGAAGAA CAATCCATCC ACAAATTACA |
| 781 | CAACACTGCC AGACGAAATT ATGAACCAGA AATTGTCGCC CTTCATGAGA AAGGGAATTA |
| 841 | CAGGAGACTG GAAAAATCAC TTTACAGTAG CCCTGAATGA AAAATTTGAT AAACATTATG |
| 901 | AGCAGCAAAT GAAGGAATCT ACACTGAAGT TTCGAACTGA GATCTAAGAA GGTCTT |

GENBANK ID: AAA63210.1
DEFINITION HUMAN KERATINOCYTE GROWTH FACTOR MRNA, COMPLETE CDS.
CDS 446..1030
/CODON_START=1

| | |
|---|---|
| 1 | ACGCGCTCAC ACACAGAGAG AAAATCCTTC TGCCTGTTGA TTTATGGAAA CAATTATGAT |
| 61 | TCTGCTGGAG AACTTTTCAG CTGAGAAATA GTTTGTAGCT ACAGTAGAAA GGCTCAAGTT |
| 121 | GCACCAGGCA GACAACAGAC ATGGAATTCT TATATATCCA GCTGTTAGCA ACAAAACAAA |
| 181 | AGTCAAATAG CAAACAGCGT CACAGCAACT GAACTTACTA CGAACTGTTT TTATGAGGAT |
| 241 | TTATCAACAG AGTTATTTAA GGAGGAATCC TGTGTTGTTA TCAGGAACTA AAAGGATAAG |
| 301 | GCTAACAATT TGGAAAGAGC AAGTACTCTT TCTTAAATCA ATCTACAATT CACAGATAGG |
| 361 | AAGAGGTCAA TGACCTAGGA GTAACAATCA ACTCAAGATT CATTTTCATT ATGTTATTCA |
| 421 | TGAACACCCG GAGCACTACA CTATAATGCA CAAATGGATA CTGACATGGA TCCTGCCAAC |
| 481 | TTTCCTCTAC AGATCATGCT TTCACATTAT CTGTCTAGTG GGTACTATAT CTTTAGCTTG |
| 541 | CAATGACATG ACTCCAGAGC AAATGGCTAC AAATGTGAAC TGTTCCAGCC CTGAGCGACA |
| 601 | CACAAGAAGT TATGATTACA TGGAAGGAGG GGATATAAGA GTGAGAAGAC TCTTCTGTCG |
| 661 | AACACAGTGG TACCTGAGGA TCGATAAAAG AGGCAAAGTA AAAGGGACCC AAGACATGAA |
| 721 | GAATAATTAC AATATCATGG AAATCAGGAC AGTGGCAGTT GGAATTGTGG CAATCAAAGG |
| 781 | GGTGGAAAGT GAATTCTATC TTGCAATGAA CAAGGAAGGA AAACTCTATG CAAAGAAAGA |
| 841 | ATGCAATGAA GATTGTAACT TCAAAGAACT AATTCTGGAA AACCATTACA ACACATATGC |
| 901 | ATCAGCTAAA TGGACACACA ACGGAGGGCA AATGTTTCTT GCCTTAAATC AAAAGCCCAT |
| 961 | TCCTGTAAGA GGAAAAAAAA CGAAGAAAGA ACAAAAAACA GCCCACTTTC TTCCTATGGC |
| 1021 | AATAACTTAA TTGCATATGG TATATAAAGA ACCCAGTTCC AGCAGGGAGA TTTCTTTAAG |
| 1081 | TGGACTGTTT TCTTTCTTCT CAAAATTTTC TTTCCATTTA TTTTTTAGTA ATCAAGAAAG |
| 1141 | GCTGGAAAAA CTACTGAAAA ACTGATCAAG CTGGACTTGT GCATTTATGT TTGTTTTAAG |
| 1201 | ACACTGCATT AAAGAAAGAT TTGAAAAGTA TACACAAAAA TCAGATTTAG TAACTAAAGG |
| 1261 | TTGTAAAAAA TTGTAAAACT GGTTGTACAA TCATGATGTT AGTAACACTA ATTTTTTTCT |
| 1321 | TAAATTAATT TACCCTTAAG AGTATGTTAG ATTTGATTAT CTGATAATGA TTATTTAAAT |
| 1381 | ATTCCTATCT GCTTATAAAA TGGCTGCTAT AATAATAATA ATACAGATGT TGTTATATAA |
| 1441 | CCTATATCAG ACCTACAGGC TTCTGGCAGG ATTTGTCAGA TAATCAACCC ACACTAACTA |
| 1501 | TCGAAAATGA GCAGCATTTT AAATGCTTTC TAGTGAAAAA TTATAATCTA CTTAAACTCT |
| 1561 | AATCAGAAAA AAAATTCTCA AAAAAACTAT TATGAAAGTC AATAAAATAG ATAATTTAAC |
| 1621 | AAAAGTACAG GATTAGAACA TGCTTATACC TATAAATAAG AACAAAATTT CTAATCCTGC |
| 1681 | TCAAGTGGAA AGGGTATTGC TAAAAGGATG TTTCCAAAAA TCTTGTATAT AAGATAGCAA |
| 1741 | CAGTGATTGA TGATAATACT GTACTTCATC TTACTTGCCA CAAAATAACA TTTTATAAAT |
| 1801 | CCTCAAAGTA AAATTGAGAA ATCTTTAACT TTTTTTCAAC TAACATAATC TATCTTTGTA |
| 1861 | TAATTCATAT TTGGGAATAT GGCTTTTAAT AATGTTCTTC CCACAAATAA TCATGCTTTT |
| 1921 | TTCCTATGGT TACAGCATTA AACTCTATTT TAAGTTGTTT TTGAACTTTA TTGTTTTGTT |
| 1981 | ATTTAAGTTT ATGTTATTTA TAAAAAAAAA ACCTTAATAA GCTGTATCTG TTTCATATGC |
| 2041 | TTTTAATTTT AAAGGAATAA CAAAACTGTC TGGCTCAACG GCAAGTTTCC CTCCCTTTTC |
| 2101 | TGACTGACAC TAAGTCTACC ACACAGCACT TGGGCCAGCA AATCCTCGAA GCAGACAAAA |
| 2161 | ATAAGAGCCT GAAGCAATGC TTACAATAGA TGTCTCACAC AGAACAATAC AAATATGTAA |
| 2221 | AAACTCTTTC ACCACATATT CTTGCCAATT AATTGGATCA TATAAGTAAA ATCATTACAA |
| 2281 | ATATAAGTAT TTACAGGATT TTAAAGTTAG AATATATTTG AATGCATGGG TAGAAAATAT |
| 2341 | CATATTTTAA AACTATGTAT ATTTAAATTT AGTAATTTTC TAATCTCTAG AAATCTCTGC |
| 2401 | TGTTCAAAAG GTGGCAGCAC TGAAAGTTGT TTTCCTGTTA GATGCCAAGA GCACAATGCC |
| 2461 | CAAAATAGAA GATGCAGTTA AGAATAAGGG GCCCTGAATG TCATGAAGGC TTGAGGTCAG |
| 2521 | CCTACAGATA ACAGGATTAT TACAAGGATG AATTTCCACT TCAAAAGTCT TTCATTGGCA |
| 2581 | GATCTTGGTA GCACTTTATA TGTTCACCAA TGGGAGGTCA ATATTTATCT AATTTAAAAG |
| 2641 | GTATGCTAAC CACTGTGCTT TTAATTTCAA AATATTTGTC ATTCAAGTCC CTTTACATAA |
| 2701 | ATAGTATTTC GTAATACATT TATAGATGAG ACTTATATGA AAAGGCTAGG TCAACAAAAA |
| 2761 | CAATAGATTC ATTTAATTTT CCTGTGGTTA ACCTATACGA CCAGGATGTA GAAAACTAGA |
| 2821 | AAGAACTGCC CTTCCTCACA TATACTCTTG CGAGAGAGCA TGAATGCTAT TCTGAACTAT |
| 2881 | CACCTGATTC AAGGACTTTG CTAGCTAGGT TTTGAGGTCA GGCTTCAGTA ACTGTAGTCT |
| 2941 | TGTGAGCATA TTGAGGGCAG AGGAGGACTT AGTTTTTCAT ATGTGTTTCC TTAGTGCCTA |
| 3001 | GCAGACTATC TGTTCATAAT CAGTTTTCAG TGTGAATTCA CTGAATGTTT ATAGACAAAA |
| 3061 | GAAAATACAC ACTAAAACTA ATCTTCATTT TAAAAGGGTA AAACATGACT ATACAGAAAT |
| 3121 | TTAAATAGAA ATAGTGTATA TACATATAAA ATACAAGCTA TGTTAGGACC AAATGCTCTT |
| 3181 | TGTCTATGGA GTTATACTTC CATCAAATTA CATAGCAATG CTGAATTAGG CAAAACCAAC |
| 3241 | ATTTAGTGGT AAATCCATTC CTGGTAGTAT AAGTCACCTA AAAAAGACTT CTAGAAATAT |
| 3301 | GTACTTTAAT TATTTGTTTT TCTCCTATTT TTAAATTTAT TATGCAAATT TTAGAAAATA |
| 3361 | AAATTTGCTC TAGTTACACA CCTTTAGAAT TCTAGAATAT TAAAACTGTA AGGGGCCTCC |
| 3421 | ATCCCTCTTA CTCATTTGTA GTCTAGGAAA TTGAGATTTT GATACACCTA AGGTCACGCA |
| 3481 | GCTGGCTAGA TATACACCTG TCACAAGAGT CTAGATCAGT TAGCACATGC TTTCTACTCT |
| 3541 | TCGATTATTA GTATTATTAG CTAATGGTCT TTGGCATGTT TTTGTTTTTT ATTTCTGTTG |
| 3601 | AGATATAGCC TTTACATTTG TACACAAATG TGACTATGTC TTGGCAATGC ACTTCATACA |
| 3661 | CAATGACTAA TGTATACTGT GATGATTTGA CTCAAAAGGA GAAAAGAAAT TATGTAGTTT |
| 3721 | TCAATTCTGA TTCCTATTCA CCTTTTGTTT ATGAATGGAA AGCTTTGTGC AAAATATACA |
| 3781 | TATAAGCAGA CTAAGCCTTT TAAAAATGTT CTTTGAAAGA TAAAATTAAA TACATGAGTT |
| 3841 | TCTAACAATT ACA |

GENBANK ID: AAA62202.1
DEFINITION HUMAN ENDOTHELIAL-MONOCYTE ACTIVATING POLYPEPTIDE II MRNA,
COMPLETE
CDS.
VERSION U10117.1 GI:498909
MRNA 1..1057
CDS 50..988
/CODON_START=1

| | |
|---|---|
| 1 | GGAACCCGTG GTCCTCCGCT TCATGATTTT CTGCCGTCTC TTGGCAAAAA TGGCAAATAA |
| 61 | TGATGCTGTT CTGAAGAGAC TGGAGCAGAA GGGTGCAGAC GCAGATCAAA TCATTGAATA |
| 121 | TCTTAAGCAG CAAGTTTCTC TACTTAAGGA GAAAGCAATT TTGCAGGCAA CTTTGAGGGA |
| 181 | AGAGAAGAAA CTTCGAGTTG AAAATGCTAA ACTGAAGAAA GAAATTGAAG AACTGAAACA |
| 241 | AGAGCTAATT CAGGCAGAAA TTCAAAATGG AGTGAAGCAA ATAGCATTPC CATCTGGTAC |
| 301 | TCCACTGCAC GCTAATTCTA TGGTTTCTGA AAATGTGATA CAGTCTACAG CAGTAACAAC |
| 361 | CGTATCTTCT GGTACCAAAG AACAGATAAA AGGAGGAACA GGAGACGAAA AGAAAGCGAA |
| 421 | AGAGAAAATT GAAAAGAAAG GAGAGAAGAA GGAGAAAAAA CAGCAATCAA TAGCTGGAAG |
| 481 | TGCCGACTCT AAGCCAATAG ATGTTTCCCG TCTGGATCTT CGAATTGGTT GCATCATAAC |
| 541 | TGCTAGAAAA CACCCTGATG CAGATTCTTT GTATGTCGAA GAAGTAGATG TCGGAGAAAT |
| 601 | AGCCCCAAGG ACAGTTGTCA GTGGCCTGGT GAATCATGTT CCTCTTGAAC AGATGCAAAA |
| 661 | TCGGATGGTG ATTTTACTTT GTAACCTGAA ACCTGCAAAG ATGAGGGGAG TATTATCTCA |
| 721 | AGCAATGGTC ATGTGTGCTA GTTCACCAGA GAAAATTGAA ATCTTGGCTC CTCCAAATGG |
| 781 | GTCTGTTCCT GGAGACAGAA TTACTTTTGA TGCTTTCCCA GGAGAGCCTG ACAAGGAGCT |
| 841 | GAATCCTAAG AAGAAGATTT GGGAGCAGAT CCAGCCTGAT CTTCACACTA ATGATGAGTG |
| 901 | TGTGGCTACA TACAAAGGAG TTCCCTTTGA GGTGAAAGGG AAGGGAGTAT GTAGGGCTCA |
| 961 | AACCATGAGC AACAGTGGAA TCAAATAAAA TGCTTCCACT ACCAAAAGAC ATTAGAGAAA |
| 1021 | ACCTTAAAAG TAATAAAGAG AAATATATTT GTCACTT |

GENBANK ID: P17936
DEFINITION HUMAN ACIDIC FIBROELAST GROWTH FACTOR MRNA, 5′END, CLONE
LA64BDA-MJ36.
CDS 358..>478
/CODON_START=1

| | |
|---|---|
| 1 | TCCCCAAGGC TAGGAGGCCA ACCTACTAAC AGGTGGGTGG GTATGGTGTG TGGTTTCACT |
| 61 | CAGTTCTTCT CATGGGGTTT CTCTGAGCTC CATTCATACC AGAAAGGGAG CAGGAGAGAG |
| 121 | AGGACAAGTG GATCCAACAG CCTTCGCTCC AGGGGAATCA GGGCATCGCC TCCTTTTCTG |
| 181 | GGAGGACACT CCCTTCTGAT GGTGAATGGG AACTCCCTTC CTCCTGCAGC AGCCTGCCTG |
| 241 | CAGCTGTCCT GGTAGAACAG TGTGGACATT GCAGAAGCTG TCACTGCCCC AGAAAGAAAG |
| 301 | CACCCCAGAG CCAAGGCAAA GAGTCTTGAA AGCGCCACAA GCAGCAGCTG CTGAGCCATG |
| 361 | GCTGAAGGGG AAATCACCAC CTTCACAGCC CTGACCGAGA AGTTTAATCT GCCTCCAGGG |
| 421 | AATTACAAGA AGCCCAAACT CCTCTACTGT AGCAACGGGG GCCACTTCCT GAGGATCC |

GENBANK ID:
U76376. 1
VERSION U76376.1 GI:1923234
MCPCPLHRGRGPPAVCACSAGRLGLRSSAAQLTAARLKALGDEL
HQRTMWRRRARSRRAPAPGALPTYWPWLCAAAQVAALAAWLLGRRNL
GENBANK ID: Y00638
VERSION Y00638.1 GI:34280
MYLWLKLLAFGFAFLDTEVFVTGQSPTPSPTGLTTAKNPSVPLS
SDPLPTHTTAFSPASTFERENDFSETTTSLSPDNTSTQVSPDSLDNASAFNTTGVSSV
QTPHLPTHADSQTPSAGTDTQTFSGSAANAKLNFTPGSNAISDVPGERSTASTFPTDP
VSPLTTTLSLAHHSSAALPARTSNTTITANTSDAYLNASETTTLSPSGSAVISTTTIA
TTPSKPTCDEKYANITVDYLYNKETKLFTAKLNVNENVECGNNTCTNNEVHNLTECKN
ASVSISHNSCTAPDKTLILDVPPGVEKFQLHDCTQVEKADTTICLKWKNIETFTCDTQ
NITYRFQCGNNIFDNKEIKLENLEPEHEYKCDSEILYNNHKFTNASKIIKTDFGSPGE
PQIIFCRSEAAHQGVITWNPPQRSFHNFTLCYIKETEKDCLNLDKNLIKYDLQNLKPY
TKYVLSLHAYIIAKVQRNGSAAMCHFTTKSAPPSQVWNMTVSMTSDNSMHVKCRPPRD
RNGPHERYHLEVEAGNTLVRNESHKNCDFRVKDLQYSTDYTFKAYFHNGDYFGEPFIL
HHSTSYNSKALIAFLAFLIIVTSIALLVVLYKIYDLHKKRSCNLDEQQELVERDDEKQ
LMNVEPIHADILLETYKRKIADEGRPFLAEFQSIPRVFSKFPIKEARKPFNQNKNRYV
DILPYDYNRVELSEINGDAGSNYINASYIDGFKEPRKYIAAQGPRDETVDDFWRMIWE
QKATVIVNVTRCEEGNRNKCAEYWPSMEEGTRAFGDVVVKINQHKRCPDYIIQKLNIV
NKKEKATGREVTHIQFTSWPDHGVPEDPHLLLKLRRRVNAFSNFFSGPIVVHCSAGVG
RTGTYIGIDAMLEGLEAENKVDVYGYVVKLRRQRCLMVQVEAQYILIHQALVEYNQFG
ETEVNLSELHPYLHNNKKRDPPSEPSPLEAEFQRLPSYRSWRTQHIGNQEENKSKNRN
SNVIPYDYNRVPLKHELEMSKESEHDSDESSDDDSDSEEPSKYINASFIMSYWKPEVM
IAAQGPLKETIGDFWQMIFGRKVKVIVNLTELKHGDDEICAQYWGEGKQTYGDIEVDL
KDTDKSSTYTLRVFELRHSKRKDSRTVYQYQYTNWSVEDLPAEPKELISNIQVVKQKL
PQKNSSEGNKHHKSTPLLTHCRDGSQQTCIFCALLNLLESAETEEVVDIFQVVRALRK
ARLGMVSTFEQYQFLYDVIASTYPAQNQQVKKNNHQEDKIEFDNEVDKVKQDANCVNP
LGAPEKLPEAKEQAEGSEPTSGTEGPENSVNGPASPALNQGS
GENBANK ID: AF001383.1
VERSION AF001383.1 GI:2199534
MAEMGSKGVTAGKIASNVQKKLTRAQEKVLQKLDKADETKDEQF
EQCVQNFNKQLTECTRLQKDLRTYLASVKANHEASKKLNECLQEVYEPDWFGRDEANK
IAENNDLLNMDYHQKLVDQALLTMDTYLGQFPDIKSRIAKRQRKLVDYDSARHHYESL
QTAKKKDEAKIAKAEEELIKAQKVFEENNVDLQEELPSLWNSRVGFYVNTEQSIAGLE
ENFHKEMSKLNQNLNDVLVGLEKQNGSNTFTVKAQPSDNAPAKGNKSPSPEDGSPAAT
PEIRVNHEPEPAGGATPGATLFKSPSQLRKGPPVPPPFKHTPSKEVKQEQILSLFEDT
FVPEISVTTPSQPAEASEVAGGTQPAAGAQEPCETAASEAASSSLPAVVVETFPATVN
GTVEGGSGAGRLDLPPGFMFKVQAQEDYTATDTDELQLKAGDVVLVIPFQNFEEQDEG
WLMGVKESDWNQHKELEKCRGVFFENFTERVP
GENBANK ID: XM_038595.3
VERSION XM_038595.3 GI:18590923
MAPPSEETPLIPQRSCSLLSTEAGALHVLLPARGPGPPQRLSES
FGDHLAEDLCVQAAKASGILPVYRSLFALATEDLSCWFPPSHIESVEDASTQVLLYRI
REYEPNWEGLEKCNREGLRKDLASAILDLPVLEHLFAQERSDLVSGRLPVGLSLKEQG
ECLSLAVLDLARMAREQAQRPGELLKTVSYKACLPPSLRDLIQGLSPVTRRRIRRTVR
RALRRVAACQADRHSLMAKYIMDLERLDPAGAAETEHVGLPGALGGHDGLGLLRVAGD
GGIAWTQGEQEVLQPECDEFEIVDISIKQAPRVGPAGEHRLVTVTRTDNQILEAEFPG
LPEALSFVALVDGYFRLTTDSQHEFCKEVAPPRLLEEVAEQCHGPITLDFAINKLKTG
GSRPGSYVLRRSPQDEDSELLTVCVQNPLGPDYKGCLIRRSPTGTELLVGLSRPHSSL
RELLATCNDGGLHVDGVAVTLTSCCIPRPKEKSNLIVVQRGHSPPTSSLVQPQSQVQL
SQMTFEKIFADSLEWHENLGHGSETKIYRGCRHEVVDDEARKTEVLLKVMDAKHKNCM
ESFLEAASLMSQVSYRHLVLLHGVCNAGDSTNVQEFVHLGAIDNYLRKRCHLVPASWK
LQVVKQLAYALNYLEDKGLPNCNVSARKVLLAREGADGSPEFIELSDPCVSPAVLSLE
MLTDRIPWVAPECLREAQTLSLEADKNGFGATVWEVFSGVTNPISALDPAKKLQFYED
RQQLPAPKNTELALLIQQCNAYEPVQRPSERAVIRDLNSLISSDVELLSDPTPGALAP
RDGLWNGAQLYACQDPTIFEERHLKYISQLGKGNEQSVELCRVDFLGDNTGALVAVKQ
LQHSGPDQQRDEQREIQILKALHSDFIVKYRGVSYGPGRQSLRLVNEYLPSGCLRDEL
QRHRARLDASRLLLYSSQICKGNEYLGSRRCVHRDLAARNILVESEAHVKIADFGLAK
LLPLDKDYYVVREPGQSPIFWYAFESLSDNIESRQSDVWSFGVVLVELFTYCDKSCSP
SAEFLRMNGCERDVPALCRLLELLEEGQRLPAPPACPAEVEELMKLCWAPSPQDRPSF
SALDPQLDNLNSCSRGCETSAFTANPEGKHESLSFS
GENEANK ID: M32292.1
VERSION M32292.1 GI:181492
MENSLRCVWVPKLAEVLECASLLSAHLQVTCFQIKAETALRELS
EPSDAVTMRCGNVLLDCSAESDRGVFVIKWKKDGIHLALCMDERKQQLSNCSLLIQNI
LHSREHKPDEGLYQCEASLGDSGSIISRTAKVAVAGPLRELSQTESVTAENGDTVLLK
CEVIGEPMPTINNQKNQQDLTPIPGDSRVVVLPSGALQISRLQPGDIGIVRCSARNPA
SSRTGNEAEVRICSDPGLHRQLYFLQRPSNVVAIEGKDAVLECCVSGVPPPSFTWLRG
EEVIQLRSKKYSLLGGSNLLISNVTDDDSGMVTCVVTYKNENISASAELTVLVPPWFL
NHPSNLYAYESNDIEFECTVSCKPVPTVNWMKNCDVVIPSDYFQIVCCSNLRILGVVK
SDEGEYQCVAENEAGNAGTSAQLIVPKPAIPSSSVLPSAPRDVVPVLVSSREVRLSWR
PPAEAKCNIQTETVFFSREGDNRERALNTTQPGSLQLTVCNLKPEANVTFRVVAYNEW
GPGESSQPIKVATQPELQVPGPVENLQAVSTSPTSILITWEPPAVANGPVQCVRLFCT
EVSTGKEQNIEVDGLSYKLEGLKKFTEYSLRELAYNRYGPGVSTDDITVVTLSDVPSA
PPQNVSLEVVNSRSIKVSWLPPPSGTQNGEITGYKIRHRKTTRRGENETLEPNNLWYL
FTGLEKGSQYSFQVSANTVNCTCPPSNWYTAETPENDLDESQVPDQFSSLHVRPQTNC
IIMSWTPPLN
GENBANK ID: X06318
VERSION X06318.1 GI:35488
MADPAACFFPSEGEESTVRFARKGALRQKNVHEVKNHKETAREF
KQPTFCSHCTDFIWCFGKQGEQCQVCCFVVHKRCNEFVTFSCPGADKGPASDDPRSKH
KEKIHTYSSPTECDHCGSLLYGLIHQGMKCDTCMNNVHKRCVNNVPSLCCTDHTERRG
RIYIQAHIDRDVLIVLVRDAKNLVPNDPNGLSDPYVKLELIPDPKSESKQETETIKCS
LNPEWNETFRFQLKESDKDRRLSVETWDWDLTSRNDFMGSLSFGISELQKASVDGWFK
LLSQEEGEYFNVPVPPEGSEANEELRQKFERAKISQGTKVFEEKTTNTVSKFDNNGNR
DRMKLTDFNFLMVLGKGSFGKVMLSERKGTDELYAVKTLKKDVVTQDDDVECTMVEKR
VLALPGKPPFLTQLHSCFQTMDRLYFVMEYVNGGDLNYNIQQVGRFKEPHAVFYAAEI
AIGLFFLQSKGIIYRDLKLDNVNLDSEGHIRIADFCNCKENINDGVTTKTFCGTPDYI
APEIIAYQFYGKSVDWWAFGVLLYEMLAGQAFFEGEDEDELFQSIMEHNVAYFKSMSK
EAVAICKGLMTKHFGKRLGCGPEGERDIKEHAFFRYIDWEKLERKEIQFPYKPKARDK
RDTSNFDKEFTRQPVELTFTDKLFIMNLDQNEFAGFSYTNPEFVINV
GENBANK ID: J04122.1
VERSION J04132.1 GI:623041
MKWKALFTAAILQAQLFITEAQSFGLLDPELCYLLDGILFIYGV
ILTALFLRVKFSRSAEPPAYQQGQNQLYNELNLGRREEYDVLDKRRGRDPEMGGKPRR
KNPQEGLYNELQKDKMAEAYSEIGNKGERRRGKGHDGLYQGLSTATKDTYDALHNQAL
PPR
GENBANK ID: 004313.1
VERSION 004313.1 GI:453368
MDALQLANSAFAVDLFKQLCEKEPLGNVLFSPICLSTSLSLAQV
GAKGDTANEIGQVLHFENVKDIPFGFQTVTSDVNKLSSFYSLKLIKRLYVDKSLNLST
EFISSTKRPYAKELETVDFKDKLEETKGQINNSIKDLTDGHFENILADNSVNDQTKIL
VVNAAYFVGKWNKKFPESETKECPFRLNKTDTKPVGMNNMEATFCMGNIDSINCKIIE
LPFQNKHLSMFILLPKDVEDESTGLEKIEKQLNSESLSQWTNPSTMANAKVKLSIPKF
KVEKMIDPKACLENLGLKHIFSEDTSDFSGNSETKGVALSNVINKVCLEITEDGGDSI
EVPGARILQHKDELNADHFFIYIIRHNKTRNIIFFGKFCSP
GENBANK ID: X68968.1
VERSION X68968.1 GI:452315
MDEPSPLAQFLELNQHSRFIIGSVSEDNSEGEISNLVKLDLLEE
KEGSLSPASVGSDTLSDLGISSLQDGLALHIRSSNSGLELVKQGRDRKKIDSQRDFTV
ASPAEFVTRFGGNKVIEKVLIANNGIAAVECMRSIRRWSYENFRNERAIRFVVMVTPE
DLKANAEYIKMADHYVPVFGGFNNNNYANVELILDIAKRIFVQAVWAGWGHASENPKL
PELLLKNGIAFNGPPSQANNALGDKIASSIVAQTAGIFTLFWSGSGLRVDWQENDFSK
RILNVFQELYEKGYVKDVDDGLQAAEEVGYPVNIKASEGGGGEGIRKVNNAGDFPNLF
RQVQAEVPGSPIFVMRLAKQSRHLEVQILADQYGNAISLFGRDCSVQRRHQKIIEEAP
ATIATFAVFEHMEQCAVKLAKMVGYVSAGTVEYLYSQDRSFYFLELNPRLQVEHFCTE
MVADVNLFAAQLQIAMGIFLYRIKDIRMMYGVSFWGDSFIDFEDSAHVFCPRGHVIAA
RITSENFDEGFKPSSGTVQELNFRSNKNVWGYFSVAAAGGLHEFADSQFGHCFSWGES
REEAISNNVVALKELSIRGDFRTTVEYLIKLLETESFQMNRIDTGWLDRLIAEKVQAE
RPDTMLGVVCGALHVADVSLRNSVSNFLHSLERGQVLEAHTLLNTVDVELIYEGVKYV
LKVTRQSPNSYVVINNGSCVEVDVHRLSDGGLLLSYDGSSYTTYNKEEVDRYRITIGN
KTCVFEKENDPSVMRSFSAGKLIQYIVEDGGHVLAGQCYAEIEVMKMVMTLTAVESGC
IHYVKRPGAALDPGCVLAKMQLDNPSKVQQAELHTGSLPRIQSTALRGEKLHRVFHYV
LDNLVNVMNGYCLFDFFSSSKVKDWVERLNKTLRDPSLPLLELQDIMTSVSGRIPPNV
EKSIKKEMAQYASNITSVLCQFPSQQIANILDSHAATLNRKSEREVFFNNTQSIVQLV
QRYRSGIRGHMKAVVMDLLRGYLRVETQFQNGHYDKCVFALREENKSDMNTVLNYIFS
HAQVTKKNLLVTMLIDQLCGRDPTLTDELLNILTELTQLSKTTNAKVALRARQVLIAS
HLPSYELRHNQVESIFLSAIDMYGHQFCIENLQKLILSEISIFDVLPNFFYNSNQVVR
MAALEVYVRRAYIAYELNSVQHRQLKDNTCVVEFQFNLPTSNPNRGNIPTLNRMSFSS
NLNHYGMTHVASVSDVLLDNSFTPPCQRMCGMVSFRTFEDFVRIFDEVNGCFSDSPPQ
SPTFPEAGHTSLYDEDKVPRDEPIHILNVAIKTDCDIEDDRLAANFREFTQQNKATLV
DHGIRELTFLVAQKDFRKQVNYEVDRRFHREFPKFFTFRARDKFEEDRIYRELEPALA
FQLELNRMRNFDLTAIPCANEKMHLYLGAAKVEVGTEVTDYRFFVRAIIRHSDLVTKE
ASFEYLQNEGERLLLEANDELEVAFNNTNVRTDCNHIFLNFVPTVIMDFSKIEESVRY
MVMRYGSRLWKLRVLQAEVKINIRQTTTGSAVPIRLFITNESGYYLDISLYKEVTDSR
SGNIMFHSFGNKQGFQHGNLINTPYVTEDLLQAKREGAQTLGTTYIYDFFEMFRQALF
KLWGSPDKYPKDILTYTELVLDSQGQLVENNRLFGGNEVGNVAFKMRFKTQEYFEGRD
VIVIGNDITFRIGSFGPGEDLLYLRASEMARAEAIFKIYVAANSGARIGMAEEIKHMF
HVAWVDPEDFHKGFKYLYLTPQDYTRISSLNSVHCKHIEEGGESRYMITDIIGKDDGL
GVENLRGSGMIAGESSLAYEEIVTISLVTCRAIGIGAYLVRLGQRVIQVENSHIILTG
ASALNKVLGREVYTSNNQLGGVQINNYNGVSEITVPDDFEGVYTILEWLSYMPKDNHS
PVPIITPTDPIDREIEFLFSRAPYDPRWNLAGRFHFTLKGTWQSGFFDHGSFKEIMAP
WAQTVVTGRARLGGIPVGVIAVETRTVEVAVPADPANLDSEAKIIQQAGQVWFPDSAY
KTAQAIKDFNREKLFLMIEANWRGFSGGNKDNYDQVLRFGAYTVDGLRQYKQPTLIYI
RPMRELRGGSWVVIDATINFLCIENYADKESRGGVLEFEGTVEIKFRKEDLIKSMRRI
DPAYKKLNEQLGEPDLSDKDRKDLEGRLKAREDLLLPIYHQVAVQFADFHDTPGEMLE
KGVISDILEWKTARTFLYWRLRRLLLEDQVKQEILQASGELSEVHIQSMLRRWFVETE
GAVRAYLWDNNQVVVQWLEQHWQAGDGPRSTIRENITYLKHDSVLKTIRGLVEENPEV
AVDCVIYLSQHISFAERAQVVHLLSTMDSPAST
GENBANK ID: L03840.1
VERSION L03840.1 GI:182570
MRLLLALLGVLLSVPGPPVLSLEASEEVELEPCLAPSLEQQEQE
LTVALGQPVRLCCGRAERGGHWYKEGSRLAPAGRVRGWRGRLEIASELFEDAGRYLCL
ARGSMIVLQNLTLITGDSLTSSNDDEGPKSNRDPSNRNSYPQQAPYNTHPQRMEKKLH
AVPAGNTVKFRCFAAGNPTPTIRWLKGGQAFHGENRIGGIRLRHQEWSLVMESVVPSD
RGTYTCLVENAVGSIRYNYLLDVLERSPHRPILQAGLPANTTAVVGSDVELLCKVYSD
AQPHIQWLKHIVINGSSFGADGFPYVQVLKTADINSSEVEVLYLRNVSAEDAGEYTCL
AGNSIGLSYQSAWLTVLPEEDPTWTAAAPEARYTDIILYASGSLALAVLLLLAGLYRG
QALNGRHPRPPATVQKLSRFPLARQFSLESGSSGKSSSSLVRGVRLSSSGPALLAGLV
SLDLPLDPLWEFPRDRLVLGKPLGEGCFGQVVRAEAFGMDPARPDQASTVAVKMLKDN
ASDKDLADLVSEMEVMKLIGRHENIINLLGVCTQEGPLYVIVECAAKGNLREFLRARR
PPGPDLSPDGPRSSEGPLSFPVLVSCAYQVARGNQYLESRKCIHRDLAARNVLVTEDN
VMKIADFGLARGVHNIDYYKKTSNGRLPVKWMAPEALFDRVYTHQSDVWSFGILLWEI
FTLGGSPYPGIPVEELFSLLREGHRMDRFFHCPFELYGLMRECWHAAPSQRPTFKQLV
EALDKVLLAVSEEYLDLRLTFGPYSPSGGDASSTCSSSDSVFSHDPLPLGSSSFPFGS
GVQT
GENBANK ID: AF043342.1
VERSION AF043342.1 GI:2905633
VRSSSRTPSDKPVANVVANPQAEGQLQWLNRRANALLANGVELR
DNQLVVPSEGLYLIYSQVLFKGQGCPSTHVLLTHITSRIAVSYQTKVNLLSAIKSPCQ
RETPRGAEAKPWYEPIYLCGVFQLEKGDRLSAEINRPDYLDFAESGQVYFGITAL
GENBANK ID: NM_000735.2
VERSION NM_000735.2 GI:10800407
MDYYRKYAAIFLVTLSVFLNVLNSAPDVQDCPECTLQENPFFSQ
PGAPILQCMGCCFSRAYPTPLRSKKTNLVQKNVTSESTCCVAKSYNRVTVN0GFKVEN
HTACNCSTCYYNKS
GENBANK ID: M83533.1
VERSION M83533.1 GI:178541
LRKHNIETYLIKQPEDSLLSLPEDIVKESVSSSDRRNSGATFTE
GSWSPELPFDNIVGKQNTLAALTRNSINLLPNHLAQALHVQSGPEEINKRIEHTIDLR
SGDKLRRENIKPFSLNFKDSSLENKYSQMRDEVFKSNLVCAFIVLLFITAIQSLLPSS
RVMPMTIQFSILIMLHSALVLITTAEDYKCLPLILRKTCCWINETYLARNVIIFASIL
INFLGAILNILWCDFDKSIPLKNLTFNSSAVFTDICSYPEYFVFTGVLANVTCAVFLR
LNSVLKLAVLLIMIAIYALLTETVYA0LFLRYDNLNESGEDFLGTKEVSLLLMANFLL
AVFYHGQQLEYTARLDFLWRVQAKEEINEMKELREHNENMLRNILPSHVARNELEKDR
DNEELYSQSYDAVGVMFASIPGFADFYSQTEMNNQGVECLRLLNEIIADFDELLGEDR
FQDIEKIKTIGSTYNAVSGLSPEKQQCEDKWGHLCALADFSLALTESIQEINKHSFNN
FELRIGISNGSVVAGVIGAKKPQYDIMGKTVNLASRMDSTGVSGRIQVPEETYLILKD
QGFAFDYRGEIYVEGISEQEGKIKTYFLLGRVQPNPFILPPRRLPGQYSLAAVVLGLV
QSLNRQRQKQLLNENNNTGIIKGNYNRRTLLSPSGTEPGAQAEGTDKSDLP
GENBANK ID: M31767.1
VERSION M31767.1 GI:181615
MDKDCEMKRTTLDSPLGKLELSGCEQGLNEIKLLGKGTSAADAV
EVPAPAAVLGGPEPLMQCTAWLNAYFHQPEAIEEFPVPALHHPVFQQESFTRQVLWKL
LKVVKFGEVISYQQLAALAGNPKATRAVGGAMRGNPVPILIPCHRVVCSSGAVGNYSG
GLAVKENLLANEGNRLGKPGLGGSSGLAGAWLKGAGATSGSPPAGRN
GENBANK ID: X14723
VERSION X14723.1 GI:30250
MMKTLLLFVGLLLTWESGQVLGDQTVSDNELQEMSNQGSKYVNK
EIQNAVNGVKQTKTLIEKTNEERKTLLSNLEEAKKKKEDALNETRESETKLKELPGVC
NETMMALWEECKPCLKQTCMKFYARVCRSGSGLVGRQLEEFLNQSSFFYFWNNGDRID
SLLENDRQQTHMLDVMQDHFSRASSIIDELFQDRFFTREPQDTYHYLPFSLPHRRPHF
FFPKSRIVRSLNPFSPYEPLNFHAMFQPFLEMTHEAQQAMDIHFESPAFQNPPTEFIR
EGDDDRTVCREIRHNSTGCLRMKDQCDKCREILSVDCSTNNPSQAKLRRELDESLQVA
ERLTRKYNELLKSYQWKMLNTSSLLEQLNEQFNWVSRLANLTQGEDQYYLRVTTVASH
TSDSDVPSGVTEVVVKLFDSDPITVTVEVEVSRKNFKFMETVAEKALQEYRKKHREE
GENBANK ID: X04391.1
VERSION X04391.1 GI:37186
MPMGSLQPLATLYLLGNLVASCLGRLSWYDPDFQARLTRSNSKC
QGQLEVYLKDGWHMVCSQSWGRSSKQWEDESQASKVCQRLNCGVPLSLGPFLVTYTPQ
SSIICYGQLGSFSNCSESRNDMCESLGLTCLEPQKTTFFTTRPPPTTTPEPTAPPRLQ
LVAQSGGQHCAGVVEFYSGSLGGTISYEAQDKTQDLENFLCNNLQCGSFLKHLPETEA
GRAQDPGEFRENQPLPIQWKIQNSSCTSLEHCFRKIKPQKSGRVLALLCSGFQPKVQS
RLVGGSSICEGTVEVRQGAQWAALCDSSSARSSLRWEEVCREQQCGSVNSYRVLDAGD
PTSRGLFCFHQKLSQCEELWERNSYCKKVFVTCQDPNPAGLAAGTVASIILALVLLVV
LLVVCGPLAYKKLVKKFRGKKQRQWIGPTGNNQNNSFHRNETATVRSHAENPTASHVD
NEYSQFPRNSRLSAYFALEGVLNRSSNQPDNSSDSDYDLHGAQRL
GENBANK ID: S78187.1
VERSION S78187.1 GI:243485
MEVPQPEPAPGSALSPAGVCGGAQRPGHLPGLLLGSHGLLGSPV
RAAASSFVTTLTQTMHDLAGLGSRSRLTELSLSRRASESSLSSESSESSDAGLCMDSP
SPMDPHMAEQTFEQAIQAASRIIRNEQFAIRRFQSNPVRLLGHSPVLRNITNSQAPDG
RRKSEAGSGAASSSGEDKENDGFVFKMPWKPTHPSSTHALAEWASRREAFAQRPSSAP
DLMCLSPDRKNEVEELSPLALGRFSLTFAEGDTEEDDGFVDILESDLKDDDAVPPGME
SLISAPLVKTLEKEEEKDLVMYSKCQRLFRSFSNPCSVIRPILKRLERPQGRDTPVQN
KRRRSVTPPEEQQEAEEEKARVLRSKSLCHDEIENDLLSDHRELIGDYSKAFLLQTVD
GKHQDLKYISPETNVALLTGKFSNIVDKFVIVDCRYPYEYEGGHIKTAVNLPLERDAE
SFLLKSPIAPCSLDKRVILIFNCEFSSERGPRMCRFIRERDRAVNDYFSLYYPEMYIL
KGGYKEFFPQHPNFCEPQDYRPNNHEAFKDELKTFRLKTRSWAGERSRRELCDRLQDQ
GENBANK ID: Y00096.1
VERSION Y00096.1 GI:30455
MREAAFMYSTAVAIFLVILVAALQGSAPRESPLPYEIPLDPEGS
LELSWNVSYTQEAIHFQLLVRRLKAGVLFGMSDRQELENADLVVLWTDGDTAYFADAW
SDQKGQIHLDFQQDYQLLQVQRTPEGLTLLFKRPFGTCDPKDYLIEDGTVHLVYGILE
EPFRSLEAINGSGLQMGLQRVQLLKFNIFEPELFSDTCTNEVQAFNIQIESQETTYWC
YIKELPKGFSRHHIIKYEPIVTKGNEALVHHMEVFQCAPEMDSVPHFSGPCDSKMKPD
RLNYCRHVLAAWALGAKAFYYPEEAGLAFGGPGSSRVLRLEVHYHNPLVIEGRNDSSG
IRLYYTAKLRRFNAGINELGLVYIPVMAIFPRETAFILTGYCTDKCTQLALPPSGIHI
FASQLHTHLTGRKVVTVLVRDGREWEIVNQDNHYSFHFQEIRMLKKVVSVHFGDVLIT
SCTYNTEDRELATVGGFGILEEMCVNYVHYYPQTQLELCKTAVDAGFLQKYFHLINRF
NNEDVCTCFQASVSQQFTSVFWNSFNCDVLKALYSFAPISMHCNKSSAVRFQGEWNLQ
PLPKVISTLEEPTPQCPTSQGRSPAGPTVVSIGGGKG
GENBANK ID: XM_055551.3
VERSION XM_055551.3 GI:18557356
MKETQKSTYYITGESKEQVANSAFVERVRKQGFEVVYNTEPIDE
YCVQQLKEFDGKSLVSVTKEGLELPEDEEEKKKMEESKEKFENLCKLMKEILDKKVEK
VTISNRLVSSPCCIVTSTYGWTANMEQIMKAQALRDNSTMGYMNAKKHLEINPDHPIM
ETLRQKAEADKNDKAVKDLVVLLFETALLSSGFSLEDPQTHSNHIYHMIKLGLGTDED
EVAAEEPSDAVPDEIFFLEQDEDASRMEEVD
GENBANK ID: M84711.1
VERSION M84711.1 GI:182774
MAVGKNKRLTKGGKKGAKKKVVDPFSKKDWYDVKAPAMFNIRNI
GKTLVTRTQGTKIASDGLKCRVFEVSLADLQNDEVAFRKFKLITEDVQGKNCLTNFHG
MDLTRDKMCSMVKKWQTNIEAHVDVKTTDGYLLRLFCVGFTKKRNNQIRKTSYAQEQQ
VRQIRKKMMEIMTREVQTNDLKEVVNKLTPDSIGKDIEKACQSIYPLHDVFVRKVKML
KKPKFELGKLMELRGEGSSSGKATGDETGAKVERADGYEPPVQESV
GENBANK ID: X53505
VERSION X53505.1 GI:36145
MAEEGIAAGGVMDVNTALQEVLKTALIHDGLARGIREAAKALDK
RQAHLCVQASNCDEPMYVKLVEALLAEHQINLIKVDDNKKLGEWVGLCKIDREGNPRK
VVGCSCVVVKDYGKESQAKDVIEEYFKCKK
GENBANK ID: X06617
VERSION X06617.1 GI:26143
MADIQTERAYQKQPTIFQNKKRVLLGETGKEKLFRYYKNIGLGF
KTPKEAIEGTYIDKKCPFTGNVSIRGRILSGVVTKMKMQRTIVIRRDYLHYIRKYNRF
EKRHKNMSVHLSPCFRDVQIGDIVTVGECRFLSKTVRFNVLKVTKAAGTKKQFQKF
GENBANK: M55040.1
VERSION M55040.1 GI:177974
MRPPQCLLHTPSLASPLLLLLLWLLGGGVGAEGREDAELLVTVR
GGRLRGIRLKTPGGPVSAFLGIPFAEPPNGPRRFLPPEPKQPWSGVVDATTFQSVCYQ
YVDTLYPGFEGTEMNNPNRELSEDCLYLNVWTPYPRPTSPTBVLVWIYGGGFYSGASS
LDVYDGRFLVQAERTVLVSNNYRVGAFGFLALPGSREAPGNVGLLDGRLALQWVQENV
AAFGGDPTSVTLFGESAGAASVGMHLLSPPSRGLFHRAVLQSGAPNGPWATVGNGEAR
RRATQLAHLVGCPBGGTGGNDTELVACLRTRPAQVLVNKEWHVLPQESVFRFSFVPVV
DGDFLSDTPEALINAGDFHGLQVLVGVVKDEGSYFLVYGAPGFSKDNESLISRAEFLA
GVRVGVPQVSDLAAEAVVLHYTDWLHPEDFARLREALSDVVGDHNVVCPVAQLAGRLA
AQGARVYAYVFEHRASTLSWPLWMGVPHGYEIEFIFGIFLDPSRNYTAEEKIFAQRLM
RYWANFARTGDPNEPRDPKAPQWPPYTAGAQQYVSLDLRPLEVRRGLRAQACAFWNRF
LPKLLSATDTLDEAERQWKAEFHRWSSYMVHWKNQFDHYSKQDRCSDL
GENBANK ID: NM_000717.2
VERSION NM_000717.2 GI:9951925
MRMLLALLALSAARPSASAESHWCYEVQAESSNYPCLVPVKNGG
NCQKDRQSPINIVTTKAKVDKKLGRFFFSGYDKKQTWTVQNNGHSVMMLLENKASISG
GGLDAPYQAKQLHLHWSDLPYKGSEHSLDGEHFANEMNIVHEKEKGTSRNVKEAQDPE
DEIAVLAFLVEAGTQVNEGFQPLVEALSNIPKPEMSTTMAESSLLDLLFKEEKLRHYF
RYLGSLTTPTCDEKVVWTVFREPIQLRREQILAFSQKLYYDKEQTVSMKDNVRPLQQL
GQRTVIKSGAPGRPLPWALPALLGPMLACLLAGFLR
GENBANK ID: S70S87.1
VERSION S70587.1 GI:546848
MTALFLMSNLFGLACGQAMSFCIPTEYTMHIERRECAYCLTINT
TMCAGYCNTRDINGKLFLPKYALSQDVCTYRDFIYRTVEIPGCPLHVAPYFSYPVALS
CKCGKCNTDYSDCIHEAIKTNYCTKPQKSYLVGFSV
GENBANK ID: M34057
VERSION M34057.1 GI:339547
MDTKLMCLLFFFSLFPLLVSNHTGRIKVVFTPSICKVTCTKGSC
QNSCEKGNTTTLISENGHAADTLTATNFRVVICHLPCNNGGQCSSRDKCQCFFNFTGK
LCQIPVHGASVPKLYQNSQGFGKALGTHVIHSTHTLPLTVTSGQGVKVKFPPNIVNIH
VKHPPEASVQIHQVSRIDGPTGQKTKEAQPGQSQVSYQGLPVQKTQTIHSTYSHQQVI
PHVYPVAAKTQLGRCFQETIGSQCGKALPGLSKQEDCCGTVGTSWGFNKCQKCPKKPS
YHGYNQMMECLPGYKRVNNTFCQDINECQLQGVCFNGECLNTMGSYRCTCKIGFGPDP
TFSSCVPDPPVISEEKGPCYRLVSSGRQCMYPLSVHLTKQLCCCSVGKAGPHCEKCPL
PGTAAFKEICPGGMGYTVSGVHRRRPIHHHVGKGPVFVKPKNTQPVAKSTHPPPLPAK
EEPVEALTFSREHGARSAEPEVATAPPEKEIFSLDQEKTKLEPGQPQLSPGISAIHLH
PQFPVVIEKTSPFVPVEVAPEASTSSASQVIAPTQVTEINECTVNPDICGAGHCINLP
VRYTCICYEGYRFSEQQRKCVDIDECTQVQNLCSQGRCENTEGSFLCICPAGFMASEE
GTNCIDVDECLRPDVCGEGHCVNTVGAFRCEYCDSCYRMTQRGRCEDIDECLNPSTCP
DEQCVNSPGSYQCVPCTEGFRGWNGQCLDVDECLEFNVCANGDCSNLEGSYMCSCHKG
YTRTPDHKHCRDIDECQQCNLCVNGQCKNTECSFRCTCGQGYQLSAAKDQCEDIDECQ
NRHLCAHGQCRNTEGSFQCVCDQGYRASGLCDHCEDINECLEDKSVCQRGDCINTAGS
YDCTCPDGFQLDDNKTCQDINECEHPGLCGPQGECLNTEGSEHCVCQQGFSISADGRT
CEDIDECVNNTVCDSHGFCDNTAGSFRCLCYQGFQAPQDGQGCVDVNECELLSGVCGE
AFCENVEGSFLCVCADENQEYSPMTGQCRSRTSTDLDVDVDQPKEEKKECYYNLNDAS
LCDNVLAPNVTKQECCCTSGAGWGDNCEIFPCPVLCTAEFTEMCPKGKGFVPAGESSS
EAGGENYKDADECLLFGQEICKNGFCLNTRPGYECYCKQGTYYDFVKLQCFDNDECQD
PSSCIDGQCVNTEGSYNCFCTHPMVLDASEKRCIRFAESNEQIEETDVYQDLCWEHLS
DEYVCSRPLVGKQTTYTECCCLYGEAWGNQCALCPLKDSDDYAQLCNIPVTGRRQPYG
RDALVDFSEQYTFEADPYFIQDRFLNSFEELQAEECGILNGCENGRCVRVQEGYTCDC
LDGYHLDTAKNTCFDVNECDELNNRNSLCKNAKCINTDGSYKCLCLPGYVPSDKPNYC
TPLNTALNLEKDSDLE
GENBANK ID: AF257099.1
VERSION AF257099.1 GI:8037944
MSDAAVDTSSEITTEDLKEKKEVVEEAENGRDAFANGNANEENG
EPEADNEVDEEEEEGGEEEGDGEEEDGDEDEGAESATGKRAAEDDEDDDVDTQKQKTD
EDD
GENBANK ID: L06505.1
VERSION L06505.1 GI:186799
MPFKFDFNEIKVVYLRCTGGEVGATSALAPKIGPLGLSPKKVGD
DIAKATGDWKGLRITVKLTIQNRQAQIEVVPSASALIIKALKEPPRDRKKQKNIKHSG
NITFDEIVNIARQMRHRSLARELSGTIKEILGTAQSVGCNVDGRRHPHDIIDDINSGAV
ECPAS
GENBANK ID: X79234.1
VERSION X79234.1 GI:495125
MAQDQGEKENPMRELRIRKLCLNICVGESGGRLTRAAKVLEQLT
GQTPVFSKARYTVRSFGIRRNEKIAVECAVRGAKAEEILEKGLKVRELELRKNNFSDT
GNFGFGIQEHIDLGIEYDFSIGIYGLDFYVVLGRFGFSIADKKRRTGCIGAKHRISKE
EAMRWFQQKYDGIILPGK
GENBANK ID: X59932.1
VERSION X59932.1 GI:30255
MASIQAAWPSGTECIAKYNFHGTAEQDLPECKGDVLTIVAVIKD
PNWYKAKNKVGREGIIFANYVGKREGVKAGTKLSLNPWFHGKITREQAERLLYPPETG
LFLVRESTNYFGDYTLCVSCDGKVEHYRINYHASKLSIDEEVYFENLNQLVEHYTSDA
DGLCTRLIKFKVNEGTVAAQDEFYRSGWALNMKELKLLQTIGKGEFGDVNLGDYRGNK
VAVKCIKNDATAQAFLAEASVNTQLRHSNLVQLLGVIVEEKGGLYIVTEYMAKGSLVD
YLRSRGRSVLGGDCLLKFSLDVCEANEYLEGNNFVHRDLAARNVLVSEDNVAKVSDFG
LTKEASSTQDTGKLPVKWTAPEALREKKESTKSDVWSFGILLWEIYSFGRVPYPRIPL
KDVVFRVEKGYKNDAFDGCPFAVYEVNKNCWHLDAANRFSFLQLREQLEHIKTHELHL
GENBANK ID: AAA98616.1
VERSION AAA98616.1 GI:178428

| | |
|---|---|
| 1 | MQGPWVLLLL GLRLQLSLGI IPVEEENPDF WNRQAAEALG AAKKLQPAQT AAKNLIMFLG |
| 61 | DGMGVSTVTA ARILKGQKKD KLGPETFLAM DRFPYVALSK TYSVDKHVPD SGATATAYLC |
| 121 | GVKGNFQTIG LSAAARFNQC NTTRGNEVIS VVNRAKKAGK SVGVVTTTRV QHASPAGTYA |
| 181 | HTVNRNWYSD ADVPASARQE GCQDIATQLI SNMDIDVILG GGRKYMFPMG TPDPEYFDDY |
| 241 | SQGGTRLDGK NLVQEWLAKH QGARYVWNRT ELLQASLDPS VTHLMGLFEP GDMKYEIHRD |
| 301 | STLDPSLMEM TEAALLLLSR NPRGFFLFVE GGRIDHGNHE SRAYRALTET IMFDDATERA |
| 361 | GQLTSEEDTL SLVTADHSHV FSFGGYPLRG SSIFGLAPGK ARGRKAYTVL LYGNGRGYVL |
| 421 | KDGARFDVTE SESGSPEYRQ QSAVFLDGET HAGEDVAVFA RGPQAHLVHG VQEQTFIAHV |
| 481 | MAFAACLEPY TACDLAPPAG TTDAAHPGPS VVPALLPLLA GTLLLLGTAT AP |

GENBANK ID: XM_041507.1
VERSION XM_041507.1 GI:14727457
MGCTVSAEDKAAAERSKMIDKNLREDGEKAAREVKLLLLGAGES
GKSTIVEQEKIIREDGVSEEECRQYRAVVYSNTIQSIEAIVKAMGNLQIDFADPSRAD
DARQLFALSCTAEEQGVLFDDLSGVIRRLWADHGVQACFCRSREYQLNDSAAYYLNDL
ERIAQSDYIPTQQDVLRTRVKTTGIVETHFTFKDLHFKEFDVGGQRSERKKWIHCFEG
VIAIIFCVALSAYDLVLAEDEEMNRNNESMKLFDSICNNKWFTDTSIILFLNKKDLFE
EKITHSPLTICFPEVTGANKYDEAASYIQSKFEDLNKRKDTKEIVTHFTCATDTKNVQ
FVFDAVTDVIIKNNLKDCGLF
GENBANK ID: NM_001032.2
VERSION NM_001032.2 GI:13904868
MGHQQLYESHPRKFGQGSRSCRVCSNRHGLIRKVGLNMCRQCFR
QYAKDIGFIKLD
GENBANK ID: M22430.1
VERSION M22430.1 GI:190888
MKTLLLLAVIMIFGLLQAHGNLVNFHRNIKLTTGKEAALSVGFY
GCHCGVGGRGSPKDATDRCCVTNDCCYKRLEKRGCGTKFLSYKFSNSGSRITCAKQDS
CRSQLCECDKAAATCFARNKTTYNKKYQYYSNKHCRGSTPRC
GENBANK ID: X63527
VERSION X63527.1 GI:36127
MSNLRLQKRLASSVLRC0KKKVELDPNETNEIANANSRQQIRKL
IRDGLIIRKPVTVHSRARCRKNTLARRKGRHMGIGKRKGTANARMPEKVTENRRMRIL
RRLLRRYRESKKIDRHNYHSLYLKVK0NVFKNKRILNEHIHKLKADKARKKLLADQAE
ARRSKTKEARKRREERLQAKKEEIIKTLSKEEETKK
GENBANK ID: AF099644.1
VERSION AF099644.1 GI:4323527
MAQFAFESDLHSLLQLDAPIPNAPPARWGRKAKEAAGFAFSPMR
AANRSHSAGRTPGRTPGKSSSKVQTTPSKFGGDRVIFHRSAAQMEVASFLLSKENQFE
NSQTPTKKEHQKAWALNLNGFDVEEAKILRLSGKPQNAPEGYQNRLKVLYSQKATPGS
SRKICRVIPSLPDRILDAPEIRNDYYLNLVDWSSGNVLAVALDNSVVLWSASSGDILQ
LLQMEQPGEYISSVAWIKEGNYLAVGTSSAEVQLWDVQQQKRLRNMTSHSARVGSLSW
NSYILSSGSRSGHIHHHDVRVAEHHVATLSGHSGEVCGLRWAPDGRHLASGGNDNLVN
VWPSAPGEGGEVFLQTFTQHQGAVKAVAECPWQSNVLATGGGTSDRHIRIENVCSGAC
LSAVDAHSQVCSILWSPHYKELISGHGFAQNQLVIWKYPTMAKVAELKGHTSRVLSLT
MSPDGATVASAAADETLRLWRCFELDPARRREREKASAAKSSLIHQGIR
GENBANK ID: X51466
VERSION X51466.1 GI:31105
MVNFTVDQIRAIMDKKANIRNMSVIAHVDHGKSTLTDSLVCKAG
IIASARAGETRFTDTRKDEQERCITIKSTAISLFVELSENDLNFIKQSKDGAGFLINL
IDSPGMVDFSSEVTAALRVTDGALVVVDCVSGVCVQTETVLRQAIAERIKPVLMMNKM
DRALLELQLEPEELYQTFQRIVENVNVIISTYGEGESGPMGNIMIDPVLGTVGFGSGL
HGMAFTLKQFAEMYVAKFAAKGEGQLGPAERAKKVEDMMKKLWGDRYFDFANGKFSKS
ATSPEGKKLPRTFCQLILDPIFKVFDAIMNFKKEETAKLIEKLDIKLDSEDKDKEGKP
LLKAVMRRWLPAGDALLQMITIHLPSPVTAQKYRCELLYEGPPDDEAAMGIKSCDPKG
PLMMVISKNVPTSDKGRFYAFGRVFSGLVSTGLKVRIMGFNYTFGKKEDLYLKPIQRT
ILMMGRYVEPIEDVFCGNIVGLVGVDGFLVKTGTITTFEHAHNMRVMKFSVSPVVRVA
VEAKNPADLPKLVEGLKRLAKSDPMVQCIIEESGEHIIAGAGELHLEICLKDLEEDHA
CIPIKKSDPVVSYRETVSEESNVLCLSKSPNKHNRLYMKARPFPDGLAEDIDKGEVSA
RQELKQRARVLAEKVEWDVAEARKIMCFGPDGTGPNILTDITKGVQYLNEIKDSVVAG
FQWATKEGALCEENMRGVRFDVMDVTLMADAIHRGGGQIIPTARRCLYASVLTAQPRL
MEPIVLVEIQCPEQVVGGIVGVLNRKRGHVFEESQVAGTPMFVVKAYLPVNESFGFTA
DLRSNTGGQAFPQCVFDHWQILPGDPFDNSSRPSQVVAETRKRKGLKKGIPALDNFLD
GENBANK ID: M15661
VERSION M15661.1 GI:337577
MVNVPKTRRTFCKKCGKHQPHKVTQYKKGKDSLYAQGRRRYDRK
QSGYGGQTKPIFRKKAKTTKKIVLRLECVEPNCRSKRMLAIKRCKHFELGGDKKRKGQ
VIQF
GENEANK ID: J04823.1
VERSION J04823.1 GI:1311703
MSVLTFLLLRGLTGSARRLPVPRAKIHSLPPEGKLGINELAVGL
TSCFVTFLLPAGWILSHLETYRRPE
GENBANK ID: NM_001760.2
VERSION NM_001760.2 GI:16950657
MELLCCEGTRHAPRAGPDPRLLGDQRVLQSLLRLEERYVPRASY
FQCVQREIKPHMRKMLAYWMLEVCEEQRCEEEVFPLANNYLDRYLSCVPTRKAQLQLL
GAVCMLLASKLRETTPLTIEKLCIYTDHAVSFRQLRDWEVLVLGKLKWDLAAVIAHDF
LAFILHRLSLPRDRQALVKKHAQTFLALCATDYTFAMYPPSMIATGSIGAAVQGLGAC
SMSGDELTELLAGITGTEVDCLRACQEQIEAALRESLREASQTSSSPAPKABRGSSSQ
GPSQTSTPTDVTAIHL
GENBANK ID: NM_002625.1
DEFINITION HOMO SAPIENS 6-PHOSPHOFRUCTO-2-KINASEFRUCTOSE-2,6-BIPHOSPHA-
TASE 1
(PFKFB1), MRNA.
VERSION NM_002625.1 GI:4505744
CDS 80..1495

| | |
|---|---|
| 1 | GAATTCCGGA CAGGTAGTAA GATAGGAAGT GAGGCCAGGT ACCTTGTGGG CAGTGATGTC |
| 61 | ATTCGGTGCG ACTCCTAAGA TGTCTCCAGA GATGGGAGAG CTCACCCAAA CCAGGTTGCA |
| 121 | GAAGATCTGG ATTCCACACA GCAGCGGCAG CAGCAGGCTG CAACGGAGAA GGGGCTCATC |
| 181 | CATACCCCAG TTTACCAATT CCCCCACAAT GGTGATCATG GTGGGTTTAC CAGCTCGAGG |
| 241 | CAAGACCTAT ATCTCCACAA AGCTCACACG ATATCTCAAC TGGATAGGAA CACCAACTAA |
| 301 | AGTGTTTAAT TTAGGCCAGT ATCGACGAGA GGCAGTGAGC TACAAGAACT ATGAATTCTT |
| 361 | TCTTCCAGAC AACATGGAAG CCCTGCAAAT CAGGAAGCAG TGCGCCCTGG CAGCCCTGAA |
| 421 | GGATGTTCAC AACTATCTCA GCCATGAGGA AGGTCATGTT GCGGTTTTTG ATGCCACCAA |
| 481 | CACTACCAGA GPACGACGGT CACTGATCCT GCAGTTTGCA AAAGAACATG GTTACAAGGT |
| 541 | GTTTTTCATT GAGTCCATTT GTAATGACCC TGGCATAATT GCAGAAAACA TCAGGCAAGT |
| 601 | GAAACTTGGC AGCCCTGATT ATATAGACTG TGACCGGGAA AAGGTTCTGG AAGACTTTCT |
| 661 | AAAGAGAATT GAGTGCTATG AGGTCAACTA CCAACCCTTG GATGAGGAAC TGGACAGCCA |
| 721 | CCTGTCCTAC ATCAAGATCT TCGACGTGGG CACACGCTAC ATGGTGAACC GAGTGCAGGA |
| 781 | TCACATCCAG AGCCGCACAG TCTACTACCT CATGAATATC CATGTCACAC CTCGCTCCAT |
| 841 | CTACCTTTGC CGACATGGCG AGAGTGAACT CAACATCAGA GGCCGCATCG GAGGTGACTC |
| 901 | TGGCCTCTCA GTTCGCGGCA AGCAGTATGC CTATGCCCTG GCCAACTTCA TTCAGTCCCA |
| 961 | GGGCATCAGC TCCCTGAAGG TGTGGACCAG TCGCATGAAG AGGACCATCC AGACAGCTGA |
| 1021 | GGCCCTGGGT GTCCCCTATG AGCAGTGGAA GGCCCTGAAT GAGATTGATG CGGGTGTCTG |
| 1081 | TGAGGAGATG ACCTATGAAG AAATCCAGGA ACATTACCCT GAAGAATTTG CACTGCGAGA |
| 1141 | CCAAGATAAA TATCGCTACC GCTATCCCAA GGGAGAGTCC TATGAGGATC TGGTTCAGCG |
| 1201 | TCTGGAGCCA GTGATAATGG AGCTAGAACG ACAGGAGAAT GTACTGGTGA TCTGCCACCA |
| 1261 | GGCTGTCATG CGGTGCCTCC TGGCCTATTT CCTGGATAAA AGTTCAGATG AGCTTCCATA |
| 1321 | TCTCAAGTGC CCTCTGCACA CAGTGCTCAA ACTCACTCCT GTGGCTTATG GCTGCAAAGT |
| 1381 | GGAATCCATC TACCTGAATG TGGAGGCCGT GAACACACAC CGGGAGAAGC CTGAGAATGT |
| 1441 | GGACATCACC CGGGAACCTG AGGAAGCCCT GGATACTGTC CCAGCCCACT ACTGAGCGCT |
| 1501 | TTCCAAGAAG TCAAACTGCC TGTGTCCTCA TCGCCTTCCA CCTTTAGGAA ATGCTATCTT |
| 1561 | TGCTCTTCTC CTACTCTGCC TTGGCCTCAC TGAGGCACCC CACTTCCAGT GAAGAAGTCC |
| 1621 | TCCGCAACTC CCAAACAAGC CTCGCTTGCT GGCCGCAACC AAGGAGCTAT CTAGCTCTGG |
| 1681 | AGGAAACTTT CTTTCTTAAT TCCTATTCTC TGACGAATAA AGACTTACTG CCTACAAGAG |
| 1741 | G |

GENBANK ID: D00760
DEFINITION HUMAN MRNA FOR PROTEASOME SUBUNIT HC3.
VERSION D00760.1 GI:220023
CDS 1..705
/CODON_START=1

| | |
|---|---|
| 1 | ATGGCGGAGC GCGGGTACAG CTTTTCGCTG ACTACATTCA GCCCGTCTGG TAAACTTGTC |
| 61 | CAGATTGAAT ATGCTTTGGC TGCTCTAGCT GGAGGAGCCC CGTCCGTGGG AATTAAAGCT |
| 121 | GCAAATGCTG TGGTATTAGC AACTGAGAAA AAACAGAAAT CCATTCTGTA TGATGACCGA |
| 181 | AGTGTACACA AAGTAGAACC AATTACCAAG CATATAGGTT TGGTGTACAG TGGCATGGGC |
| 241 | CCCGATTACA GAGTGCTTGT GCACAGAGCT CGAAAACTAG CTCAACAATA CTATCTTGTG |
| 301 | TACCAAGAAC CCATTCCTAC AGCTCAGCTG GTACAGAGAG TAGCTTCTGP CATGCAAGAA |
| 361 | TATACTCAGT CAGGTGGTGT TCGTCCATTT GGAGTTTCTT TACTTATTTG TGGTTGGAAT |
| 421 | GACGGACCAC CATATTTATT TCAGTCAGAT CCATCTGGAG CTTACTTTGC CTGGAAAGCT |
| 481 | ACACCAATGG GAAAGAACTA TGTGAATCGG AAGACTTTCC TTGAGAAAAG ATATAATGAA |
| 541 | GATCTGGAAC TTGAAGATGC CATTCATACA GCCATCTTAA CCCTAAAGGA AAGCTTTGAA |
| 601 | GGGCAAATGA CAGAGGATAA CATAGAAGTT GGAATCTGCA ATGAAGCTGG ATTTAGGAGG |
| 661 | CTTACTCCAA CTGAAGTTAA GGATTACTTG GCTGCCATAG CATAACAATG AAGTGACTGA |
| 721 | AAAATCCAGA ATTTCAGATA ATCTATCTAC TTAAACATGT TTAAAGTATG TTTTGTTTTG |
| 781 | CAGACTTTTT GCATACTTAT TTCTACATGG TTTAAATCGA CTGTTTTTAA AATGACACTT |
| 841 | ATAAATCCTA ATAAACTGTT AAACCC |

GENBANK ID: P10644
GENBANK ID: XM_043948.2
DEFINITION HOMO SAPIENS ALDOLASE A, FRUCTOSE-BISPHOSPHATE (ALDOA), MRNA.
VERSION XM_043948.2 GI:18585537
CDS 243..1349
/CODON_START=1

| | |
|---|---|
| 1 | AAAAACCAGG GCTCCAGAGA ATCAGAACAG CCACCATCAC CGCAGGGAGT CAAGGGAGGA |
| 61 | GGGAGATTAG AGAAGGAGCC AGGGAGGGTG GCAGGGAGGC CACGTGATCC GAGTCCCCTC |
| 121 | ACCCCTTTCC TTCCCACAGG TCCCTGGCCA AAGATTTATT TCTCTTGACA ACCAAGGGCC |
| 181 | TCCGTCTGGA TTTCCAAGGA AGAATTTCCT CTGAAGCACC GGAACTTGCT ACTACCAGCA |
| 241 | CCATGCCCTA CCAATATCCA GCACTGACCC CGGAGCAGAA GAAGGAGCTG TCTGACATCG |
| 301 | CTCACCGCAT CGTGGCACCT GGCAAGGGCA TCCTGGCTGC AGATGAGTCC ACTGGGAGCA |
| 361 | TTGCCAAGCG GCTGCAGTCC ATTGGCACCG AGAACACCGA GGAGAACCGG CGCTTCTACC |
| 421 | GCCAGCTGCT GCTGACAGCT GACGACCGCG TGAACCCCTG CATTGGGGGT GTCATCCTCT |
| 481 | TCCATGAGAC ACTCTACCAG AAGGCGGATG ATGGGCGTCC CTTCCCCCAA GTTATCAAAT |
| 541 | CCAAGGGCGG TGTTGTGGGC ATGAAGGTAG ACAAGGGCGT GGTCCCCCTG GCAGGGACAA |
| 601 | ATGGCGAGAC TACCACCCAA GGGTTGGATG GGCTGTCTGA GCGCTGTGCC CAGTACAAGA |
| 661 | AGGACGGAGC TGACTTCGCC AAGTGGCGTT GTGTGCTGAA GATTGGGGAA CACACCCCCT |
| 721 | CAGCCCTCGC CATCATGGAA AATGCCAATG TTCTGGCCCG TTATGCCAGT ATCTGCCAGC |
| 781 | AGGTGGGCCT GCAGAATGGC ATTGTGCCCA TCGTGGAGCC TGAGATCCTC CCTGATGGGG |
| 841 | ACCATGACTT GAAGCGCTGC CAGTATGTGA CCGAGAAGGT GCTGGCTGCT GTCTACAAGG |
| 901 | CTCTGAGTGA CCACCACATC TACCTGGAAG GCACCTTGCT GAAGCCCAAC ATGGTCACCC |
| 961 | CAGGCCATGC TTGCACTCAG AAGTTTTCTC ATGAGGAGAT TGCCATGGCG ACCGTCACAG |
| 1021 | CGCTGCGCCG CACAGTGCCC CCCGCTGTCA CTGGGATCAC CTTCCTGTCT GGAGGCCAGA |
| 1081 | GTGAGGAGGA GGCGTCCATC AACCTCAATG CCATTAACAA GTGCCCCCTG CTGAAGCCCT |
| 1141 | GGGCCCTGAC CTTCTCCTAC GGCCGAGCCC TGCAGGCCTC TGCCCTGAAG GCCTGGGGCG |
| 1201 | GGAAGAAGGA GAACCTGAAG GCTGCGCAGG AGGAGTATGT CAAGCGAGCC CTGGCCAACA |
| 1261 | GCCTTGCCTG TCAAGGAAAG TACACTCCGA GCGGTCAGGC TGGGGCTGCT GCCAGCGAGT |
| 1321 | CCCTCTTCGT CTCTAACCAC GCCTATTAAG CGGAGGTGTT CCCAGGCTGC CCCCAACACT |
| 1381 | CCAGGCCCTG CCCCCTCCCA CTCTTGAAGA GGAGGCCGCC TCCTCGGGGC TCCAGGCTGG |
| 1441 | CTTGCCCGCG CTCTTTCTTC CCTCGTGACA GTGGTGTGTG GTGTCGTCTG TGAATGCTAA |
| 1501 | GTCCATCACC CTTTCCGGCA CACTGCCAAA TAAACAGCTA TTTAAGGGGG |

GENBANK ID: NM_005175.1
DEFINITION HOMO SAPIENS ATP SYNTHASE, H+TRANSPORTING, MITOCHONDRIAL F0
COMPLEX, SUBUNIT C (SUBUNIT 9), ISOFORM 1 (ATP5G1), MRNA.
VERSION NM_005175.1 GI:4885080
CDS 120..530
/CODON_START=1

| | |
|---|---|
| 1 | GGGGAAGCTG AGGGCTGAGA CCAAGGGCTA AAGCTGGGAG GTGAGTCTGT CACCTTGAGC |
| 61 | CGGGCGAGCG CTGTGGGCCA AGCAGGGGTT GCAGGGCAGT AGGAGTGCAG ACTGAAAAAA |
| 121 | TGCAGACCGC CGGGGCATTA TTCATTTCTC CAGCTCTGAT CCGCTGTTGT ACCAGGGGTC |
| 181 | TAATCAGGCC TGTGTCTGCC TCCTTCTTGA ATAGCCCAGT GAATTCATCT AAACAGCCTT |
| 241 | CCTACAGCAA CTTCCCACTC CAGGTGGCCA GACGGGAGTT CCAGACCAGT GTTGTCTCCC |
| 301 | GGGACATTGA CACAGCAGCC AAGTTTATTG GTGCTGGGGC AGCCACAGTT GGTGTGGCTG |
| 361 | GTTCAGGGGC TGGCATTGGA ACCGTGTTTG GCAGCTTGAT CATTGGCTAT GCCAGGAACC |
| 421 | CGTCTCTCAA GCAGCAGCTC TTCTCCTATG CCATTCTTGG CTTTGCCCTG TCTGAGGCCA |
| 481 | TGGGGCTTTT CTGTTTGATG GTCGCCTTCC TCATCCTCTT CGCCATGTGA GGCTCCATGG |
| 541 | GGGGTCACCG GCCTGTTGCT ACTGCAACTC CACACCATTC TTGGTGCTGC GGTGTGTTAA |
| 601 | GCTTTACCAT TAAACACAAC GTTTCTCTAA A |

GENBANK ID: M20496.1
DNA LINEAR
DEFINITION HUMAN CATHEPSIN L GENE, COMPLETE CDS.
VERSION M20496.1 GI:809235
CDS 134..1135
/CODON_START=1

| | |
|---|---|
| 1 | ACCTCCACGT GCCCTGTTTT TCTGGAGGCA CATCCTTGGC CTCTTCCACA GTCCTTGGGT |
| 61 | AAATGCTTGG GAGAATAATT TAAATATTTT TATTGTACCA TGGTGGCCCT AATTTTTCAG |
| 121 | GGGGCAGTAA GATATGAATC CTACACTCAT CCTTGCTGCC TTTTGCCTGG GAATTGCCTC |
| 181 | AGCTACTCTA ACATTTGATC ACAGTTTAGA GGCACAGTGG ACCAAGTGCA AGGCGATGCA |
| 241 | CAACAGATTA TACGGCATGA ATGAAGAAGG ATGGAGGAGA GCAGTGTGGG AGAAGAACAT |
| 301 | GAAGATGATT GAACTGCACA ATCAGGAATA CAGGGAAGGG AAACACAGCT TCACAATGGC |
| 361 | CATGAACGCC TTTGGAGACA TGACCAGTGA AGAATTCAGG CAGGTGATGA ATGGCTTTCA |
| 421 | AAACCGTAAG CCCAGGAAGG GGAAAGTGTT CCAGGAACCT CTGTTTTATG AGGCCGCCAG |
| 481 | ATCTGTGGAT TGGAGAGAGA AAGGCTACGT GACTCCTGTG AAGAATCAGG GTCAGTGTGG |
| 541 | TTCTTGTTGG GCTTTTAGTG CTACTGGTGC TCTTGAAGGA CAGATGTTCC GGAAAACTGC |
| 601 | GAGGCTTATC TCACTGAGTG AGCAGAATCT GGTAGACTGC TCTGGGCCTC AAGGCAATGA |
| 661 | AGGCTGCAAT GGTGGCCTAA TGGATTATGC TTTCCAGTAT GTTCAGGATA ATGGAGGCCT |
| 721 | GGACTCTGAG GAATCCTATC CATATGAGGC AACAGAAGAA TCCTGTAAGT ACAATCCCAA |
| 781 | GTATTCTGTT GCTAATGACA CCGGCTTTGT GGACATCCCT AAGCAGGAGA AGGCCCTGAT |
| 841 | GAAGGCAGTT GCAACTGTGG GGCCCATTTC TGTTGCTATT GATGCAGGTC ATGAGTCCTT |
| 901 | CCTGTTCTAT AAAGAAGGCA TTTATTTTGA GCCAGACTGT AGCAGTGAAG ACATGGATCA |
| 961 | TGGTGTGCTG GTGGTTGGCT ACGGATTTGA AAGCACAGAA TCAGATAACA ATAAATATTG |
| 1021 | GCTGGTGAAG AACAGCTGGG GTGAAGAATG GGGCATGGGT GGCTACGTAA AGATGGCCAA |
| 1081 | AGACCGGAGA AACCATTGTG GAATTGCCTC AGCAGCCAGC TACCCCACTG TGTGAGCTGT |
| 1141 | GGACGGTGAT GAGGAAGGAC TTGACTGGGG ATGGCGCATG CATGGGAGGA ATTCTTCAGT |
| 1201 | CTACCAGCCC CCGCTGTGTC GGATACACAC TCGAATCATT GAAGATCCGA GTGTGATTTG |
| 1261 | AATTCTGTGA TATTTTCACA CTGGTAAATG TTACCTCTAT TTTAATTACT GCTATAAATA |
| 1321 | GGTTTATATT ATTGATTCAC TTACTGACTT TGCATTTTCG TTTTTAAAAG GATGTATAAA |
| 1381 | TTTTTACCTG TTTAAATAAA ATCG |

GENBANK ID: XM_031596.3
DEFINITION HOMO SAPIENS ANNEXIN A4 (ANXA4), MRNA.
VERSION XM_031596.3 GI:18553329
CDS 48..770
/CODON_START=1

| | |
|---|---|
| 1 | GAAGAACTTC TGCTTGGGTG GCTGAACTCT GATCTTGACC TAGAGTCATG GCCATGGCAA |
| 61 | CCAAAGGAGG TACTGTCAAA GCTGCTTCAG GATTCAATGC CATGGAAGAT GCCCAGACCC |
| 121 | TGAGGAAGGC CATGAAAGGG CTCGGCACCG ATGAAGACGC CATTATTAGC GTCCTTGCCT |
| 181 | ACCGCAACAC CGCCCAGCGC CAGGAGATCA GGACAGCCTA CAAGAGCACC ATCGGCAGGG |
| 241 | ACTTGATAGA CGACCTGAAG TCAGAACTGA GTGGCAACTT CGAGCAGGTG ATTGTGGGGA |
| 301 | TGATGACGCC CACGGTGCTG TATGACGTGC AAGAGCTGCG AAGGGCCATG AAGGGAGCCG |
| 361 | GCACTGATGA GGGCTGCCTA ATTGAGATCC TGGCCTCCCG GACCCCTGAG GAGATCCGGC |
| 421 | GCATAAGCCA AACCTACCAG CAGCAATATG GACGGAGCCT TGAAGATGAC ATTCGCTCTG |
| 481 | ACACATCGTT CATGTTCCAG CGAGTGCTGG TGTCTCTGTC AGCTGGTGGG AGGGATGAAG |
| 541 | GAAATTATCT GGACGATGCT CTCGTGAGAC AGGATGCCCA GGACCTGTAT GAGGCTGGAG |
| 601 | AGAAGAAATG GGGGACAGAT GAGGTGAAAT TTCTAACTGT TCTCTGTTCC CGGAACCGAA |
| 661 | ATCACCTGTT GCATGGTTTG ATGAATACAA AAGGATATCA CAGAAGGATA TTGAACAGAG |
| 721 | TATTAAATCT GAAACATCTG GTAGCTTTGA AGATGCTCTG CTGGCTATAG TAAAGTGCAT |
| 781 | GAGGAACAAA TCTGCATATT TTGCTGAAAA GCTCTATAAA TCGATGAAGG GCTTGGGCAC |
| 841 | CGATGATAAC ACCCTCATCA GAGTGATGGT TTCTCGAGCA GAAATTGACA TGTTGGATAT |
| 901 | CCGGGCACAC TTCAAGAGAC TCTATGGAAA GTCTCTGTAC TCGTTCATCA AGGGTGACAC |
| 961 | ATCTGGAGAC TACAGGAAAG TACTGCTTGT TCTCTGTGGA GGAGATGATT AAAATAAAAA |
| 1021 | TCCCAGAAGG ACAGGAGGAT TCTCAACACT TTGAATTTTT TTAACTTCAT TTTTCTACAC |
| 1081 | TGCTATTATC ATTATCTCAG AATGCTTATT TCCAATTAAA ACCGCTACAG CTGCCTCCTA |
| 1141 | GAATATAGAC TGTCTGTATT ATTATTGACG TATAATTAGT CATTATGATG CTTTAAAGCT |
| 1201 | GTACTTGCAT TTCAAAGCTT ATAAGATATA AATGGAGATT TTAAAGTAGA AATAAATATG |
| 1261 | TATTCCATGT TTTTAAAAGA TTACTTTCTA CTTTGTCTTT CACAGACATT GAATATATTA |
| 1321 | AATTATTCCA TATTTTCTTT TCAGTGAAAA ATTTTTTAAA TGGAAGACTG TTCTAAAATC |
| 1381 | ACTTTTTTCC CTAATCCAAT TTTTAGAGTG GCTAGTAGTT TCTTCATTTG AAATTGTAAG |
| 1441 | CATCCGGTCA GTAAGAATGC CCATCCAGTT TTCTATATTT CATAGTCAAA GCCTTGAAAG |
| 1501 | CATCTACAAA TCTCTTTTTT TAGGTTTTGT CCATAGCATC AGTTGATCCT TACTAAGTTT |
| 1561 | TTCATGGGAG ACTTCCTTCA TCACATCTTA TGTTGAAATC ACTTTCTGTA GTCAAAGTAT |
| 1621 | ACCAAAACCA ATTTATCTGA ACTAAATTCT AAAGTATGGT TATACAAACC ATATACATCT |
| 1681 | GGTTACCAAA CATAAATGCT GAACATTCCA TATTATTATA GTTAATGTCT TAATCCAGCT |
| 1741 | TGCAAGTGAA TGGAAAAAAA AATAAGCTTC AAACTAGGTA TTCTGGGAAT GATGTAATGC |
| 1801 | TCTGAATTTA GTATGATATA AAGAAAACTT TTTTGTGCTA AAAATACTTT TTAAAATCAA |
| 1861 | TTTTGTTGAT TGTAGTAATT TCTATTTGCA CTGTGCCTTT CAACTCCAGA AACATTCTGA |
| 1921 | AGATGTACTT GGATTTAATT AAAAAGTTCA CTTTGT |

GENBANK ID: M22865.1
DEFINITION HUMAN CYTOCHROME B5 MRNA, COMPLETE CDS.
VERSION M22865.1 GI:181226
53..457
CODON_START=1

| | |
|---|---|
| 1 | CAGCCAGCTC GACGGGGCTG TGTGTGCTGG GCCTGGCTCG CGGCGAACCG AGATGGCAGA |
| 61 | GCAGTCGGAC GAGGCCGTGA ACTACTACAC CCTAGAGGAG ATTCAGAAGC ACAACCACAG |
| 121 | CAAGAGCACC TGGCTGATCC TGCACCACAA GGTGTACGAT TTGACCAAAT TTCTGGAAGA |
| 181 | GCATCCTGGT GGGGAAGAAG TTTTAAGGGA ACAAGCTGGA GGTGACGCTA CTGAGAACTT |
| 241 | TGAGGATGTC GGGCACTCTA CAGATGCCAG GGAAATGTCC AAAACATTCA TCATTGGGGA |
| 301 | GCTCCATCCA GATGACAGAC CAAAGTTAAA CAAGCCTCCG GAAACTCTTA TCACTACTAT |
| 361 | TGATTCTAGT TCCAGTTGGT GGACCAACTG GGTGATCCCT CCCATCTCTG CAGTGGCCGT |
| 421 | CGCCTTGATG TATCGCCTAT ACATGGCAGA GGACTGAACA CCTCCTCAGA AGTCAGCGCA |
| 481 | GGCCGAGCCT GCTTTGGACA CGGGAGAAAA GAAGCCATTG CTAACTACTT CAACTGACAG |
| 541 | AAACCTTCAC TTGAAAACAA TGATTTTAAT ATATCTCTTT CTTTTTCTTC CGACATTAGA |
| 601 | AACAAAACAA AAAGAACTGT CCTTTCTGCG CTCAAATTTT TCGAGTGTGC CTTTTTATTC |
| 661 | ATCTACTTTA TTTTGATGTT TCCTTAATGT GTAATTTACT TATTATAAGC ATCATCTTTT |
| 721 | AAAAATATAT TTGGCTTTTA AAG |

GENBANK ID: M14362.1
DEFINITION HUMAN T-CELL SURFACE ANTIGEN CD2 (T11) MRNA, COMPLETE CDS.
VERSION M14362.1 GI:179133
CDS 10..1065
/CODON_START=1

| | |
|---|---|
| 1 | ACCCCTAAGA TGAGCTTTCC ATGTAAATTT GTAGCCAGCT TCCTTCTGAT TTTCAATGTT |
| 61 | TCTTCCAAAG GTGCAGTCTC CAAAGAGATT ACGAATGCCT TGGAAACCTG GGGTGCCTTG |
| 121 | GGTCAGGACA TCAACTTGGA CATTCCTAGT TTTCAAATGA GTGATGATAT TGACGATATA |
| 181 | AAATGGGAAA AAACTTCAGA CAAGAAAAAG ATTGCACAAT TCAGAAAAGA GAAAGAGACT |
| 241 | TTCAAGGAAA AAGATACATA TAAGCTATTT AAAAATGGAA CTCTGAAAAT TAAGCATCTG |
| 301 | AAGACCGATG ATCAGGATAT CTACAAGGTA TCAATATATG ATACAAAAGG AAAAAATGTG |
| 361 | TTGGAAAAAA TATTTGATTT GAAGATTCAA GAGAGGGTCT CAAAACCAAA GATCTCCTGG |
| 421 | ACTTGTATCA ACACAACCCT GACCTGTGAG GTAATGAATG GAACTGACCC CGAATTAAAC |
| 481 | CTGTATCAAG ATGGGAAACA TCTAAAACTT TCTCAGAGGG TCATCACACA CAAGTGGACC |
| 541 | ACCAGCCTGA GTGCAAAATT CAAGTGCACA GCAGGGAACA AAGTCAGCAA GGAATCCAGT |
| 601 | GTCGAGCCTG TCAGCTGTCC AGAGAAAGGT CTGGACATCT ATCTCATCAT TGGCATATGT |
| 661 | GGAGGAGGCA GCCTCTTGAT GGTCTTTGTG GCACTGCTCG TTTTCTATAT CACCAAAAGG |
| 721 | AAAAAACAGA GGAGTCGGAG AAATGATGAG GAGCTGGAGA CAAGAGCCCA CAGAGTAGCT |
| 781 | ACTGAAGAAA GGGGCCGGAA GCCCCACCAA ATTCCAGCTT CAACCCCTCA GAATCCAGCA |
| 841 | ACTTCCCAAC ATCCTCCTCC ACCACCTGGT CATCGTTCCC AGGCACCTAG TCATCGTCCC |
| 901 | CCGCCTCCTG GACACCGTGT TCAGCACCAG CCTCAGAAGA GGCCTCCTGC TCCGTCGGGC |
| 961 | ACACAAGTTC ACCAGCAGAA AGGCCCGCCC CTCCCCAGAC CTCGAGTTCA GCCAAAACCT |
| 1021 | CCCCATGGGG CAGCAGAAAA CTCATTGTCC CCTTCCTCTA ATTAAAAAAG ATAGAAACTG |
| 1081 | TATTTTTCAA TAAAAAGCAC TGTGGATTTC TGCCCTCCTG ATGTGCATAT CCGTACTTCC |
| 1141 | ATGAGGTGTT TTCTGTGTGC AGAACATTGT CACCTCCTGA GGCTGTGGGC CACAGCCACC |
| 1201 | TCTGCATCTT CGAACTCAGC CATGTGGTCA ACATCTGGAG TTTTTGGTCT CCTCAGAGAG |
| 1261 | CTCCATCACA CCAGTAAGGA GAAGCAATAT AAGTGTGATT GCAAGAATGG TAGAGGACCG |
| 1321 | AGCACAGAAA TCTTAGAGAT TTCCTGTCCC CTCTCAGGTC ATGTGTAGAT GCGATAAATC |
| 1381 | AAGTGATTGG TGTGCCTGGG TCTCACTACA AGCAGCCTAT CTGCTTAAGA GACTCTGGAG |
| 1441 | TTTCTTATGT GCCCTGGTGG ACACTTGCCC ACCATCCTGT GAGTAAAAGT GAAATAAAAG |
| 1501 | CTTTGACTAG |

GENBANK ID: XM_087746.1
DEFINITION HOMO SAPIENS SIMILAR TO KIDNEY ANINOPEPTIDASE N; LEUCINE
ARYLANINOPEPTIDASE 1 (LOC153726), MRNA.
VERSION XM_087746.1 GI:18561749
CDS 262..639
/CODON_START=1

| | |
|---|---|
| 1 | GAGTTCCATG CCACCTCCCC GCCCTTTACA GACATGCTAT AAGGTCCCCA GCCCAGTCAC |
| 61 | TCCGCAGTGC CTCTCTCTTC CTCCCCATGG ACTATACACA GGCCCTGCTT GTCCTGGAGG |
| 121 | AAAGTTTGGA CGTCATTATA TAGAICAGGA GACTGAAGTA CTGAAAGGTT AAATGACTTG |
| 181 | CCAAAGAATG AGATCTTTTT TTCTAACATT TTACATAATA TCCTCAGAGA AGATCACGCC |
| 241 | CTGGTGACTA GAGCTGTGGC CATGAAGGTG GAAAATTTCA AAACAAGTGA AATACAGGAA |
| 301 | CTCTTTGACA TATTTACTTA CAGCAAGGGA GCGTCTATGG CCCGGATGCT TTCTTGTTTC |
| 361 | TTGAATGAGC ATTTATTTGT CAGTGCACTC AAGTCATATT TGAAGACATT TTCCTACTCA |
| 421 | AACGCTGAGC AAGATGATCT ATGGAGGCAT TTTCAAATGG CCATAGATGA GCAGACTACA |
| 481 | GTTATTTTGC CAGCAACAAT AAAAAACATA ATGGACAGGT GGACACACCA GAGTGGTTTT |
| 541 | CCAGTGATCA CTTTAAATGT GTCTACTGGC GTCATGAAAC AGGAGCCATT TTATCTTGAA |
| 601 | AACATTAAAA ATCGGACTCT TCTAACCAGC AATAAGTGAC ACATGGATTG TCCCTATTCT |
| 661 | TTGGATAAAA AATGGAACTA CACAACCTTT AGTCTGGCTA GA |

GENBANK ID: P31749
DEFINITION DICTYOSTELION DISCOIDEUM RAC-ALPHA SERINETHREONINE KINASE
HOMOLOG
MRNA, COMPLETE CDS.
VERSION PU15210.1 GI:1000068
CDS 1..1335
/CODON_START=1

| | |
|---|---|
| 1 | ATGTCAACAG CACCAATTAA ACATGAAGGT TTCCTCACTA AAGAAGGTGG TGGTTTCAAA |
| 61 | AGTTGGAAAA AGAGATGGTT CATTCTCAAA GGTGGTGATT TAAGTTATTA TAAAACAAAA |
| 121 | GGTGAACTTG TACCATTAGG AGTTATTCAT TTAAATACAT CAGGTCATAT TAAAAATTCT |
| 181 | GATCGTAAGA AAAGAGTTAA TGGATTTGAA GTACAAACAC CATCACGTAC ATATTTCTTA |
| 241 | TGTTCAGAGA CAGAGGAAGA ACGTGCAAAA TGGATAGAGA TATTAATTAA TGAAAGAGAA |
| 301 | TTATTATTGA ATGGTGGTAA ACAACCAAAG AAATCGGAAA AGGTAGGAGT TGCAGATTTT |
| 361 | GAATTATTGA ATTTAGTTGG TAAAGGTAGT TTTGGTAAAG TTATTCAAGT TAGAAAGAAA |
| 421 | GATACTGGTG AAGTGTATGC AATGAAAGTT TTATCAAAGA AACATATCGT AGAGCATAAC |
| 481 | GAAGTCGAAC ATACATTGAG TGAGCGTAAT ATTCTTCAAA AGATCAATCA CCCATTTTTG |
| 541 | GTTAATCTCA ACTACAGTTT TCAAACAGAG GATAAGCTTT ACTTTATCTT GGATTATGTT |
| 601 | AATGGTGGTG AGTTATTCTA TCATCTTCAA AAGGACAAAA AGTTTACAGA GGATCGTGTC |
| 661 | CGTTATTATG GCGCAGAGAT CGTATTGGCA TTGGAACATT TACATTTGTC GGGTGTCATC |
| 721 | TATAGAGATT TGAAACCAGA GAATTTACTA CTCACCAACG AGGGTCACAT TTGCATGACC |
| 781 | GATTTCGGTC TTTGCAAAGA GGGTCTATTG ACACCAACCG ACAAAACTGG TACTTTCTGT |
| 841 | GGTACTCCTG AATATTTAGC ACCCGAAGTA CTTCAAGGCA ATGGTTATGG TAAACAAGTG |
| 901 | GATTGGTGGA GTTTTGGTTC TCTCCTCTAT GAAATGCTCA CTGGTTTACC ACCATTCTAC |
| 961 | AATCAAGACG TCCAAGAGAT GTATCGTAAG ATCATGATGG AGAAATTATC TTTCCCACAT |
| 1021 | TTCATTTCTC CAGATGCTCG TTCCCTCTTG GAACAACTCT TGGAAAGAGA TCCTGAAAAA |
| 1081 | AGACTTGCCG ATCCAAATCT TATTAAAAGA CATCCTTTCT TCCGTTCCAT CGATTGGGAA |
| 1141 | CAATTATTCC AAAAGAATAT TCCACCACCA TTCATTCCAA ATGTTAAAGG TTCTGCTGAT |
| 1201 | ACCTCTCAAA TTGATCCAGT TTTCACTGAT GAAGCTCCTT CTTTAACTAT GGCTGGTGAA |
| 1261 | TGTGCTTTAA ATCCGCAACA ACAAAAAGAT TTTGAAGGAT TTACATATGT CGCTGAATCT |
| 1321 | GAACATTTAA GATAA |

GENBANK ID: NM_000102.2
DEFINITION HOMO SAPIENS CYTOCEROME P450, SUBFANILY XVII (STEROID
17-ALPHA--HYDROXYLASE), ADRENAL HYPERPLASIA (CYP17), MRNA.
VERSION NM_000102.2 GI:13904854
CDS 61..1587

| | |
|---|---|
| 1 | GAGTTGCCAC AGCTCTTCTA CTCCACTGCT GTCTATCTTG CCTGCCGGCA CCCAGCCACC |
| 61 | ATGTGGGAGC TCGTGGCTCT CTTGCTGCTT ACCCTAGCTT ATTTGTTTTG GCCCAAGAGA |
| 121 | AGGTGCCCTG GTGCCAAGTA CCCCAAGAGC CTCCTGTCCC TGCCCCTGGT GGGCAGCCTG |
| 181 | CCATTCCTCC CCAGACATGG CCATATGCAT AACAACTTCT TCAAGCTGCA GAAAAAATAT |
| 241 | GGCCCCATCT ATTCTGTTCG TATGGGCACC AAGACTACAG TGATTGTCGG CCACCACCAG |
| 301 | CTGGCCAAGG AGGTGCTTAT TAAGAAGGGC AAGGACTTCT CTGGGCGGCC TCAAATGGCA |
| 361 | ACTCTAGACA TCGCGTCCAA CAACCGTAAG GGTATCGCCT TCGCTGACTC TGGCGCACAC |
| 421 | TGGCAGCTGC ATCGAAGGCT GGCGATGGCC ACCTTTGCCC TGTTCAAGGA TGGCGATCAG |
| 481 | AAGCTGGAGA AGATCATTTG TCAGGAAATC ACTACATTGT GTGATATGCT GGCCACCCAC |
| 541 | AACGGACAGT CCATAGACAT CTCCTTTCCT GTCTTCGTGG CGGTAACCAA TGTCATCTCC |
| 601 | TTGATCTGCT TCAATACCTC CTACAAGAAT GGCGACCCTG AGTTGAATGT CATACAGAAT |
| 661 | TACAATGAAG GCATCATAGA CAACCTCAGC AAACACAGCC TGCTGGACCT AGTCCCCTGG |
| 721 | TTGAAGATTT TCCCCAACAA AACCCTGGAA AAATTAAAGA GCCATGTTAA AATACGAAAT |
| 781 | GATCTGCTGA ATAAAATACT TGAAAATTAC AAGGAGAAAT TCCGGAGTGA CTCTATCACC |
| 841 | AACATGCTGG ACACACTGAT CCAAGCCAAG ATGAACTCAG ATAATGGCAA TGCTGGCCCA |
| 901 | GATCAAGATT CAGAGGTGCT TTCAGATAAC CACATTCTCA CCACCATAGG GGACATCTTT |
| 961 | GGGGCTGGCG TGGAGACCAC CACCTCTGTG GTTAAATGGA CCCTGGCCTT CCTGCTGCAC |
| 1021 | AATCCTCAGG TGAAGAAGAA GCTCTACGAG GAGATTGACC AGAATGTGGG TTTCAGCCGC |
| 1081 | ACACCAACTA TCAGTGACCG TAACCGTCTC CTCCTGCTGG AGGCCACCAT CGGAGAGGTG |
| 1141 | CTTCGCCTCA GGCCCGTGGC CCCTATGCTC ATCCCCCACA AGGCCAACGT TGACTCCAGC |
| 1201 | ATCGGTGAGT TTGCTGTGGA CAAGGGCACA GAAGTTATCA TCAATCTGTG GGCGCTGCAT |
| 1261 | CACAATGAGA AGGAGTGGCA CCAGCCGGAT CAGTTCATGC CTGAGCGTTT CTTGAATCCA |
| 1321 | GCGGGGACCC AGCTCATCTC ACCGTCAGTA AGCTATTTGC CCTTCGGAGC AGGACCTCGC |
| 1381 | TCCTGTATAG GTGAGATCCT GGCCCGCCAG GAGCTCTTCC TCATCATGGC CTGGCTGCTG |
| 1441 | CAGAGGTTCG ACCTGGAGGT GCCAGATGAT GGGCAGCTGC CCTCCCTGGA AGGCATCCCC |
| 1501 | AAGGTGGTCT TTCTGATCGA CTCTTTCAAA GTGAAGATCA AGGTGCGCCA GGCCTGGAGG |
| 1561 | GAAGCCCAGG CTGAGGGTAG CACCTAAAGG CTGTAACTCA CAGCCCCTGT CCACCCTATG |
| 1621 | TGGCCCCACA ACACAGATTT AGAGATACAA CCCCCCACCC TTCTCCGCCA TTCTTCCCTA |
| 1681 | CTCCCAACCC ACTCTGCCTT CTTTTTCAGC TTGTGGCAAT GCCAGTGATG TGCATAAACA |
| 1741 | GTTTTTTTTT TTTCC |

GENBANK ID: NM_001662
DEFINITION HOMO SAPIENS ADP-RIBOSYLATION FACTOR 5 (ARF5), MRNA.
VERSION NM_001662.2 GI:6995999
CDS 37..579
/CODON_START=1

| | |
|---|---|
| 1 | CCGCGTCGGT GCCCGCGCCC CTCCCCGGGC CCCGCCATGG GCCTCACCGT GTCCGCGCTC |
| 61 | TTTTCGCGGA TCTTCGGGAA GAAGCAGATG CGGATTCTCA TGGTTGGCTT GGATGCGGCT |
| 121 | GGCAAGACCA CAATCCTGTA CAAACTGAAG TTGGGGGAGA TTGTCACCAC CATCCCAACC |
| 181 | ATAGGCTTCA ATGTAGAAAC AGTGGAATAT AAGAACATCT GTTTCACAGT CTGGGACGTG |
| 241 | GGAGGCCAGG ACAAGATTCG GCCTCTGTGG CGGCACTACT TCCAGAACAC TCAGGGCCTC |
| 301 | ATCTTTGTGG TGGACAGTAA TGACCGGGAG CGGGTCCAAG AATCTGCTGA TGAACTCCAG |
| 361 | AAGATGCTGC AGGAGGACGA GCTGCGGGAT GCAGTGCTGC TGGTATTTGC CAACAAGCAG |
| 421 | GACATGCCCA ACGCCATGCC CGTGAGCGAG CTGACTGACA AGCTGGGGCT ACAGCACTTA |
| 481 | CGCAGCCGCA CGTGGTATGT CCAGGCCACC TGTGCCACCC AAGGCACAGG TCTGTACGAT |
| 541 | GGTCTGGACT GGCTGTCCCA CGAGCTGTCA AAGCGCTAAC CAGCCAGGGG CAGGCCCCTG |
| 601 | ATGCCCGGAA GCTCCTGCGT GCATCCCCGG GATGACCAGA CTCCCGGACT CCTCAGGCAG |
| 661 | TGCCCTTTCC TCCCACTTTT CCTCCCCCAT AGCCACAGGC CTCTGCTCCT GCTCCTGCCT |
| 721 | GCATGTTCTC TCTGTTGTTG GAGCCTGGAG CCTTGCTCTC TGGGCACAGA GGGGTCCACT |
| 781 | CTCCTGCCTG CTGGGACCTA TGGAAGGGGC TTCCTGGCCA AGGCCCCCTC TTCCAGAGGA |
| 841 | GGAGCAGGGA TCTGGGTTTC CTTTTTTTTT TCTGTTTTGG GTGTACTCTA GGGGCCAGGT |
| 901 | TGGGAGGGGG AAGGTGAGGG CTTCGGGTGG TGCTATAATG TGGCACTGGA TCTTGAGTAA |
| 961 | TAAATTTGCT GTGGTTTG |

GENBANK 10: XM_048886.3
DEFINITION HOMO SAPIENS MICROSOMAL GLUTATHIONE 5-TRANSFERASE 1
(MGST1), MRNA.
VERSION XM_048886.3 GI:18580621
CDS 89..556
/CODON_START=1

| | |
|---|---|
| 1 | AGTCCCTGCA TTGCGCGCGA CCCGGCGGCG GGACAGGCTT GCTGCTTCCT CCTCCTCGGC |
| 61 | CTCACCATTC CAGACCAAAA TTGAAAAAAT GGTTGACCTC ACCCAGGTAA TGGATGATGA |
| 121 | AGTATTCATG GCTTTTGCAT CCTATGCAAC AATTATTCTT TCAAAAATGA TGCTTATGAG |
| 181 | TACTGCAACT GCATTCTATA GATTGACAAG AAAGGTTTTT GCCAATCCAG AAGACTGTGT |
| 241 | AGCATTTGGC AAAGGAGAAA ATGCCAAGAA GTATCTTCGA ACAGATGACA GAGTAGAACG |
| 301 | TGTACGCAGA GCCCACCTGA ATGACCTTGA AAATATTATT CCATTTCTTG GAATTGGCCT |
| 361 | CCTGTATTCC TTGAGTGGTC CCGACCCCTC TACAGCCATC CTGCACTTCA GACTATTTGT |
| 421 | CGGAGCACGG ATCTACCACA CCATTGCATA TTTGACACCC CTTCCCCAGC CAAATAGAGC |
| 481 | TTTGAGTTTT TTTGTTGGAT ATGGAGTTAC TCTTTCCATG GCTTACAGGT TGCTGAAAAG |
| 541 | TAAATTGTAC CTGTAAAGAA AATCATACAA CTCAGCATCC AGTTGGCTTT TTAAGAATTC |
| 601 | TGTACTTCCA ATTTATAATG AATACTTTCT TAGATTTTAG GTAGGAGGGG AGCAGAGGAA |
| 661 | TTATGAACTG GGGTAAACCC ATTTTGAATA TTAGCATTGC CAATATCCTG TATTCTTGTT |
| 721 | TTACATTTGG ATTAGAAATT TAACATAGTA ATTCTTAAGT CTTTTGTCTG ATTTTTAAAG |
| 781 | TACTTTCTTA TAAATTTGGA TCATGTTATG ATTTGTAACA TTCACACAAC ACCTCACTTT |
| 841 | TGAATCTATA AAAGAATTGC ACGTATGAGA AACCTATATT TCAATACTGC TGAAACAGAC |
| 901 | ATGAAATAAA GAATTTAAAG AATG |

GENBANK ID: X02162
DEFINITION HUMAN MRNA FOR AFOLIPOPROTEIN AI (APO AI)=.
VERSION X02162.1 GI:28771
CDS 87..890
/CODON_START=1

| | |
|---|---|
| 1 | GAATTCAAAA AAAAAAGAAA AAAAAAAAAA AAAAAAAAAA AAAAAAAGAG AGACTGCGAG |
| 61 | AAGGAGGTCC CCCACGGCCC TTCAGGATGA AAGCTGCGGT GCTGACCTTG GCCGTGCTCT |
| 121 | TCCTGACGGG GAGCCAGGCT CGGCATTTCT GGCAGCAAGA TGAACCCCCC CAGAGCCCCT |
| 181 | GGGATCGAGT GAAGGACCTG GCCACTGTGT ACGTGGATCT GCTCAAAGAC AGCGGCAGAG |
| 241 | ACTATGTGTC CCAGTTTGAA GGCTCCGCCT TGGGAAAACA GCTAAACCTA AAGCTCCTTG |
| 301 | ACAACTGCGA CAGCCTGACC TCCACCTTCA GCAAGCTGCG CGAACAGCTC GGCCCTGTGA |
| 361 | CCCAGGAGTT CTGGGATAAC CTGGAAAAGG AGACAGAGGG CCTGAGGCAG GAGATGAGCA |
| 421 | AGGATCTGGA GGAGGTGAAG GCCAAGGTGC AGCCCTACCT GGACGACTTC CAGAAGAAGT |
| 481 | GGCAGGAGGA GATGGAGCTC TACCGCCAGA AGGTGGAGCC GCTGCGCGCA GAGCTCCAAG |
| 541 | AGGGCGCGCG CCAGAAGCTG CACGAGCTGC AAGAGAAGCT GAGCCCACTG GGCGAGGAGA |
| 601 | TGCGCGACCG CGCGCGCGCC CATCTGGACG CGCTGCGCAC GCATCTGGCC CCCTACAGCG |
| 661 | ACGAGCTGCG CCAGCGCTTG GCCGCGCGCC TTGAGCCTCT CAAGGACAAC GGCGGCGCCA |
| 721 | GACTGGCCGA GTACCACGCC AAGGCCACCG AGCATCTGAG CACGCTCAGC GAGAAGGCCA |
| 781 | AGCCCGCGCT CGAGGACCTC CGCCAAGCCC TCCTGCCCGT GCTGGAGAGC TTCAAGGTCA |
| 841 | GCTTCCTGAG CGCTCTCGAC GACTACACTA AGAAGCTCAA CACCCAGTGA GCCGCCCGCC |
| 901 | GCCGCCCCCC TTCCCGGTGC TCAGAATAAA CGTTTCCAAA GTGGGAAAAA AAAAAAAAAG |
| 961 | AATTC |

GENBANK ID: XM_007441.1
DEFINITION HOMO SAPIENS PRESENILIN 1 (ALZHEIS2ER DISEASE 3) (PSEN1),
MRNA.
VERSION XM_007441.1 GI:11435041
CDS 249..1652
/CODON_START=1

| | |
|---|---|
| 1 | TGGGACAGGC AGCTCCGGGG TCCGCGGTTT CACATCGGAA ACAAAACAGC GGCTGGTCTG |
| 61 | GAAGGAACCT GAGCTACGAG CCGCGGCGGC AGCGGGGCGG CGGGGAAGCG TATACCTAAT |
| 121 | CTGGGAGCCT GCAAGTGACA ACAGCCTTTG CGGTCCTTAG ACAGGTTGGG CTGGAGGAGA |
| 181 | ACACATGAAA GAAAGAACCT CAAGAGGCTT TGTTTTCTGT GAAACAGTAT TTCTATACAG |
| 241 | TTGCTCCAAT GACAGAGTTA CCTGCACCGT TGTCCTACTT CCAGAATGCA CAGATGTCTG |
| 301 | AGGACAACCA CCTGAGCAAT ACTGTACGTA GCCAGAATGA CAATAGAGAA CGGCAGGAGC |
| 361 | ACAACGACAG ACGGAGCCTT GGCCACCCTG AGCCATTATC TAATGGACGA GCGCAGGGTA |
| 421 | ACTCCCGGCA GGTGGTGGAG CAAGATGAGG AAGAAGATGA GGAGCTGACA TTGAAATATG |
| 481 | GCGCCAAGCA TGTGATCATG CTCTTTGTCC CTGTGACTCT CTGCATGGTG GTGGTCGTGG |
| 541 | CTACCATTAA GTCAGTCAGC TTTTATACCC GGAAGGATGG GCAGCTAATC TATACCCCAT |
| 601 | TCACAGAAGA TACCGAGACT GTGGGCCAGA GAGCCCTGCA CTCAATTCTG AATGCTGCCA |
| 661 | TCATGATCAG TGTCATTGTT GTCATGACTA TCCTCCTGGT GGTTCTGTAT AAATACAGGT |
| 721 | GCTATAAGGT CATCCATGCC TGGCTTATTA TATCATCTCT ATTGTTGCTG TTCTTTTTTT |
| 781 | CATTCATTTA CTTGGGGGAA GTGTTTAAAA CCTATAACGT TGCTGTGGAC TACATTACTG |
| 841 | TTGCACTCCT GATCTGGAAT TTTGGTGTGG TGGGAATGAT TTCCATTCAC TGGAAAGGTC |
| 901 | CACTTCGACT CCAGCAGGCA TATCTCATTA TGATTAGTGC CCTCATGGCC CTGGTGTTTA |
| 961 | TCAAGTACCT CCCTGAATGG ACTGCGTGGC TCATCTTGGC TGTGATTTCA GTATATGATT |
| 1021 | TAGTGGCTGT TTTGTGTCCG AAAGGTCCAC TTCGTATGCT GGTTGAAACA GCTCAGGAGA |
| 1081 | GAAATGAAAC GCTTTTTCCA GCTCTCATTT ACTCCTCAAC AATGGTGTGG TTGGTGAATA |
| 1141 | TGGCAGAAGG AGACCCGGAA GCTCAAAGGA GAGTATCCAA AAATTCCAAG TATAATGCAG |
| 1201 | AAAGCACAGA AAGGGAGTCA CAAGACACTG TTGCAGAGAA TGATGATGGC GGGTTCAGTG |
| 1261 | AGGAATGGGA AGCCCAGAGG GACAGTCATC TAGGGCCTCA TCGCTCTACA CCTGAGTCAC |
| 1321 | GAGCTGCTGT CCAGGAACTT TCCAGCAGTA TCCTCGCTGG TGAAGACCCA GAGGAAAGGG |
| 1381 | GAGTAAAACT TGGATTGGGA GATTTCATTT TCTACAGTGT TCTGGTTGGT AAAGCCTCAG |
| 1441 | CAACAGCCAG TGGAGACTGG AACACAACCA TAGCCTGTTT CGTAGCCATA TTAATTGGTT |
| 1501 | TGTGCCTTAC ATTATTACTC CTTGCCATTT TCAAGAAAGC ATTGCCAGCT CTTCCAATCT |
| 1561 | CCATCACCTT TGGGCTTGTT TTCTACTTTG CCACAGATTA TCTTGTACAG CCTTTTATGG |
| 1621 | ACCAATTAGC ATTCCATCAA TTTTATATCT AGCATATTTG CGGTTAGAAT CCCATGGATG |
| 1681 | TTTCTTCTTT GACTATAACA AAATCTGGGG AGGACAAAGG TGATTTTCCT GTGTCCACAT |
| 1741 | CTAACAAAGT CAAGATTCCC GGCTGGACTT TTGCAGCTTC CTTCCAAGTC TTCCTGACCA |
| 1801 | CCTTGCACTA TTGGACTTTG GAAGGAGGTG CCTATAGAAA ACGATTTTGA ACATACTTCA |
| 1861 | TCGCAGTGGA CTGTGTCCCT CGGTGCAGAA ACTACCAGAT TTGAGGGACG AGGTCAAGGA |
| 1921 | GATATGATAG GCCCGGAAGT TGCTGTGCCC CATCAGCAGC TTGACGCGTG GTCACAGGAC |
| 1981 | GATTTCACTG ACACTGCGAA CTCTCAGGAC TACCGTTACC AAGAGGTTAG GTGAAGTGGT |
| 2041 | TTAAACCAAA CGGAACTCTT CATCTTAAAC TACACGTTGA AAATCAACCC AATAATTCTG |
| 2101 | TATTAACTGA ATTCTGAACT TTTCAGGAGG TACTGTGAGC AAGAGCAGGC ACCAGCAGCA |
| 2161 | GAATGGGGAA TCGACAGGTG GGCAGCGGTT CCAGCTTCCC TTTGATTTTT TGCTGCAGAC |
| 2221 | TCATCCTTTT TAAATGAGAC TTGTTTTCCC CTCTCTTTGA GTCAAGTCAA ATATGTAGAT |
| 2281 | TGCCTTTGGC AATTCTTCTT CTCAAGCACT GACACTCATT ACCGTCTGTG ATTGCCATTT |
| 2341 | CTTCCCAAGG CCAGTCTGAA CCTGAGGTTG CTTTATCCTA AAAGTTTTAA CCTCAGGTTC |
| 2401 | CAAATTCAGT AAATTTTGGA AACAGTACAG CTATTTCTCA TCAATTCTCT ATCATGTTGA |
| 2461 | AGTCAAATTT GGATTTTCCA CCAAATTCTG AATTTCTAGA CATACTTGTA CGCTCACTTG |
| 2521 | CCCCAGATGC CTCCTCTGTC CTCATTCTTC TCTCCCACAC AAGCAGTCTT TTTCTACAGC |
| 2581 | CAGTAAGGCA GCTCTGTCGT GGTAGCAGAT GGTCCCATTA TTCTAGGGTC TTACTCTTTG |
| 2641 | TATGATGAAA AGAATGTGTT ATGAATCGGT GCTGTCAGCC CTGCTGTCAG ACCTTCTTCC |
| 2701 | ACAGCAAATG AGATGTATGC CCAAAGACGG TAGAATTAAA GAAGAGTAAA ATGGCTGTTG |
| 2761 | AAGC |

GENBANK ID: XM_087242.1
DEFINITION HOMO SAPIENS ARGINYL AMINOPEPTIDASE (AMINOPEPTIDASE B) -LIKE
1(RNPEPL1), MRNA.
VERSION XM_087242.1 GI:18600482
CDS 700..1764
/CODON_START=1

| | |
|---|---|
| 1 | GTGGACCCGT TCACCGACTA CGGCTCCTCC CTCACCGTCA CGCTGCCGCC CGAGCTGCAG |
| 61 | GCGCACCAGC CCTTCCAGGT CATCCTGCGG TACACCTCGA CCGACGCCCC CGCCATCTGG |
| 121 | TGGCTGGACC CAGAGCTGAC CTATGGCTGC GCCAAGCCCT TCGTCTTCAC CCAGGGCCAC |
| 181 | TCCGTGTGCA ACCGCTCCTT CTTCCCGTGC TTCGACACAC CTGCCGTGAA GTGCACCTAC |
| 241 | TCTGCCGTCG TCAAGGCGCC ATCGGGGGTG CAGGTGCTGA TGAGTGCCAC CCGGAGTGCA |
| 301 | TACATGGAGG AAGAAGGCGT CTTCCACTTC CACATGGAGC ACCCCGTGCC CGCCTACCTC |
| 361 | GTGGCCCTGG TGGCCGGAGA CCTCAAGCCG GCAGACATCG GGCCCAGGAG CCGCGTGTGG |
| 421 | GCCGAGCCAT GCCTCCTGCC CACGGCCACC AGCAAGCTGT CGGGCGCAGT GGAGCAGTGG |
| 481 | CTGAGTGCAG CTGAGCGGCT GTATGGGCCC TACATGTGGG GCAGGTACGA CATTGTCTTC |
| 541 | CTGCCACCCT CCTTCCCCAT CGTGGCCATG GAGAACCCCT GCCTCACCTT CATCATCTCC |
| 601 | TCCATCCTGG AGAGCGATGA GTTCCTGGTC ATCGATGTCA TCCACGAGGT GGCCCACAGT |
| 661 | TGGTTCGGCA ACGCTGTCAC CAACGCCACG TGGGAAGAGA TGTGGCTGAG CGAGGGCCTG |
| 721 | GCCACCTATG CCCAGCGCCG TATCACCACC GAGACCTACG GTGCTGCCTT CACCTGCCTG |
| 781 | GAGACTGCCT TCCGCCTGGA CGCCCTGCAC CGGCAGATGA AGCTTCTGGG AGAGGACAGG |
| 841 | CCGGTCAGCA AACTGCAGGT CAAGCTGGAG CCAGGAGTGA ATCCCAGCCA CCTGATGAAC |
| 901 | CTGTTCACCT ACGAGAAGGG CTACTGCTTC GTGTACTACC TGTCCCAGCT CTGCGGAGAC |
| 961 | CCACAGCGCT TTGATGACTT TCTCCGAGCC TATGTGGAGA AGTACAAGTT CACCAGCGTG |
| 1021 | GTGGCCCAGG ACCTGCTGGA CTCCTTCCTG AGCTTCTTCC CGGAGCTGAA GGAGCAGAGC |
| 1081 | GTGGACTGCC GGGCAGGGCT GGAATTCGAG CGCTGGCTCA ATGCCACAGG CCCGCCGCTG |
| 1141 | GCTGAGCCGG ACCTGTCTCA GGGATCCAGC CTGACCCGGC CCGTGGAGGC CCTTTTCCAG |
| 1201 | CTGTGGACCG CAGAACCTCT GGACCAGGCA GCTGCCTCGG CCAGCGCCAT TGACATCTCC |
| 1261 | AAGTGGAGGA CCTTCCAGAC AGCACTCTTC CTGGACCGGC TCCTGGATGG GTCCCCGCTG |
| 1321 | CCGCAGGAGG TGGTGATGAG CCTGTCCAAG TGCTACTCCT CCCTGCTGGA CTCGATGAAC |
| 1381 | GCTGAGATCC GCATCCGCTG GCTGCAGATT GAGGTCCGCA ACGACTACTA TCCTGACCTC |
| 1441 | CACAGGGTGC GGCGCTTCCT GGAGAGCCAG ATGTCACGCA TGTACACCAT CCCGCTGTAC |
| 1501 | GAGGACCTCT GCACCGGTGC CCTCAAGTCC TTCGCGCTGG AGGTCTTCTA CCAGACGCAG |
| 1561 | GGCCGGCTGC ACCCCAACCT GCGCAGAGCC ATCCAGCAGA TCCTGTCCCA GGGCCTGGGC |
| 1621 | TCCAGCACAG AGCCCGCCTC AGAGCCCAGC ACGGAGCTGG GCAAGGCTGA AGCAGACACA |
| 1681 | GACTCGGACG CACAGGCCCT GCTGCTTGGG GACGAGGCCC CCAGCAGTGC CATCTCTCTC |
| 1741 | AGGGACGTCA ATGTGTCTGC CTAGCCCTGT TGGCGGGCTG ACCCTCGACC TCCCAGACAC |
| 1801 | CACAATTGTG CCTTCTGTGG GCCAGGCCTG CCATGACTGC GTCTCGGCTC TGGCCATGAG |
| 1861 | CTCTGCCCAG GCCCACAAGC CCCTCCCCTG GGCTCTCCCA GGCAGGGAGA ATGGGGAGAG |
| 1921 | GGACCTCCTT GTGTCTGGCA GAGACCTGTG GACCTGGCCT CCCCACTCCC AGCTCTCTTG |
| 1981 | CACTGCAGGC CCTGGGGCCA GCCCGCACAC ACCATGCCTC CTGTCTCAAC ACTGACAGCT |
| 2041 | GTGCCTAGCC CCGGATGCCA GCACCTGCCA GGTGCCGCCC CGGGGCAAGG GCCCCAGCAG |
| 2101 | CCCTATGGTG ACCGCCACAC TGTGCCTTAA TGTCTGCCGG GGGCCCAGGC TGTGCTGTCC |
| 2161 | CTGCAGCACG CCTCCTTGCA GGGATCTGAG CCACCCTCCC CGCACAGCCC TGCACCCCGC |
| 2221 | CCCTAGGGTT GGCAGCCTCA GTTGGCCCCT GGCAGAGGAA CAAGGACACA GACATTCCCT |
| 2281 | CAGTGTGGGG GGCAGGGGAC ACAGGGAGAG GATGGTTGTC CCTGGGGAGG GCCCTCTGGC |
| 2341 | CCCAGGCAAC CTTAGCCCCT CAGAACAGGG AGTCCCAGGA CCCAGGGAGA GTGTGGGGAC |
| 2401 | AGGACAGCCT GTCTCTTGTA GCTTCCTGGG GTGGGAGGCA CAGGGGCAAA GCAATACCCC |
| 2461 | AGGGAAAGTG GGAGGTGGTG CTGGTGCTCT CTCCAGGCCC ACCATGCTGG GAGAGGCGGC |
| 2521 | CAGAGCCTGG GGCCTCCAGC CTGGGACTGC TGTGATGGGG TATCACGGTG ATGGTCCCAT |
| 2581 | TAAACTTCCA CTCTGCAAAC CTG |

GENBANK ID: S90469
DEFINITION CYTOCHROME P450 REDUCTASE [HUMAN, PLACENTA, MRNA PARTIAL, 2403
NT].
VERSION S90469.1 GI:247306
CDS 1..2031
CODO_START=1

| | |
|---|---|
| 1 | GGAGACTCCC ACGTGGACAC CAGCTCCACC GTGTCCGAGG CGGTCGCCGA AGAAGTATCT |
| 61 | CTTTTCAGCA TGACGCACAT GATTCTGTTT TCGCTCATCG TGGGTCTCCT AACCTACTGG |
| 121 | TTCCTCTTCA GAAAGAAAAA AGAAGAAGTC CCCGACTTCA CCAAAATTCA GACATTGACC |
| 181 | TCCTCTGTCA GAGAGAGGAG CTTTGTGGAA AAGATGAAGA AAACGGGGAG GAACATCATC |
| 241 | GTGTTCTACG GCTCCCAGAC GGGGACTGCA GAGGAGTTTG CCAACCGCCT GTCCAAGGAC |
| 301 | GCCCACCGCT ACGGGATGCG AGGCATGTCA GCGGACCCTG AGGAGTATGA GCTGCCGGAC |
| 361 | CTGAGCAGCC TGCCAGAGAT CGACAACGCC CTGGTGGTTT TCTGCATGGC CACGTACGCT |
| 421 | GAGGGAGACC CCACCGACAA TGCCCAGGAC TTCTACGACT GGCTGCAGGA GACAGACGTG |
| 481 | GATCTCTCTG GGGTCAAGTT CGCGGTGTTT GGTCTTGGGA ACAAGACCTA CGAGCACTTC |
| 541 | AATGCCATGG GCAAGTACGT GGACAAGCGG CTGGAGCAGC TCGGCGCCCA GCGCATCTTT |
| 601 | GAGCTGGGGT TGGGCGACGA CGATGGGAAC TTGGAGGAGG ACTTCATCAC CTGGCGAGAG |
| 661 | CAGTTCTGGC CGGCCGTGTG TGAACACTTT GGGGTGGAAG CCACTGGCGA GGAGTCCAGC |
| 721 | ATTCGCCAGT ACGAGCTTGT GGTCCACACC GACATAGATG CGGCCAAGGT GTACATGGGG |
| 781 | GAGATGGGCC GGCTGAAGAG CTACGAGAAC CAGAAGCCCC CCTTTGATGC CAAGAATCCG |
| 841 | TTCCTGGCTG CAGTCACCAC CAACCGGAAG CTGAACCAGG GAACCGAGCG CCACCTCATG |
| 901 | CACCTGGAAT TGGACATCTC GGACTCCAAA ATCAGGTATG AATCTGGGGA CCACGTGGCT |
| 961 | GTGTACCCAG CCAACGACTC TGCTCTCGTC AACCAGCTGG GCAAAATCCT GGGTGCCGAC |
| 1021 | CTGGACGTCG TCATGTCCCT GAACAACCTG GATGAGGAGT CCAACAAGAA GCACCCATTC |
| 1081 | CCGTGCCCTA CGTCCTACCG CACGGCCCTC ACCTACTACC TGGACATGAC CAACCCGCCG |
| 1141 | CGTACCAACG TGCTGTACGA GCTGGCGCAG TACGCCTCGG ACCGCTGGGA GCAGGAGCTG |
| 1201 | CTGCGCAAGA TGGCCTCCTC CTCCGGCGAG GGCAAGGAGC TGTACCTGAG CTGGGTGGTG |
| 1261 | GAGGGCGGGA GGCACATCCT GGCCATCCTG CAGGACTGCC CGTCCCTGCG GCCCCCCATC |
| 1321 | GACCACCTGT GTGAGCTGCT GCCGCGCCTG CAGGCCCGCT ACTACTCCAT CGCCTCATCC |
| 1381 | TCCAAGGTCC ACCCCAACTC TGTGCACATC TGTGCGGTGG TTGTGGAGTA CGAGACCAAG |
| 1441 | GCCGGCCGCA TCAACAAGGG CGTGGCCACC AACTGGCTGC GGGCCAAGGA GCCTGTCGGG |
| 1501 | GAGAACGGCG GCCGTGCGCT GGTGCCCATG TTCGTGCGCA AGTCCCAGTT ACGCCTGCCC |
| 1561 | TTCAAGGCCA CCACGCCTGT CATCATGGTG GGCCCCGGCA CCGGGTGGCA CCCTTTCATA |
| 1621 | GGCTTCATCC AGGAGCGGGC CTGGCTGCGA CAGCAGGGCA AGGAGGTGGG GGAGACGCTG |
| 1681 | CTGTACTACG GCTGCCGCCG CTCGGATGAG GACTACCTGT ACGGGGAGGA GCTGGCGCAG |
| 1741 | TTCCACAGGG ACGGTGCGCT CACCCAGCTC AACGTGGCCT TCTCCCGGGA GCAGTCCCAC |
| 1801 | AAGGTCTACG TCCAGCACCT GCTAAAGCAA GACCGAGAGC ACCTGTGGAA GTTGATCGAA |
| 1861 | GGCGGTGCCC ACATCTACGT CTGTGGGGAT GCACGGAACA TGGCCAGGGA TGTGCAGAAC |
| 1921 | ACCTTCTACG ACATCGTGGC TGAGCTCGGG GCCATGGAGC ACGCGCAGGC GGTGGACTAC |
| 1981 | ATCAAGAAAC TGATGACCAA GGGCCGCTAC TCCCTGGACG TGTGGAGCTA GGGGCCTGCC |
| 2041 | TGCCCCACCC ACCGCACAGA CTCCGGCCTG TAATCAGCTC TCCTGGCTCC CTCCCGTAGT |
| 2101 | CTCCTGGGTG TGTTTGGCTT GGCCTTGGCA TGGGCGCAGG CCCAGTGACA AAGACTCCTC |
| 2161 | TGGGCCTGGG GTGCATCCTC CTCAGCCCCC AGGCCAGGTG AGGTCCACCG GCCCCTGGCA |
| 2221 | GCACAGCCCA GGGCCTGCAT GGGGGCACCG GGCTCCATGC CTCTGGAGCC TCTGGCCCTC |
| 2281 | GGTGGCTGCA CAGAAGGGCT CTTTCTCTCT GCTGAGCTGG CCCAGCCCCT CCACGTGATT |
| 2341 | TCCAGTGAGT GTAAATAATT TTAAATAACC TCTGGCCCTT GGAATAAAGT TCTGTTTTCT |
| 2401 | GTA |

GENBANK ID: NM_006254.1
DEFINITION HOMO SAPIENS PROTEIN KINASE C, DELTA (PRKCD), MRNA.
VERSION NM_006254.1 GI:5453969
CDS 59..2089
/CODON_START=1

| | |
|---|---|
| 1 | TGCCGCCGCG ACCCTTGGCG CCTGCCCCTG CAACGGGAGC CCCACTGCAG GCCCCACCAT |
| 61 | GGCGCCGTTC CTGCGCATCG CCTTCAACTC CTATGAGCTG GGCTCCCTGC AGGCCGAGGA |
| 121 | CGAGGCGAAC CAGCCCTTCT GTGCCGTGAA GATGAAGGAG GCGCTCAGCA CAGAGCGTGG |
| 181 | GAAAACACTG GTGCAGAAGA AGCCGACCAT GTATCCTGAG TGGAAGTCGA CGTTCGATGC |
| 241 | CCACATCTAT GAGGGGCGCG TCATCCAGAT TGTGCTAATG CGGGCAGCAG AGGAGCCAGT |
| 301 | GTCTGAGGTG ACCGTGGGTG TGTCGGTGCT GGCCGAGCGC TGCAAGAAGA ACAATGGCAA |
| 361 | GGCTGAGTTC TGGCTGGACC TGCAGCCTCA GGCCAAGGTG TTGATGTCTG TTCAGTATTT |
| 421 | CCTGGAGGAC GTGGATTGCA AACAATCTAT GCGCAGTGAG GACGAGGCCA AGTTCCCAAC |
| 481 | GATGAACCGC CGCGGAGCCA TCAAACAGGC CAAAATCCAC TACATCAAGA ACCATGAGTT |
| 541 | TATCGCCACC TTCTTTGGGC AACCCACCTT CTGTTCTGTG TGCAAAGACT TTGTCTGGGG |
| 601 | CCTCAACAAG CAAGGCTACA AATGCAGGCA ATGTAACGCT GCCATCCACA AGAAATGCAT |
| 661 | CGACAAGATC ATCCGCAGAT CCACTGGCAC CGCCGCCAAC AGGCGGGACA CTATATTCCA |
| 721 | GAAAGAACGC TTCAACATCC ACATGCCGCA CCGCTTCAAG GTTCACAACT ACATGAGCCC |
| 781 | CACCTTCTGT GACCACTGCG GCAGCCTGCT CTGGGGACTG GTGAAGCAGG GATTAAAGTG |
| 841 | TGAAGACTGC GGCATGAATG TGCACCATAA ATGCCGGGAG AAGGTGGCCA ACCTCTGCGG |
| 901 | CATCAACCAG AAGCTTTTGG CTGAGGCCTT GAACCAAGTC ACGCAGAGAG CCTCCCGGAG |
| 961 | ATCAGACTCA GCCTCCTCAG AGCCTGTTGG GATATATCAG GGTTTCGAGA AGAAGACCGG |
| 1021 | AGTTGCTGGG GAGGACATGC AAGACAACAG TGGGACCTAC GGCAAGATCT GGGAGGGCAG |
| 1081 | CAGCAAGTGC AACATCAACA ACTTCATCTT CCACAAGGTC CTGGGCAAAG GCAGCTTCGG |
| 1141 | GAAGGTGCTG CTTGGAGAGC TGAAGGGCAG AGGAGACTAC TCTGCCATCA AGGCCCTCAA |
| 1201 | GAAGGATGTG GTCCTGATCG ACGACGACGT GGAGTGCACC ATGGTTGAGA AGCGGGTGCT |
| 1261 | GACACTTGCC GCAGAGAATC CCTTTCTCAC CCACCTCATC TGCACCTTCC AGACCAAGGA |
| 1321 | CCACCTGTTC TTTGTGATGG AGTTCCTCAA CGGGGGGGAC CTGATGTACC ACATCCAGGA |
| 1381 | CAAAGGCCGC TTTGAACTCT ACCGTGCCAC GTTTTATGCC GCTGAGATAA TGTGTGGACT |
| 1441 | GCAGTTTCTA CACAGCAAGG GCATCATTTA CAGGGACCTC AAACTGGACA ATGTGCTGTT |
| 1501 | GGACCGGGAT GGCCACATCA AGATTGCCGA CTTTGGGATG TGCAAAGAGA ACATATTCGG |
| 1561 | GGAGAGCCGG GCCAGCACCT TCTGCGGCAC CCCTGACTAT ATCGCCCCTG AGATCCTACA |
| 1621 | GGGCCTGAAG TACACATTCT CTGTGGACTG GTGGTCTTTC GGGGTCCTTC TGTACGAGAT |
| 1681 | GCTCATTGGC CAGTCCCCCT TCCATGGTGA TGATGAGGAT GAACTCTTCG AGTCCATCCG |
| 1741 | TGTGGACACG CCACATTATC CCCGCTGGAT CACCAAGGAG TCCAAGGACA TCCTGGAGAA |
| 1801 | GCTCTTTGAA AGGGAACCAA CCAAGAGGCT GGGAATGACG GGAAACATCA AAATCCACCC |
| 1861 | CTTCTTCAAG ACCATAAACT GGACTCTGCT GGAAAAGCGG AGGTTGGAGC CACCCTTCAG |
| 1921 | GCCCAAAGTG AAGTCACCCA GAGACTACAG TAACTTTGAC CAGGAGTTCC TGAACGAGAA |
| 1981 | GGCGCGCCTC TCCTACAGCG ACAAGAACCT CATCGACTCC ATGGACCAGT CTGCATTCGC |
| 2041 | TGGCTTCTCC TTTGTGAACC CCAAATTCGA GCACCTCCTG GAAGATTGAG GTTCCTGGAC |
| 2101 | AGAT |

GENBANK ID: X61971.1
DEFINITION H. SAPIENS MRNA FOR MACROPAIN SUBUNIT DELTA.
VERSION X61971.1 GI:296733
CDS <1..543
/CODON_START=1

| | |
|---|---|
| 1 | ATCGCCAATC GAGTGACTGA CAAGCTGACA CCTATTCACG ACCGCATTTT CTGCTGTCGC |
| 61 | TCAGGCTCAG CTGCTGATAC CCAGGCAGTA GCTGATGCTG TCACCTACCA GCTCGGTTTC |
| 121 | CACAGCATTG AACTGAATGA GCCTCCACTG GTCCACACAG CAGCCAGCCT CTTTAAGGAG |
| 181 | ATGTGTTACC GATACCGGGA AGACCTGATG GCGGGAATCA TCATCGCAGG CTGGGACCCT |
| 241 | CAAGAAGGAG GGCAGGTCTA CTCAGTGCCT ATGGGGGGTA TGATGGTAAG GCAGTCCTTT |
| 301 | GCCATTGGAG GCTCCGGGAG CTCCTACATC TATGGCTATG TTGATGCTAC CTACCGGGAA |
| 361 | GGCATGACCA AGGAAGAGTG TCTGCAATTC ACGGCCAATG CTCTCGCTTT GGCCATGGAG |
| 421 | CGGGATGGCT CCAGTGGAGG AGTGATCCGC CTGGCAGCCA TTGCAGAGTC AGGGGTAGAG |
| 481 | CGGCAAGTAC TTTTGGGAGA CCAGATACCC AAATTCGCCG TTGCCACTTT ACCACCCGCC |
| 541 | TGAATCCTGG GATTCTAGTA TGCAATAAGA GATGCCCTGT ACTGATGCAA AATTTAATAA |
| 601 | AGTTTGTCAC AGAGAAAAAA AAAA |

GENBANK ID: AH005909.1
GENBANK ID: XM_088424.1
DEFINITION HOMO SAPIENS RETINOID X RECEPTOR, ALPHA (RXRA), MRNA.
VERSION XM_088424.1 GI:18571706
CDS 519..1016
/CODON_START=1

| | |
|---|---|
| 1 | AAGCAGAACC TGGCCTCCCT GGCCACAGCA GCCTTACCCA CCGCTCTACG TGTCCCGGGC |
| 61 | ACTTCCCGCA GCCTTCCCGT CCCTTTCTCA TCGGCCTTGT AGTTGTACAG TGCTGTTGGT |
| 121 | TTGAAAAGGT GATGTGTGGG GAGTGCGGCT CATCACTGAG TAGAGAGGTA GAATTTCTAT |
| 181 | TTAACCAGAC CTGTAGTAGT ATTACCAATC CAGTTCAATT AAGGTGATTT TTTGTAATTA |
| 241 | TTATTATTTT GGTGGGACAA TCTTTAATTT TCTAAAGATA GCACTAACAT CAGCTCATTA |
| 301 | GCCACCTGTG CCTGTCCCCG CCTTGGCCCG GCTGGATGAA GCGGCTTCCC CGCAGGGCCC |
| 361 | CCACTTCCCA GTGGCTGCTT CCTGGGGACC CAGGGCACCC CGGCACCTTC AGGCACGCTC |
| 421 | CTCAGCTGGT CACCTCCCGG CTTTGCCGTT CAGATGGGGC TCCTGAGGCT CAGGAGTGAA |
| 481 | GATGCCACAG AGCCGGGCTC CCCTAGGCTG CGTCGGGCAT GCTTGGAAGC TGGCCTGCCA |
| 541 | GGACCTTCCA CCCTGGGGCC TGTGTCAGCC GCCGGCCCTC CGCACCCTGG AAGCACACGG |
| 601 | CCTCTGGGAA GGACAGCCCT GACCTTCGGT TTTCCGAGCA CGGTGTTTCC CAAGAATTCT |
| 661 | GGGCTTGCCG CCTGGTGGCA GTGCTGGAGA TGACCCCGAG CCCCTCCCCG TGGGGCACCC |
| 721 | AGGAGGGCCC TGCCGAAATG TGCAGCCTGT GGGTAGTCGG CTGGTGTCCC TGTCGTGGAG |
| 781 | CTGGGGTGCG TGATCTGGTG CTCGTCCACG CAGGTGTGTG GTGTAAACAT GTATGTGCTG |
| 841 | TACAGACAGA CGCGTGTGGA GAGAGCCGCA CACCAGCGCC ACCCAGGAAA GGCGGAGCGG |
| 901 | TTACCAGTGT TTTGTGTTTA TTTTTAATCA AGACGTTTCC CCTGTTTTCC TATAAATTTG |
| 961 | CTTCGTGTAA GCAAGTACAT AAGGACCCTC CTTTGGTGAA ATCCGGGTTC GAATGAATAT |
| 1021 | CTCAAGGCAG GAGATGCATC TATTTTAAGA TGCTTTGGAG CAGACAGCTT TAGCCGTTCC |
| 1081 | CAATCCTTAG CAATGCCTTA GCTGGGACGC ATAGCTAATA CTTTAGAGAG GATGACAGAT |
| 1141 | CCATAAAGAG AGTAAAGATA AGAGAAAATG TCTAAAGCAT CTGGAAAGGT AAAAAAAAAA |
| 1201 | AATCTATTTT TGTACAAATG TAATTTTATC CCTCATGTAT ACTTGGATAT GGCGGGGGGA |
| 1261 | GGGGTCGGAC TGTTTCGTTT CTGCTTCTAG AGATTGAGGT GAAAGCTTCG TCCGAGAAAC |
| 1321 | GCCAGGACAG ACGATGGCAG AGGAGAGGGC TCCTGTGACG GGGGCCAGGG TTGGGAGGAA |
| 1381 | ACCGCCGCAA TGGGGGTGTC TTCCCTCGGG GGAGGAGGGT GGGCCTGAGG CTTTCAAGGG |
| 1441 | TTTTCTTCCC TTTCGAGTAA TTTTTAAAGC CTTGCTCTGT TGTGTCCTGT TGCCGGCTCT |
| 1501 | GGCCTTCCTG TGACTGACTG TGAAGTGGCT TCTCCGTACG ATTGTCTCTG AAACATCGTG |
| 1561 | GCCGCAGGTG CCAGGGTTTG ATGGACAGTA GCATTAGAAT TGTGGAAAAG GAACACGCAA |
| 1621 | AGGGAGAAGT GTGAGAGGAG AAACAAAATA TGAGCGTTTA AAATACATCG CCATTCAG |

GENBANK ID: U41745.1
DEFINITION HUMAN PDGF ASSOCIATED PROTEIN MRNA, COMPLETE CDS.
VERSION U41745.1 GI:1136583
CDS 22..567
/CODON_START=1

| | |
|---|---|
| 1 | GAATTCCGCG GCGGCGCCTC AATGCCTAAA GGAGGAAGAA AGGGAGGCCA CAAAGGCCGG |
| 61 | GCGAGGCAGT ATACAAGCCC TGAGGAGATC GACGCGCAGC TGCAGGCTGA GAAGCAGAAG |
| 121 | GCCAGGGAAG AAGAGGAGCA AAAAGAAGGT GGAGATGGGG CTGCAGGTGA CCCCAAAAAG |
| 181 | GAGAAGAAAT CTCTAGACTC AGATGAGAGT GAGGATGAAG AAGATGACTA CCAGCAAAAG |
| 241 | CGCAAAGGCG TTGAAGGGCT CATCGACATC GAGAACCCCA ACCGGGTGGC ACAGACAACC |
| 301 | AAAAAGGTCA CACAACTGGA TCTGGACGGG CCAAAGGAGC TTTCGAGGAG AGAACGAGAA |
| 361 | GAGATTGAGA AGCAGAAGGC AAAAGAGCGT TACATGAAAA TGCACTTGGC CGGGAAGACA |
| 421 | GAGCAAGCCA AGGCTGACCT GGCCCGGCTG GCCATCATCC GGAAACAGCG GGAGGAGGCT |
| 481 | GCCCGGAAGA AGGAAGAGGA AAGGAAAGCA AAAGACGATG CCACATTGTC AGGAAAACGA |
| 541 | ATGCAGTCAC TCTCCCTGAA TAAGTAACTG CGACCCGTGG GAGGAGATGC CGGGGACCTG |
| 601 | GGCCGCGCTG CCAGGACCTC TGCTGTGTCT CGCCCACCCT GTGCCCTGGC GCCGCTGCAA |
| 661 | CAGCCCCTCA TGGCCAGGAG CCCCCCATGC CTGGGCCTCC TCTTCATCTT GGCACAGAAA |
| 721 | TTGTTTGGGG GATGGGGGGG GGACTGGGGG AGGGGTAGCT GCTATCTTTG AGACAGAAAG |
| 781 | ATGCAGGACA GCATTTCATA TGTAACCATT TGAATGTTTT TGCTGTTTTT AGAATTC |

GENBANK ID: AH002617.1
GENBANK ID: XM_034862.1
DEFINITION HOMO SAPIENS INTERFERON REGULATORY FACTOR 1 (IRF1), MRNA.
VERSION XM_034862.1 GI:14726087
CDS 197..1174
/CODON_START=1

| | |
|---|---|
| 1 | CGAGCCCCGC CGAACCGAGG CCACCCGGAG CCGTGCCCAG TCCACGCCGG CCGTGCCCGG |
| 61 | CGGCCTTAAG AACCCGGCAA CCTCTGCCTT CTTCCCTCTT CCACTCGGAG TCGCGCTCCG |
| 121 | CGCGCCCTCA CTGCAGCCCC TGCGTCGCCG GGACCCTCGC GCGCGACCGC CGAATCGCTC |
| 181 | CTGCAGCAGA GCCAACATGC CCATCACTCG GATGCGCATG AGACCCTGGC TAGAGATGCA |
| 241 | GATTAATTCC AACCAAATCC CGGGGCTCAT CTGGATTAAT AAAGAGGAGA TGATCTTCCA |
| 301 | GATCCCATGG AAGCATGCTG CCAAGCATGG CTGGGACATC AACAAGGATG CCTGTTTGTT |
| 361 | CCGGAGCTGG GCCATTCACA CAGGCCGATA CAAAGCAGGG GAAAAGGAGC CAGATCCCAA |
| 421 | GACGTGGAAG GCCAACTTTC GCTGTGCCAT GAACTCCCTG CCAGATATCG AGGAGGTGAA |
| 481 | AGACCAGAGC AGGAACAAGG GCAGCTCAGC TGTGCGAGTG TACCGGATGC TTCCACCTCT |
| 541 | CACCAAGAAC CAGAGAAAAG AAAGAAAGTC GAAGTCCAGC CGAGATGCTA AGAGCAAGGC |
| 601 | CAAGAGGAAG TCATGTGGGG ATTCCAGCCC TGATACCTTC TCTGATGGAC TCAGCAGCTC |
| 661 | CACTCTGCCT GATGACCACA GCAGCTACAC AGTTCCAGGC TACATGCAGG ACTTGGAGGT |
| 721 | GGAGCAGGCC CTGACTCCAG CACTGTCGCC ATGTGCTGTC AGCAGCACTC TCCCCGACTG |
| 781 | GCACATCCCA GTGGAAGTTG TGCCGGACAG CACCAGTGAT CTGTACAACT TCCAGGTGTC |
| 841 | ACCCATGCCC TCCACCTCTG AAGCTACAAC AGATGAGGAT GAGGAAGGGA AATTACCTGA |
| 901 | GGACATCATG AAGCTCTTGG AGCAGTCGGA GTGGCAGCCA ACAAACGTGG ATGGGAAGGG |
| 961 | GTACCTACTC AATGAACCTG GAGTCCAGCC CACCTCTGTC TATGGAGACT TTAGCTGTAA |
| 1021 | GGAGGAGCCA GAAATTGACA GCCCAGGGGG GGATATTGGG CTGAGTCTAC AGCGTGTCTT |
| 1081 | CACAGATCTG AAGAACATGG ATGCCACCTG GCTGGACAGC CTGCTGACCC CAGTCCGGTT |
| 1141 | GCCCTCCATC CAGGCCATTC CCTGTGCACC GTAGCAGGGC CCCTGGGCCC CTCTTATTCC |
| 1201 | TCTAGGCAAG CAGCACCTGG CATCATGGTG GATATCGTGC ACAGAAGCTG GACTTCTCTG |
| 1261 | GGCCCCTCAA CAGCCAAGTG TGACCCCACT CCCAAGTGGG GATGGGGCCT CCCTCCTTGG |
| 1321 | GTCATTGACC TCTCAGGGCC TGGCAGGCCA GTGTCTGCGT TTTTCTTGTC GTCTAAAGCT |
| 1381 | GGCCCTGCCT CCTGGCAAGA TGAGGTTCTG AGACCAGTGT ATCAGGTCAG GCACTTGGAC |
| 1441 | AGGAGTCAGT GTCTGGCTTT TTCCTCTGAG CCCAGCTGCC TGGAGAGGGT CTCGCTGTCA |
| 1501 | CTGGGTGGCT CCTAGGGGAA CAGACCAGTG ACCCCAGAAA AGCATAACAC CAATCCCAGG |
| 1561 | GCTGGCTCTG CACTAAGAGA AAATTGCACT AAATCAATCT CGTTCCCAAA GAACTACCCC |
| 1621 | CTTTTCAGCT CAGCCCTGGG GACTGTTCCA AAGCCAGTGA AATGTGAAGG AAAGTGCGGT |
| 1681 | CCTTCGGGGC GATGCTGGCT CAGCCTCAGA GGAGCTCTAC CCTGCTCCCT GCTTTGGCTG |
| 1741 | AGGGGCTTGG GAAAAAAACT TGGCACTTTT TCGTGTGGAT CTTGCCACAT TTCTGATCAG |
| 1801 | AGGTGTACAC TAACATTTCC CCCGAGCTCT TGGCCTTTCC ATTTATTTAT ACAGTGCCTT |
| 1861 | GCTCGGCGCC CACCACCCCC TCAAGCCCCA GCAGCCCTCA ACAGGGCCAG GGAGGGAAGT |
| 1921 | GTGAGCGCCT TGGTATGACT TAAAATTGGA AATGTCATCT AACCATTAAG TCATGTGTGA |
| 1981 | ACACATAGGA CGTGTGTATA TATGTACATT TGTCTTTTTA TAAAAAGTAA ATTGTT |

GENBANK ID: AJ310549.1
DEFINITION HOMO SAPIENS MRNA FOR CLP-36 PROTEIN.
VERSION AJ310549.1 GI:13160404
CDS 1..990
/CODON_START=1

| | |
|---|---|
| 1 | ATGACCACCC ACCAGATAGA CCTCCAGGGC CCGGGGCCGT GGGGCTTCCG CCTCGTGGGC |
| 61 | GGCAAGGACT TCGAGCAGCC TCTCGCCATT TCCCGGGTCA CTCCTGGAAG CAAGGCGGCT |
| 121 | CTAGCTAATT TATGTATTGG AGATGTAATC ACAGCCATTG ATGGGGAAAA TACTAGCAAT |
| 181 | ATGACACACT TGGAAGCTCA GAACAGAATC AAAGGCTGCA CAGACAACTT GACTCTCACT |
| 241 | GTAGCCAGAT CTGAACATAA ACTGTGGTCT CCTCTGGTGA CGGAGGAAGG GAAGCGTCAT |
| 301 | CCATACAAGA TGAATTTAGC CTCTGAACCC CAGGAGGTCC TGCACATAGG AAGCGCCCAC |
| 361 | AACCGAAGTG CCATGCCCTT TACCGCCTCG CCTGCCTCCA GCACTACTGC CAGGGTCATC |
| 421 | ACAAACCAGT ACAACAACCC AGCTGGCCTC TACTCTTCTG AAAATATCTC CAACTTCAAC |
| 481 | AATGCCCTGG AGTCAAAGAC TGCTGCCAGC GGGGTGGAGG CGAACAGCAG ACCCTTAGAC |
| 541 | CATGCTCAGC CTCCAAGCAG CCTTGTCATC GACAAAGAAT CTGAAGTTTA CAAGATGCTT |
| 601 | CAGGAGAAAC AGGAGTTGAA TGAGCCCCCG AAACAGTCCA CGTCTTTCTT GGTTTTGCAG |
| 661 | GAAATCCTGG AGTCTGAAGA AAAAGGGGAT CCCAACAAGC CCTCAGGATT CAGAAGTGTT |
| 721 | AAAGCTCCTG TCACTAAAGT GGCTGCGTCG ATTGGAAATG CTCAGAAGTT GCCTATGTGT |
| 781 | GACAAATGTG GCACTGGGAT TGTTGGTGTG TTTGTGAAGC TGCGGGACCG TCACCGCCAC |
| 841 | CCTGAGTGTT ATGTGTGCAC TGACTGTGGC ACCAACCTGA AACAGAAGGG CCATTTCTTT |
| 901 | GTGGAGGATC AAATCTACTG TGAGAAGCAT GCCCGGGAGC GAGTCACACC ACCTGAGGGT |
| 961 | TATGAAGTGG TCACTGTGTT CCCCAAGTGA GCCAGCAGAT CTGACCACTG TTCTCCAGCA |
| 1021 | GGCCTCTGCT GCAGCTTTTT CTCTCAGTGT TCTGGCCCTC TCCTCTCTTG AAAGTTCTCT |
| 1081 | GCTTACTTTG GTT |

GENBANK ID: XM_016642.3
DEFINITION HOMO SAPIENS ADENYLATE KINASE 3 (AK3), MRNA.
VERSION XM_016642.3 GI:16163712
CDS 145..816
/CODON_START=1

| | |
|---|---|
| 1 | CCTTCCCCCT GTAGGGCCGG CCGGCGAGTC CCAGTGAGAG CGGAGGGTGC CAGAGGTAGG |
| 61 | GGGCCGAGAA ACAAAGTTCC CGGGGCTCCC TCCGGGGCCG CGGTCGGGGC TGCGCGTTTG |
| 121 | ACCGCCCCCC TCCTCGCGAA GGCAATGGCT TCCAAACTCC TGCGCGCGGT CATCCTCGGG |
| 181 | CCGCCCGGCT CGGGCAAGGG CACCGTGTGC CAGAGGATCG CCCAGAACTT TGGTCTCCAG |
| 241 | CATCTCTCCA GCGGCCACTT CTTGCGGGAG AACATCAAGG CCAGCACCGA AGTTGGTGAG |
| 301 | GTGGCAAAGC AGTATATAGA GAAAAGTCTT TTGGTTCCAG ACCATGTGAT CACACGCCTA |
| 361 | ATGATGTCCG AGTTGGAGAA TAGGCGTGGC CAGCACTGGC TCCTTGATGG TTTTCCTAGG |
| 421 | ACATTAGGAC AAGCCGAGGC CCTGGACAAA ATCTGTGAAG TGGATCTAGT GATCAGTTTG |
| 481 | AATATTCCAT TTGAAACACT TAAAGATCGT CTCAGCCGCC GTTGGATTCA CCCTCCTAGC |
| 541 | GGAAGGGTAT ATAACCTGGA CTTCAATCCA CCTCATGTAC ATGGTATTGA TGACGTCACT |
| 601 | GGTGAACCAT TAGTCCAGCA GGAGGAIGAT AAACCCGAAG CAGTTGCTGC CAGGCTAAGA |
| 661 | CAGTACAAAG ACGCGGCAAA GCCAGTCATT GAATTATACA AGAGCCGAGG AGTGCTCCAC |
| 721 | CAATTTTCCG GAACGGAGAC GAACAAAATC TGGCCCTACG TTTACACACT TTTCTCAAAC |
| 781 | AAGATCACAC CTATTCAGTC CAAAGAAGCA TATTGACCCT GCCCAATGGA AGAACCAGGA |
| 841 | AGATGTGGTC ATTCATTCAA TAGTGTGTGT AGTATTGGTG CTGTGTCCAA ATTAGAAGCT |
| 901 | AGCTGAGGTA GCTTGCAGCA TCTTTTCTAG TTGAAATGGT GAACTGATAG CAAAACAAAT |
| 961 | GAGTAGAAAG AGTTCATGAA GAGGCCCTCC TCTGCCTTTC AAAAGGGTGG TCACCTACAC |
| 1021 | ATGTTTAAGG TGTCTCTGCA CATGTCTCAA GCCCATCACA AGAAAGCAAG TACAGTGTGG |
| 1081 | ATTTCAAATG GTGTGTAACT TCAGCTCCAG CTGGTTTTTG ACAGCTGTTG CTGTGGTAAT |
| 1141 | ATTTTTTACA TGTGATGGTG ATAGTCTCTG GTTCTCCCCA TCCCCACAAA GGCTGTTGAA |
| 1201 | CCACACCACC AGGAAGCCTG AGAATGAATC CTGAGGGCTC TACCGCAGGC TTTGTCCCAG |
| 1261 | GCTTTCTGGT GTGTGCCCTC CTGGTAACAG TGAAATTGAA GCTACTTACT CATAGTGGTT |
| 1321 | GTTTCTCTGG TCTTGAGTGA CTGTGTCCAC AGTTCATTTT TTTCCGGTAG GAATAACTCC |
| 1381 | TTTTCTACAT CCACACTCCA TAGAGTCTCT CCTTTTCAGA TATCCTGGGA TGAAAGAATT |
| 1441 | TGGCTTTTTT TTTTTTTTTT TTTTTGACAT CTGTTTTCAC TCTTAGGCTT TTAAACAATA |
| 1501 | GTTATTGCTC TTATCCCTCT CAGATTCTAA TAACTGAGAG TGATGGGGCT ATATTGAATC |
| 1561 | TCTGTATGCA CTGAGAACTG AGCTATGAAG AGGATCTTAT TAAACTGCTG GTCTGACTTT |
| 1621 | ATGGATTGAC ACTGTTCCTT TCTTTTATTG TG |

GENBANK ID: Z35307.1
DEFINITION H. SAPIENS MRNA FOR ENDOTHELIN-CONVERTING-ENZYME 1.
VERSION Z35307.1 GI:535181
CDS 38..2299
/CODON_START=1

| | |
|---|---|
| 1 | CGCCCCCCCG GTGTCCGCCC TGCTGTCGGC GCTGGGGATG TCGACGTACA AGCGGGCCAC |
| 61 | GCTGGACGAG GAGGACCTGG TGGACTCGCT CTCCGAGGGC GACGCATACC CCAACGGCCT |
| 121 | GCAGGTGAAC TTCCACAGCC CCCGGAGTGG CCAGAGGTGC TGGGCTGCAC GGACCCAGGT |
| 181 | GGAGAAGCGG CTGGTGGTGT TGGTGGTACT TCTGGCGGCA GGACTGGTGG CCTGCTTGGC |
| 241 | AGCACTGGGC ATCCAGTACC AGACAAGATC CCCCTCTGTG TGCCTGAGCG AAGCTTGTGT |
| 301 | CTCAGTGACC AGCTCCATCT TGAGCTCCAT GGACCGCACA GTGGACCCCT GCCATGACTT |
| 361 | CTTCAGCTAC GCCTGTGGGG GCTGGATCAA GGCCAACCCA GTCCCTGATG GCCACTCACG |
| 421 | CTGGGGGACC TTCAGCAACC TCTGGGAACA CAACCAAGCA ATCATCAAGC ACCTCCTCGA |
| 481 | AAACTCCACG GCCAGCGTGA GCGAGGCAGA GAGAAAGGCG CAAGTATACT ACCGTGCGTG |
| 541 | CATGAACGAG ACCAGGATCG AGGAGCTCAG GGCCAAACCT CTAATGGAGT TGATTGAGAG |
| 601 | GCTCGGGGGC TGGAACATCA CAGGTCCCTG GGCCAAGGAC AACTTCCAGG ACACCCTGCA |
| 661 | GGTGGTCACC GCCCACTACC GCACCTCACC CTTCTTCTCT GTCTATGTCA GTGCCGATTC |
| 721 | CAAGAACTCC AACAGCAACG TGATCCAGGT GGACCAGTCT GGCCTGGGCT TGCGCTGGAG |
| 781 | AGACTATTAC CTGAACAAAA CTGAAAACGA GAAGGTGCTG ACCGGATATC TGAACTACAT |
| 841 | GGTCCAGCTG GGGAAGCTGC TGGGCGGCGG GGACGAGGAG GCCATCCGGC GCCAGATGCA |
| 901 | GCAGATCTTG GACTTTGAGA CGGCACTGGC CAACATCACC ATCCCACAGG AGAAGCGCCG |
| 961 | TGATGAGGAG CTCATCTACC ACAAAGTGAC GGCAGCCGAG CTGCAGACCT TGGCACCCGC |
| 1021 | CATCAACTGG TTGCCTTTTC TCAACACCAT CTTCTACCCC GTGGAGATCA ATGAATCCGA |
| 1081 | GCCTATTGTG GTCTATGACA AGGAATACCT TGAGCAGATC TCCACTCTCA TCAACACCAC |
| 1141 | CGACAGATGC CTGCTCAACA ACTACATGAT CTGGAACCTG GTGCGGAAAA CAAGCTCCTT |
| 1201 | CCTTGACCAG CGCTTTCAGG ACGCCGATGA GAAGTTCATG GAAGTCATGT ACGGGACCAA |
| 1261 | GAAGACCTGT CTTCCTCGCT GGAAGTTTTG CGTGAGTGAC ACAGAAAACA ACCTGGGCTT |
| 1321 | TGCGTTGGGC CCCATGTTTG TCAAAGCAAC CTTCGCCGAG GACAGCAAGA GCATAGCCAC |
| 1381 | CGAGATCATC CTGGAGATTA AGAAGGCATT TGAGGAAAGC CTGAGCACCC TGAAGTGGAT |
| 1441 | GGATGAGGAA ACCCGAAAAT CAGCCAAGGA AAAGGCCGAT GCCATCTACA ACATGATAGG |
| 1501 | ATACCCCAAC TTCATCATGG ATCCCAAGGA GCTGGACAAA GTGTTTAATG ACTACACTGC |
| 1561 | AGTTCCAGAC CTCTACTTTG AAAATGCCAT GCGGTTTTTC AACTTCTCAT GGAGGGTCAC |
| 1621 | TGCCGATCAG CTCAGGAAAG CCCCCAACAG AGATCAGTGG AGCATGACCC CGCCCATGGT |
| 1681 | GAACGCCTAC TACTCGCCCA CCAAGAATGA GATTGTGTTT CCGGCCGGGA TCCTGCAGGC |
| 1741 | ACCATTCTAC ACACGCTCCT CACCCAAGGC CTTAAACTTT GGTGGCATAG GTGTCGTCGT |
| 1801 | GGGCCATGAG CTGACTCATG CTTTTGATGA TCAAGGACGG GAGTATGACA AGGACGGGAA |
| 1861 | CCTCCGGCCA TGGTGGAAGA ACTCATCCGT GGAGGCCTTC AAGCGTCAGA CCGAGTGCAT |
| 1921 | GGTAGAGCAG TACAGCAACT ACAGCGTGAA CGGGGAGCCG GTGAACGGGC GGCACACCCT |
| 1981 | GGGGGAGAAC ATCGCCGACA ACGGGGGTCT CAAGGCGGCC TATCGGGCTT ACCAGAACTG |
| 2041 | GGTGAAGAAG AACGGGGCTG AGCACTCGCT CCCCACCCTG GGCCTCACCA ATAACCAGCT |
| 2101 | CTTCTTCCTG GGCTTTGCAC AGGTCTGGTG CTCCGTCCGC ACACCTGAGA GCTCCCACGA |
| 2161 | AGGCCTCATC ACCGATCCCC ACAGCCCCTC TCGCTTCCGG GTCATCGGCT CCCTCTCCAA |
| 2221 | TTCCAAGGAG TTCTCAGAAC ACTTCCGCTG CCCACCTGGC TCACCCATGA ACCCGCCTCA |
| 2281 | CAAGTGCGAA GTCTGGTAAG GACGAAGCGG AGAGAGCCAA GACGGAGGAG GGGAAGGGGC |
| 2341 | TGAGGACGAG ACCCCCATCC AGCCTCCAGG GCATTGCTCA GCCCGCTTGG CCACCCGGGG |
| 2401 | CCCTGCTTCC TCACACTGGC GGGTTTTCAG CCGGAACCGA GCCCATGGTG TTGGCTCTCA |
| 2461 | ACGTGACCCG CAGTCTGATC CCCTGTGAAG AGCCGGACAT CCCAGGCACA CGTGTGCGCC |
| 2521 | ACCTTCAGCA GGCATTCGGG TGCTGGGCTG GTGGCTCATC AGGCCTGGGC CCCACACTGA |
| 2581 | CAAGCGCCAG ATACGCCACA AATACCACTG TGTCAAATGC TTTCAAGATA TATTTTTGGG |
| 2641 | GAAACTATTT TTTAAACACT GTGGAATACA CTGGAAATCT TCAGGGAAAA ACACATTTAA |
| 2701 | ACACTTTTTT TTTTAAGCCC |

GENBANK ID: J02683
DEFINITION HUMAN ADPATP CARRIER PROTEIN MRNA, COMPLETE CDS.
VERSION J02683.1 GI:179246
CDS 70..966
/CODON_START=1

| | |
|---|---|
| 1 | CCGCAGCGCC GTAGTCAAAC CGAACCCGGC CCAGTCCCGT CCTGCAGCAG TCTGCCTCCT |
| 61 | TCTTTCAACA TGACAGATGC CGCATTGTCC TTCGCCAAGG ACTTCCTGGC AGGTGGAGTG |
| 121 | GCCGCAGCCA TCTCCAAGAC GGCGGTAGCG CCCATCGAGC CGGTCAAGCT GCTGCTGCAG |
| 181 | GTGCAGCATG CCAGCAAGCA GATCACTGCA GATAAGCAAT ACAAAGGCAT TATAGACTGC |
| 241 | GTGGTCCGTA TTCCCAAGGA GCACCAAGTT CTGTCCTTCT GGCGCGGTAA CCTGGCCAAT |
| 301 | GTCATCAGAT ACTTCCCCAC CCACGCTCTT AACTTCGCCT TCAAAGATAA ATACAAGCAG |
| 361 | ATCTTCCTCG GTGGTGTGGA CAAGAGAACC CAGTTTTGGC GCTACTTTGC AGGGAATCTG |
| 421 | GCATCGGGTG CTGCCGCAGG GGCCACATGC CTGTGTTTTC TGTACCCTCT TGATTTTGCC |
| 481 | CGTACCCGTC TAGCAGCTGA TGTGGGTAAA GCTGGAGCTG AAAGGGAATT CCGAGGCCTC |
| 541 | GGTGACTGCC TGGTTAAGAT CTACAAATCT GATGGGATTA AGGGCCTGTA CCAAGGCTTT |
| 601 | AACGTGTCTG TGCAGGGTAT TATCATCTAC CGAGCCGCCT ACTTCGGTAT CTATGACACT |
| 661 | GCAAAGGGAA TGCTTCCGGA TCCCAAGAAC ACTCACATCG TCATCAGCTG GATGATCGCA |
| 721 | CAGACTGTCA CTGCTGTTGC CGGGTTGACT TCCTATCCAT TTGACACCGT TCGCCGCCGC |
| 781 | ATGATGATGC AGTCAGGGCG CAAAGGAACT GACATCATGT ACACAGGCAC GCTTGACTGC |
| 841 | TGGCGGAAGA TTGCTCGTGA TGAAGGAGGC AAAGCTTTTT TCAAGGGTGC ATGGTCCAAT |
| 901 | GTTCTCAGAG GCATGGGTGG TGCTTTTGTG CTTGTCTTGT ATGATGAAAT CAAGAAGTAC |
| 961 | ACATAAGTTA TTTCCTAGGA TTTTTCCCCC TGTGAACAGG CATGTTGTAT TCTATAACAC |
| 1021 | AATCTTGAGC ATTGTTGACA GACTCCTGGC TGTCAGTTTC TCAGTGGCAA CTACTTTACT |
| 1081 | GGTTGAAAAT GGGAAGCAAT AATATTCATC TGACCAGTTT TCTCTTAAAG CCATTTCCAT |
| 1141 | GCATGATGAT GATGGGACTC AATTGTATTT TTTATTTCAG TCACTCCTGA CTAAATAACA |
| 1201 | ATTTGGAGAA ATAAAAATAG TCTAAAAT |

GENBANK ID: M22760.1
DEFINITION HOMO SAPIENS NUCLEAR-ENCODED MITOCHONDRIAL CYTOCHROME C OXI-
DASE VA
SUBUNIT MRNA, COMPLETE CDS.
VERSION M22760.1 GI:695359
20..472
/CODON_START=1

| | |
|---|---|
| 1 | GGGCGCCGCC ATCGCCGTCA TGCTGGGCGC CGCTCTCCGC CGCTGCGCTG TGGCCGCAAC |
| 61 | CACCCGGGCC GACCCTCGAG GCCTCCTGCA CTCCGCCCGG ACCCCCGGCC CCGCCGTGGC |
| 121 | TATCCAGTCA GTTCGCTGCT ATTCCCATGG GTCACAGGAG ACAGATGAGG AGTTTGATGC |
| 181 | TCGCTGGGTA ACATACTTCA ACAAGCCAGA TATAGATGCC TGGGAATTGC GTAAAGGGAT |
| 241 | AAACACACTT GTTACCTATG ATATGGTTCC AGAGCCCAAA ATCATTGATG CTGCTTTGCG |
| 301 | GGCATGCAGA CGGTTAAATG ATTTTGCTAG TCTAGTTCGA ATCCTAGAGG TTGTTAAGGA |
| 361 | CAAAGCAGGA CCTCATAAGG AAATCTACCC CTATGTCATC CAGGAACTTA GACCAACTTT |
| 421 | AAATGAACTG GGAATCTCCA CTCCGGAGGA ACTGGGCCTT GACAAAGTGT AAACCGCATG |
| 481 | GATGGGCTTC CCCAAGGATT TATTGACATT GCTACTTGAG TGTGAACAGT TACCTGGAAA |
| 541 | TACTGATGAT AACATATTAC CTTATTTTGA ACAAGTTTCC CTTTATTGAG TACCAAGCCA |
| 601 | TGTAATGGTA ACTTGGACTT TAATAAAAGG GAAATGAGTT TGAACTG |

GENBANK ID: 5118079
DNA LINEAR
DEFINITION HUMAN, INTESTINAL FATTY ACID BINDING PROTEIN GENE, COMPLETE
CDS, AND AN ALU REPETITIVE ELEMENT.
VERSION M18079.1 GI:182351

| | |
|---|---|
| 1 | GTAATATCTT GGGCAAGCCC TAGAGCTTCT TTCCTGACCC TTAGTTAATA AGATGTTATC |
| 61 | TGGTCACATT CAGTCACAAT AATAGACTCA TTTTAGTAAT AAACATCTTA AGACTAGTAA |
| 121 | TTAAAACTCT TTACTTCACA CCAAGTTTCC TCCCCAAGCT TGGCCTGTTC CTGGCTGGCA |
| 181 | GCCTGAAGTA GGGAAAGGAG AGATATGGTG ACCTTTTCTT TGTACCTTTC TAGCTACCCT |
| 241 | CTATACCCTG ACCCCACATA CATAATTGAG CTGTGGCTTC TGACTCTACT GGGTTTGGGG |
| 301 | ATGAGAGGCA GTGAGAGTAA AATGAAGGAG TGGTTTTAAT TAATGGCACA GCTAAAACTG |
| 361 | GATTTTGTTC TCTCTGCACA TGGCAGATGT TTAAAGCTCA TTCTTTCTTT TATGCAAGTT |
| 421 | TTTACACCAT CCAGCCTCAT TTGTACCTCT TGAATTTTTG CTCAGTGGCC TATCACCATT |
| 481 | CAGGATCAAG ACAAAAATCA ATGAGCACTT ATTGTGTGTC ATGCACCCTA CAAAGTGCCA |
| 541 | GGATATTTAT CCAAACTCCT GGCAATGCTA AACACAATGC AAAAAGACAT ATTAGAAAAC |
| 601 | GAATCTTATT AACTTTAGCT TTTCAACTGT ATTTCATCAT AAAGTCTTAC TTTACAAGAT |
| 661 | AATTGCTGTT GTGAAAAAGG GAAAGGTCAT GGTCTCATTT CCCAGATGTT ATTTGATATA |
| 721 | TGCTATAAAT TATATTACCT CCAACATAGT CTGCACTTTG AACTTAGAAA AACAATCTTC |
| 781 | AGACGGCATG CATTCTAATT CTTGAAATAA GTATGCCCAC AAACTGTAGT TTAAGACAGA |
| 841 | ATAGGTATGC TTCTCATGTT TTAATTCAGT TGAATTTCAG AAGATCTCAG GAATGTACAG |
| 901 | AACGAGAATT AAGAATTAAT AAGAATAAGA ATTAATTAAT TGCTTGACAT AGAGTAGTTA |
| 961 | GGTGATTTCC TGAACTTTAA GCTTCCACAT CACACTATGA AGTTGGTTCA AGATAAGAAA |
| 1021 | TATAATAAAT TCTCGCCCAA GGACAGACCT GAATCTCTAG CTGCCTAGAG GCTGACTCAA |
| 1081 | CTGAAATCAT GGCGTTTGAC AGCACTTCCA AGGTAGACCG GAGTGAAAAC TATGACAAGT |
| 1141 | TCATGGAAAA AATGGGTAAA GACTTTATTT CTTTGTGCCT CATTCTTTGC TTTCTTACAA |
| 1201 | ACATTTTTCT TTCTAACTCC TAAATCTCTA GGAGATTACA GATAGCTTAC AGATAGCTCC |
| 1261 | TGATGTGGTA GAGACGGATC CAGAAGATGT TCAGAGGAGG GAAACCATAT TTTCCCTTCT |
| 1321 | TACATTAGGA AGAATCCACT ATCTCACTAA TGCAAGAAAA GATTCTTTGA GTGCTGTTCT |
| 1381 | CTGAAACACA CCAAAAAGAT CCAGAAATGT TTCCTTCACT CTTTAACTGA AAAATGACTT |
| 1441 | TTTTTCTTGT TTACAGTAAG AAAATGGCAG CGTGTAATGA TAACTTCCAG ATCTGAAAAT |
| 1501 | GTTAAATTCT ACGAGATGGA AAAACAAAGA CCATATAACA AAGTAATGGA AAAAGTTCTC |
| 1561 | TTAAAATTTA TAGCTCTGAA TAAGTTAGAT TTAATTCTGA TTTCTTCTAA CTTAAAAAAG |
| 1621 | TTTTGGAATA ATCTTGAGAA GCTGTGTAGT TTTCTCCAGG GCGTTTAATT TAACTGATTT |
| 1681 | ATAATTTGAT ACCAATACTC TGGCAGCCCA TATACTATAC AAGATAGGCA AACAAATTTG |
| 1741 | TGTCATTCCC CTAAAAGAAA AATCTGCATC AATTATAGCT TACAGTTTAG GAACTCTAAG |
| 1801 | TTTAAATTTA TAAAAGTTGT AGATTCTTAT AGTGATTTTG GCTTAATATT TGCTAATTTT |
| 1861 | CTCATTTTTG TGTCAGAAAG AAATGCCACA AGAAGCAAAT AGAACTATAA AGTTCAAAAT |
| 1921 | GTTAAAGCCA CTAAGAAAAA CAAAGGGGCA TTTAAGAAAA AAGAATACTG TATATGTGGA |
| 1981 | ATTAAAGATG TGCTTCCTTA TAAATATATG AATATACATT TTAATCCTTC ATTTAATATT |
| 2041 | TCTAGAATTT GATTTACTTA ACACTGAAAT GAACAGTTTG TTAATCTTAT TAAGGTTGCT |
| 2101 | CAGCTCTAAG ATTCTATAAT TCTGTACTCT ACTTAATTTT TCTCAAGTTA TGGAAAAACA |
| 2161 | ACTTTAATCA GTTCTCTTGA TCGGATTGAA CCTGAACTTC TATAGAAGCA ATCTGAATGT |
| 2221 | TCTTGTGCAA AGGCAATGCT ACCGAGTTTT CTTCCCACCC TCAAAATAAA CAAACAAAAC |
| 2281 | ATAACTTGGA AAAATAAACA CTTCCTATGG GATTTGACTT TATTTTCTCC ATTGTCTTAC |
| 2341 | CTTTTACAGG TGTTAATATA GTGAAAAGGA AGCTTGCAGC TCATGACAAT TTGAAGCTGA |
| 2401 | CAATTACACA AGAAGGAAAT AAATTCACAG TCAAAGAATC AAGCGCTTTT CGAAACATTG |
| 2461 | AAGTTGTTTT TGAACTTGGT GTCACCTTTA ATTACAACCT AGCAGACGGA ACTGAACTCA |
| 2521 | GGGTAAGAAT TTTTTTTTTT ATGAGCAATG CATTCTTGAT TTTTCTACCC AATATTAAAA |
| 2581 | TGATTTCTGC TCTATTTCAT TGGATGGTTT AATTAATGCA GGTCTCCTTC ACTAACTGAA |
| 2641 | GAAGCCAATG AAGTTTGTCT ACATTATATA TTACACAAAT TGGCAGGGTA TTTAAATATG |
| 2701 | CTTTTATTTT TATACGCATC TGTGAAGAAT CTGAATTGAA CAGTAAGAAT TAGAAAACTA |
| 2761 | TCTTTTGAAT GACTGAATAT AGACCTATTC ATAAAGAAAT TTAAAACTGT GTTTTTAAAC |
| 2821 | AGTACAGCAA AACAAGCCTT TAGAGTTAAT ATGTAACTTA ACTGTAACAT GTTGAAATAA |
| 2881 | TAAAAGAAAT GAATAGATGA ACAAATGACT GAGTTACCAA ATGGAAAGAT TTGATGTATT |
| 2941 | GTAGGTCATT GGCAGTGTAC CTTTTCATGT TTAAGATAAC ACATTTTAGG AAGTCATCAT |
| 3001 | TTTCAACAAA TTTTTTAAAA ACTTTTTTTA GCCTCAACAT TTTTCTATTT AAATTACATG |
| 3061 | TTTGTAATGA CAATTTAACT ACTGAATGTT TTATCGTAAG TTATCTCTTT CCTTAATTAG |
| 3121 | TACCACAATC ACACAAATTA AAACAAGCAC AGGTTATTAA CATCTCCGTG AAACTAATTT |
| 3181 | TAACCATGAC TATATTTCTG GACACGTAAC ATGAAAGATT CAGAAAGAAG TGCTGCTCAT |
| 3241 | CTGCCTTAAA ATTCAGCGTA TGCAAATTAT TGAAGAGAAC AAGCATAATG GTTATCAACA |
| 3301 | CATACTCTGT AGCCCAATGG CCTAGGTTCA ATCCTCACTC TGTGACTTTA GGTGAATCAC |
| 3361 | TGTGCCATTT TACAGTCTCC TCTTCTGCAA ACTAGAGATA GTAGTATCAG TTTCATAGGG |
| 3421 | TCACCATGAA GATTAAATGA AAAAGTGTGT CTACAGAACT CAGAACAGTG CCTGACATGT |
| 3481 | GTAAGACCCT AATAAATGCC ATTATTATTA TTATTATTAT TATTATTATT ATTATTATTA |
| 3541 | TGTAGGGGAC CTGGAGCCTT GAGGGAAATA AACTTATTGG AAAATTCAAA GGGACAGACA |
| 3601 | ATGGAAACGA ACTGAATACT GTCCGAGAAA TTATAGGTGA TGAACTAGTC CAGGTGAGTT |
| 3661 | GTCAAATTTA TAGCTATTTT CAAAAGGCAA AAATTACTAC AAAACAATAA TTTTTGTCAC |
| 3721 | TGCTGAGCCA GATCTTCAGT AAACTGACTA CTTCTTTTCT CATAAATCTT ACTGATTTTA |
| 3781 | AAAATATTGT ATAGCTATTT TCTGATGCCT ATTTACTAAA GACAACTTAT ATATGTCAAA |
| 3841 | TAATCAATGC CTATTTTAAC TGAAAATATA AATGACTACA AACCAACATG TGTTTTAAAA |
| 3901 | TGGCTGTATC CCATATCTGT ATAAATCTTG CTATCAAGTA CAAGAAAAAA TTGTATAAAC |
| 3961 | TCATACTCAT ATAATATATA TGAATATATA ATATAAAAAT AGTATAAACT CATATACTAT |
| 4021 | AAAACTATAA TACTACTTTT TCTTAACTTA GATGTAAACC TTAAAGATAA ATTCTTCTGT |
| 4081 | TTGTTAACAC CTTTCAGACT TATGTGTATG AAGGAGTAGA AGCCAAAAGG ATCTTTAAAA |
| 4141 | AGGATTGAGC ATTATTCTTG GCGCACAGTC CAAAATACAA ATTGGACAGA AGATCTATAT |
| 4201 | TGTACCAGAA CTGTTTATTT CACCCCATCA AGTATAAGGT TACTGATTGA TTGGTCCTTT |
| 4261 | TATAAACATT GGTATATTTC CATTCATGCC AAAGCAAAAG AAGTAAAAGC TAATTAGGAT |
| 4321 | TTAATTTGTT TTATATTCTC TAAGATATAT ATTTACTAAA AGAATTTGTG ACATTTTAAA |
| 4381 | AAACAAAAAT AAATATTGCA TCCATGTTGC TTTATATGTA GCCTTGCCTT TTAAAAGAAA |
| 4441 | AAGTATGTGA ATATGAATTG ACAGATTGTT TTCGTAGAGA GAGGGTCTTA CTCTTTCACT |
| 4501 | CAGGCTGGAA TGCAGTGGAG AGATCATAGC TCACTGTAAC CTCAAACTCC TGGACTCATG |
| 4561 | CAATCTTCCT GCCTCAGGCT TCTGAGTAGC TAGGACTATG GGTACATTCC ACAGTGCCCA |
| 4621 | GCTAATTTTT GTTTTGTTTT CTTTTTATTT TTTTTAGAGA TGGGGTCTTG CTATATTGCC |
| 4681 | CAGGCTGGTC TTGAACCCCT GGCCTCAAGC AATCCTCCTG CCTCAGCCTC TCAAGTTGTT |
| 4741 | TTTTTCTTTA CATTTGATAA ACTAAAAGCA TAGGCTGCAT ATGAGTCTTT AACATCTTGA |
| 4801 | ACTGGTTGTG AATAATTTTC TGGCACTGGT TGTAAGTAAT ATCTATTATT ATAAAAATAA |
| 4861 | TATATGCTCA ACCAGAAAAC TTAGAAATAA GAAACACAAA TGTAAAATAA GTATTTCCAT |
| 4921 | AACTCATAAT CCAGAGATAA TTGCCATTCT GATTTTGATA GATATCCTCT CAGCTCTCTT |
| 4981 | CCCTGGGGGC AGATATTTCC CAATACATAC CACTTTGAAT AGGATGATAG GAAATAAATG |
| 5041 | ATGTACTACA TTAAATTAAA TTATTGTATT ACATTTTTGT ACACATCAGT CATTCCCAGG |
| 5101 | CTTGGCTGAA AATCAGGATC ATCTGAGAAA CTTAAACAAT TTCTGCATTC TTAATCTCCA |
| 5161 | CTGTTATTCT ATTATATCAG AATCGCTAAT AGAACCAAGA ATTC |

GENBANK ID: X16277
DNA LINEAR
DEFINITION HUMAN GENE FOR ORNITHINE DECARBOXYLASE ODC (EC 4.1.1.17).
VERSION X16277.1 GI:35137
MRNA JOIN(795..1001,3858..3967,4073..4191,4475..4648,
4855..5027,5286..5420,5551..5632,5809..5892,6948..7110,
7193..7305,7399..7613,8254..8740)

| | |
|---|---|
| 1 | GGATCCGGGT CCCCTCACGC TCCTGGCTGA GTCCCTGGCT TCACAGGGGA AACTACCTCC |
| 61 | GCAGGCCAGG ACCCATCTAG TTACACGATA CCTCGATGTT ACAAAGACGA GGCTTCCAGC |
| 121 | GCGGGGGCGT GGAGGCGGCT GCCAGCCCTG CCCGCAGCGT GCTGGCGACC CCCGGGACGC |
| 181 | CCCTTCCCTC CCGCGCCTCT GCTCCCTAGC TGGTGGGAGC AGAGCGCACC GGGATCACTT |
| 241 | CCAGGTCCCT TGCACCGGAG GAATGGGCGG CAGCAGGGTC CGGAGTCGGC CCGGCGGGGC |
| 301 | CCACGTGGCC AGCACATCGG TCCTCCGCTC GCGATTTCCC TTTTCCGCTC TCGGGCACGA |
| 361 | GGTACTGAAC GCCAGGTGGA AGCACAGCTG TGCAGGTACA GGCTCTGCCG TTCAGCTGCC |
| 421 | GCGGGCCGGG GCCGGGGCCT GCGGCGTCGT GCGCGTGCGC GGACCAGTTC CAGGCGGGCG |
| 481 | AGACCGCCGC AGGGCGGGGC GGGGCGAGGC GGCCGCAGGG CGGGGAGGGC GGGGAGAGGC |
| 541 | GGCCGCAGGG CGGGGAGGGC GGGGCGCGAA GCCGGGGGCG GGGGCCACGC GTGGGGCAGG |
| 601 | CGGTGCTCGG CTCGGCTGAC GTCGGCCCGC CGGCGCCCCA CCAGCTCCGC GCGGGCCCGG |
| 661 | GTTGGCCACC GCCGGGCCCC CGCCCCTCCC CCGGCCGTGT CCCGGCCGGA ACCGATCGTG |
| 721 | GCTGGTTTGA GCTGGTGCGT CTCCATGGCG ACCCGCCGGT GCTATAAGTA GGGACCGGGG |
| 781 | TGCCGTGGGG CTTTGTCAGT CCCTCCTGTA GCCGCCGCCG CCGCCGCCCG CCGCCCCTCT |
| 841 | GGCACCAGGT CGGGCGCCAC CTCGGGCCGG CGTCTCCGGC GGGCGGGAGC CAGGCGCTGA |
| 901 | CGGGCGCGGC GGGGGCGGCC GAGCGCTCCT GCGGCTGCGA CTCAGGCTCC GGCGTCTGCG |
| 961 | CTTCCCCATG GGGCTGGCCT GCGGCGCCTG GGCGCTCTGA GGTGAGGGAC TCCCCGGCCG |
| 1021 | CGGAGGAAGG GAGGGAGCGA GGGCGGGAGC CGGGGCGGGC TGCGGGCCCC GGGCCCCGGG |
| 1081 | CACGTGTGCG GCGCGCCTCG CCGGCCTGCA GAGACACGTG GTCGCCGAGC GGGCCACGAC |
| 1141 | CTTGAGGCGC CGCTTCCTCC CGGCCCGGGG TTCTCCCGCG GCTGGATAAG GGTGATCCGG |
| 1201 | GCGCCTCGTT CTGCCCCCGT CTTCACAGCT CGGGGCTGGA GGGGCCTAGG GGAGACCCAC |
| 1261 | CCGGAGACCC TGCGGCCCCG CGCCGGCCTC TTTCCCAACC CTTCGGCGGC CGCGCGCTGG |
| 1321 | CCGGGGAGCC GTTGGGGAGG CCCTGGCGGC CGCGCAGCAG GTGCAGGGGC GCAGAGCCTG |
| 1381 | GGCTCGCCTT GGTACAGACG AGCGGCCCCG GCCTTGGCGC CTTCAGTTTC CTTCCAGTTT |
| 1441 | TTATTTTCGC TGTGTCTACA GAGCAGATGA CACCAATTTG GAAACCCGCG AGAGTGGGTA |
| 1501 | GAGCTAAGAT AGTCTTGCTG TAGTAGCTGT GATATTAGAT GCTCGGCCAT GACTTAGAGG |
| 1561 | TGTTTATTTA AGGACTGTGA ATGACTCGGT GATTTCGGAA AAGCTTGGCT TAGATGAACG |
| 1621 | GACATACACA GGGGAGACAG CCCTAAGGTT TGCAGAAAAG GCTGATTGTG CTGTTTGCGA |
| 1681 | AGTCGAAATA ATTGGTGAAA GTGTAGAAGG CAGAACCTCT CAGGAATGTC TGOGGAGGAC |
| 1741 | AAAGAATGTG TTGGCTGACT TTGTTTAAAC ATAAAATTGG GCAGACTTTA ATTGATTTGT |
| 1801 | GAAATTTTTT TCAAAGTTTG TTTGAATTAG CCCCTATCTC TTCTAACATT ATCCTCTTGT |
| 1861 | GCTAATTGAT TGACCATTTT AAATAACTTA GGTGTTACAG AAAGACCGAA AGGTGTTCTT |
| 1921 | CAGTAAAATA TATTCAAGTA AGTTACTTAA GTAACGCCTT AAAAGATACA GAAAAGCAAA |
| 1981 | AAAGTATTGG CGTATTAAAA AGAAATCAAA ACTTTCCAAG TTTAGGCCTG AACATTGCCT |
| 2041 | TAAAAATATT TAATAAGGCC TCAAATGACC CAGTCCGAGA CTGCATGAGC CTATTTATTA |
| 2101 | TTAAATTGTA AATATTCTTC ATATAAACAA AAATATATAA CCATGTCTGT AACAAAAATG |
| 2161 | GTTTTGCTAG CGTTGTTACT CTCTTCCCTT CTCCGAGGGG TGATTTAGGC AACTTCGGAG |
| 2221 | GTTGACAATG CCAAGCAGTC ACAATAGATA GAGCTTTAAA GCAAATTCTA TGCATGGGTT |
| 2281 | TGGATTTATG ACAGGCCCGT CACCCTGGGC CTGTCATAGT ACCCCATGCC AGAGCAAACT |
| 2341 | GTGTCCCCGA ACCATTGCCT GGCCTCTGTG CCCGTAGGCT GCTGGCACTG AAGTGGGTTG |
| 2401 | CACAGTGGAA AAGAAGAAAG CTCTACCTGG CAGAAATTTT TAAAGGTTAA AATAAATAAT |
| 2461 | TTTAAGAAAG CTGGTTCACA AGGTGCCACA TTTGATGAAA GCAAAATACA GTGGCTTTTA |
| 2521 | TTGTTACTAG AGTGATGTTC TTGCTTGTTT TTCTTTTTTG GTGAAGTTAG CCCCAAATTA |
| 2581 | TTCTCATAGC TAAGCAAATA CGAGAGTGAC TGTAAGGACA GTTGGCATTC CCGGAATTGC |
| 2641 | TAAACTTGGT AGGCAACGCT GGTTTAAGAA TACTGAGTTC TAGCCGGGCG TGGTGGCTCA |
| 2701 | CGCCTGTAAT CCCAACACTT TGGGAGGCTG AGGCAGGCGG ATCACCTGAG GTCGGGAGTT |
| 2761 | GGAGACCAGC CTGACTAACA TGGAGAAACG CCATCTCCAC TAAAAATATA AAATTAGCCA |
| 2821 | GGCCCCGGGT GTGGTGGCAC ATGCCGGTAA TCCCAGCTAC TCGGGAGACT GAGGCAGGAG |
| 2881 | AATCGCTTGA ACCCAGGAGG CGGAGGTTGA GGTGAGCCGA GATCATGCCA TTGCACTCCA |
| 2941 | GCCTGGGCAA CAAGAGTAAA ACTCTGTCTC AAAAAAAAAA AAAAAAAAAT ACTGAATTCT |
| 3001 | GATCAGGTAA CAGCAACTGT AATACAATGT GATAAGTTGA CTTGAAGATT ACAGTTTTTA |
| 3061 | AGAAGTATAT ACCCAGCTAA TACATGAAAA TTAACTCCTA AAATCTCAAA TGCTCCAGAC |
| 3121 | ATTTCCATGA TGCCTGTTGG TCAGTAAAAA TCATTCTAAG ACTTAGTGGA AGTAGGAAAT |
| 3181 | GTTTGTATGG CTGTGTATAA AGGCTATAAT GTAATCCCAG CACTTTGGAA GACCGAGGCG |
| 3241 | GGACGATCAC CTGGGGTCAG GAGTTTGACA CCCACCTGGA CAACGTGGTG AAATCCTGTC |
| 3301 | TCTACTAAAA ACACAAAAAT TAGCCGGGCA TGGTGCCAGG CGCCTGTAAT CCCAGCTGCT |
| 3361 | GGGGAGGCTG AGGCAGGAGA ATCGCTTGAA CCCGGGAGGC AGAGGTTGCA CTGAGCCAAG |
| 3421 | ATTGCACCGC TCCACTCCAG CCTGGGTGAC AACCTGAGAC TCTGTCTCAA AAAAAATAAA |
| 3481 | AAAGTCTATA ATGCTATTTT AAGTTTCTAA GGAACTGAAA CTGCTCTGAA ATAAATCAGA |
| 3541 | CCATTATAAG ACTTTTTTCC ATATCAGTGA GCTAAGTGCA GATAAGCTTC TGAAACTTCC |
| 3601 | ATGCTAGATT TTTTTGGTAC AAATATTTGA AATGCTTAGT GTCCTGCCTT GGAAAAACCT |
| 3661 | GGTATTTTTT GTTGTCTCCT TATACTGCCA AGGTTTATGC AATCATGTAC CTTATGCCAA |
| 3721 | GTAATAATTA GGATGACCAG GCCAGTGAGT GGTTCATATC CCGGGCATGA TTAGCTCTGC |
| 3781 | GTGTGCTCAG CCAGTGCCCC ATCTTCAACT CGATGTGTTC CTAACGTAGA CACCAAATTC |
| 3841 | CCTATTTTAT TTCTCAGATT GTCACTGCTG TTCCAAGGCC ACACGCAGAG GGATTTGCAA |
| 3901 | TTCCTGGAGA GTTGCCTTTG TGAGAAGCTG CAAATATTTC TTTCAAITCC ATCTCTTAGT |
| 3961 | TTTCCATGTA AGTATTCAGT TTACATTTAT GTTGCAGGTT AATCTTAAGA ATTGTATTGC |
| 4021 | TAAGGCTTCT AAGTGAATTT CTCCACTCTA TTTGCATTTT GTTGCATTTC AGAGGAACAT |
| 4081 | CAAGAAATCA TGAACAACTT TGGTAATGAA GAGTTTGACT GCCACTTCCT CGATCAAGGT |
| 4141 | TTTACTGCCA AGCACATTCT GGACCAGAAA ATTAATGAAG TTTCTTCTTC TGTAAGTATA |
| 4201 | TGAGGCCCAT CCTGGCAGTG CAGCTGAGAG TCCCAGGCAA GTGGAAAACT TTGGCAAGGT |
| 4261 | CTAAGGAAGA GCAATGAGGC TTACATGTCT TGTTATGGAA TGTAGAAATT AATTCACTGG |
| 4321 | TGGTAAATTA ATAGTGATAA TGCTGATACT CATATCAGTC GCTAGACTCA AAAGAGCAGG |
| 4381 | ATTCATTGTG ACTGATGGGA ATGAAGGTCG CTGGCTATTG GTGTGGTGTG TGGTGAGGCT |
| 4441 | GCTAGTGAGT CACCTGTGAC CACTCTTGTT TCAGGATGAT AAGGATGCCT TCTATGTGGC |
| 4501 | AGACCTGGGA GACATTCTAA AGAAACATCT GAGGTCGTTA AAAGCTCTCC CTCGTGTCAC |
| 4561 | CCCCTTTTAT GCACTCAAAT GTAATGATAG CAAAGCCATC CTGAAGACCC TTGCTGCTAC |
| 4621 | CGGGACAGGA TTTGACTGTG CTAGCAAGGT AAGCGATAGC AGCAGGCCTC AAAAGCGTTG |
| 4681 | TATAAAATGC GCCTGGTATT CCCCACGAGG CACATACAAG TTGTGTTTTT TGGGCAATAA |
| 4741 | ATGCTCACTA AAGGCAAATG GGCCGGGGGG GTACATGACA ACTTCCCATG CTTTTCTGTT |
| 4801 | TATTCCACGT GTTAAGCCAC ATATGGATAG CATGACACCA CTCTTCTTTT TCAGACTGAA |
| 4861 | ATACAGTTGG TGCAGAGTCT GGCGGTGCCT CCAGAGAGGA TTATCTATGC AAATCCTTGT |
| 4921 | AAACAAGTAT CTCAAATTAA GTATCCTGCT AATAATGGAG TCCAGATGAT GACTTTTCAT |
| 4981 | AGTCAAGTTG AGTTGATGAA AGTTGCCAGA GCACATCCCA AAGCAAAGTG AGTTATTCCC |
| 5041 | CCATCTGAGG GCAAGATCGG GAGCATAAGA TATCTGGATT CTTATCAAAC AAACTTAAAT |
| 5101 | TTCTGATTAT TATATTTCTA TACTTTAGTA GAAAGTAGTT GAAACCCCCA TTGAGTCATG |
| 5161 | AAGCCTGGGA CTCAAACTAC AGAATATATC AGCGACAGTA TTTAGAACAG GATTGTTTTT |
| 5221 | ATTTTAATTG TGGCTATAAG TGAACATCTA TCATGAGACA TTTGCTGCAC TTTCCTTGCT |
| 5281 | TGTAGGTTGG TTTTGCGGAT TGCCACTGAT GATTCCAAAG CAGTCTGTCG TCTCAGTGTG |
| 5341 | AAATTCGGTG CCACCCTCAG AACCAGCAGG CTCCTTTTGG AACGGGCGAA AGAGCTAAAT |
| 5401 | ATCGATGTTG TTGGTGTCAG GTGAGATTTT GGTGGGATAG CTAGAGGTCA AGACATTGAA |
| 5461 | CAGTTTGAGT TTTACAGGCT TTCTCCTAGT GTTTGCTATT A |
| TTTAAGAA ATACTAAGAC | |
| 5521 | ACAGTGTCTC GTCTCTTTAT TTTACCCCAC CTTCCATCTA GGAACCGGCT GTACCGATCC |
| 5581 | TGAGACCTTC CTGCAGGCAA TCTCTGATGC CCGCTGTGTT TTTGACATGC GGGTGAGTAT |
| 5641 | ACGTGACCCT GTTAGGCAAG GGCGGGACAC AACTGACAAT AACTAGTCTT AATTCTAGAG |
| 5701 | TTAACTTTTT A2GGCAGTTG GTTCTCTATT ACATGGGTTT CAGCCTATCT GCTGCATACA |
| 5761 | TTTTTGTTAT TACCTGTGGA TCTGGCTGAC TTATTTTCTT CATTCTAGGC TCAGGTTGGT |
| 5821 | TTCAGCATGT ATCTCCTTGA TATTGGCGGT GGCTTTCCTG CATCTGAGGA TGTGAAACTT |
| 5881 | AAATTTCAAG AGGTAATTTA GAACAAAACT GTAATACTCA GTAGCCGTTC TAATAAATTC |
| 5941 | CTTTTTGGAA TATTTCAAAA TTTAAGTGTC TTAACTAATA CCACAATGGG CTGAAGTGTC |
| 6001 | TTGGTGTGAT ATTTTGAGTG ATTTCTTTGT GCTGTCTGAC ATTACACTTG ATACCATTTG |
| 6061 | GTTTTCTAAA GTGTGAATCA GCTTTCCCAG AAGTCTTGGA TAAATGGTTA CATTGGAAAT |
| 6121 | CATGGCTCAC ACCTGTAATC CACCACTTGG GCAGGCCAAG GTGCTAGGAT CACTTGAGCC |
| 6181 | CAGGAGTTTG AGACCAGCCT GGGCAACACA GTGAGACCCG ATCTCTACAA AAAAAATTTT |
| 6241 | AAAATTAGCC TGGTGTGGTG GCCGGCACCT GTAATCCCAG CTACTTGGAA GGCTGAGGTG |
| 6301 | CGAGGATCAC TTGAGCCCAG GAGGTTGAGG CTGCAGTGAG CCATGATCAT GCCACTGCAC |
| 6361 | TCAGCCTGGG CTACAGAGTG AGACCCTGTC TCAAAAAAAA AAAAGAAAAA GCATGTTGCT |
| 6421 | GTGGGCTTCC TAGAGAATAT GCTGACTGTA GCACATCATC ACCCCAAATG TGCTTTCCTA |
| 6481 | GACCTATGCT TCCTCTCCTT AAAATACTTG AAATCTTTAG TCACTTACCA AGTTAACCCA |
| 6541 | TTATATTGGT GCTTGAATTT ATAAAATACA TCCACATGGT TTGTTAAAAT CATGACGTAG |
| 6601 | GCAGAATAGG ATTTTTATCC TGTTGGCATC TATTTGTTAA AATGTTTTCA CATCTTGATC |
| 6661 | CCTTCCTAGG TAGTAGTTAG TTGCGTACTG TTCTTTGATA AAAATCATAC CCATAACATC |
| 6721 | CTAAAGGACA TAGGGTGCCT GGAGGGGAAT GAAAACGAGC CACGTGGGAT ATGTAGCCTG |
| 6781 | GTTTTCAGGG AGATGTTGAT GTTTTTTTGC TTTTGTTACT TTAATGATAA ACCTGTCTGT |
| 6841 | TGATGCCTGG TCTCATGATG TCATGTCACA AGGCCCTGTG ATGTTACTCC CCCATGTGAA |
| 6901 | TTTCCCACAA TGAAGGCTGC TCTTTCTTTT CTGTTTCACT CTCTTAGATC ACCGGCGTAA |
| 6961 | TCAACCCAGC GTTGGACAAA TACTTTCCGT CAGACTCTGG AGTGAGAATC ATAGCTGAGC |
| 7021 | CCGGCAGATA CTATGTTGCA TCAGCTTTCA CGCTTGCAGT TAATATCATT GCCAAGAAAA |
| 7081 | TTGTATTAAA GGAACAGACG GCCTCTGATG GTATGTATAA AGGACGAATC ACTTCATGTA |
| 7141 | TAACTGAAAG CTGATGCAAA AAGTCATTAA GATTGTTGAT CTGCCTTTCT AGACGAACAT |
| 7201 | GACTCGAGTG AGCAGACCTT TATGTATTAT GTGAATGATG GCGTCTATGG ATCATTTAAT |
| 7261 | TGCATACTCT ATGACCACGC ACATGTAAAG CCCCTTCTGC AAAAGGTAAT TTCTGAGCAT |
| 7321 | ACTGTATAAA ACAATTAAGA GGACTGGTCA CAACACCTGT AATTAACTAC TACTTCCTCT |
| 7381 | CTCCGTCTCT TTATATAGAG ACCTAAACCA GATGAGAAGT ATTATTCATC CAGCATATGG |
| 7441 | GGACCAACAT GTGATGCCCT CGATCGGATT GTTGAGCGCT CTGACCTGCC TCAAATGCAT |
| 7501 | GTGGGTGATT GGATGCTCTE TGAAAACATG GGCGCTTACA CTGTTGCTGC TGCCTCTACG |
| 7561 | TTCAATGGCT TGCAGAGGGC GACCATGTAC TATGTGATGT CAGGGCCTGC GTGGTAAGTA |
| 7621 | AGCCATGCAT GTTGATGGTG CTGCCAAGAA TAGGCACCTT CTTGGATGTC TGCTTCTTGT |
| 7681 | CTAGACGAAT AAGAAATTGT CTTGCCTAAG ATTAAATATA TATGGATATT TTTCCTAAGA |
| 7741 | AAAGTTTTAG AAAAGACTGA TGAGTGTATT TCTATGTAAT TGCAATATAT TTAAGTTCAT |
| 7801 | GCCATGTGTC TTGTGGTTTC CTTATTACCA AAACGGTGAC TGAAGAAACG CTTGCTTTAG |
| 7861 | AAATACATTG AATTGGCCAG GTGTGCTGGC TCACACCTGA AATCACAACA CATTGGGAGG |
| 7921 | CCAAGGCAGA AGGATCACTT GAGCCCAGGA GTTCGAGCCT GGGCAACATA GTGAGACCCT |
| 7981 | GTCTCTACAA AAAATTAAAA AATTAGTTGG CCATGGTAGT GGGCGCCTGT AGTCCCAGCT |
| 8041 | GCTTGGCTAA GGTGAGAGGT TTGCTTGAGC CTGGGAGGTT GAGGCTGCGG TGAGCTATGA |
| 8101 | TAGCACCATT GTATTCCAGC CTGAGTAACA GAGAAAGACC CTGTCTCAGA AAAAAAAAAA |
| 8161 | ATACATTGAA TTGTTTCCTG ATGGGAAGTA AATACTCTCA TGCCCAGTTA GGAGTGAGTC |
| 8221 | AGGGTTTTTA ATATGCCACT TTTTCTTTCT CAGGCAACTC ATGCAGCAAT TCCAGAACCC |
| 8281 | CGACTTCCCA CCCGAAGTAG AGGAACAGGA TGCCAGCACC CTGCCTGTGT CTTGTGCCTG |
| 8341 | GGAGAGTGGG ATGAAACGCC ACAGAGCAGC CTGTGCTTCG GCTAGTATTA ATGTGTAGAT |
| 8401 | AGCACTCTGG TAGCTGTTAA CTGCAAGTTT AGCTTGAATT AAGGGATTTG GGGGGACCAT |
| 8461 | GTAACTTAAT TACTGCTAGT TTTGAAATGT CTTTGTAAGA GTAGGGTCGC CATGATGCAG |
| 8521 | CCATATGGAA GACTAGGATA TGGGTCACAC TTATCTGTGT TCCTATGGAA ACTATTTGAA |
| 8581 | TATTTGTTTT ATATGGATTT TTATTCACTC TTCAGACACG CTACTCAAGA GTGCCCCTCA |
| 8641 | GCTGCTGAAC AAGCATTTGT AGCTTGTACA ATGGCAGAAT GGGCCAAAAG CTTAGTGTTG |
| 8701 | TGACCTGTTT TTAAAATAAA GTATCTTGAA ATAATTAGGC ATTGGGACGT TTTTATGGTG |
| 8761 | TGTTCATTCC AGACAGTTCA CGAATCCCGT ATAGCTCGCT CTGATTCTCA GAGAACAATG |
| 8821 | AGTGGGTCCA CCCACACACA GGTAGGAGGA CAGGTGAGAC GGAAGCCCCA TCCTCCCATG |
| 8881 | TGGACGGTGC ACATCTGCTC AGCCCACCCC ACATGTCCAG AGTTGGCTGC AAACTCCTTG |
| 8941 | TCCAGAGCCT CTGGTGGTGG GACCTACTTA AGTCTGACGG ACCTGTCCTG TCCAGGCCAG |
| 9001 | TGCCCAGGGA AGGTGTGGGA GGCCCTTTGA GCCTGGCCTG CAG |

GENBANK ID: X57522
DEFINITION H. SAPIENS RING4 CDNA.
VERSION X57522.1 GI:36060
CDS 31..2457
/CODON_START=1

| | |
|---|---|
| 1 | GCGGCCGCTT TCGATTTCGC TTTCCCCTAA ATGGCTGAGC TTCTCGCCAG CGCAGGATCA |
| 61 | GCCTGTTCCT GGGACTTTCC GAGAGCCCCG CCCTCGTTCC CTCCCCCAGC CGCCAGTAGG |
| 121 | GGAGGACTCG GCGGTACCCG GAGCTTCAGG CCCCACCGGG GCGCGGAGAG TCCCAGACCC |
| 181 | GGCCGGGACC GGGACGGCGT CCGAGTGCCA ATGGCTAGCT CTAGGTGTCC CGCTCCCCGC |
| 241 | GGGTGCCGCT GCCTCCCCGG AGCTTCTCTC GCATGGCTGG GGACAGTACT GCTACTTCTC |
| 301 | GCCGACTGGG TGCTGCTCCG GACCGCGCTG CCCCGCATAT TCTCCCTGCT GGTGCCCACC |
| 361 | GCGCTGCCAC TGCTCCGGGT CTGGGCGGTG GGCCTGAGCC GCTGGGCCGT GCTCTGGCTG |
| 421 | GGGGCCTGCG GGGTCCTCAG GGCAACGGTT GGCTCCAAGA GCGAAAACGC AGGTGCCCAG |
| 481 | GGCTGGCTGG CTGCTTTGAA GCCATTAGCT GCGGCACTGG GCTTGGCCCT GCCGGGACTT |
| 541 | GCCTTGTTCC GAGAGCTGAT CTCATGGGGA GCCCCCGGGT CCGCGGATAG CACCAGGCTA |
| 601 | CTGCACTGGG GAAGTCACCC TACCGCCTTC GTTGTCAGTT ATGCAGCGGC ACTGCCCGCA |
| 661 | GCAGCCCTGT GGCACAAACT CGGGAGCCTC TGGGTGCCCG GCGGTCAGGG CGGCTCTGGA |
| 721 | AACCCTGTGC GTCGGCTTCT AGGCTGCCTG GGCTCGGAGA CGCGCCGCCT CTCGCTGTTC |
| 781 | CTGGTCCTGG TGGTCCTCTC CTCTCTTGGG GAGATGGCCA TTCCATTCTT TACGGGCCGC |
| 841 | CTCACTGACT GGATTCTACA AGATGGCTCA GCCGATACCT TCACTCGAAA CTTAACTCTC |
| 901 | ATGTCCATTC TCACCATAGC CAGTGCAGTG CTGGAGTTCG TGGGTGACGG GATCTATAAC |
| 961 | AACACCATGG GCCACGTGCA CAGCCACTTG CAGGGAGAGG TGTTTGGGGC TGTCCTGCGC |
| 1021 | CAGGAGACGG AGTTTTTCCA ACAGAACCAG ACAGGTAACA TCATGTCTCG GGTAACAGAG |
| 1081 | GACACGTCCA CCCTGAGTGA TTCTCTGAGT GAGAATCTGA GCTTATTTCT GTGGTACCTG |
| 1141 | GTGCGAGGCC TATGTCTCTT GGGGATCATG CTCTGGGGAT CAGTGTCCCT CACCATGGTC |
| 1201 | ACCCTGATCA CCCTGCCTCT GCTTTTCCTT CTGCCCAAGA AGGTGGGAAA ATGGTACCAG |
| 1261 | TTGCTGGAAG TGCAGGTGCG GGAATCTCTG GCAAAGTCCA GCCAGGTGGC CATTGAGGCT |
| 1321 | CTGTCGGCCA TGCCTACAGT TCGAAGCTTT GCCAACGAGG AGGGCGAAGC CCAGAAGTTT |
| 1381 | AGGGAAAAGC TGCAAGAAAT AAAGACACTC AACCAGAAGG AGGCTGTGGC CTATGCAGTC |
| 1441 | AACTCCTGGA CCACTAGTAT TTCAGGTATG CTGCTGAAAG TGGGAATCCT CTACATTGGT |
| 1501 | GGGCAGCTGG TGACCAGTGG GGCTGTAAGC ACTCCGAACC TTCTCACATT TCTTCTCTAC |
| 1561 | CAGATCCAGT TCACCCAGGC TGTGGAGGTA CTGCTCTCCA TCTACCCCAG AGTACAGAAG |
| 1621 | GCTGTGGGCT CCTCAGAGAA AATATTTGAG TACCTGGACC GCACCCCTCG CTGCCCACCC |
| 1681 | AGTGGTCTGT TGACTCCCTT ACACTTGGAG GGCCTTGTCC AGTTCCAAGA TGTCTCCTTT |
| 1741 | GCCTACCCAA ACCGCCCAGA TGTCTTAGTG CTACAGGGGC TCACATTCAC CCTACGCCCT |
| 1801 | GGCGAGGTGA CGGCGCTGGT GCGACCCAAT GGGTCTGGGA AGAGCACAGT CGCTGCCCTG |
| 1861 | CTGCACAATC TGTACCAGCC CACCGGGCGA CAGCTGCTGT TGGATGGGAA GCCCCTTCCC |
| 1921 | CAATATGAGC ACCGCTACCT GCACAGGCAG GTGGCTGCAG TGGGACAAGA GCCACAGGTA |
| 1981 | TTTGGAAGAA GTCTTCAAGA AAATATTGCC TATGGCCTGA CCCAGAAGCC AACTATGGAG |
| 2041 | GAAATCACAG CTGCTGCAGT AAACTCTGGG GCCCATACTT TCATCTCTGG ACTCCCTCAG |
| 2101 | GGCTATGACA CAGAGGTAGA CGAGGCTGGG AGCCAGCTGT CAGGGGCTCA GCGACAGGCA |
| 2161 | GTGGCGTTGG CCCGAGCATT GATCCGGAAA CCCTGTGTAC TTATCCTGGA TGATGCCACC |
| 2221 | AGTGCCCTGG ATGCAAACAG CCAGTTACAG GTGGAGCAGC TCCTGTACGA AAGCCCTGAG |
| 2281 | CGGTACTCCC GCTCAGTGCT TCTCATCACC CAGCACCTCA GCCTGGTGGA GCAGGCTGAC |
| 2341 | CACATCCTCT TTCTGGAAGG AGGCGCTATC CGGGAGCGGG GAACCCACCA GCAGCTCATG |
| 2401 | GAGAAAAAGG GGTGCTACTG GGCCATGGTG CAGGCTCCTG CAGATGCTCC AGAATCAAAG |
| 2461 | CCTTCTCAGA CCTGCGCACT CCATCTCCCT CCCTTTTCTT CTCTCTGTGG TGGAGAACCA |
| 2521 | CAGCTGCAGA GTAGCAGCTC CCTCCAGGAT GAGTTACTTG AAATTTGCCT TGAGTGTGTT |
| 2581 | ACCTCCTTTC CAAGCTCCTC GTGATAATGC AGACTTCCTG GAGTACAAAC ACAGGATTTG |
| 2641 | TAATTCCTAC TGTAACGGAG TTTAGAGCCA GGGCTGATGC TTTGGTGTGG CCAGCACTCT |
| 2701 | GAAACTGAGA AATGTTCAGA ATGTACGGAA AGATGATCAC CTATTTTCAA CATAACTGAA |
| 2761 | GGCATATGCT GGCCCATAAA CACCCTGTAG GTTCTTGATA TTTATAATAA AATTGGTGTT |
| 2821 | TTGT |

GENBANK ID: D00017.1
DEFINITION HOMO SAPIENS MRNA FOR LIPOCORTIN II, COMPLETE CDS.
VERSION D00017.1 GI:219909
CDS 50..1069
/CODON_START=1

| | |
|---|---|
| 1 | CATTTGGGGA CGCTCTCAGC TCTCGGCGCA CGGCCCAGCT TCCTTCAAAA TGTCTACTGT |
| 61 | TCACGAAATC CTGTGCAAGC TCAGCTTGGA GGGTGATCAC TCTACACCCC CAAGTGCATA |
| 121 | TGCGTCTGTC AAAGCCTATA CTAACTTTGA TGCTGCGCGG GATGCTTTGA ACATTGAAAC |
| 181 | AGCCATCAAC ACCAAACGTG TGGATGAGGT CACCATTGTC AACATTTTGA CCAACCGCAG |
| 241 | CAATGCACAG AGACAGGATA TTGCCTTCGC CTACCAGAGA AGGACCAAAA AGGAACTTGC |
| 301 | ATCAGCACTG AAGTCAGCCT TATCTGGCCA CCTGGAGACG GTGATTTTGG GCCTATTGAA |
| 361 | GACACCTGCT CAGTATGACG CTTCTGAGCT AAAAGCTTCC ATGAAGGGGC TGGGAACCGA |
| 421 | CGAGGACTCT CTCATTGAGA TCATCTGCTC CAGAACCAAC CAGGAGCTGC AGGAAATTAA |
| 481 | CAGAGTCTAC AAGGAAATGT ACAAGACTGA TCTGGAGAAG GACATTATTT CGGACACATC |
| 541 | TGGTGACTTC CGCAAGCTGA TGGTTGCCCT GGCAAAGGGT AGAAGAGCAG AGCATGGCTC |
| 601 | TGTCATTGAT TATGAACTGA TTGACCAAGA TGCTCGGGAT CTCTATGACG CTGGAGTGAA |
| 661 | GAGGAAAGGA ACTGATGTTC CCAAGTGGAT CAGCATCATG ACCGAGGGGA GCGTGCCCCA |
| 721 | CCTCCAGAAA GTATTTGATA GGTACAAGAG TTACAGCCCT TATGACATGT TGGAAAGCAT |
| 781 | CAGGAAAGAG GTTAAAGGAG ACCTGGAAAA TGCTTTCCTG AACCTGGTTC AGTGCATTCA |
| 841 | GAACAAGCCC CTGTATTTTG CTGATCGGCT GTATGACTCC ATGAAGGGCA AGGGGACGCG |
| 901 | AGATAAGGTC CTGATCAGAA TCATGGTCTC CCGCAGTGAA GTGGACATGT TGAAAATTAG |
| 961 | GTCTGAATTC AAGAGAPAGT ACGGCAAGTC CCTGTACTAT TATATGCAGG AAGACACTAA |
| 1021 | GGGCGACTAC CAGAAAGCGC TGCTGTACCT GTGTGGTGGA GATGACTGAA GCCCGACACG |
| 1081 | GCCTGAGCGT CCAGAAATGG TGCTCACCAT GCTTCCAGCT AACAGGTCTA GAAAACCAGC |
| 1141 | TTGCGAATAA CAGTCCCCGT GGCCATCCCT GTGAGGGTGA CGTTAGCATT ACCCCCAACC |
| 1201 | TCATTTTAGT TGCCTAAGCA TTGCCTGGCC TTCCTGTCTA GTCTCTCCTG TAAGCCAAAG |
| 1261 | AAATGAACAT TCCAAGGAGT TGGAAGTGAA GTCTATGATG TGAAACACTT TGCCTCCTGT |
| 1321 | GTACTGTGTC ATAAACAGAT GAATAAACTG AATTTGTACT TT |

GENBANK ID: M10277.1
DNA LINEAR
DEFINITION HUMAN CYTOPLASMIC BETA-ACTIN GENE, COMPLETE CDS.
VERSION M10277.1 GI:177967

| | |
|---|---|
| 1 | GCCCAGCACC CCAAGGCGGC CAACGCCAAA ACTCTCCCTC CTCCTCTTCC TCAATCTCGC |
| 61 | TCTCGCTCTT TTTTTTTTTC GCAAAAGGAG GGGAGAGGGG GTAAAAAAAT GCTGCACTGT |
| 121 | GCGGCGAAGC CGGTGAGTGA GCGGCGCGGG GCCAATCAGC GTGCGCCGTT CCGAAAGTTG |
| 181 | CCTTTTATGG CTCGAGCGGC CGCGGCGGCG CCCTATAAAA CCCAGCGGCG CGACGCGCCA |
| 241 | CCACCGCCGA GACCGCGTCC GCCCGCGAGC ACAGAGCCTC GCCTTTGCCG ATCCGCCGCC |
| 301 | CGTCCACACC CGCCGCCAGG TAAGCCCGGC CAGCCGACCG GGGCATGCGG CCGCGGCCCT |
| 361 | TCGCCCGTGC AGAGCCGCCG TCTGGGCCGC AGCGGGGGGC GCATGGGGCG GAACCGGACC |
| 421 | GCCGTGGGGG GCGCGGGAGA AGCCCCTGGG CCTCCGGAGA TGGGGGACAC CCCACGCCAG |
| 481 | TTCGCAGGCG CGAGCCCGCG CTCGGGCGGG CGCGCTCCGG GCGTGCCGCT CTCGGGGCGG |
| 541 | GGGCAACCGG CGGCGTCTTT GTCTGAGCCG GCCTCTTGCC AATGGGGATC GCACCCTGGG |
| 601 | CGCGGCGTAG CCCCCGTCAG GCCCGGTGGG GGCTCGGGCG CCATGCGCGT GCGCGCTGGT |
| 661 | CCTTTGGGCG CTAACTGCCT GCGCGCTGGG AATTGCCGCT AATTGCCCGT GCGCGCTGCG |
| 721 | ACTCAATGCC GCTAATCGCG CGTCCGTTCT GGGGCCCGGG CCCTTGCGCC ACTTCCTGCC |
| 781 | CGAGCCGCTG GCGCCCGAGG GTGTGGCCGC TGCGTGCGCG CCCGCGACCC GGTCGCTGTT |
| 841 | TCAACCGGGC GGAGGCCGGG CTCCCGCCCC GTTCGGAGGG GGTTGGGGCC TGGCTTCCTG |
| 901 | CCGCCCGCCG CGGGCACGCC TCCGACCAGT GTTTGCCTTT TATGGTAATA ACGCGGCCGG |
| 961 | CCCCCCTTCC TTTGTCCCCA ATCTCGCCGC GCGCCGGCGC CCCCTGGCGG CCTAAGGACT |
| 1021 | CGGCGCGCCG GAAGTGGCCA GGGCGGGGGC GACTTCGGCT CACAGCGCGC CCGGCTATTC |
| 1081 | TCGCAGCTCA CCATGGATGA TGATATCGCC GCGCTCGTCG TCGACAACGG CTCCGGCATG |
| 1141 | TGCAAGGCCG GCTTCCCGGG CCACGATGCC CCCCGGGCCG TCTTCCCCTC CATCGTGGGG |
| 1201 | CGCCCCAGGC ACCAGGTAGG GGAGCTGGCT GGGTGGGGCA GCCCCGGGAG CCGGCGGGAG |
| 1261 | GCAAGGGCGC TTTCTCTGCA CAGGAGGCTG CCGGTTTCCG GGGTGGGCTG CGCCCGTGCT |
| 1321 | CAGGGCTTCT TGTCCTTTCC TTCCCAGGGC GTGATGGTGG GCATGGGTCA GAAGGATTCC |
| 1381 | TATGTGGGCG ACGAGGCCCA GAGCAAGAGA GGCATCCTCA CCCTGAAGTA CCCCATCGAG |
| 1441 | CACGCCATGG TCACCAACTG GGACGACATG GAGAAAATCT GGCACCACAC CTTCTACAAT |
| 1501 | GAGCTGCGTG TGGCTCCCGA GGAGCACCCC GTGCTGCTGA CCGAGGCCCC CCTGAACCCC |
| 1561 | AAGGCCAACC GCGAGAAGAT GACCCAGGTG AGTGGCCCGC TACCTCTTCT GGTGGCCGCC |
| 1621 | TCCCTCCTTC CTGGCCTCCC GGAGCTGCGC CCTTTCTCAC TGGTTCTCTC TTCTGCCGTT |
| 1681 | TTCCGTAGGA CTCTCTTCTC TGACCTGAGT CTCCTTTGGA ACTCTGCAGG TTCTATTTGC |
| 1741 | TTTTTCCCAG ATGAGCTCTT TTTCTGGTGT TTGTCTCTCT GACTAGGTGT CTGAGACAGT |
| 1801 | GTTGTGGGTG TAGGTACTAA CACTGGCTCG TGTGACAAGG CCATGAGGCT GGTGTAAAGC |
| 1861 | GGCCTTGGAG TGTGTATTAA GTAGGCGCAC AGTAGGTCTG AACAGACTCC CCATCCCAAG |
| 1921 | ACCCCAGCAC ACTTAGCCGT GTTCTTTGCA CTTTCTGCAT GTCCCCCGTC TGGCCTGGCT |
| 1981 | GTCCCCAGTG GCTTCCCCAG TGTGACATGG TGCATCTCTG CCTTACAGAT CATGTTTGAG |
| 2041 | ACCTTCAACA CCCCAGCCAT GTACGTTGCT ATCCAGGCTG TGCTATCCCT GTACGCCTCT |
| 2101 | GGCCGTACCA CTGGCATCGT GATGGACTCC GGTGACGGGG TCACCCACAC TGTGCCCATC |
| 2161 | TACGAGGGGT ATGCCCTCCC CCATGCCATC CTGCGTCTGG ACCTGGCTGG CCGGGACCTG |
| 2221 | ACTGACTACC TCATGAAGAT CCTCACCGAG CGCGGCTACA GCTTCACCAC CACGGCCGAG |
| 2281 | CGGGAAATCG TGCGTGACAT TAAGGAGAAG CTGTGCTACG TCGCCCTGGA CTTCGAGCAA |
| 2341 | GAGATGGCCA CGGCTGCTTC CAGCTCCTCC CTGGAGAAGA GCTACGAGCT GCCTGACGGC |
| 2401 | CAGGTCATCA CCATTGGCAA TGAGCGGTTC CGCTGCCCTG AGGCACTCTT CCAGCCTTCC |
| 2461 | TTCCTGGGTG AGTGGAGACT GTCTCCCGGC TCTGCCTGAC ATGAGGGTTA CCCCTCGGGG |
| 2521 | CTGTGCTGTG GAAGCTAAGT CCTGCCCTCA TTTCCCTCTC AGGCATGGAG TCCTGTGGCA |
| 2581 | TCCACGAAAC TACCTTCAAC TCCATCATGA AGTGTGACGT GGACATCCGC AAAGACCTGT |
| 2641 | ACGCCAACAC AGTGCTGTCT GGCGGCACCA CCATGTACCC TGGCATTGCC GACAGGATGC |
| 2701 | AGAAGGAGAT CACTGCCCTG GCACCCAGCA CAATGAAGAT CAAGGTGGGT GTCTTTCCTG |
| 2761 | CCTGAGCTGA CCTGGGCAGG TCAGCTGTGG GGTCCTGTGG TGTGTGGGGA GCTGTCACAT |
| 2821 | CCAGGGTCCT CACTGCCTGT CCCCTTCCCT CCTCAGATCA TTGCTCCTCC TGAGCGCAAG |
| 2881 | TACTCCGTGT GGATCGGCGG CTCCATCCTG GCCTCGCTGT CCACCTTCCA GCAGATGTGG |
| 2941 | ATCAGCAAGC AGGAGTATGA CGAGTCCGGC CCCTCCATCG TCCACCGCAA ATGCTTCTAG |
| 3001 | GCGGACTATG ACTTAGTTGC GTTACACCCT TTCTTGACAA AACCTAACTT GCGCAGAAAA |
| 3061 | CAAGATGAGA TTGGCATGGC TTTATTTGTT TTTTTTGTTT TGTTTTGGTT TTTTTTTTTT |
| 3121 | TTTTGGCTTG ACTCAGGATT TAAAAACTGG AACGGTGAAG GTGACAGCAG TCGGTTGGAG |
| 3181 | CGAGCATCCC CCAAAGTTCA CAATGTGGCC GAGGACTTTG ATTGCATTGT TGTTTTTTTA |
| 3241 | ATAGTCATTC CAAATATGAG ATGCATTGTT ACAGGAAGTC CCTTGCCATC CTAAAAGCCA |
| 3301 | CCCCACTTCT CTCTAAGGAG AATGGCCCAG TCCTCTCCCA AGTCCACACA GGGGAGGTGA |
| 3361 | TAGCATTGCT TTCGTGTAAA TTATGTAATG CAAAATTTTT TTAATCTTCG CCTTAATACT |
| 3421 | TTTTTATTTT GTTTTATTTT GAATGATGAG CCTTCGTGCC CCCCCTTCCC CCTTTTTGTC |
| 3481 | CCCCAACTTG AGATGTATGA AGGCTTTTGG TCTCCCTGGG AGTGGGTGGA GGCAGCCAGG |
| 3541 | GCTTACCTGT ACACTGACTT GAGACCAGTT GAATAAAAGT GCACACCTTA AAAATGAGGC |
| 3601 | CAAGTGTGAC TTTGTGGTGT GGCTGGGTTG GGGGCAGCAG AGGGTG |

GENBANK ID: XM_042788.1
DEFINITION HOMO SAPIENS ALDOLASE B, FRUCTOSE-BISPHOSPHATE (ALDOB), MRNA.
VERSION XM_042788.1 GI:14738248
CDS 126..1220
/CODON_START=1

| | |
|---|---|
| 1 | AAAAACATGA TGAGAAGTCT ATAAAAATTG TGTGCTACCA AAGATCTGTC TTATTTGGCA |
| 61 | GCTGCTGCCT CACCCACAGC TTTTGATATC TAGGAGGACT CTTCTCTCCC AAACTACCTG |
| 121 | TCACCATGGC CCACCGATTT CCAGCCCTCA CCCAGGAGCA GAAGAAGGAG CTCTCAGAAA |
| 181 | TTGCCCAGAG CATTGTTGCC AATGGAAAGG GGATCCTGGC TGCAGATGAA TCTGTAGGTA |
| 241 | CCATGGGGAA CCGCCTGCAG AGGATCAAGG TGGAAAACAC TGAAGAGAAC CGCCGGCAGT |
| 301 | TCCGAGAAAT CCTCTTCTCT GTGGACACTT CCATCAACCA GAGCATCGGG GGTGTCATCC |
| 361 | TTTTCCACGA GACCCTCTAC CAGAAAGACA GCCAGGGAAA GCTGTTCACA AACATCCTCA |
| 421 | AGGAAAAGGG GATCGTGGAG CGAATCAAGT TAGACCAAGG AGGTGCTCCT CTTGCAGGAA |
| 481 | CAAACAAAGA AACCACCATT CAAGGGCTTG ATGGCCTCTC AGAGCGCTGT GCTCACTACA |
| 541 | AGAAAGATGG TGTTGACTTT GGGAAGTGGC GTGCTGTGCT GAGGATTGCC GACCAGTGTC |
| 601 | CATCCAGCCT CGCTATCCAG CAAAACGCCA ACGCCCTGGC TCGCTACGCC AGCATCTGTC |
| 661 | AGCAGAATGG ACTGGTACCT ATTGTTGAAC CAGAGGTAAT TCCTGATGGA GACCATGACC |
| 721 | TGGAACACTG CCAGTATGTT ACTGAGAAGG TCCTGGCTGC TGTCTACAAG GCCCTGAATG |
| 781 | ACCATCATGT TTACCTGGAG GGCACCCTGC TAAAGCCCAA CATGGTGACT GCTGGACATG |
| 841 | CCTGCACCAA GAAGTATACT CCAGAACAAG TAGGTATGGC CACCGTAACA GCTCTCCACC |
| 901 | GTACTGTTCC TGCAGCTGTT CCTGGCATCT GCTTTTTGTC TGGTGGCATG AGTGAAGAGG |
| 961 | ATGCCACTCT CAACCTCAAT GCTATCAACC TTTGCCCTCT ACCAAAGCCC TGGAAACTAA |
| 1021 | GTTTCTCTTA TGGACGGGCC CTGCAGGCCA GTGCACTGGC TGCCTGGGGT GGCAAGGCTG |
| 1081 | CAAACAAGGA GGCAACCCAG GAGGCTTTTA TGAAGCGGGC CATGGCTAAC TGCCAGGCGG |
| 1141 | CCAAAGGACA GTATGTTCAC ACGGGTTCTT CTGGCGCTGC TTCCACCCAG TCCCTCTTCA |
| 1201 | CAGGCTGCTA TACCTACTAC GGTCCAATGC CCGCCAGCCT ACCTCCAGTG CTTCTACTAG |
| 1261 | GAGGGCTGAA AGGGAGCAAC TTTTCCTCCA ATCCTGGAAA TTCGACACAA TTAGATTTGA |
| 1321 | ACTGCTGGAA ATACAACACA TGTTAAATCT TAAGTACAAG GCGGAAAAAA TAAATCAGTT |
| 1381 | ATTGAAACAT AAAAATGAAT ACCAAGGACC TGATCAAATT TCACACAGCA GTTTCCTTGC |
| 1441 | AACACTTTCA GCTCCCCATG CTCCAGAATA CCCACCCAAG AAAATAATAG GCTTTAAAAC |
| 1501 | AATATCGGCT CCTCATCCAA AGAACAACTG CTGATTGAAA CACCTCATTA GCTGAGTGTA |
| 1561 | GAGAAGTGCA TCTTATGAAA CAGTCTTAGC AGTGGTAGGT TGGGAAGGAG ATAGCTCCAA |
| 1621 | CCAAAAAAGA AATAAATATT CTATAAACCT TC |

GENBANK ID: NM_005317.2
DEFINITION HOMO SAPIENS GRANZYME N (LYMPHOCYTE MET-ASE 1) (GZMM), MRNA.
VERSION NM_005317.2 GI:7108347
CDS 46..819
/CODON_START=1

| | |
|---|---|
| 1 | GGCTCGGGGC CGGGGCCAGC ACCCACACTG GGTCTCCACA GCGGCATGGA GGCCTGCGTG |
| 61 | TCTTCACTGC TGGTGCTGGC CCTGGGGGCC CTGTCAGTAG GCAGCTCCTT TGGGACCCAG |
| 121 | ATCATCGGGG GCCGGGAGGT GATCCCCCAC TCGCGCCCGT ACATGGCCTC ACTGCAGAGA |
| 181 | AATGGCTCCC ACCTGTGCGG GGGTGTCCTG GTGCACCCAA AGTGGGTGCT GACGGCTGCC |
| 241 | CACTGCCTGG CCCAGCGGAT GGCCCAGCTG AGGCTGGTGC TGGGGCTCCA CACGCTGGAC |
| 301 | AGCCCCGGTC TCACCTTCCA CATCAAGGCA GCCATCCAGC ACCGTGGCTA CAAGCCCGTC |
| 361 | CCTGCCCTGG AGAACGACCT CGCGCTGCTT CAGCTGGACG GGAAAGTGAA GCCCAGCCGG |
| 421 | ACCATCCGGC CGTTGGCCCT GCCCAGTAAG CGCCAGGTGG TGGCAGCAGG GACTCGGTGC |
| 481 | AGGATGCCGG GCTGGGGGCT GACCGACCAG GGCGGGCGCC TGTCCCGGGT GCTGCGGGAG |
| 541 | CTGGACCTCC AAGTGCTGGA CACCCGCATG TGTAACAACA GCCGCTTCTG GAACGGCAGC |
| 601 | CTCTCCCCCA GCATGGTCTG CCTGGCGGGC GACTCCAAGG ACCAGGCTCC CTGCAAGGGT |
| 661 | GACTCGGGCG GGCCCCTGGT GTGTGGCAAA GGCCGGGTGT TGGCCGGAGT CCTGTCCTTC |
| 721 | AGCTCCAGGG TCTGCACTGA CATCTTCAAG CCTCCCGTGG CCACCGCTGT GGCGCCTTAC |
| 781 | GTGTCCTGGA TCAGGAAGGT CACCGGCCGA TCGGCCTGAT GCCCTGGGGT GATGGGGACC |
| 841 | CCCTCGCTGT CTCCACAGGA CCCTTCCCCT CCAGGGGTGC AGTGGGGTGG GTGAGGACGG |
| 901 | GTGGGAGGGA CAGGGAGGGA CCAATAAATC ATAATGAAGA AACGCTC |

GENBANK ID: XM_003595.2
DEFINITION HOMO SAPIENS GLOTAMYL AMINOPEPTIDASE (ANINOPEPTIDASE A)
(ENPEP) ,MRNA.
VERSION XM_003595.2 GI:13647140
CDS 1401..2957
/CODON_START=1

| | |
|---|---|
| 1 | TCCAATTTAA AAAGGAAGTC TGCTGACGTT AGTTAGTTAA ATTTAACATC TTTTTATGTG |
| 61 | TAACACTTGA CTTTGGAAGC AAAAATGAAC TTTGCGGAGA GAGAGGGCTC TAAGAGATAC |
| 121 | TGCATTCAAA CGAAACATGT GGCCATTCTC TGTGCGGTGG TGGTGGGTGT AGGATTAATA |
| 181 | GTGGGACTTG CCGTGGGCTT GACCAGATGG TGTGACTCCA GCGGGGACGG CGGGCCGGGC |
| 241 | ACTGCGCCAG CTCCTTCCCA CCTGCCTTCT TCCACGGCCA GCCCCTCAGG TCCTCCTGCC |
| 301 | CAGGACCAGG ACATCTGCCC GGCCAGTGAG GATGAGAGCG GACAGTGGAA AAACTTTCGA |
| 361 | CTGCCGGACT TCGTCAACCC AGTCCACTAC GACCTGCACG TGAAGCCCCT GTTGGAGGAG |
| 421 | GACACGTACA CGGGCACCGT GAGGATGTGC ATCAACCTGA GCGCTGCCAC CCGGTACCTG |
| 481 | TGGCTGCACC TCCGGGAGAC CAGGATGACG CGGCTCCCGG AGCTGAAGAG GCCCTCTGGG |
| 541 | GACCAGGTGC AAGTCCGGAG GTGTTTCGAG TACAAAAAGC AGGAGTACGT GGTGGTCGAG |
| 601 | GCGGAGGAAG AGCTTACCCC CAGCAGTGGA GATGGCCTGT ATCTCCTGAC CATGGAGTTC |
| 661 | GCCGGCTGGC TGAACGGCTC CCTCGAGGGA TTTTATAGAA CCACCTACAC GGAGAACGGA |
| 721 | CAAGTCAACA GCATAGTGGC CACCGATCAT GAACCAACAG ATCCCAGGAA ATCTTTTCCT |
| 781 | TGTTTTGATG AGCCCAACAA AAAGGCAACT TATACAATAT CTATCACCCA TCCCAAAGAA |
| 841 | TACGGACCAC TTTCAAATAT GCCAGTGGCG AAACAACAGT CAGTGGATGA TAAATGGACT |
| 901 | CGAACAACTT TTGAGAAGTC TGTCCCCATG AGCACGTACC TGGTGTGCTT TGCTGTACAT |
| 961 | CAATTTGACT CTGTAAAGAG AATATCAAAT AGTGGAAAAC CTCTTACAAT TTATGTCCAC |
| 1021 | CCAGAGCAAA AGCACACAGC CGAATATGCT GCAAACATAA CTAAAAGTGT GTTTGATTAT |
| 1081 | TTTGAAGAAT ACTTTGCTAT GAATTATTCT CTTCCTAAAT TAGATAAAAT CGCTATTCCA |
| 1141 | GATTTTGGCA CTGGTGCCAT GGAGAACTGG CGACTCATCA CGTACAGAGA AACGAACCTG |
| 1201 | CTTTATGACC CTAAGGAATC AGCCTCATCA AACCAACAGA GGGTGGCCAC TGTGGTTGCC |
| 1261 | CATGAACTTG TGCATCAGTG GTTTCGAAAT ATTGTGACCA TGGACTGGTG GGAAGACTTG |
| 1321 | TGCCTAAATG AAGGATTTGC TTCTTTCTTT GAGTTTCTGG GAGTAAACCA TGCAGAAACA |
| 1381 | GACTGGCAAA TGGTGACCAA ATGTTACTTG AAGATGTATT ACCTGTTCAA GAGGATGATT |
| 1441 | CTTTGATGTC TTCCCATCCA ATTATTGTGA CTGTGACAAC CCCTGATGAA ATAACATCTC |
| 1501 | TTTTTGATGG AATATCCTAT AGCAAGGGAT CTTCTATTTT GAGAATGCTT GAAGACTGGA |
| 1561 | TAAAACCAGA GAATTTTCAA AAAGGATGTC AGATGTACTT GGAAAAATAC CAATTCAAGA |
| 1621 | ATGCAAAAAC TTCTGATTTT TGGGCAGCAC TGGAAGAGGC AAGTAGGCTA CCAGTGAAAG |
| 1681 | AAGTAATGGA CACCTGCACC AGACAGATGG CTTATCCTGT GCTTAACGTG AACGGTGTCA |
| 1741 | AGAACATCAC ACAGAAACGC TTTTTGTTGC ACCCAAGAGC TAACCCTTCT CAGCCCCCTT |
| 1801 | CAGATCTTCG TTATACATGG AATATCCCAG TTAAATGGAC TGAAGATAAT ATAACAAGCA |
| 1861 | GTCTGTTATT TAATAGGTCA GAAAAAGAAG GAATGACTTT GAACTCCTCT AATCCTAGTG |
| 1921 | GAAATGCTTT TCTCAAAATA AACCCACATC ATATTGGGTT TTATCGTGTA AATTATGAAG |
| 1981 | TAGCAACTTG GGACTCGATA CCTACAGCGC TCTCCTTGAA CCACAAGACA TTTTCTTCAG |
| 2041 | CAGATCGTCC AAGTCTTATT GATGATGCTT TTGCCTTGGC AAGAGCTCAA CTTCTACATT |
| 2101 | ATAAGGTGGC TTTGAACTTG ACCAAGTATC TCAAAACGGA AGAGAATTTT TTACCATGGC |
| 2161 | AGAGAGTAAT TTCAGCTGTA ACCTACATCA TTAGCATGTT TGAAGATGAT AAAGAGCTAT |
| 2221 | ATCCTATGAT TGAGGAATAC TTCCAAGGTC AAGTGAAGCC TATTGCAGAT TCTCTGGGAT |
| 2281 | GGAATGATGC TGGAGACCAT GTCACAAAGT TACTCCGTTC CTCCGTGTTA GGGTTTGCGT |
| 2341 | GCAAGATGGG AGACAGAGAA GCCTTGAACA ATGCTTCCTC GTTATTTGAG CAGTGGCTAA |
| 2401 | ATGCCACTGT AAGCCTTCCC GTAAATCTCA GGCTTCTGGT GTATCGGTAT GGGATGCAGA |
| 2461 | ACTCTGGCAA TGAGATTTCA TGGAACTACA CTCTTGAGCA ATACCAGAAA ACTTCATTAG |
| 2521 | CTCAAGAAAA AGAAAAACTG CTGTATGGAT TAGCATCAGT GAAGAACGTT ACTCTTTTGT |
| 2581 | CAAGGTATTT GGATTTGCTC AAGGACACGA ACCTTATTAA AACTCAGGAT GTGTTTACAG |
| 2641 | TCATTCGATA TATCTCATAT AACAGCTATG GGAAGAACAT GGCCTGGAAT TGGATACAAC |
| 2701 | TCAACTGGCA CTATCTAGTC AACAGATATA CACTCAATAA CAGAAACGTT GGCCGAATTG |
| 2761 | TCACAATAGC AGAGCCATTC AACACTGAAC TGCAACTGTG GCAGATGGAG AGCTTTTTTG |
| 2821 | CAAAATATCC ACAAGCTGGA GCAGGAGAAA AACCTAGGGA ACAAGTGCTG GAAACAGTGA |
| 2881 | AAAACAATAT AGAGTGGCTA AAACAACATA GAAACACCAT CAGAGAATGG TTTTTTAATT |
| 2941 | TACTTGAGAG TGGTTAATGT ATTCAAATGT TAGAGTTTAA TTTTGTGAAT CTATTGTTTC |

GENBANK ID: U34683.1
DEFINITION HUMAN GLUTATHIONE SYNTHETASE MRNA, COMPLETE CDS.
VERSION 034683.1 GI:1236349
CDS 41..1465
/CODON_START=1

| | |
|---|---|
| 1 | GGGAGAACCG TTCGCGGAGG AAAGGCGAAC TAGTGTTGGG ATGGCCACCA ACTGGGGGAG |
| 61 | CCTCTTGCAG GATAAACAGC AGCTAGAGGA GCTGGCACGG CAGGCCGTGG ACCGGGCCCT |
| 121 | GGCTGAGGGA GTATTGCTGA GGACCTCACA GGAGCCCACT TCCTCGGAGG TGGTGAGCTA |
| 181 | TGCCCCATTC ACGCTCTTCC CCTCACTGGT CCCCAGTGCC CTGCTGGAGC AAGCCTATGC |
| 241 | TGTGCAGATG GACTTCAACC TGCTAGTGGA TGCTGTCAGC CAGAACGCTG CCTTCCTGGA |
| 301 | GCAAACTCTT TCCAGCACCA TCAAACAGGA TGACTTTACC GCTCGTCTCT TTGACATCCA |
| 361 | CAAGCAAGTC CTAAAAGAGG GCATTGCCCA GACTGTGTTC CTGGGCCTGA ATCGCTCAGA |
| 421 | CTACATGTTC CAGCGCAGCG CAGATGGCTC CCCAGCCCTG AAACAGATCG AAATCAACAC |
| 481 | CATCTCTGCC AGCTTTGGGG GCCTGGCCTC CCGGACCCCA GCTGTGCACC GACATGTTCT |
| 541 | CAGTGTCCTG AGTAAGACCA AAGAAGCTGG CAAGATCCTC TCTAATAATC CCAGCAAGGG |
| 601 | ACTGGCCCTG GGAATTGCCA AAGCCTGGGA GCTCTACGGC TCACCCAATG CTCTGGTGCT |
| 661 | ACTGATTGCT CAAGAGAAGG AAAGAAACAT ATTTGACCAG CGTGCCATAG AGAATGAGCT |
| 721 | ACTGGCCAGG AACATCCATG TGATCCGACG AACATTTGAA GATATCTCTG AAAAGGGGTC |
| 781 | TCTGGACCAA GACCGAAGGC TGTTTGTGGA TGGCCAGGAA ATTGCTGTGG TTTACTTCCG |
| 841 | GGATGGCTAC ATGCCTCGTC AGTACAGTCT ACAGAATTGG GAAGCACGTC TACTGCTGGA |
| 901 | GAGGTCACAT GCTGCCAAGT GCCCAGACAT TGCCACCCAG CTGGCTGGGA CTAAGAAGGT |
| 961 | GCAGCAGGAG CTAAGCAGGC CGGGCATGCT GGAGATGTTG CTCCCTGGCC AGCCTGAGGC |
| 1021 | TGTGGCCCGC CTCCGCGCCA CCTTTGCTGG CCTCTACTCA CTGGATGTGG GTGAAGAAGG |
| 1081 | GGACCAGGCC ATCGCCGAGG CCCTTGCTGC CCCTAGCCGG TTTGTGCTAA AGCCCCAGAG |
| 1141 | AGAGGGTGGA GGTAACAACC TATATGGGGA GGAAATGGTA CAGGCCCTGA AACAGCTGAA |
| 1201 | GCACACTGAG GAGAGGGCCT CCTACATCCT CATGGAGAAG ATCGAACCTG AGCCTTTTGA |
| 1261 | GAATTGCCTG CTACGGCCTG GCAGCCCTGC CCGAGTGGTC CAGTGCATTT CAGAGCTGGG |
| 1321 | CATCTTTGGG GTCTATGTCA GGCAGGAAAA GACACTCGTG ATGAACAAGC ACGTGGGGCA |
| 1381 | TCTACTTCGA ACCAAAGCCA TCGAGCATGC AGATGCTGGT GTGGCACCGG GAGTGGCAGT |
| 1441 | CCTGGACAAC CCATACCCTG TGTGAGGGCA CAACCAGGCC ACGGGACCTT CTATCCTCTG |
| 1501 | TATTTGTCAT TCCTCTCCTA GCCCTCCTGA GGGGTATCCT CCTAAAGACC TCCAAAGTTT |
| 1561 | TTATGGAAGG GTAAATACTG CTACCTTCCC CCAGCTTTCC ATCTGACGAC CAGAAAACTT |
| 1621 | GTGTCTCCCT TACATGACAT CTAGACGCCC CCAAATCCTT CAGATGTGGG TATAGCTCAG |
| 1681 | GCTAAGCTGC TCTGAGGTAA AGGTCCATGA ACCCTGCCCC ACTCCTGTCA GCCCCTCATC |
| 1741 | AGCCTTTTCA CCAGGTTCCA GTCCCTGACT TGGGATAGCA CTCAGTGCTA CCACCAGGGC |
| 1801 | GAGTGGAGGG GCATAGCCTT TCCCTAATTC TGCCTTAAAT AAAACTGCAT TGCTGT |

GENBANK ID: AF035429.1
DNA LINEAR
DEFINITION HOMO SAPIENS CYTOCHEOME OXIDASE SUBUNIT I (001) AND SUBUNIT II
(CCII) PSEUDOGENES, COMPLETE SEQUENCE.
VERSION AF035429.1 GI:2665724

| | |
|---|---|
| 1 | AATATGAAAA TCACCTCGGA GCTGGTAAAA AGAGGCTTAA CCCCTGTCTT TAGATTTACA |
| 61 | GTCCAATGCT TCACTCAGCC ATTTTACCTC ACCCCCACTG ATGTTCGCCG ACCGTTGACT |
| 121 | ATTCTCTACA AACCACAAAG ACATTGGAAC ACTATACCTA TTATTCGGCG CATGAGCTGG |
| 181 | AGTCCTACGC ACAGCTCTAA GCCTCCTTAT TCGAGCCGAA CTGGGCCAGC CAGGCAACCT |
| 241 | TCTAGGTAAC GACCACATCT ACAACGTTAT CGTCACAGCC CATGCATTTG TAATAATCTT |
| 301 | CTTCATAGTA ATACCCATCA TAATCGGAGG CTTTGGCAAC TGACTAGTTC CCCTAATAAT |
| 361 | CGGTCCCCCC GATATGGCGT TTCCCCGCAT AAACAACATA AGCTTCTCAC TCTTACCCCC |
| 421 | CTCTCTCCTA CTCCTGCTTG CATCTCCTAT AGTGGAGGCC GGCGCAGGAA CAGGTTGAAC |
| 481 | AGTCTACCCT CCCTTGGCAG GGAACTACTC CCACCCTGGA GGCTGCGTAG ACCTAACCAT |
| 541 | CTTCTCCTTA CACCTAGCAG GTATCTCCTC TATCTTAGGA GCCATCAATT TCATCACAAC |
| 601 | AATTATTAAT ATAAAACCCC CTGCCATAAC CCAATACCAA ACGCCCCTTT TCGTCTGATC |
| 661 | CGTCCTAATC ACAGCAGTCT TACTTCTCCT ATCTCTCCCA GTCCTAGCCG CTGGCATCAC |
| 721 | TATACTACTA ACAGACCGTA ACCTCAACAC CACCTTCTTC GACCCAGCCG GAGGAGGAGA |
| 781 | CCCCATTCTA TACCAACACC TATTCTGATT TTTCGGTCAC CCTGAAGTTT ATATTGTGAT |
| 841 | CCTACCAGGC TTCGGAATAA TCTCCCATAT TGTAACTTAC TACTCCGGAA AAAAAGAACC |
| 801 | ATTTGGATAC ATAGGTATGG TCTGAGCTAT GATATCAATT GGCTTCCTAG GGTTTATCGT |
| 961 | GTGAGCACAC CATATATTTA CAGTAGGAAT AGACGTAGAC ACACGAGCAT ATTTCACCTC |
| 1021 | CGCTACCATA ATCATCGCTA TCCCCACCGG CGTCAAAGTA TTTAGCTGAC TCGCCACACT |
| 1081 | CCACGGAAGC AATATGAAAT GATCTGCTGC AGTGCTCTGA GCCCTAGGAT TTATTTTTCT |
| 1141 | TTTGACCGTA GGTGGGCTGA CTGGCATTGT ATTAGCAAAC TCATCACTAG ACATCGTACT |
| 1201 | ACACGACACG TACTACGTTG TAGCCCACTT CCACTATGTC CTATCAATAG GAGCTGTATT |
| 1261 | TGCCATCATA GGAGGCTTCA TTCACTGATT TCCCCTATTC TCAGGCTACA CCCTAGACCA |
| 1321 | AACCTACGCC AAAATCCATT TCGCTATCAT ATTCATCGGC GTAAATCTAA CTTTCTTCCC |
| 1381 | ACAACACTTT CTCGGCCTAT CCGGAATGCC CCGACGTTAC TCGGACTATC CCGATGCATA |
| 1441 | CACCACATGA AATATCCTAT CATCTGTAGG CTCATTCATT TCTCTAACAG CAGTAATATT |
| 1501 | AATAATTTTC ATAATTTGAG AAGCCTTCGC TTCGAAGCGA AAAGTCCTAA TAGTAGAAGA |
| 1561 | ACCCTCCATA AACCTGGAGT GACTATATGG ATGCCCCCCA CGCTACCACA CATTCGAAGA |
| 1621 | ACCCGTATAC ATAAAATCTA GACAAAAAAG GAAGGAATCG AACCCCCCAA AGCTGGTTTC |
| 1681 | AAGCCAACCC CATGGCCTCC ATGACTTTTT CAAAAAGATA TTAGAAAAAC CATTTCATAA |
| 1741 | CTTTGTCAAA GTTAAATTAT AGGCTAAATC CTATATATCT TAATGGCACA TGCAGCGCAA |
| 1801 | GTAGGTCTAC AAAACGCTAC TTCCCCTATC ATAGAAGAGC TTATCATCTT TCATGATCAC |
| 1861 | GCCCTCATAA TCATTTTCCT TATCTGCTTC CTAGTCCTGT ACGCCCTTTT CCTAACACTC |
| 1921 | ACAACAAAAC TAACTAATAC TAACATCTCA GACGCTCAGG AAATAGAAAC CGTCTGAACT |
| 1981 | ATCCTGCCCG CCATCATCCT AGTCCTTATC GGGCTGCCAT CCCTACGCAT CCTTTACATA |
| 2041 | ACAGACGAGG TCAACGATCC CTCCTTTACC ATCAAATCAA TTGGCCATCA ATGGTACTGA |
| 2101 | ACCTACGAAT ACACGGACTA CGGCGGACTA ATCTTCAACT CCTACATACT TCCCCCATTA |
| 2161 | TTCCTAGAAC CAGGCGACCT GCGACTCCTT GACGTTGACA ATCGAGTAGT ACTCCCGGTT |
| 2221 | GAAGCCCCCA TTCGTATAAT AATTACATCA CAAGACGTCT TACACTCATG AGCTGTCCCC |
| 2281 | ACATTAGGCT TAAAAACAGA TGCAATTCCC GGACGTCTAA ACCAAACCAC TTTCACTGCT |
| 2341 | ACACGACCAG GGGTATACTA CGGCCAATGC TCTGAAATCT GTGGAGCAAA CCAGTTTTAT |
| 2401 | GCCCATCGTC CTAGAATTAA TTCCCCTAAA AATCTTTGAA ATAGGGCCTG TATTTACCCT |
| 2461 | ATAGCACCCC CTCTACCCCC TCTAGAGCCC ACTGTAAAGC TAACTTAGCA TTAACCTTTT |
| 2521 | AAGTTAAAGA TTAAGAGAAC CAACACCTCT TTACAGTGAA ATGCCCCAAC TAAATACTA |

GENBANK ID: NM_024409.1
DEFINITION HOMO SAPIENS NATRIURETIC PEPTIDE PRECURSOR C (NPPC), MRNA.
VERSION NM_024409.1 GI:13249345
CDS 1..381
/CODON_START=1

| | |
|---|---|
| 1 | ATGCATCTCT CCCAGCTGCT GGCCTGCGCC CTGCTGCTCA CGCTGCTCTC CCTCCGGCCC |
| 61 | TCCGAAGCCA AGCCCGGGGC GCCGCCGAAG GTCCCGCGAA CCCCGCCGGC AGAGGAGCTG |
| 121 | GCCGAGCCGC AGGCTGCGGG CGGCGGTCAG AAGAAGGGCG ACAAGGCTCC CGGGGGCGGG |
| 181 | GGCGCCAATC TCAAGGGCGA CCGGTCGCGA CTGCTCCGGG ACCTGCGCGT GGACACCAAG |
| 241 | TCGCGGGCAG CGTGGGCTCG CCTTCTGCAA GACCACCGCA ACGCGCGCAA ATACAARGGA |
| 301 | GCCAACAAGA AGGGCTTGTC CAAGGGCTGC TTCGGCCTCA AGCTGGACCG AATCGGCTCC |
| 361 | ATGAGCGGCC TGGGATGTTA G |

GENBANK ID: M37763.1
DNA LINEAR
DEFINITION HUMAN NEUROTROFHIN-3 (NT-3) GENE, COMPLETE CDS.
VERSION M37763.1 GI:189300
CDS 76..849
/CODON_START=1
GENE 76..849
MAT PEPTIDE 130..846

| | |
|---|---|
| 1 | TAACACAGAC TCAGCTGCCA GAGCCTGCTC TTAACACCTG TGTTTCCTTT TCAGATCTTA |
| 61 | CAGGTGAACA AGGTGATGTC CATCTTGTTT TATGTGATAT TTCTCGCTTA TCTCCGTGGC |
| 121 | ATCCAAGGTA ACAACATGGA TCAAAGGAGT TTGCCAGAAG ACTCGCTCAA TTCCCTCATT |
| 181 | ATTAAGCTGA TGCAGGCAGA TATTTTGAAA AACAAGCTCT CCAAGCAGAT GGTGGACGTT |
| 241 | AAGGAAAATT ACCAGAGCAC CCTGCCCAAA GCTGAGGCTC CCCGAGAGCC GGAGCGGGGA |
| 301 | GGGCCCGCCA AGTCAGCATT CCAGCCGGTG ATTGCAATGG ACACCGAACT GCTGCGACAA |
| 361 | CAGAGACGCT ACAACTCACC GCGGGTCCTG CTGAGCGACA GCACCCCCTT GGAGCCCCCG |
| 421 | CCCTTGTATC TCATGGAGGA TTACGTGGGC AGCCCCGTGG TGGCGAACAG ARCATCACGG |
| 481 | CGGAAACGGT ACGCGGAGCA TAAGAGTCAC CGAGGGGAGT ACTCGGTATG TGACAGTGAG |
| 541 | AGTCTGTGGG TGACCGACAA GTCATCGGCC ATCGACATTC GGGGACACCA GGTCACGGTG |
| 601 | CTGGGGGAGA TCAAAACGGG CAACTCTCCC GTCAAACAAT ATTTTTATGA AACGCGATGT |
| 661 | AAGGAAGCCA GGCCGGTCAA AAACGGTTGC AGGGGTATTG ATGATAAACA CTGGAACTCT |
| 721 | CAGTGCAAAA CATCCCAAAC CTACGTCCGA GCACTGACTT CAGAGAACAA TAAACTCGTG |
| 781 | GGCTGGCGGT GGATACGGAT AGACACGTCC TGTGTGTGTG CCTTGTCGAG AAAAATCGGA |
| 841 | AGAACATGAA TTGGCATCTC TCCCCATATA TAAATTATTA CTTTAAATTA TATGATATGC |
| 901 | ATGTAGCATA TAAATGTTTA TATTGTTTTT ATATATTATA AGTTGACCTT TATTTATTAA |
| 961 | ACTTCAGCAA CCCTACAGTA TATAAGCTTT TTTCTCAATA AAATCAGTGT GCTTGCCTTC |

GENBANK ID: NM_000932.1
DEFINITION HOMO SAPIENS PHOSPHOLIPASE C, BETA 3
(PHOSPHATIDYLINOSITOL-SPECIFIC) (PLCB3), MRNA.
VERSION NM_000932.1 GI:11386138
CDS 1..3705

| | |
|---|---|
| 1 | ATGGCGGGCG CCCAGCCCGG CGTCCACGCG CTGCAGTTGG AGCCGCCCAC CGTGGTGGAG |
| 61 | ACCCTGCGGC GCGGGAGTAA GTTCATCAAA TGGGACGAGG AGACCTCCAG TCGGAACCTG |
| 121 | GTGACCCTGC GTGTGGACCC CAATGGCTTC TTCTTGTACT GGACGGGCCC CAACATGGAG |
| 181 | GTGGACACAC TGGACATCAG TTCCATCAGG GACACACGGA CAGGCCGGTA CGCCCGCCTG |
| 241 | CCCAAGGACC CCAAGATCCG GGAAGTTCTG GGCTTTGGGG GTCCCGATGC CCGGCTGGAG |
| 301 | GAGAAGCTGA TGACGGTGGT GTCTGGGCCA GACCCGGTGA ACACAGTGTT CTTGAACTTC |
| 361 | ATGGCCGTGC AGGATGACAC AGCCAAGGTC TGGTCTGAGG AGCTATTCAA GCTGGCTATG |
| 421 | AACATCCTGG CTCAGAACGC CTCCCGGAAC ACCTTCCTGC GCAAAGCATA CACGAAGCTG |
| 481 | AAGCTGCAGG TGAACCAGGA TGGTCGGATC CCCGTCAAGA ACATCCTGAA GATGTTCTCA |
| 541 | GCAGACAAGA AGCGGGTGGA GACTGCGCTG GAATCCTGTG GCCTCAAATT CAACCGGAGT |
| 601 | GAGTCCATCC GGCCTGATGA GTTTTCCTTG GAAATCTTTG AGCGGTTCCT GAACAAGCTG |
| 661 | TGTCTGCGGC CGGACATTGA CAAGATCCTG CTGGAGATAG GCGCCAAGGG CAAGCCATAC |
| 721 | CTGACGCTGG AGCAGCTCAT GGACTTCATC AACCAGAAGC AACGCGACCC GAGACTCAAC |
| 781 | GAAGTGCTGT ACCCGCCCCT GCGGCCCTCC CAGGCCCGGC TGCTCATCGA AAAGTATGAG |
| 841 | CCCAACCAGC AGTTTCTGGA GCGAGACCAG ATGTCCATGG AGGGCTTTAG CCGCTACCTG |
| 901 | GGAGGCGAGG AGAATGGCAT CCTGCCCCTG GAAGCCCTGG ATCTGAGCAC GGACATGACC |
| 961 | CAGCCACTGA GTGCCTACTT CATCAACTCC TCGCATAACA CCTATCTCAC TGCGGGGCAG |
| 1021 | CTGGCTGGGA CCTCGTCGGT GGAGATGTAC CGCCAGGCAC TACTATGGGG CTGCCGCTGC |
| 1081 | GTGGAGCTGG ACGTGTGGAA GGGACGGCCG CCTGAGGAGG AACCCTTCAT TACCCACGGC |
| 1141 | TTCACCATGA CCACAGAGGT GCCTCTGCGC GACGTGCTGG AGGCCATTGC CGAGACTGCC |
| 1201 | TTCAAGACCT CGCCCTACCC CGTCATCCTC TCCTTCGAGA ACCATGTGGA CTCGGCAAAG |
| 1261 | CAACAGGCAA AGATGGCTGA GTACTGCCGC TCCATCTTTG GAGACGCGCT ACTCATCGAG |
| 1321 | CCTCTGGACA AGTACCCGCT GGCCCCAGGC GTTCCCCTGC CCAGCCCCCA GGACCTGATG |
| 1381 | GGCCGTATCC TGGTGAAGAA CAAGAAGCCG CACCGACGCA GCGCAGGTGG CCCAGACAGC |
| 1441 | GCCGGGCGCA AGCGGCCCCT GGAGCAGAGC AATTCTGCCC TGAGCGAGAG CTCCGCGGCC |
| 1501 | ACCGAGCCCT CCTCCCCGCA GCTGCGGTCT CCCAGCTCTG ACAGCTGCCC AGGCCTGAGC |
| 1561 | AATGGGGAGG AGGTAGGCCT TCACAAGCCC ACCCTGCAGC CTCAGAAGTC TCTCGGTGAC |
| 1621 | GAGCGCCTGA ACCGAGGCCC CTATGTTCTT GGACCTGCTG ACCGTGAGGA TGAGGAGGAA |
| 1681 | GATGAGCAAG AGCAGGAACA GACAGACCCC AAAAAGCCAA CTACAGATGA GGGGACAGCC |
| 1741 | AGCAGCGAGG TGAATGCCAC TGAGGAGATG TCCACGCTTG TCAACTACAT CGAACCTGTC |
| 1801 | AAGTTCAAGT CCTTTGAGGC TGCTCGAAAG AGGAACAAAT GCTTCGAGAT GTCGTCCTTT |
| 1861 | GTGGAGACCA AGGCCATGGA GCAACTGACC AAGAGCCCCA TGGAGTTTGT GGAATACAAC |
| 1921 | AAGCAGCAGC TCAGCCGCAT CTACCCCAAG GGCACCCGCG TGGACTCCTC CAACTACATG |
| 1981 | CCCCAGCTCT TCTGGAACGT AGGGTGCCAG CTTGTTGCGC TCAACTTCCA GACCCTCGAT |
| 2041 | GTGGCGATGC AGCTCAACGC GGGCGTTTTT GAGTACAACG GGCGCAGCGG GTACCTGCTC |
| 2101 | AAGCCGGAGT TCATGCGGCG GCCGGACAAG TCCTTCGACC CCTTCACTGA GGTCATCGTG |
| 2161 | GATGGCATCG TGGCCAATGC CTTGCGGGTC AAGGTGATCT CAGGGCAGTT CCTGTCCGAC |
| 2221 | AGGAAGGTGG GCATCTACGT GGAGGTGGAC ATGTTTGGCC TCCCTGTTGA TACGCGGCGC |
| 2281 | AAGTACCGCA CCCGGACCTC TCAGGGGAAC TCGTTCAACC CCGTGTGGGA CGAAGAGCCC |
| 2341 | TTCGACTTCC CCAAGGTGGT GCTGCCCACG CTGGCTTCAC TTCGCATTGC AGCCTTTGAG |
| 2401 | GAGGGGGGTA AATTCGTAGG GCACCGGATC CTGCCTGTCT CTGCCATCCG CTCCGGATAC |
| 2461 | CACTACGTGT GCCTGCGGAA CGAGGCCAAC CAACCGCTGT GCCTGCCGGC CCTGCTCATC |
| 2521 | TACACCGAAG CCTCGGACTA CATTCCTGAC GACCACCAGG ACTATGCGGA GGCCCTGATC |
| 2581 | AACCCCATTA AGCACGTCAG CCTGATGGAC CAGAGGGCCC GGCAGCTGGC CGCCCTCATT |
| 2641 | GGGGAGAGTG AGGGTGAGGC TGGCCAAGAG ACGTGCCAGG ACACCCAGTC TCAGCAGCTG |
| 2701 | GGGTCTCAGC CGTCCTCAAA CCCCACCCCC AGCCCACTGG ATGCCTCCCC CCGCCGGCCC |
| 2761 | CCTGGCCCCA CCACCTCCCC TGCCAGCACC TCCCTCAGCA GCCCAGGGCA GCGTGATGAT |
| 2821 | CTCATCGCCA GCATCCTCTC AGAGGTGGCC CCCACCCCGC TGGATGAGCT CCGAGGTCAC |
| 2881 | AAGGCTCTGG TCAAGCTCCG GAGCCGGCAA GAGCGAGACC TGCGGGAGCT GCGCAAGAAG |
| 2941 | CATGAGGGGA AGGCAGTCAC CCTCACCCGC CGCCTGCTGG ATGGCCTGGC TCAGGCACAG |
| 3001 | GCTGAGGGCA GGTGCCGGCT GCGGCCAGGT GCCCTAGGTG GGGCCGCTGA TGTGGAGGAC |
| 3061 | ACGAAGGAGG GGGAGGACGA GGCAAAGCGG TATCAGGAGT TCCAGAACAG ACAGGTGCAG |
| 3121 | AGCCTGCTGG AGCTGCGGGA GGCCCAGGTG GACGCAGAGG CCCAGCGGAG GCTGGAACAC |
| 3181 | CTGAGACAGG CTCTGCAGCG GCTCAGGGAG GTCGTCCTTG ATGCAAACAC AACTCAGTTC |
| 3241 | AAGAGGCTGA AAGAGATGAA CGAGAGGGAG AAGAAGGAGC TGCAGAAGAT CCTGGACAGA |
| 3301 | AAGCGCCATA ACAGCATCTC GGAGGCCAAG ATGAGGGACA AGCATAAGAA GGAGGCGGAA |
| 3361 | CTGACGGAGA TTAACCGTCG GCACATCACT GAGTCAGTCA ACTCCATCCG TCGGCTGGAG |
| 3421 | GAGGCCCAGA AGCAGCGGCA TGACCGTCTT GTGGCTGGGC AGCAGCAGGT CCTGCAACAG |
| 3481 | CTGGCAGAAG AGGAGCCCAA GCTGCTGGCC CAGCTGGCCC AGGAGTGTCA GGAGCAGCGG |
| 3541 | GCGAGGCTCC CCCAGGAGAT CCGCCGGAGC CTGCTGGGCG AGATGCCGGA GGGGCTGGGG |
| 3601 | GACGGGCCTC TGGTGGCCTG TGCCAGCAAC GGTCACGCAC CCGGGAGCAG CGGGCACCTG |
| 3661 | TCGGGCGCTG ACTCGGAGAG CCAGGAGGAG AACACGCAGC TCTGA |

GENBANK ID: D13119.1
DEFINITION HOMO SAPIENS P2 MRNA FOR ATP SYNTHASE SUBUNIT C, COMPLETE CDS.
VERSION D13119.1 GI:285909
CDS 31..456
/CODON_START=1

| | |
|---|---|
| 1 | TCTCCTGCCA CAGCTCCTCA CCCCCTGAAA ATGTTCGCCT GCTCCAAGTT TGTCTCCACT |
| 61 | CCCTCCTTGG TCAAGAGCAC CTCACAGCTG CTGAGCCGTC CGCTATCTGC AGTGGTGCTG |
| 121 | AAACGACCGG AGATACTGAC AGATGAGAGC CTCAGCAGCT TGGCAGTCTC ATGTCCCCTT |
| 181 | ACCTCACTTG TCTCTAGCCG CAGCTTCCAA ACCAGCGCCA TTTCAAGGGA CATCGACACA |
| 241 | GCAGCCAAGT TCATTGGAGC TGGGGCTGCC ACAGTTGGGG TGGCTGGTTC TGGGGCTGGG |
| 301 | ATTGGAACTG TGTTTGGGAG CCTCATCATT GGTTATGCCA GGAACCCTTC TCTGAAGCAA |
| 361 | CAGCTCTTCT CCTACGCCAT TCTGGGCTTT GCCCTCTCGG AGGCCATGGG GCTCTTTTGT |
| 421 | CTGATGGTAG CCTTTCTCAT CCTCTTTGCC ATGTGAAGGA GCCGTCTCCA CCTCCCATAG |
| 481 | TTCTCCCGCG TCTGGTTGGC CCCGTGTGTT CCTTTTCCTA TACCTCCCCA GGCAGCCTGG |
| 541 | GGAACGTGGT TGGCTCAGGG TTTGACAGAG AAAAGACAAA TAAATACTGT ATTAATAAG |

GENBANK ID: NM_004530.1
DEFINITION HOMO SAPIENS MATRIX METALLOPROTEINASE 2 (GELATINASE A, 72KD
GELATINASE, 72KD TYPE IV COLLAGENASE) (MMP2), MRNA.
VERSION NM_004530.1 GI:11342665
CDS 290..2272
/CODON_START=1

| | |
|---|---|
| 1 | TGTTTCCGCT GCATCCAGAC TTCCTCAGGC GGTGGCTGGA GGCTGCGCAT CTGGGGCTTT |
| 61 | AAACATACAA AGCGATTGCC AGGACCTGCG GCGGCGGCGG CGGCGGCGGG GGCTGGGGCG |
| 121 | CGGGGGCCGG ACCATGAGCC GCTCAGCCGG GCAAACCCCA GGCCACCGAG CCAGCGGACC |
| 181 | CTCGGAGCGC AGCCCTGCGC CGCGGACCAG GCTCCAACCA GGCGGCGAGG CGGCCACACG |
| 241 | CACCGAGCCA GCGACCCCCG GGCGACGCGC GGGGCCAGGG AGCGCTACGA TGGAGGCGCT |
| 301 | AATGGCCCGG GGCGCGCTCA CGCGTCCCCT GAGGGCGCTC TGTCTCCTGG GCTGCCTGCT |
| 361 | GAGCCACGCC GCCGCCGCGC CGTCGCCCAT CATCAAGTTC CCCGGCGATG TCGCCCCCAA |
| 421 | AACGGACAAA GAGTTGGCAG TGCAATACCT GAACACCTTC TATGGCTGCC CCAAGGAGAG |
| 481 | CTGCAACCTG TTTGTGCTGA AGGACACACT AAAGAAGATG CAGAAGTTCT TTGGACTGCC |
| 541 | GCAGACAGCT GATCTTGACC AGAATACCAT CGAGACCATG CGGAAGCCAC GCTGCGGCAA |
| 601 | CCCAGATGTG GCCAACTACA ACTTCTTCCC TCGCAAGCCC AAGTGGGACA AGAACCAGAT |
| 661 | CACATACAGG ATCATTGGCT ACACACCTGA TCTGGACCCA GAGACAGTGG ATGATGCCTT |
| 721 | TGCTCGTGCC TTCCAAGTCT GGAGCGATGT GACCCCACTG CGGTTTTCTC GAATCCATGA |
| 781 | TGGAGAGGCA GACATCATGA TCAACTTTCG CCGCTGGGAG CATGGCGATG GATACCCCTT |
| 841 | TGACGGTAAG GACGGACTCC TGGCTCATCC CTTCGCCCCA GGCACTGGTG TTGGGGGAGA |
| 901 | CTCCCATTTT GATGACGATG AGCTATGGAC CTTGGGAGAA GGCCAAGTGG TCCGTGTGAA |
| 961 | GTATGGCAAC GCCGATGGGG AGTACTGCAA GTTCCCCTTC TTGTTCAATG GCAAGGAGTA |
| 1021 | CAACAGCTGC ACTGATACTG GCCGCAGCGA TGGCTTCCTC TGGTCCTCCA CCACCTACAA |
| 1081 | CTTTGAGAAG GATGGCAAGT ACGGCTTCTG TCCCCATGAA GCCCTGTTCA CCATGGGCGG |
| 1141 | CAACGCTGAA GGACAGCCCT GCAAGTTTCC ATTCCGCTTC CAGGGCACAT CCTATGACAG |
| 1201 | CTGCACCACT GAGGGCCGCA CCGATGGCTA CCGCTGGTGC GGCACCACTG AGGACTACGA |
| 1261 | CCGCGACAAG AAGTATGGCT TCTGCCCTGA GACCGCCATG TCCACTGTTG GTGGGAACTC |
| 1321 | AGAAGGTGCC CCCTGTGTCT TCCCCTTCAC TTTCCTGGGC AACAAATATG AGAGCTGCAC |
| 1381 | CAGCGCCGGC CGCAGTGACG GAAAGATGTG GTGTGCGACC ACAGCCAACT ACGATGACGA |
| 1441 | CCGCAAGTGG GGCTTCTGCC CTGACCAAGG GTACAGCCTG TTCCTCGTGG CAGCCCACGA |
| 1501 | GTTTGGCCAC GCCATGGGGC TGCAGCACTC CCAAGACCCT GGGGCCCTGA TGGCACCCAT |
| 1561 | TTACACCTAC ACCAAGAACT TCCGTCTGTC CCAGGATGAC ATCAAGGGCA TTCAGGAGCT |
| 1621 | CTATGGGGCC TCTCCTGACA TTGACCTTGG CACCGGCCCC ACCCCCACAC TGGGCCCTGT |
| 1681 | CACTCCTGAG ATCTGCAAAC AGGACATTGT ATTTGATGGC ATCGCTCAGA TCCGTGGTGA |
| 1741 | GATCTTCTTC TTCAAGGACC GGTTCATTTG GCGGACTGTG ACGCCACGTG ACAAGCCCAT |
| 1801 | GGGGCCCCTG CTGGTGGCCA CATTCTGGCC TGAGCTCCCG GAAAAGATTG ATGCGGTATA |
| 1861 | CGAGGCCCCA CAGGAGGAGA AGGCTGTGTT CTTTGCAGGG AATGAATACT GGATCTACTC |
| 1921 | AGCCAGCACC CTGGAGCGAG GGTACCCCAA GCCACTGACC AGCCTGGGAC TGCCCCCTGA |
| 1981 | TGTCCAGCGA GTGGATGCCG CCTTTAACTG GAGCAAAAAC AAGAAGACAT ACATCTTTGC |
| 2041 | TGGAGACAAA TTCTGGAGAT ACAATGAGGT GAAGAAGAAA ATGGATCCTG GCTTTCCCAA |
| 2101 | GCTCATCGCA GATGCCTGGA ATGCCATCCC CGATAACCTG GATGCCGTCG TGGACCTGCA |
| 2161 | GGGCGGCGGT CACAGCTACT TCTTCAAGGG TGCCTATTAC CTGAAGCTGG AGAACCAAAG |
| 2221 | TCTGAAGAGC GTGAAGTTTG GAAGCATCAA ATCCGACTGG CTAGGCTGCT GAGCTGGCCC |
| 2281 | TGGCTCCCAC AGGCCCTTCC TCTCCACTGC CTTCGATACA CCGGGCCTGG AGAACTAGAG |
| 2341 | AAGGACCCGG AGGGGCCTGG CAGCCGTGCC TTCAGCTCTA CAGCTAATCA GCATTCTCAC |
| 2401 | TCCTACCTGG TAATTTAAGA TTCCAGAGAG TGGCTCCTCC CGGTGCCCAA GAATAGATGC |
| 2461 | TGACTGTACT CCTCCCAGGC GCCCCTTCCC CCTCCAATCC CACCAACCCT CAGAGCCACC |
| 2521 | CCTAAAGAGA TCCTTTGATA TTTTCAACGC AGCCCTGCTT TGGGCTGCCC TGGTGCTGCC |
| 2581 | ACACTTCAGG CTCTTCTCCT TTCACAACCT TCTGTGGCTC ACAGAACCCT TGGAGCCAAT |
| 2641 | GGAGACTGTC TCAAGAGGGC ACTGGTGGCC CGACAGCCTG GCACAGGGCA GTGGGACAGG |
| 2701 | GCATGGCCAG GTGGCCACTC CAGACCCCTG GCTTTTCACT GCTGGCTGCC TTAGAACCTT |
| 2761 | TCTTACATTA GCAGTTTGCT TTGTATGCAC TTTGTTTTTT TCTTTGGGTC TTGTTTTTTT |
| 2821 | TTTCCACTTA GAAATTGCAT TTCCTGACAG AAGGACTCAG GTTGTCTGAA GTCACTGCAC |
| 2881 | AGTGCATCTC AGCCCACATA GTGATGGTTC CCCTGTTCAC TCTACTTAGC ATGTCCCTAC |
| 2941 | CGAGTCTCTT CTCCACTGGA TGGAGGAAAA CCAAGCCGTG GCTTCCCGCT CAGCCCTCCC |
| 3001 | TGCCCCTCCC TTCAACCATT CCCCATGGGA AATGTCAACA AGTATGAATA AAGACACCTA |
| 3061 | CTGAGTGGC |

GENBANK ID: NN_000852.2
DEFINITION HOMO SAPIENS GLUTATHIONE S-TRANSFERASE PI (GSTP1), MRNA.
VERSION NM_000852.2 GI:6552334
CDS 30..662

| | |
|---|---|
| 1 | GGAGTTTCGC CGCCGCAGTC TTCGCCACCA TGCCGCCCTA CACCGTGGTC TATTTCCCAG |
| 61 | TTCGAGGCCG CTGCGCGGCC CTGCGCATGC TGCTGGCAGA TCAGGGCCAG AGCTGGAAGG |
| 121 | AGGAGGTGGT GACCGTGGAG ACGTGGCAGG AGGGCTCACT CAAAGCCTCC TGCCTATACG |
| 181 | GGCAGCTCCC CAAGTTCCAG GACGGAGACC TCACCCTGTA CCAGTCCAAT ACCATCCTGC |
| 241 | GTCACCTGGG CCGCACCCTT GGGCTCTATG GGAAGGACCA GCAGGAGGCA GCCCTGGTGG |
| 301 | ACATGGTGAA TGACGCCGTG GAGGACCTCC GCTGCAAATA CATCTCCCTC ATCTACACCA |
| 361 | ACTATGAGGC GGGCAAGGAT GACTATGTGA AGGCACTGCC CGGGCAACTG AAGCCTTTTG |
| 421 | AGACCCTGCT GTCCCAGAAC CAGGGAGGCA AGACCTTCAT TGTGGGAGAC CAGATCTCCT |
| 481 | TCGCTGACTA CAAGCTGCTG GACTTGCTGC TGATCCATGA GGTCCTAGCC CCTGGCTGCC |
| 541 | TGGATGCGTT CCCCCTGCTC TCAGCATATG TGGGCCGCCT CACCGCCCGG CCCAAGCTCA |
| 601 | AGGCCTTCCT GGCCTCCCCT CAGTACGTGA ACCTCCCCAT CAATGGCAAC GGGAAACAGT |
| 661 | GAGGGTTGGG GGGACTCTGA GCGGGAGGCA GAGTTTGCCT TCCTTTCTCC AGGACCAATA |
| 721 | AAATTTCTAA GAGAGCT |

GENBANK ID: XM_016524.4
DEFINITION HOMO SAPIENS CREATINE KINASE, MITOCHONDRIAL 1 (UBIQUITOUS)
(CENT1) ,MRNA.
VERSION XM_016524.4 GI:17477504
CDS 358..1704
/CODON_START=1

| | |
|---|---|
| 1 | CGCGCGAGTC TCAGGTCCCG CTAATTACCT GGCGGGTGCT GCCCACCCCT GCCCTCGCGC |
| 61 | ACCTACCGCG GTGGCAGGCG GGAAGGCGGG GCCTGGGGGA GCCCCACCCC TGGAGACTGC |
| 121 | GGCTGGGGCC TCCCTCTCCT CCGCCCGCCC GCCTGCCACT AGCTCATTGC GCCTCTCCTG |
| 181 | CAGTCTGATT GGCACCGGCT CCCATTCCGG CTCCAGCCTC CAATCCGACC CCCATTTCGG |
| 241 | CTGCAGCCTC GGACCTAGCT CCGGCCCTCG GTCTATCCGG TTGCATCCTC CCTCCCTGTT |
| 301 | CCGGATCTTA TCTTGCGCCA GCGCCTACTC CAGGATCCCG TACCGAGACG TCAAGCCATG |
| 361 | GCTGGTCCCT TCTCCCGTCT GCTGTCCGCC CGCCGGGGAC TCAGGCTCCT GGCTTTGGCC |
| 421 | GGAGCGGGGT CTCTAGCCGC TGGGTTTCTG CTCCGACCGG AACCTGTACG AGCTGCCAGT |
| 481 | GAACGACGGA GGCTGTATCC CCCGAGCCAG ACATGGCCAA CTGGACAGCT CCCAGGTAAC |
| 541 | TGCACTAGGT CTAGGCGTCT GTGCCCTCCC TCCATGGTTA CTGGGTACCC CCTCCCCAGC |
| 601 | GCTGAGTACC CAGACCTCCG AAAGCACAAC AACTGCATGG CCAGTCACCT GACCCCAGCA |
| 661 | GTCTATGCAC GGCTCTGCGA CAAGACCACA CCCACTGGTT GGACGCTAGA TCAGTGTATC |
| 721 | CAGACTGGCG TGGACAACCC TGGCCACCCC TTCATCAAGA CTGTGGGCAT GGTGGCTGGA |
| 781 | GATGAGGAGA CCTATGAGGT ATTTGCTGAC CTGTTTGACC CTGTGATCCA AGAGGGACAC |
| 841 | AATGGATATG ACCCCCGGAC AATGAAGCAC ACCACGGATC TAGATGCCAG TAAAATCCGT |
| 901 | TCTGGCTACT TTGATGAGAG GTATGTATTG TCCTCTAGAG TCAGAACTGG CCGAAGCATC |
| 961 | CGAGGACTCA GTCTGCCTCC AGCTTGCACT CGAGCAGAGC GACGAGAGGT GGAACGTGTT |
| 1021 | GTGGTGGATG CACTGAGTGG CCTGAAGGGT GACCTGGCTG GACGTTACTA TAGGCTCAGT |
| 1081 | GAGATGACAG AGGCTGAACA GCAGCAGCTT ATTGATGACC ACTTTCTGTT TGATAAGCCT |
| 1141 | GTGTCCCCGT TGCTGACTGC AGCAGGAATG GCTCGAGACT GGCCAGATGC TCGTGGAATT |
| 1201 | TGGCACAACA ATGAGAAGAG CTTCCTGATC TGGGTGAATG AGGAGGATCA TACACGGGTG |
| 1261 | ATCTCCATGG AGAAGGGTGG TAACATGAAG AGAGTGTTTG AAAGATTCTG CCGAGGCCTC |
| 1321 | AAAGAGGTGG AGAGACTTAT CCAAGAACGT GGCTGGGAGT TCATGTGGAA TGAGCGTTTG |
| 1381 | GGATACATCT TGACCTGTCC ATCTAACCTG GGCACTGGAC TTCGGGCAGG AGTGCACATC |
| 1441 | AAACTGCCCC TGCTAAGCAA AGATAGCCGC TTCCCAAAGA TCCTGGAGAA CCTAAGACTC |
| 1501 | CAAAAACGTG GTACTGGAGG AGTGGACACT GCTGCTACAG GCGGTGTCTT TGATATTTCT |
| 1561 | AATTTGGACC GACTAGGCAA ATCAGAGGTG GAGCTGGTGC AACTGGTCAT CGATGGAGTA |
| 1621 | AACTATTTGA TTGATTGTGA ACGGCGTCTG GAGAGAGGUC AGGATATCCG CATCCCCACA |
| 1681 | CCTGTCATCC ACACCAAGCA TTAACTCCCC ATCGCCAGCT GATGACTCAA GATTCCCAGG |
| 1741 | AGTTCTGCTC ATTCTAATGA TGGCCCATTC TACTTGCTCT GGACCTGCCC CCGCATCCCC |
| 1801 | TGCCTCCATC CTAGTAAAGA CTCCTTGCTA TGCTGC |

GENBANK ID: NM_001443.1
DEFINITION HOMO SAPIENS FATTY ACID BINDING PROTEIN 1, LIVER (FABP1),
MRNA. VERSION NM_001443.1 GI:4557576
CDS 43..426

| | |
|---|---|
| 1 | AGAGCCGCAG GTCAGTCGTG AAGAGGGAGC TCTATTGCCA CCATGAGTTT CTCCGGCAAG |
| 61 | TACCAACTGC AGAGCCAGGA AAACTTTGAA GCCTTCATGA AGGCAATCGG TCTGCCGGAA |
| 121 | GAGCTCATCC AGAAGGGGAA GGATATCAAG GGGGTGTCGG AAATCGTGCA GAATGGGAAG |
| 181 | CACTTCAAGT TCACCATCAC CGCTGGGTCC AAAGTGATCC AAAACGAATT CACGGTGGGG |
| 241 | GAGGAATGTG AGCTGGAGAC AATGACAGGG GAGAAAGTCA AGACAGTGGT TCAGTTGGAA |
| 301 | GGTGACAATA AACTGGTGAC AACTTTCAAA AACATCAAGT CTGTGACCGA ACTCAACGGC |
| 361 | GACATAATCA CCAATACCAT GACATTGGGT GACATTGTCT TCAAGAGAAT CAGCAAGAGA |
| 421 | ATTTAAACAA GTCTGCATTT CATATTATTT TAGTGTGTAA AATTAATGTA ATAAAGTGAA |
| 481 | CTTTGTTTT |

GENBANK ID: NM_001220.1
DEFINITION HOMO SAPIENS CALCIUMCALNODULINDEPENDENT PROTEIN KINASE (CAN
KINASE) II BETA (CZANK2B), MRNA.
VERSION NM_001220.1 GI:10835005
CDS 47..1675

| | |
|---|---|
| 1 | GCGGCCCGCG TCGACCGAGC GCACGCCCAG CCCCTCCGCC GCCGCCATGG CCACCACGGT |
| 61 | GACCTGCACC CGCTTCACCG ACGAGTACCA GCTCTACGAG GATATTGGCA AGGCGGCTTT |
| 121 | CTCTGTGGTC CGACGCTGTG TCAAGCTCTG CACCGGCCAT GACTATGCAG CCAAGATCAT |
| 181 | CAACACCAAG AAGCTGTCAG CCAGAGATCA CCAGAAGCTG GAGAGACAGG CTCGGATCTG |
| 241 | CCGCCTTGTG AAGCATTCCA ACATCGTGCG TCTCCACGAC AGCATCTCCG AGGAGGGCTT |
| 301 | CCACTACCTG GTCTTCGATC TGGTCACTGG TGGGGAGCTC TTTGAAGACA TTGTGGCGAG |
| 361 | AGAGTACTAC AGCGAGGCTG ATGCCAGTCA CTGTATCCAG CAGATCCTGK AGGCCGTTCT |
| 421 | CCATTGTCAC CAAATGGCCG TCCTCCACAG AGACCTCAAG CCGSAGAACC TGCTTCTGGC |
| 481 | CAGCAAGTGC AAAGGGGCTG CAGTGAAGCT GGCAGACTTC GGCCTAGCTA TCGAGGTGCA |
| 541 | GGGGGACCAG CAGGCATGGT TTGGTTTCGC TGGCACACCA GCCTACCTGT CCCCTGAGGT |
| 601 | CCTTCGCAAA GAGGCGTACG GCAAGCCCGT GGACATCTGG GCATGTGGGG TGATCCTGTA |
| 661 | CATCCTGCTC GTGGGCTACC CACCCTTCTG GGACGAGGAC CAGCACAAGC TGTACCAGCA |
| 721 | GATCAAGGCT GGTGCCTATG ACTTCCCGTC CCCTGAGTGG GACACCGTCA CTCCTGAAGC |
| 781 | CAAAAACCTC ATCAACCAGA TGCTGACCAT CAACCCTGCC AAGCGCATCA CAGCCCATGA |
| 841 | GGCCCTGAAG CACCCGTGGG TCTGCCAACG CTCCACGGTA GCATCCATGA TGCACAGACA |
| 901 | GGAGACTGTG GAGTGTCTGA AAAAGTTCAA TGCCAGGAGA AAGCTCAAGG GAGCCATCCT |
| 961 | CACCACCATG CTGGCCACAC GGAATTTCTC AGTGGGCAGA CAGACCACCG CTCCGGCCAC |
| 1021 | AATGTCCACC GCGGCCTCCG GCACCACCAT GGGGCTGGTG GAACAAGCCA AGAGTTTACT |
| 1081 | CAACAAGAAA GCAGATGGAG TCAAGCCCCA GACGAATAGT ACCAAAAACA GTGCAGCCGC |
| 1141 | CACCAGCCCC AAAGGGACGC TTCCTCCTGC CGCCCTGGAG CCTCAAACCA CCGTCATCCA |
| 1201 | TAACCCAGTG GACGGGATTA AGGAGTCTTC TGACAGTGCC AATACCACCA TAGAGGATGA |
| 1281 | AGACGCTAAA GCCCGGAAGC AGGAGATCAT TAAGACCACG GAGCAGCTCA TCGAGGCCGT |
| 1321 | CAACAACGGT GACTTTGAGG CCTACGCGAA AATCTGTGAC CCAGGGCTGA CCTCGTTTGA |
| 1381 | GCCTGAAGCA CTGGGCAACC TGGTTGAAGG GATGGACTTC CACAGATTCT ACTTCGAGAA |
| 1441 | CCTGCTGGCC AAGAACAGCA AGCCGATCCA CACGACCATC CTGAACCCAC ACGTGCACGT |
| 1501 | CATTGGAGAG GATGCCGCCT GCATCGCTTA CATCCGGCTC ACGCAGTACA TTGACGGGCA |
| 1561 | GGGCCGGCCC CGCACCAGCC AGTCTGAGGA GACCCGCGTG TGGCACCGCC GCGACGGCAA |
| 1621 | GTGGCAGAAC GTGCACTTCC ACTGCTCGGG CGCGCCTGTG GCCCCGCTGC AGTGAAGAGC |
| 1681 | TGCGCCCTGG TTTCGCCGGA CAGAGTTGGT GTTTGGAGCC CGACTGCCCT CGGGCACACG |
| 1741 | GCCTGCCTGT CGCATGTTTG TGTCTGCCTC GTTCCCTCCC CTGGTTCCTG TGTGTGCAGA |
| 1801 | AAAACAAGAC CAGATGTGAT TTGTT |

GENBANK ID: NM_001677.1
DEFINITION HOMO SAPIENS ATPASE, NA+K+TRANSPORTING, BETA 1 POLYPEPTIDE
(ATP1B1), MRNA.
VERSION NM_001677.1 GI:4502276
CDS 127..1038

| | |
|---|---|
| 1 | GAATTCATGC TAAATTGCTG GAAGGCTGCG TCTCTGCTGT GGTGTCAGTT CCGGATGCCT |
| 61 | CATCGCCAGG GGCGCGCCGC AGCCACCCAC CCTCCGGACC GCGGCAGCTG CTGACCCGCC |
| 121 | ATCGCCATGG CCCGCGGGAA AGCCAAGGAG GAGGGCAGCT GGAAGAAATT CATCTGGAAC |
| 181 | TCAGAGAAGA AGGAGTTTCT GGGCAGGACC GGTGGCAGTT GGTTTAAGAT CCTTCTATTC |
| 241 | TACGTAATAT TTTATGGCTG CCTGGCTGGC ATCTTCATCG GAACCATCCA AGTGATGCTG |
| 301 | CTCACCATCA GTGAATTTAA GCCCACATAT CAGGACCGAG TGGCCCCGCC AGGATTAACA |
| 361 | CAGATTCCTC AGATCCAGAA GACTGAAATT TCCTTTCGTC CTAATGATCC CAAGAGCTAT |
| 421 | GAGGCATATG TACTGAACAT AGTTAGGTTC CTGGAAAAGT ACAAAGATTC AGCCCAGAGG |
| 481 | GATGACATGA TTTTTGAAGA TTGTGGCGAT GTGCCCAGTG AACCGAAAGA ACGAGGAGAC |
| 541 | TTTAATCATG AACGAGGAGA GCGAAAGGTC TGCAGATTCA AGCTTGAATG GCTGGGAAAT |
| 601 | TGCTCTGGAT TAAATGATGA AACTTATGGC TACAAAGAGG GCAAACCGTG CATTATTATA |
| 661 | AAGCTCAACC GAGTTCTAGG CTTCAAACCT AAGCCTCCCA AGAATGAGTC CTTGGAGACT |
| 721 | TACCCAGTGA TGAAGTATAA CCCAAATGTC CTTCCCGTTC AGTGCACTGG CAAGCGAGAT |
| 781 | GAAGATAAGG ATAAAGTTGG AAATGTGGAG TATTTTGGAC TGGGCAACTC CCCTGGTTTT |
| 841 | CCTCTGCAGT ATTATCCGTA CTATGGCAAA CTCCTGCAGC CCAAATACCT GCAGCCCCTG |
| 901 | CTGGCCGTAC AGTTCACCAA TCTTACCATG GACACTGAAA TTCGCATAGA GTGTAAGGCG |
| 961 | TACCGTGAGA ACATTGGGTA CAGTGAGAAA GACCGTTTTC AGGGACGTTT TGATGTAAAA |
| 1021 | ATTGAAGTTA AGAGCTGATC ACAAGCACAA ATCTTTCCCA CTAGCCATTT AATAAGTTAA |
| 1081 | AAAAAGATAC AAAAACAAAA ACCTACTAGT CTTGAACAAA CTGTCATACG TATGGGACCT |
| 1141 | ACACTTAATC TATATGCTTT ACACTAGCTT TCTGCATTTA ATAGGTTAGA ATGTAAATTA |
| 1201 | AAGTGTAGCA ATAGCAACAA AATATTTATT CTACTGTAAA TGACAAAAGA AAAAGAAAAA |
| 1261 | TTGAGCCTTG GGACGTGCCC ATTTTTACTG TAAATTATGA TTCCGTAACT GACCTTGTAG |
| 1321 | TAAGCAGTGT TTCTGGCCCC TAAGTATTGC TGCCTTGTGT ATTTTATTTA GTGTACAGTA |
| 1381 | CTACAGGTGC ATACTCTGGT CATTTTTCAA GCCATGTTTT ATTGTATCTG TTTTCTACTT |
| 1441 | TATGTGAGCA AGGTTTGCTG TCCAAGGTGT AAATATTCAA CGGGAATAAA ACTGGCATGG |
| 1501 | TAATTTTTTT TTTTTGTTTG TTTTTTGTTT TTTGGCTCTT TCAAAGGTAA TGGCCCATCG |
| 1561 | ATGAGCATTT TTAACATACT CCATAGTCTT TTCCTGTGGT GTTAGGTCTT TATTTTTATT |
| 1621 | TTTTTCCTGG GGGCTGGGGT CGGGGTTTGT CATGGGGGAA CTGCCCTTTA AATTTTAAGT |
| 1681 | GACACTACAG AAAAACACAA AAAGGTGATG GGTTGTGTTA TGCTTGTATT GAATCCTGTC |
| 1741 | TTGACATCTC TTGCCTTGTC CTCCGGTATG TTCTAAAGCT GTGTCTGAGA TCTGGATCTG |
| 1801 | CCCATCACTT TGGCCTAGGG ACAGGGCTAA TTAATTTGCT TTATACATTT TCTTTTACTT |
| 1861 | TCCTTTTTTC CTTTCTGGAG GCATCACATG CTGGTGCTGT GTCTTTATGA ATGTTTTAAC |
| 1921 | CATTTTCATG GTGGAAGAAT TTTATATTTA TGCAGTTGTA CAATTTTATT TTTTTCTGCA |
| 1981 | AGAAAAAGTG TAATGTATGA AATAAACCAA AGTCACTTGT TTGAAAATAA ATCTTTATTT |
| 2041 | TGAACTTTAT AAAAGCAATG CAGTACCCCA TAGACTGGTG TTAAATGTTG TCTACACTGC |
| 2101 | AAAATCCATG TTCTAACATA TGTAATAATT GCCAGGAGTA CAGTGCTCTT GTTGATCTTG |
| 2161 | TATTCAGTCA GGTTAAAACA ACGGACAATA AAAGAATGAA CCGAATTC |

GENBANK ID: U82535.1
VERSION U82535.1 GI:2149155
CDS 36..1775
/CODON_START=1
MVQYELWAALPGASGVALACCFVAAAVALRWSGRRTARGAVVRA
RQKQRAGLENMDRAAQRFRLQNPDLDSEALLALPLFQLVQKLHSRELAPEAVLFTYVG
KAWEVNKGTNCVTSYLADCETQLSQAPRQGLLYGVPVSLKECFTYKGQDSTLGLSLNE
GVPAECDSVVVHVLKLQGAVPFVHTNVPQSMFSYDCSNPLFGQTVNPWKSSKSPGGSS
GGEGALIGSGGSPLGLGTDIGGSIRFPSSFCGICGLKPTGNRLSKSGLKGCVYGQEAV
RLSVGPNARDVESLALCLRALLCEDMFRLDPTVPPLPFREEVYTSSQPLRVGYYETDN
YTMPSPAMRRAVLETKQSLEAAGHTLVPFLPSNIPHALETLSTGGLFSDGGHTFLQNF
KGDFVDPCLGDLVSILKLPQWLKGLLAFLVKPLLPRLSAFLSNMKSRSAGKLWELQHE
IEVYRKTVIAQWRALDLDVVLTPMLAPALDLNAPGRATGAVSYTMLYNCLDFPAGVVP
VTTVTAEDEAQMEHYRGYFGDINDKNLQKGMKKSVGLPVAVQCVALPWQEELCLRFMR
EVERLMTPEKQSS
GENBANK ID: NM_000789.1
DEFINITION HOMO SAPIENS DIPEPTIDYL CARBOXYPEPTIDASE 1 (ANGIOTENSIN I
CONVERTING ENZYME) (ACE), MRNA.
VERSION NM_000789.1 GI:4503272
CDS 23..3943

| | |
|---|---|
| 1 | GCCGAGCACC GCGCACCGCG TCATGGGGGC CGCCTCGGGC CGCCGGGGGC CGGGGCTGCT |
| 61 | GCTGCCGCTG CCGCTGCTGT TGCTGCTGCC GCCGCAGCCC GCCCTGGCGT TGGACCCCGG |
| 121 | GCTGCAGCCC GGCAACTTTT CTGCTGACGA GGCCGGGGCG CAGCTCTTCG CGCAGAGCTA |
| 181 | CAACTCCAGC GCCGAACAGG TGCTGTTCCA GAGCGTGGCC GCCAGCTGGG CGCACGACAC |
| 241 | CAACATCACC GCGGAGAATG CAAGGCGCCA GGAGGAAGCA GCCCTGCTCA GCCAGGAGTT |
| 301 | TGCGGAGGCC TGGGGCCAGA AGGCCAAGGA GCTGTATGAA CCGATCTGGC AGAACTTCAC |
| 361 | GGACCCGCAG CTGCGCAGGA TCATCGGAGC TGTGCGAACC CTGGGCTCTG CCAACCTGCC |
| 421 | CCTGGCTAAG CGGCAGCAGT ACAACGCCCT GCTAAGCAAC ATGAGCAGGA TCTACTCCAC |
| 481 | CGCCAAGGTC TGCCTCCCCA ACAAGACTGC CACCTGCTGG TCCCTGGACC CAGATCTCAC |
| 541 | CAACATCCTG GCTTCCTCGC GAAGCTACGC CATGCTCCTG TTTGCCTGGG AGGGCTGGCA |
| 601 | CAACGCTGCG GGCATCCCGC TGAAACCGCT GTACGAGGAT TTCACTGCCC TCAGCAATGA |
| 661 | AGCCTACAAG CAGGACGGCT TCACAGACAC GGGGGCCTAC TGGCGCTCCT GGTACAACTC |
| 721 | CCCCACCTTC GAGGACGATC TGGAACACCT CTACCAACAG CTAGAGCCCC TCTACCTGAA |
| 781 | CCTCCATGCC TTCGTCCGCC GCGCACTGCA TCGCCGATAC GGAGACAGAT ACATCAACCT |
| 841 | CAGGGGACGC ATCCCTGCTC ATCTGCTGGG AGACATGTGG GCCCAGAGCT GGGAAAACAT |
| 901 | CTACGACATG GTGGTGCCTT TCCCAGACAA GCCCAACCTC GATGTCACCA GTACTATGCT |
| 961 | GCAGCAGGGC TGGAACGCCA CGCACATGTT CCGGGTGGCA GAGGAGTTCT TCACCTCCCT |
| 1021 | GGAGCTCTCC CCCATGCCTC CCGAGTTCTG GGAAGGGTCG ATGCTGGAGA AGCCGGCCGA |
| 1081 | CGGGCGGGAA GTGGTGTGCC ACGCCTCGGC TTGGGACTTC TACAACAGGA AAGACTTCAG |
| 1141 | GATCAAGCAG TGCACACGGG TCACGATGGA CCAGCTCTCC ACAGTGCACC ATGAGATGGG |
| 1201 | CCATATACAG TACTACCTGC AGTACAAGGA TCTGCCCGTC TCCCTGCGTC GGGGGGCCAA |
| 1261 | CCCCGGCTTC CATGAGGCCA TTGGGGACGT GCTGGCGCTC TCGGTCTCCA CTCCTGAACA |
| 1321 | TCTGCACAAA ATCGGCCTGC TGGACCGTGT CACCAATGAC ACGGAAAGTG ACATCAATTA |
| 1381 | CTTGCTAAAA ATGGCACTGG AAAAAATTGC CTTCCTGCCC TTTGGCTACT TGGTGGACCA |
| 1441 | GTGGCGCTGG GGGGTCTTTA GTGGGCGTAC CCCCCCTTCC CGCTACAACT TCGACTGGTG |
| 1501 | GTATCTTCGA ACCAAGTATC AGGGGATCTG TCCTCCTGTT ACCCGAAACG AAACCCACTT |
| 1561 | TGATGCTGGA GCTAAGTTTC ATGTTCCAAA TGTGACACCA TACATCAGGT ACTTTGTGAG |
| 1621 | TTTTGTCCTG CAGTTCCAGT TCCATGAAGC CCTGTGCAAG GAGGCAGGCT ATGAGGGCCC |
| 1681 | ACTGCACCAG TGTGACATCT ACCGGTCCAC CAAGGCAGGG GCCAAGCTCC GGAAGGTGCT |
| 1741 | GCAGGCTGGC TCCTCCAGGC CCTGGCAGGA GGTGCTGAAG GACATGGTCG GCTTAGATGC |
| 1801 | CCTGGATGCC CAGCCGCTGC TCAAGTACTT CCAGCCAGTC ACCCAGTGGC TGCAGGAGCA |
| 1861 | GAACCAGCAG AACGGCGAGG TCCTGGGCTC GCCCGAGTAC CAGTGGCACC CGCCGTTGCC |
| 1921 | TGACAACTAC CCGGAGGGCA TAGACCTGGT GACTGATGAG GCTCAGGCCA CCAAGTTTGT |
| 1981 | GGAGGAATAT GACCGGACAT CCCAGGTGGT GTGGAACGAG TATGCCGAGG CCAACTGGAA |
| 2041 | CTACAACACC AACATCACCA CAGACACCAG CAAGATTCTG CTGCAGAAGA ACATGCAAAT |
| 2101 | AGCCAACCAC ACCCTGAAGT ACGGCACCCA GGCCAGGAAG TTTGATGTGA ACCAGTTGCA |
| 2161 | GAACACCACT ATCAAGCGGA TCATAAAGAA GGTTCAGGAC CTAGAACGGG CAGCGCTGCC |
| 2221 | TGCCCAGGAG CTGGAGGAGT ACAACAAGAT CCTGTTGGAT ATGGAAACCA CCTACAGCGT |
| 2281 | GGCCACTGTG TGCCACCCGA ATGGCAGCTG CCTGCAGCTC GAGCCAGATC TGACGAATGT |
| 2341 | GATGGCCACA TCCCGGAAAT ATGAAGACCT GTTATGGGCA TGGGAGGGCT GGCGAGACAA |
| 2401 | GGCGGGGAGA GCCATCCTCC AGTTTTACCC GAAATACGTG GAACTCATCA ACCAGGCTGC |
| 2461 | CCGGCTCAAT GGCTATGTAG ATGCAGGGGA CTCGTGGAGG TCTATGTACG AGACACCATC |
| 2521 | CCTGGAGCAA GACCTGGAGC GGCTCTTCCA GGAGCTGCAG CCACTCTACC TCAACCTGCA |
| 2581 | TGCCTACGTG CGCCGGGCCC TGCACCGTCA CTACGGGGCC CAGCACATCA ACCTGGAGGG |
| 2641 | GCCCATTCCT GCTCACCTGC TGGGGAACAT GTGGGCGCAG ACCTGGTCCA ACATCTATGA |
| 2701 | CTTGGTGGTG CCCTTCCCTT CAGCCCCCTC GATGGACACC ACAGAGGCTA TGCTAAAGCA |
| 2761 | GGGCTGGACG CCCAGGAGGA TGTTTAAGGA GGCTGATGAT TTCTTCACCT CCCTGGGGCT |
| 2821 | GCTGCCCGTG CCTCCTGAGT TCTGGAACAA GTCGATGCTG GAGAAGCCAA CCGACGGGCG |
| 2881 | GGAGGTGGTC TGCCACGCCT CGGCCTGGGA CTTCTACAAC GGCAAGGACT TCCGGATCAA |
| 2941 | GCAGTGCACC ACCGTGAACT TGGAGGACCT GGTGGTGGCC CACCACGAAA TGGGCCACAT |
| 3001 | CCAGTATTTC ATGCAGTACA AAGACTTACC TGTGGCCTTG AGGGAGGGTG CCAACCCCGG |
| 3061 | CTTCCATGAG GCCATTGGGG ACGTGCTAGC CCTCTCAGTG TCTACGCCCA AGCACCTGCA |
| 3121 | CAGTCTCAAC CTGCTGAGCA GTGAGGGTGG CAGCGACGAG CATGACATCA ACTTTCTGAT |
| 3181 | GAAGATGGCC CTTGACAAGA TCGCCTTTAT CCCCTTCAGC TACCTCGTCG ATCAGTGGCG |
| 3241 | CTGGAGGGTA TTTGATGGAA GCATCACCAA GGAGAACTAT AACCAGGAGT GGTGGAGCCT |
| 3301 | CAGGCTGAAG TACCAGGGCC TCTGCCCCCC AGTGCCCAGG ACTCAAGGTG ACTTTGACCC |
| 3361 | AGGGGCCAAG TTCCACATTC CTTCTAGCGT GCCTTACATC AGGTACTTTG TCAGCTTCAT |
| 3421 | CATCCAGTTC CAGTTCCACG AGGCACTGTG CCAGGCAGCT GGCCACACGG GCCCCCTGCA |
| 3481 | CAAGTGTGAC ATGTACCACT CCAAGGAGGC CGGGCAGCGC CTGGCGACCG CCATGAAGCT |
| 3541 | GGGCTTCAGT AGGCCGTGGC CGGAAGCCAT GCAGCTGATC ACGGGCCAGC CCAACATGAG |
| 3601 | CGCCTCGGCC ATGTTGAGCT ACTTCAAGCC GCTGCTGGAC TGGCTCCGCA CGGAGAACGA |
| 3661 | GCTGCATGGG GAGAAGCTGG GCTGGCCGCA GTACAACTGG ACGCCGAACT CCGCTCGCTC |
| 3721 | AGAAGGGCCC CTCCCAGACA GCGGCCGCGT CAGCTTCCTG GGCCTGGACC TGGATGCGCA |
| 3781 | GCAGGCCCGC GTGGGCCAGT GGCTGCTGCT CTTCCTGGGC ATCGCCCTGC TGGTAGCCAC |
| 3841 | CCTGGGCCTC AGCCAGCGGC TCTTCAGCAT CCGCCACCGC AGCCTCCACC GGCACTCCCA |
| 3901 | CGGGCCCCAG TTCGGCTCCG AGGTGGAGCT GAGACACTCC TGAGGTGACC CGGCTGGGTC |
| 3961 | GGCCCTGCCC AAGGGCCTCC CACCAGAGAC TGGGATGGGA ACACTGGTGG GCAGCTGAGG |

GENBANK ID: XM_052144.2
DEFINITION HOMO SAPIENS APOLIPOPROTEIN A-IV (APOA4), MRNA.
VERSION XM_052144.2 GI:15314431
CDS 114..1304
/CODON_START=1

| | |
|---|---|
| 1 | AGTTCCCACT GCAGCGCAGG TGAGCTCTCC TGAGGACCTC TCTGTCAGCT CCCCTGATTG |
| 61 | TAGGGAGGAT CCAGTGTGGC AAGAAACTCC TCCAGCCCAG CAAGCAGCTC AGGATGTTCC |
| 121 | TGAAGGCCGT GGTCCTGACC CTGGCCCTGG TGGCTGTCGC CGGAGCCAGG GCTGAGGTCA |
| 181 | GTGCTGACCA GGTGGCCACA GTGATGTGGG ACTACTTCAG CCAGCTGAGC AACAATGCCA |
| 241 | AGGAGGCCGT GGAACATCTC CAGAAATCTG AACTCACCCA GCAACTCAAT GCCCTCTTCC |
| 301 | AGGACAAACT TGGAGAAGTG AACACTTACG CAGGTGACCT GCAGAAGAAG CTGGTGCCCT |
| 361 | TTGCCACCGA GCTGCATGAA CGCCTGGCCA AGGACTCGGA GAAACTGAAG GAGGAGATTG |
| 421 | GGAAGGAGCT GGAGGAGCTG AGGGCCCGGC TGCTCCGCCA TGCCAATGAG GTGAGCCAGA |
| 481 | AGATGGGGGA CAACCTGCGA GAGCTTCAGC AGCGCCTGGA GCCCTACGCG GACCAGCTGC |
| 541 | GCACCCAGGT CAACACGCAG GCCGAGCAGC TGCGGCGCCA GCTGACCCCC TACGCACAGC |
| 601 | GCATGGAGAG AGTGCTGCGG GAGAACGCCG ACAGCCTGCA GGCCTCGCTG AGGCCCCACG |
| 661 | CCGACGAGCT CAAGGCCAAG ATCGACCAGA ACGTGGAGGA GCTCAAGGGA CGCCTTACGC |
| 721 | CCTACGCTGA CGAATTCAAA GTCAAGATTG ACCAGACCGT GGAGGAGCTG CGCCGCAGCC |
| 781 | TGGCTCCCTA TGCTCAGGAC ACGCAGGAGA AGCTCAACCA CCAGCTTGAG GGCCTGACCT |
| 841 | TCCAGATGAA GAAGAACGCC GAGGAGCTCA AGGCCAGGAT CTCGGCCAGT GCCGAGGAGC |
| 901 | TGCGGCAGAG GCTGGCGCCC TTGGCCGAGG ACGTGCGTGG CAACCTGAGG GGCAACACCG |
| 961 | AGGGGCTGCA GAAGTCACTG GCAGAGCTGG GTGGGCACCT GGACCAGCAG GTGGAGGAGT |
| 1021 | TCCGACGCCG GGTGGAGCCC TACGGGGAAA ACTTCAACAA AGCCCTGGTG CAGCAGATGG |
| 1081 | AACAGCTCAG GCAGAAACTG GGCCCCCATG CGGGGGACGT GGAAGGCCAC TTGAGCTTCC |
| 1141 | TGGAGAAGGA CCTGAGGGAC AAGGTCAACT CCTTCTTCAG CACCTTCAAG GAGAAAGAGA |
| 1201 | GCCAGGACAA GACTCTCTCC CTCCCTGAGC TGGAGCAACA GCAGGAACAG CAGCAGGAGC |
| 1261 | AGCAGCAGGA GCAGGTGCAG ATGCTGGCCC CTTTGGAGAG CTGAGCTGCC CCTGGTGCAC |
| 1321 | TGGCCCCACC CTCGTGGACA CCTGCCCTGC CCTGCCACCT GTCTGTCTGT CTGTCCCAAA |
| 1381 | GAAGTTCTGG TATGAACTTG AGGACACATG TCCAGTGGGA GGTGAGACCA CCTCTCAATA |
| 1441 | TTCAATAAAG CTGCTGAGAA TCTAGCCTC |

GENBANK ID: M29366.1
DEFINITION HUMAN EFIDERMAL GROWTH FACTOR RECEPTOR (ERBB3) MRNA, COMPLETE
CDS.
VERSION M29366.1 GI:181979
CDS 100..4128
/CODON_START=1

| | |
|---|---|
| 1 | ACCAATTCGC CAGCGGTTCA GGTGGCTCTT GCCTCGATGT CCTAGCCTAG GGGCCCCCGG |
| 61 | GCCGGACTTG GCTGGGCTCC CTTCACCCTC TGCGGAGTCA TGAGGGCGAA CGACGCTCTG |
| 121 | CAGGTGCTGG GCTTGCTTTT CAGCCTGGCC CGGGGCTCCG AGGTGGGCAA CTCTCAGGCA |
| 181 | GTGTGTCCTG GGACTCTGAA TGGCCTGAGT GTGACCGGCG ATGCTGAGAA CCAATACCAG |
| 241 | ACACTGTACA AGCTCTACGA GAGGTGTGAG GTGGTGATGG GGAACCTTGA GATTGTGCTC |
| 301 | ACGGGACACA ATGCCGACCT CTCCTTCCTG CAGTGGATTC GAGAAGTGAC AGGCTATGTC |
| 361 | CTCGTGGCCA TGAATGAATT CTCTACTCTA CCATTGCCCA ACCTCCGCGT GGTGCGAGGG |
| 421 | ACCCAGGTCT ACGATGGGAA GTTTGCCATC TTCGTCATGT TGAACTATAA CACCAACTCC |
| 481 | AGCCACGCTC TGCGCCAGCT CCGCTTGACT CAGCTCACCG AGATTCTGTC AGGGGGTGTT |
| 541 | TATATTGAGA AGAACGATAA GCTTTGTCAC ATGGACACAA TTGACTGGAG GGACATCGTG |
| 601 | AGGGACCGAG ATGCTGAGAT AGTGGTGAAG GACAATGGCA GAAGCTGTCC CCCCTGTCAT |
| 661 | GAGGTTTGCA AGGGGCGATG CTGGGGTCCT GGATCAGAAG ACTGCCAGAC ATTGACCAAG |
| 721 | ACCATCTGTG CTCCTCAGTG TAATGGTCAC TGCTTTGGGC CCAACCCCAA CCAGTGCTGC |
| 781 | CATGATGAGT GTGCCGGGGG CTGCTCAGGC CCTCAGGACA CAGACTGCTT TGCCTGCCGG |
| 841 | CACTTCAATG ACAGTGGAGC CTGTGTACCT CGCTGTCCAC AGCCTCTTGT CTACAACAAG |
| 901 | CTAACTTTCC AGCTGGAACC CAATCCCCAC ACCAAGTATC AGTATGGAGG AGTTTGTGTA |
| 961 | GCCAGCTGTC CCCATAACTT TGTGGTGGAT CAAACATCCT GTGTCAGGGC CTGTCCTCCT |
| 1021 | GACAAGATGG AAGTAGATAA AAATGGGCTC AAGATGTGTG AGCCTTGTGG GGGACTATGT |
| 1081 | CCCAAAGCCT GTGAGGGAAC AGGCTCTGGG AGCCGCTTCC AGACTGTGGA CTCGAGCAAC |
| 1141 | ATTGATGGAT TTGTGAACTG CACCAAGATC CTGGGCAACC TGGACTTTCT GATCACCGGC |
| 1201 | CTCAATGGAG ACCCCTGGCA CAAGATCCCT GCCCTGGACC CAGAGAAGCT CAATGTCTTC |
| 1261 | CGGACAGTAC GGGAGATCAC AGGTTACCTG AACATCCAGT CCTGGCCGCC CCACATGCAC |
| 1321 | AACTTCAGTG TTTTTTCCAA TTTGACAACC ATTGGAGGCA GAAGCCTCTA CAACCGGGGC |
| 1381 | TTCTCATTGT TGATCATGAA GAACTTGAAT GTCACATCTC TGGGCTTCCG ATCCCTGAAG |
| 1441 | GAAATTAGTG CTGGGCGTAT CTATATAAGT GCCAATAGGC AGCTCTGCTA CCACCACTCT |
| 1501 | TTGAACTGGA CCAAGGTGCT TCGGGGGCCT ACGGAAGAGC GACTAGACAT CAAGCATAAT |
| 1561 | CGGCCGCGCA GAGACTGCGT GGCAGAGGGC AAAGTGTGTG ACCCACTGTG CTCCTCTGGG |
| 1621 | GGATGCTGGG GCCCAGGCCC TGGTCAGTGC TTGTCCTGTC GAAATTATAG CCGAGGAGGT |
| 1681 | GTCTGTGTGA CCCACTGCAA CTTTCTGAAT GGGGAGCCTC GAGAATTTGC CCATGAGGCC |
| 1741 | GAATGCTTCT CCTGCCACCC GGAATGCCAA CCCATGGAGG GCACTGCCAC ATGCAATGGC |
| 1801 | TCGGGCTCTG ATACTTGTGC TCAATGTGCC CATTTTCGAG ATGGGCCCCA CTGTGTGAGC |
| 1861 | AGCTGCCCCC ATGGAGTCCT AGGTGCCAAG GGCCCAATCT ACAAGTACCC AGATGTTCAG |
| 1921 | AATGAATGTC GGCCCTGCCA TGAGAACTGC ACCCAGGGGT GTAAAGGACC AGAGCTTCAA |
| 1981 | GACTGTTTAG GACAAACACT GGTGCTGATC GGCAAAACCC ATCTCACAAT GGCTTTGACA |
| 2041 | GTGATAGCAG GATTGGTAGT GATTTTCATG ATGCTGGGCG GCACTTTTCT CTACTGGCGT |
| 2101 | GGGCGCCGGA TTCAGAATAA AAGGGCTATG AGGCGATACT TGGAACGGGG TGAGAGCATA |
| 2161 | GAGCCTCTGG ACCCCAGTGA GAAGGCTAAC AAAGTCTTGG CCAGAATCTT CAAAGAGACA |
| 2221 | GAGCTAAGGA AGCTTAAAGT GCTTGGCTCG GGTGTCTTTG GAACTGTGCA CAAAGGAGTG |
| 2281 | TGGATCCCTG AGGGTGAATC AATCAAGATT CCAGTCTGCA TTAAAGTCAT TGAGGACAAG |
| 2341 | AGTGGACGGC AGAGTTTTCA AGCTGTGACA GATCATATGC TGGCCATTGG CAGCCTGGAC |
| 2401 | CATCCCCACA TTGTAAGGCT GCTGGGACTA TGCCCAGGGT CATCTCTGCA GCTTGTCACT |
| 2461 | CAATATTTGC CTCTGGGTTC TCTGCTGGAT CATGTGAGAC AACACCGGGG GGCACTGGGG |
| 2521 | CCACAGCTGC TGCTCAACTG GGGAGTACAA ATTGCCAAGG GAATGTACTA CCTTGAGGAA |
| 2581 | CATGGTATGG TGCATAGAAA CCTGGCTGCC CGAAACGTGC TACTCAAGTC ACCCAGTCAG |
| 2641 | GTTCAGGTGG CAGATTTTGG TGTGGCTGAC CTGCTGCCTC CTGATGATAA GCAGCTGCTA |
| 2701 | TACAGTGAGG CCAAGACTCC AATTAAGTGG ATGGCCCTTG AGAGTATCCA CTTTGGGAAA |
| 2761 | TACACACACC AGAGTGATGT CTGGAGCTAT GGTGTGACAG TTTGGGAGTT GATGACCTTC |
| 2821 | GGGGCAGAGC CCTATGCAGG GCTACGATTG GCTGAAGTAC CAGACCTGCT AGAGAAGGGG |
| 2881 | GAGCGGTTGG CACAGCCCCA GATCTGCACA ATTGATGTCT ACATGGTGAT GGTCAAGTGT |
| 2941 | TGGATGATTG ATGAGAACAT TCGCCCAACC TTTAAAGAAC TAGCCAATGA GTTCACCAGG |
| 3001 | ATGGCCCGAG ACCCACCACG GTATCTGGTC ATAAAGAGAG AGAGTGGGCC TGGAATAGCC |
| 3061 | CCTGGGCCAG AGCCCCATGG TCTGACAAAC AAGAAGCTAG AGGAAGTAGA GCTGGAGCCA |
| 3121 | GAACTAGACC TAGACCTAGA CTTGGAAGCA GAGGAGGACA ACCTGGCAAC CACCACACIG |
| 3181 | GGCTCCGCCC TCAGCCTACC AGTTGGAACA CTTAATCGGC CACGTGGGAG CCAGAGCCTT |
| 3241 | TTAAGTCCAT CATCTGGATA CATGCCCATG AACCAGGGTA ATCTTGGGCA GTCTTGCCAG |
| 3301 | GAGTCTGCAG TTTCTGGGAG CAGTGAACGG TGCCCCCGTC CAGTCTCTCT ACACCCAATC |
| 3361 | CCACGGGGAT GCCTGGCATC AGAGTCATCA GAGGGGCATG TAACAGGCTC TGAGGCTGAG |
| 3421 | CTCCAGCAGA AAGTGTCAAT GTGTAGAAGC CCGAGCAGCA GGCGGAGGCG ACGGCCACGC |
| 3481 | GGAGATAGGG CCTACCATTC CCAGCGCCAC AGTCTGCTGA CTCCTGTTAC CCCACTCTCC |
| 3541 | CCACCCGGGT TAGAGGAAGA GGATGTCAAC GGTTATGTCA TGCCAGATAC ACACCTCAAA |
| 3601 | GGTACTCCCT CCTCCCGGGA AGGCACCCTT TCTTCAGTGG GTCTTAGTTC TGTCCTGGGT |
| 3661 | ACTGAAGAAG AAGATGAAGA TGAGGAGTAT GAATACATGA ACCGGAGGAG AAGGCACAGT |
| 3721 | CCACCTCATC CCCCTAGGCC AAGTTCCCTT GAGGAGCTGG GTTATGAGTA CATGGATGTG |
| 3781 | GGGTCAGACC TCAGTGCCTC TCTGGGCAGC ACACAGAGTT GCCCACTCCA CGCTGTACGC |
| 3841 | ATCATGCCCA CTGCAGGCAC AACTCCAGAT GAAGACTATG AATATATGAA TCGGCAACGA |
| 3901 | GATGGAGGTG GTCCTGGGGG TGATTATGCA GCCATGGGGG CCTGCCCAGC ATCTGAGCAA |
| 3961 | GGGTATGAAG AGATGAGAGC TTTTCAGGGG CCTGGACATC AGGCCCCCCA TGTCCATTAT |
| 4021 | GCCCGCCTAA AAACTCTACG TAGCTTAGAG GCTACAGACT CTGCCTTTGA TAACCCTGAT |
| 4081 | TACTGGCATA GCAGGCTTTT CCCCAAGGCT AATGCCCAGA GAACGTAACT CCTGCTCCCT |
| 4141 | GTGGCACTCA GGGAGCATTT AATGGCAGCT AGTGCCTTTA GAGGGTACCG TCTTCTCCCT |
| 4201 | ATTCCCTCTC TCTCCCAGGT CCCAGCCCCT TTTCCCCAGT CCCAGACAAT TCCATTCAAT |
| 4261 | CTTTGGAGGC TTTTAAACAT TTTGACACAA AATTCTTATG GTATGTAGCC AGCTGTGCAC |
| 4321 | TTTCTTCTCT TTCCCAACCC CAGGAAAGGT TTTCCTTATT TTGTGTGCTT TGCCAGTCGC |
| 4381 | ATTCCTCAGC TTCTTCACAG GCACTCCTGG AGATATGAAG GATTACTCTC CATATCCCTT |
| 4441 | CCTCTCAGGC TCTTGACTAC TTGGAACTAG GCTCTTATGT GTGCCTTTGT TTCCCATCAG |
| 4501 | ACTGTCAAGA AGAGGAAAGG GAGGAAACCT AGCAGAGGAA AGTGTAATTT TGGTTTATGA |
| 4561 | CTCTTAACCC CCTAGAAAGA CAGAAGCTTA AAATCTGTGA AGAAAGAGGT TAGGAGTAGA |
| 4621 | TATTGATTAC TATCATAATT CAGCACTTAA CTATGAGCCA GGCATCATAC TAAACTTCAC |
| 4681 | CTACATTATC TCACTTAGTC CTTTATCATC CTTAAAACAA TTCTGTGACA TACATATTAT |
| 4741 | CTCATTTTAC ACAAAGGGAA GTCGGGCATG GTGGCTCATG CCTGTAATCT CAGCACTTTG |
| 4801 | GGAGGCTGAG GCAGAAGGAT TACCTGAGGC AAGGAGTTTG AGACCAGCTT AGCCAACATA |
| 4861 | GTAAGACCCC CATCTCTTT |

GENBANK ID: X54686
VERSION X54686.1 GI:56909
MCTKMEQAFYHDDSYAAAGYGRSPGSLSLHDYKLLKPTLALNLA
DPYRGLEGPGARGPGPEGSGAGSYFSGQGSDTGASLKLASTELERLIVPNSNGVITTT
PTPPGQYPYPRGGGSGGGTGGGVTEEQEGFADGFVKALDDLQKMNHVTPPNVSLGASG
GPQAGPGGVYAGPEPPPVYTNLSSYSPASAPSGGSGTAVGTGSSYPTATISYLPHAPP
FAGGNPAQLGLSRGASAFKEEPQTVPEARSRDATPPVSPINMEDQERIKVERKRLRNR
LAATKCRKRKLERIARLEDKVKTLKAENAGLSSAAGLLREQVAQLKQKVMTHVSNGCQ
LLLGVKGHAF
GENBANK ID: D26307
VERSION D26307.1 GI:450471
METPFYGEEALSGLAAGASSVAGAAGAPGGGGPAPPGRAFPGAP
PTSSNLKKQALTLSLAEQGAAGLKPGSATAPSALRPDGAPDGLLASPDLGLLKLASPE
LERLIIQSNGLVTTTPTSTQFLYPKVAASEEQEFAEGFVKALEDLHKQSQLGAATAAT
SGAPAPPAPADLAATPGATETPVYANLSSFAGGAGPPGGAATVAFAAEPVPFPPPPGA
LGPPPPPHPPRLAALKDEPQTVPDVPSFGDSPPLSPIDMDTQERIKAERKRLRNRIAA
SKCRKRKLERISRLEEKVKTLKSQNTELASTASLLREQVAQLKQKVLSHVNSGCQLLP
QHQVPAY
GENBANK ID: NM_012747.1
VERSION NM_012747.1 GI:6981591
MAQWNQLQQLDTRYLEQLHQLYSDSFPMELRQFLAPWIESQDWA
YAASKESHATLVFHNLLGEIDQQYSRFLQESNVLYQHNLRRIKQFLQSRYLEKPMEIA
RIVARCLWEESRLLQTAATAAQQGGQANHPTAAVVTEKQQMLEQHLQDVRKRVQDLEQ
KMKVVENLQDDFDFNYKTLKSQGDMQDLNGNNQSVTRQKMQQLEQMLTALDQMRRSIV
SELAGLLSAMEYVQKTLTDEELADWKRRQQIACIGGPPNICLDRLENWITSLAESQLQ
TRQQIKKLEELQQKVSYKGDPIVQHRPMLEERIVDLFRNLMKSAFVVERQPCMPMHPD
RPLVIKTGVQFTTKVRLLVKFPELNYQLKIKVCIDKDSGDVAALRGSRKFNILGTNTK
VMNMEESNNGSLSAEFKHLTLREQRCGNGGRANCDASLIVTEELHLITFETEVYHQGL
KIDLETHSLPVVVISNICQMPNAMASILWYNMLTNNPKNVNFFTKPPIGTWDQVAEVL
SWQFSSTTKRGLSIEQLTTLAEKLLGPGVNYSGCQITWAKFCKENMAGKGFSFWVWLD
NIIDLVKKYILALWNEGYIMGFISKERERAILSTKPPGTFLLRFSESSKEGGVTPTWV
EKDISGKTQIQSVEPYTKQQLNNMSFAEIIMGYKIMDATNILVSPLVYLYPDIPKEEA
FGKYCRPESQEHPEADPGSAAPYLKTKFICVTPTTCSNTIDLPMSPRTLDSLMQFGNN
GEGAEPSAGGQFESLTFDMDLTSECATSPM
GENBANK
ID: L26267.1
VERSION L26267.1 GI:425471
REILNPPEKETQGEGPSLFMASTKTEAIAPASTMEDKEEDVGFQ
DNLFLEKALQLAKRHANALFDYAVTGDVKNLLAVQRHLTAVQDEN0DSVLHLAIIHLE
AQLVRDLLEVTSGSISDDIINMRNDLYQTPLHLAVITKQEDVVEDLLRVGADLSLLDR
WGNSVLHLAAKEGHDKILGVLLKNSKAALLINHPNGEGLNAIHIAVMSNSLSCLQLLV
AAGAEVNAQEQKSGRTALHLAVEYDNISLAGCLLLEGDALVDSTTYDGTTPLHIAAGR
GSTRLAALLKAAGADPLVENFEPLYDLDDSWEKAGEDEGVVPGTTFLDMAANWQVFDI
LNGKFYEFVFTSDDILPQGDIKQLTEDTRLQLCKLLEIFDPDKNNATLAQKLGLGILN
NAFRLSFAPSKTLMDNYEVSGGTIKELVEALRQMGYTEAIEVIQAAFRTPETTASSPV
TTAQAHLLPLSSSSTRQHIDELRDNDSVCDSGVETSFRKLSFSESLTGDGPLLSLNKM
FHNYGQDGPIEGKI
GENEANK ID:
M3435 6.1
VERSION M34356.1 GI:181042
MTMESGAENQQSGDAAVTEAENQQMTVQAQFQIATLAQVSMPAA
HATSSAPTVTLVQLPNGQTVQVHGVIQAAQFSVIQSPQVQTVQISTIAESEDSQESVD
SVTDSQKRREILSRRPSYRKILNDLSSDAFGVFRIEEEKSEEETSAFAITTVTVPTPI
YQTSSGQYIAITQGGAIQLANNGTDGVQGLQTLTMTNAAATQPGTTILQYAQTTDGQQ
ILVPSNQVVVQAASGDVQTYQIRTAPTSTIAFGVVMASSPALPTQPAEEAARKREVRL
MKNREAARECRRKKKEYVKCLENRVAVLENQNKTLIEELKALKDYCHKSD
GENBANK ID:
X68193.1
VERSION X68193.1 GI:53353
MANLERTFIAIKPDGVQRGLVGEIIKRFEQKGFRLVAMKFLRAS
EEHLEQEYIDLKDRPFFFGLVKYMNSGPVVAMVWEGLNVVKTGRVNLGETNPADSKFG
TIRGDFCIQVGRNIIHGSDSVESAEKEIHLWFKPEELIDYKSCAHDWVYE
GENBANK
ID: U29200.1
VERSION U29200.1 GI:924934
MANLERTFIAIKPDGVQR
GENBANK ID: L35572
VERSION L35572.1 GI:531219
MNVHCAGCERFILDRFLLNVLDRAWHIKCVQCCECKTNLSEKCF
SREGKLYCKNDFFRRFGTKCAGCAQGISPSDLVRKARSKVFHLNCFTCMVCNKQLSTG
EELYVIDENKFVCKDDYLSSSSLKEGSLNSVSSCTDRSLSPDLQDFLQDDPKETDNST
SSDKETANNENEEQNSGTKRRGFRTTIKAKQLETLKAAFAATFKFTRHIREQLAQETG
LNMRVIQVWFDNRRSKERRMKQLSALGARRHAFFRSPRRMRPLGGRLDESEMLGSTPY
TYYGDYGSDYVAPGGNYDFFAHGPPSQAQSPADSSFLAASGPGSTPLGALEPPLAGPH
GADNPRFTDMISHPDTPSPEPGLPGALHPNPGEVFSGGPSPPFPMSGTSGY5GPLSHP
NPELNEAAVW
GENBANK ID: X55787.1
VERSION X55797.1 GI:296022
MKAAVDLKPTLTIIKTEKVDLELFPSPENECADVPLSTPSSKEM
MSQALKSYFSGFTKEQQRRGIPKDPRQWTDTHVRDWVMAVNEFSLKGVDFHKFCMSG
AAVCALGKECFLELAPDFVGDILWEELEILQKEDVKPYQVNGANPTYPESCYTSVYFI
SYGIEHAQCVPPSEFSEPSFITESYQTLHPISSEELLSLKYENDYPSVILRDPLQTET
LQTDYFRIKQEVLTPNNMCLGRASRGKLGGQDSFESVESYDSCDRLTQSWSSQSSFNS
LQRVPSYDSFDYEDYPAALPNHKPKGTFKDYVRDRADLNKDKPVIPAAALAGYTGSGP
IQLWQFLLELLTDKSCQSFISWTGDCNEFKLSDPDEVARRWGKRKNKPSMNYEKLSRA
LRYYYDKNIIHKTAGNAYVYAFVCDLQSLLGYTPEELHAMLDVKPDAD
GENBANK ID: M11507.1
DEFINITION Human transferrln receptor
VERSION M11507.1 GI:339515
MMDQARSAFSNLFGGEFLSYTRFSLARQVDGDNSHVENKLAVDE
EENADNNTKANVTKPKRCSGSICYCTIAVIVFFLIGFMIGYLGYCKGVEPKTECERLA
GTESPVREEFGEDFFAARRLYWDDLKRKLSEKLQSTDFTSTIKLLNENSYVPREAGSQ
KDENLALYVENQFREFKLSKVWRDQHFVKTQVKDSAQNSVIIVDKNGRLVYLVENPGG
YVAYSKAATVTCKLVHANFGTKKDFEDLYTPVNCSTVIVRAGKITFAEKVPNAESLNA
IGVLIYMDQTKFFIVNAELSFFGHAHLGTGDPYTPGFPSFNHTQFPPSRSSGLPNIPV
QTISRAAAEKLFGNMEGDCPSDWKTDSTCRMVTSESKNVKLTVSNVLKEIKILNIFGV
IKCFVEPDNYVVVGAQRDAWGP0AAKSGVGTALLLKLAQMFSDNVLKQGFQFSRSIIF
ASWSAGDFGSVGATEWLEGYLSSLHLKAFTYINLDKAVLGTSNFKVSASPLLYTLIEK
TMQNVKHPVTGQFLYQDSNWASKVEKLTLDNAAFPFLAYSGIPAVSFCFCEDTDYPYL
GTTMDTYKELIERIPELNKVARAAAEVAGQFVIKLTHDVELNLDYERYNSQLLSFVRD
LNQYRADIKEMGLSLQWLYSARGDFFPATSRLTTDFGNAEKTDRFVMKKLNDRVMRVE
YHFLSPYVSPKESPFRHVFWGSGSHTLPALLENLKLRKQNNCAFNETLFRNQLALATW
TIQGAANALSGDVWDIDNEF
GENBANK ID: AAA60255
VERSION AAA60255.1 GI:190927

| | |
|---|---|
| 1 | mteyklvvvg aggvgksalt iqllqnhfvd eydptledsy rkqvvidget clldildtag |
| 61 | qeeysamrdq ymrtgegflc vfainnsksf adlnlyreqi krvkdsddvp mvlvgnkcdl |
| 121 | ptrtvdtkqa helaksygip fietsaktrq gvedafytlv reirqyrmkk lnssddgtgg |
| 181 | cmglpcvvm |

GENBANK ID: AAB38309
VERSION AAB38309.1 GI:1497931

| | |
|---|---|
| 1 | mslvlndlli ccrqlehdra terkkevekf krllrdpeti khldrhsdsk qgkylnwdav |
| 61 | frflqkyiqk eteclrlakp nvsastqasr qkknqeissl vkytikcanr raprlkcqel |
| 121 | lnyimdtvkd ssngalygad csnhllkdil svrkywceis qqqwlelfsv yfrlylkpsq |
| 181 | dvhrvlvari ihavtkgccs qtdglnskkfl dffskaiqca rqeksssgln hilaaltifi |
| 241 | ktlavnfrir vcelgdeilp tllyiwtqhr lndslkevii elfqlqiyih hpkgaktqek |
| 301 | gayestkwrs ilynlydllv neishlgsrg kyssgfrnia vkenllelma dichqvfned |
| 361 | trslelsqsy tttqressdy svpckrkkie lgwevikdhl qksqndfdlv pwlqiatqli |
| 421 | skypaslpnc elspllmils qllpqqrhge rtpyvlrclt evalcqdkrs nlessqksdl |
| 481 | lklwnkiwci tfrgisseqi qaenfgllga iiqgslvevd refwklftgs acrpscpavc |
| 541 | cltlalttsi vpgtvkmgie qnmcevnrsf slkesimkwl ltyqlegdle nstevppilh |
| 601 | snfphlvlek ilvslomknc kaamnffqsv pecehhqkdk eelsfsevee lflqttfdkm |
| 661 | dfltivrecg iekhqssigf svhqnlkesl drcllglseq llnnysseit nsetlvrcsr |
| 721 | llvgvlgcyc ymgviaeeea ykselfqkak slmqcaqesi tlfknktnee trigslrnmm |
| 781 | qlctrclsnc tkkspnkias gfflrlltsk lmndiadick slasflkkpf firgevesmed |
| 841 | dtngnlmeve dgssmnlfnd ypdssvsdan epgesqstig ainplaeeyl skgdllfldm |
| 901 | lkflclcvtt aqtntvsfra adirrkllml idsstleptk sihihmylmi lkelpgeeyp |
| 961 | lpmedvlell kplsnvcsly rrdqdvckti lnhvlhvvkn lgqsnmdsen trdaqgqflt |
| 1021 | vigafwhltk erkyifsvrm alvnclktll eadpyskwai lnvmgkdfpv nevftqflad |
| 1081 | nhhqvrmlaa esinrlfqdt kgdssrllka lplklqqtaf enaylkaqeg mremshsaen |
| 1141 | petldeiynr ksvlltliav vlscapicek qalfalcksv kenglephlv kkvlekvset |
| 1201 | fgyrrledfm ashldylvle wlnlqdteyn lsstpfilln ytniedtyrs cykvliphlv |
| 1261 | irshfdevks ianqigedwk slltdcfpki lvnilpytay egtrdsgmaq qretatkvyd |
| 1321 | mlksenllgk qidhitisni peivvellmt lhepanssas qstdlcdfsg dldpapnpph |
| 1381 | fpshvlkatf ayisnchktk ikaileilak spdsyqkill aiceqaaetn nvykkhrilk |
| 1441 | iyhlfvslll kdiksglgga watvlrdviy tlihylnqrp scimdvslrs fslccdllsq |
| 1501 | vcqtavtyck dalenhlhvl vgtliplvye qvevqkqvld llkylvidnk dnenlyitik |
| 1561 | lldpfpdhvv fkdlrltqqk ikysrgpfsl leeinhflsv svydalplcr leglkdlrrq |
| 1621 | lelhkdqmvd inrasqdnpq dglmvklvvn llqlskmain htgekevlea vgsclgevgp |
| 1681 | idfstiaiqh skdasytkal kltedkelqw tfimltylnn tlvedcvkvr saavtclkni |
| 1741 | latktghsfw eiykmttdpm laylqpfrts rkkflevprf dkenpfegld dinlwiplse |
| 1801 | nhdiwiktlt cafldsggtk ceilqllkpm cevktdfcqt vlpylihdil lqdtneswrn |
| 1861 | llsthvqgff tsclrhfsqt srsttpanld sesehffrcc ldkksgrtml avvdymrrqk |
| 1921 | rpssgtifnd atwldinyle vakvaqscaa hftallyaei yadkksmddq ekrslateeg |
| 1981 | sqsttissls ekskeetgis lqdllleiyr sigepdslyg cgggkmlqpi trlrtyehea |
| 2041 | mwgkalvtyd letaipsstr qagiiqalqn igichilavy lkgldyenkd wcpeleelhy |
| 2101 | qaawrnmqwd hctsvskeve gtsyheslyn alqslrdref stfyeslkya rvkeveemck |
| 2161 | rslesvysly ptlsrlqaig elesigelfs rsvthrqlse vyikwqkhsq llkdsdfsfq |
| 2221 | epimalrtvi leilmekemd nsqrecikdl ltkhlvelsi lartfkntql peralfqikq |
| 2281 | ynsvscgvse wqleeaqvfw akkeqslals ilkqmikkld ascaannpsl kltyteclrv |
| 2341 | cgnwlaetcl enpavimqty lekavevagn ydgessdelr ngkmkaflsl arfsdtqyqr |
| 2401 | ienymkssef enkqallkra keevgllreh klqtnrytvk vqreleldel alralkedrk |
| 2461 | rflckaveny incllsgeeh dmwvtrlcsl wlensgvsev ngmmkrdgmk iptykflplm |
| 2521 | yqlaarmgtk mmgglgfhev lnnlisrism dhphhtlfii lalananrde fltkpevarr |
| 2581 | sritknvpkq ssqldedrte aanrilctir srrpqmvrsv ealcdayill anldatqwkt |
| 2641 | qrkginipad qpitklknle dvvvptmeik vdhtgeygnl vtiqsfkaet rlaggvnlpk |
| 2701 | iidcvgsdgk errqlvkgrd dlrqdavmgq vfqmcntllq rntetrkrkl tictykvvpl |
| 2761 | sqrsgvlewc tgtvpigefl vnnedgahkr yrpndfsafq cqkkmmevqk ksfeekyevf |
| 2821 | mdvcqntqpv fryfcmekfl dpaiwfekrl aytrsvatss ivgyilglgd rhvqniline |
| 2881 | qsaelvhidl gvafeqgkil ptpetvpfrl trdivdgmgl tgvegvfrrc cektmevmrn |
| 2941 | sqetlitlve vllydplfdw tmnplkalyl qqrpedetel hptlnaddqe ckrnlsdidq |
| 3001 | sfdkvaervl mrlqeklkgv eegtvlsvgg qvnlliqqai dpknlsrlfp gwkawv |

GENBANK ID: AAA59145.1
VERSION AAA59145.1 GI:307058

| | |
|---|---|
| 1 | mlkpslpfts llflqlpllg vglnttiltp ngnedttadf flttmptdsl svstlplpev |
| 61 | qcfvfnveym nctwnsssep qptnltlhyw yknsdndkvq kcshyltsee itsgcqlqkk |
| 121 | eihlyqtfvv qlqdpreprr qatqmlklqn lvipwapenl tlhklsesql elnwnnrfln |
| 181 | hclehlvqyr tdwdhswteq svdyrhkfsl psvdgqkryt frvrsrfnpl cgsaqhwsew |
| 241 | shpihwgsnt skenptlfal eavvisvgsm gliisllovy fwlertmpri ptlknledlv |
| 301 | teyhgnfsaw sgvskglaes lqpdyserlc lvseippkgg algegpgasp cnqhspywap |
| 361 | pcytlkpet |

GENBANK ID: AAC50825.1
VERSION AAC50825.1 GI:1117984

| | |
|---|---|
| 1 | mvaprplrrv vlfyggklcs magnfwqssh ylqwildkqd llkerqkdlk flseeeywkl |
| 61 | qifftnviqa lgehlklrqq viatatvyfk rfyaryslks idpvlmaptc vflaskveef |
| 121 | gvvsntrlia aatsvlktrf syafpkefpy rmnhilecef yllelmdccl ivyhpyrpll |
| 181 | qyvqdmgged mllplawriv ndtyrtdlcl lyppfmiala clhvacvvqq kdarqwfael |
| 241 | svdmekilei irvilklyeq wkntderkem atilskmpkp kpppnsegeq gpngsqnssy |
| 301 | sqs |

GENBANK ID: AAC50473.1
VERSION AAC50473.1 GI:1314346

| | |
|---|---|
| 1 | mamssggsgg gvpeqedsvl frrgtgqsdd sdiwddtali kaydkavasf khalkngdic |
| 61 | etsgkpkttp krkpakknks qkkntaaslq qwkvgdkcsa iwsedgciyp atlasidfkr |
| 121 | etcvvvytgy gnreeqnlsd llspicevan nleqnaqene nesqvstdes ensrspgnks |
| 181 | dnikpksapw nsflpppppm pgprlgpgkp glkfngpppp pppppphlls cwlppfpsgp |
| 241 | piipppppic pdslddadal gsmliswyms gyhtgyymgf rqnqkegrcs hsln |

GENBANK ID: CAC15525
VERSION CAC15525.1 GI:11137517

| | |
|---|---|
| 1 | mvsrdqahlg pkyvglwdtk srtdeelsfr agdvfhvark eeqwwwatll deaggavaqg |
| 61 | yvphnylaer etvesepwff qcisrseavr rlqaegnatg aflirvsekp sadyvlsvrd |
| 121 | tqavrhykiw rraggrlhln eavsflslpe lvnyhraqsl shglrlaapc rkhepeplph |
| 181 | wddwerpree ftlcrklgsg yfgevfeglw kdrvqvaikv isrdnllhqq mlqseiqamk |
| 241 | klrhkhilal yavvsvgdpv yiltelmakg sllellrdsd ekvlpvsell diawqvaegm |
| 301 | cylesqnyih rdlaarnilv gentlckvgd fglarliked vylshdhnip ykwtapeals |
| 361 | rghystksdv wsfglllhem fsrgqvpypg msnheaflrv dagyrmpcpl ecppsvhklm |
| 421 | ltcwcrdpeq rpcfkalrer lssftsyenp t |

GENBANK ID: AAB84296
VERSION AAB84296.1 GI:2613135

| | |
|---|---|
| 1 | tsttvrglna strylfrvra svqqigdwsn tveettlglq saspvqesrv aedgldqqlv |
| 61 | iavvgsvsat cltiiaalla lvclrrsclh rrhtftyqsg sgeetilqfs sgtltltrrp |
| 121 | kpqpeplsyp vle |

GENBANK ID: X63594.1
VERSION X63594.1 GI:57673
MFQPAGHGQDWANEGPRDGLKKERLVDDRHDSGLDSNKDEDYEQ
MVKELREIRLQFQEAPLAAEPWKQQLTEDGDSFLHLAIIHEEKTLTMEVIGQVKGDLA
FLNFQNNLQQTPLHLAVITNQPGIAEALLKAGCDPELRDFRGNTPLHLACEQGCLASV
AVLTQTCTPQHLHSVLQATNYNGHTCLHLASIHGYLGIVEHLVTLGADVNAQEPCNGR
TALHLAVDLQNPDLVSLLLKCGADVNRVTYQGYSPYQLTWGRPSTRIQQQLGQLTLEN
LQTLPESEDEESYDTESEFTEDELPYDDCVFGGQRLTL
GENBANK ID: AAB60641
VERSION AAB60641.1 GI:516515

| | |
|---|---|
| 1 | meqqdqsmke gmgttwllst pqhwlmqqfy netyygrtge fmedfpltll wsvtvsmfpf |
| 61 | ggfigsllvg plvnkfgrkg allfnnifsi vpailmgcsr vatsfeiiii srllvgicag |
| 121 | vssnvvpmyl gelapknlrg algvvpqlfi tvgilvaqif glrnllanvd gwpillgltg |
| 181 | vgaalqllll pffpespryl liqkkdeaaa kkalqtlrgw dsvdrevaei rqedeaekaa |
| 241 | gfisvlklfr mrslrwqlls iivlmggqql sgvnaiyyya dqiylsagvp eehvqyvtag |
| 301 | tgavnvvmtf cavfvvellg rrlllllgfs icliaccvlt aalalgdtvs wmpyisivcv |
| 361 | isyvlghaig pspipallit eiflqssrps afmvggsvhw lsnftvglif pfiqeglgpy |
| 421 | sfivfavic1 lttiyifliv petkaktfie ingiftkmnk vsevypekee lkelppvtse |
| 481 | q |

GENEANK ID: M29069
VERSION M29069.1 GI:205553
MLSCTTSTMPGMICKNSDLEFDSLKFCFYPEDDDIYFGGRNSTP
PGEDIWKKEELLPTFRLSPGRALAEDSLEPANMATEMLLFEADLWSNPAEEEDIFGLK
GLSGSSSNPVVLQDCMWSGFSSREKPETVVSEKLPGGCGSLAVGAGTLVPGAAAATSA
GHARSGTAGVGRRKAAWLTELSHLDSECVDSAVIFFANKRESMPVATIPASAGPAISL
GDHQGLSSSLEDFLSNSGYVEEGGEEIYVVMLGETQFSKTVTKLPTAAHSENAALTPE
CAQSGELILKRSDLIQEDHNYAAFPLPYAEDARPLKKPRSQDPLGPLKCVLRPKAPRL
RSRSNSDLEDIERRRNHNRMERQRRDIMRSSFLNLRDLVPELVHNEKAAKVVILKKAT
EYIHTLQTDESKLLVEREKLYERKQQLLEKIKQSAVC
GENBANK ID: M29039.1
VERSION M29039.1 GI:186626
MCTKMEQPFYHDDSYTATGYGRAPGGLSLHDYKLLKPSLAVNLA
DFYRSLKAPGARGPGPEGGQGGSYFSGQGSDTGASLKLASSELERLIVFNSNGVITTT
PTPPGQYFYPRGGGSGGGAGGAGGGVTEEQEGFADGFVKALDDLHKMNMVTPFNVSLG
ATGGPPAGPGGVYAGPEPPPVYTNLSSYSPASASSGGAGAAVGTGSSYPTTTISYLPH
APPFAGGHPAQLGLGRGASTFKEEPQTVFEARSRDATPPVSPINMEDQERIKVERKRL
RNRLAATKCRKRKLERIARLEDKVKTLKAENAGLSSTAGLLREQVAQLKQKVNTMVSN
GCQLLLGVKGHAF
GENBANK ID: X56681.1
VERSION X56681.1 GI:34018
METPFYGDEALSGLGGGASGSGGTFASPGRLFPGAPPTAAAGSM
MKKDALTLSLSEQVAAALKPAPAPASYPPAADGAPSAAPPDGLLASPDLGLIKLASPE
LERLIIQSNGLVTTTFTSSQFLYFKVAASEEQEFAEGFVKALEDLHKQNQLGAGRAAA
AAAAAAGGPSGTATGSAPPGGELAPAAAAPEAPVYANLSSYAGAGGAGGAATVAFAAE
PVPFPPPPPPGALGFFRLAALKDEPQTVFDVFSFGESFFLSFIDMDTQERIKAERKRL
RNRIAASKCRKRKLERISRLEEKVKTLKSQNTELASTASLLREQVAQLKQKVLSMVNS
GCQLLPQHQVPAY
GENBANK ID: NM_003150.1
VERSION NM_003150.1 GI:4507252
MAQWNQLQQLDTRYLEQLHQLYSDSFFMELRDFLAFWIESQDWA
YAASKESHATLVFHNLLGEIDQQYSRFLQESNVLYGHNLRRIKQFLQSRYLEKFMEIA
RIVARCLWEESRLLQTAATAAQQGGQANMFTAAVVTEKQQMLEQHLQDVRKRVQQLEQ
KMKVVENLQDDFDFNYKTLKSQGDMQDLNGNNQSVTRQKMQQLEQMLTALDQMRRSIV
SELAGLLSAMEYVQKTLTGEELADWKRRQQIACIGGFFNICLDRLENWITSLAESQLQ
TRQQIKKLEELHQKVSYKGDFIVQHRFMLEERIVVELFRNLMKSAFWERQFCMFMHPD
RPLVIKTGVQFTTKVRLLVKFFELNYQLKIKVCIDKDSGDVAALRGSRKFNILGTNTK
VMNMEESNNGSLSAEFKHLTLREQRCGNGGRANCDASLIVTEELHLITFETEVYHQGL
KIDLETMSLSVVVISNICQMFNAWASILWYNMLTNNFKNVNFFTKPPIGTWDGVAEVL
SWQFSSTTKRGLSIEQLTTLAEKLLGFGVNYSGCQITWANFCKENNAGKGESYMVMLD
NIIDLVKKYILALWNEGYIMGFISKEREFAILSTKFFGTELLRESESSKEGGVTETMV
EKDISGKTQIQSVEFYTKQQLNNMSFAEIIMGYKIMDATNILLSFLVYLYFDIPKEEA
FGKYCRFESGEHFEADFGSAAFYLKTKFICVTPTTCSNTIDLPMSPRALDSLMQFGNN
GEGAEPSAGGQFESLTFDMELTSECATSPM

1. A method of increasing the levels of a protein in a Peyer's patch cell, said method comprising delivering to said cell a nucleic acid coding for a protein, wherein absent said increase, the levels of said protein or its mRNA is greater than in a non-Peyer's patch cell.

2. The method of claim 1 wherein the protein is a transcription factor or a protein that activates a transcription factor.

3. The method of claim 2 wherein the transcription factor or a protein that activates a transcription factor is selected from the group consisting of Jun-B; c-jun related TF, Jun-D; c-jun related TF, STAT 3-signal transducer and activator of transcription 3, NF-kappaβ Tf p105 subunit, S-myc proto-oncogene; myc related, Nm23-M2; nucleoside diphosphate kinase B; metastasis reducing protein, and C-est-l proto-oncogene; p54.

4. The method of claim 1 wherein the protein is a receptor, or cell surface antigen,

5. The method of claim 4 wherein the protein is a receptor or a transporter.

6. The method of claim 1 wherein the protein is selected from the group consisting of nucleoside diphosphate kinases and member of the 14-3-3 family.

7. The method of claim 1 wherein the protein is coded for by a gene with an expression Fold Change denoted by a **, *, or number greater than 2.00 in Tables 2 or 3.

8. The method of claim 1 wherein the nucleic acid coding for at least 2 proteins is delivered, each of said proteins coded for by a gene with an expression Fold Change denoted by a **, *, or number greater than 2.00 in Tables 2 or 3.

9. The method of claim 1 wherein the cell to which the nucleic acid is delivered is a human cell.

10. The method of claim 9 wherein the cell is in a Peyer's patch in a human and the nucleic acid is delivered by the oral route.

11. The method of claim 9 wherein the cell is not within the body of a human.

12. The method of claim 1 wherein the cell to which the nucleic acid is delivered is a rat cell.

13. The method of claim 1 wherein a nucleic acid coding for a tumor antigen or foreign peptide is also delivered to the Peyer's patch cell.

14. The method of claim 13 wherein the cell to which the nucleic acid is delivered is a human cell.

15. A method of decreasing the levels of a protein in a Peyer's patch cell, said method comprising delivering to said cell an anti-sense nucleic acid molecule, a ribozyme nucleic acid molecule, an RNA interference nucleic acid molecule (RNAi), said anti-sense molecule, ribozyme or RNAi nucleic acid being complementary to a sequence of at least 10 nucleotides of the mRNA for said protein, wherein absent said anti-sense molecule, ribozyme or RNAi nucleic acid, the levels of said protein or its mRNA is less than in a non-Peyer's patch cell.

16. The method of claim 15 wherein the anti-sense nucleic acid, a ribozyme nucleic acid molecule, an RNA interference nucleic acid molecule is complementary to a sequence of at least 15 nucleotides of the mRNA of the protein.

17. The method of claim 16 wherein the anti-sense nucleic acid, a ribozyme nucleic acid molecule, an RNA interference nucleic acid molecule is complementary to a sequence of at least 30 nucleotides of the mRNA of the protein.

18. The method of claim 15 wherein the protein is coded for by a gene with an expression Fold Change denoted by a “−”, or a number less that 0.5 in Tables 2 or 3.

19. The method of claim 15 comprising delivering to said cell anti-sense nucleic acid molecules, ribozyme nucleic acid molecules, RNA interference nucleic acid molecules, said anti-sense, ribozyme or RNAi nucleic acid being complementary to a sequence of at least 10 nucleotides of the mRNA for at least 5 different protein a, wherein absent said anti-sense, ribozyme or RNAi nucleic acid molecule, the levels of each of said proteins or its mRNA is less than in a non-Peyer's patch cell.

20. A method of decreasing the levels of a protein in a Peyer's patch cell, said method comprising delivering to said cell an anti-sense nucleic acid molecule, a ribozyme nucleic acid molecule, an RNA interference nucleic acid molecule said anti-sense, ribozyme or RNAi nucleic acid forming a double-stranded molecule with part or all of the mRNA for said protein, wherein absent said anti-sense, ribozyme or RNAi nucleic acid molecule, the levels of said protein or its mRNA is less than in a non-Peyer's patch cell.

21. A method of claims 1, 13, or 15 in which the Peyer's patch cell is an M cell.

22. A human cell to which the method of claims 1 has been applied, or the progeny of said human cell.

23. A human cell to which the method of claim 13 has been applied, or the progeny of said human cell.

24. A human cell to which the method of claim 15 has been applied, or the progeny of said human cell.

25. A human cell to which the method of claims 1 has been applied, or the progeny of said human cell.

26. A human cell to which the method of claim 13 has been applied, or the progeny of said human cell.

27. A human cell to which the method of claim 15 has been applied, or the progeny of said human cell.

28. A method for enhancing transport of a drug through the gastrointestinal tract, said method comprising orally administering said drug in a composition that comprises a transport-enhancing protein, said transport-enhancing protein selected from the group consisting of human serum albumin (HSA), clusterin, T-cell surface glycoprotein CD5 precursor, HSP84, and Ca2+pla2, or a homolog that has at least 80% amino acid identity with said transport-enhancing protein over a length of said transport-enhancing protein identical to the homolog.

29. A method of claim 28 wherein the homolog has at least 90% amino acid with the transport-enhancing protein over a length of the transport-enhancing protein identical to the homolog.

30. A method of claim 28 wherein the transport-enhancing protein is selected from the group consisting of human serum albumin (HSA), clusterin, T-cell surface glycoprotein CD5 precursor, HSP84, and Ca2+ pla2.

31. A method to facilitate intracellular trafficking of an antigen that has been orally delivered by itself or as part of a composition or particle, said method comprising administering a protein selected from the group consisting of calreticulin, rab family proteins and ribosomal proteins.

32. A chimeric protein comprising the amino acid sequence for calreticulin, rab family proteins and ribosomal proteins and the amino acid sequence for a second polypeptide.

33. A method of administering a polypeptide, where said polypeptide is part of a chimeric protein of claim 32, and wherein said chimeric protein is orally administered.

34. A method of delivering a vaccine to a target cell, said method comprising utilizing as the target cell a Peyer's patch cell in which a normally upregulated protein or mRNA is further upregulated.

35. A method of claim 34 wherein the Peyer's patch cell is an M Cell.

36. A method of claim 1 wherein the protein is selected from the group consisting of clusterin, T-cell surface gicoprotein CD5 precursor, HSP84, Ca2+ dependent phospholipase A2 precursor, and the ribosomal proteins, S12, S11, L12, L11, S29, S19, L21, L19, L13, L44, and L36.

37. A method of claim 34 wherein the upregulated protein is selected from the group consisting of clusterin, T-cell surface glycoprotein CD5 precursor, HSP84, and Ca2+ dependent phospholipase A2 precursor and the mRNA is for a protein selected from said group.

38. A method of claim 1 wherein the protein is selected from the group consisting of cyclin D1, PLC-L, GRB2, ERK3/MAPK6, ERK1, ERK3, JNK2, CD40, CRAF1, C-MYC, PT-α, IL-R, CD40, C-MYC, PKC-α, GSTA1, GATA-2, PLGF, ezrin, HGF activator, hepatocyte growth factor-like protein, NCAD, MNDA, LHX1, TIE-1, NCAML1, CD104, CD44, SRC1, NMDA, TKT, ephrin (type A), Sp1, RAB proteins, PKC, TIR, Jak3, EGR-1, TNK1, CAMK IV, HSP40, HSP70, HSP60, HSO27, fMLP-related receptor, HSP27, glutaredoxin, CREB, gadd153, XPG, XPD, ubiqitin-conjugating enzyme, RAD23, cadherin 2, neural cell adhesion molecule, integrin alpha 3, leukocyte adhesion glycoprotein p150, integrin beta 4, TIE, NCAML1, α3β1 integrin, CD11C antigen, CD104 antigen, CD44, NMDA, TKT, ephrin (type A), and Sp1, a RAB protein, PKC, TfR, bcl-x and caspase-9.

39. A method of claim 34 wherein the upregulated protein is selected from the group consisting of cyclin D1, PLC-L, GRB2, ERK3/MAPK6, ERK1, ERK3, JNK2, CD40, CRAF1, C-MYC, PT-α, IL-R, CD40, C-MYC, PKC-α, GSTA1, GATA-2, PLGF, ezrin, HGF activator, hepatocyte growth factor-like protein, NCAD, MNDA, LHX1, TIE-1, NCAML1, CD104, CD44, SRC1, NMDA, TKT, ephrin (type A), Sp1, RAB proteins, PKC, TIR, Jak3, EGR-1, TNK1, CAMK IV, HSP40, HSP70, HSP60, HSO27, fMLP-related receptor, HSP27, glutaredoxin, CREB, gadd153, XPG, XPD, ubiqitin-conjugating enzyme, RAD23, cadherin 2, neural cell adhesion molecule, integrin alpha 3, leukocyte adhesion glycoprotein p150, integrin beta 4, TIE, NCAML1, α3β1 integrin, CD11C antigen, CD104 antigen, CD44, NMDA, TKT, ephrin (type A), and Sp1, a RAB protein, PKC, TfR, bcl-x and caspase-9. and the mRNA is for a protein selected from said group.

40. A method of claim 1 wherein the protein is selected from the group consisting of an 1 L-2 receptor, a gamma c chain of an IL-2 receptor, intereron-γ, and a C-C chemokine.

41. A method of claim 34 wherein the upregulated protein is selected from the group consisting of an IL-2 receptor, a gamma c chain of an IL-2 receptor, intereron-γ, and a C-C chemokine and the mRNA is for a protein selected from said group.

42. A method of claim 1 wherein the protein is selected from the group consisting of cyclin D1, PLC-L, GRB2, ERK3/MAPK6, ERK1, ERK3, PKC-α, GSTA1, GATA-2, and PLGF.

43. A method of claim 34 wherein the upregulated protein is selected from the group consisting of cyclin D1, PLC-L, GRB2, ERK3/MAPK6, ERK1, ERK3, JNK2, CD40, CRAF1, C-MYC, PT-α, IL-R, PKC-α, GSTA1, GATA-2, and PLGF and the mRNA is for a protein selected from said group.

44. A method of claim 1 wherein the protein is selected from the group consisting of a RAB protein, PKC, and TfR.

45. A method of claim 34 wherein the upregulated protein is selected from the group consisting of a RAB protein, PKC, and TfR and the mRNA is for a protein selected from said group.

46. A method of claim 1 wherein the protein is selected from the group consisting of Jak 3, EGR-1, TNK1, and CAMK IV.

47. A method of claim 34 wherein the upregulated protein is selected from the group consisting of Jak 3, EGR-1, TNK1, and CAMK IV and the mRNA is for a protein selected from said group.

48. A method of claim 1 wherein the protein is selected from the group consisting of HSP40, HSP70, HSP60, HSO27, fMLP-related receptor, HSP27, glutaredoxin, CREB, gadd 153, XPG, XPD, ubiquitin, conjugating enzyme, RAD 23, and ataxia telengiectasia.

49. A method of claim 34 wherein the upregulated protein is selected from the group consisting of HSP40, HSP70, HSP60, HSO27, fMLP-related receptor, HSP27, glutaredoxin, CREB, gadd 153, XPG, XPD, ubiquitin, conjugating enzyme, RAD 23, and ataxia telengiectasia and the mRNA is for a protein selected from said group.

50. A method of decreasing the levels of a protein in a Peyer's patch cell, said method comprising delivering to said cell a DNA molecule coding for an anti-sense nucleic acid molecule, a ribozyme nucleic acid molecule, an RNA interference nucleic acid molecule (RNAi), said anti-sense molecule, ribozyme or RNAi nucleic acid being complementary to a sequence of at least 10 nucleotides of the mRNA for said protein, wherein absent said anti-sense molecule, ribozyme or RNAi nucleic acid, the levels of said protein or its mRNA is less than in a non-Peyer's patch cell.

51. A method of increasing the extent to which the function of a protein is carried out in a Peyer's patch cell, said method comprising delivering to said cell a nucleic acid coding for said protein, wherein absent said delivery, the level of said protein or its mRNA is greater in said cell than in a non-Peyer's patch cell.

52. A chimeric protein that comprises two or more segments, each of said segments enhancing a different step in the peptide transport process, said steps selected from the group consisting of binding to a cell, transporting the peptide into the cell, transporting the peptide through the cell, and transporting the peptide out of the cell.

53. A chimeric protein of claim 52 wherein one of the segments binds to the cell.

54. A chimeric protein of claim 52 wherein one of the segments is a protein that is more prevalent in a Peyer's patch cell than in a non-Peyer's patch cell.

55. A chimeric protein of claim 52 wherein the cell is a Peyer's patch cell.

56. A chimeric protein of claim 55 wherein the cell is an M cell.

57. A method of targeting a composition or delivery vehicle to a Peyer's patch cell said method comprising utilizing a composition or vehicle that contains a protein ligand that will specifically bind to a protein that is up-regulated in Peyer's patch cells.

58. The method of claim 57 wherein the composition or delivery vehicle comprises a drug or antigen.

59. A method of selecting for a ligand that will selectively bind to a target in a Peyer's patch cell, said method comprising contacting a phage library with a protein that is upregulated in Peyer's patch cells.

60. The method of claim 59 wherein the protein is attached to a solid substrate.

61. A method of claim 1 wherein the protein is selected from the group consisting of HGF activator, ezrin, NCAD, MNDA, and LHX1.

62. A method of claim 34 wherein the upregulated protein is selected from the group consisting of HGF activator, ezrin, NCAD, MNDA, and LHX1, and the mRNA is for a protein selected from said group.

63. A method of claim 1 wherein the protein is selected from the group consisting of cadherin 2, neural cell adhesion molecule, integrin alpha 3, leukocyte adhesion glycoprotein p150, integrin beta 4, TIE, NCAML1, α3β1 integrin, CD11C antigen, CD104 antigen, CD44, NMDA, TKT, ephrin (type A), and Sp1.

64. A method of claim 34 wherein the upregulated protein is selected from the group consisting of cadherin 2, neural cell adhesion molecule, integrin alpha 3, leukocyte adhesion glycoprotein p150, integrin beta 4, TIE, NCAML1, α3β1 integrin, CD11C antigen, CD104 antigen, CD44, NMDA, TKT, ephrin (type A), and Sp1, and the mRNA is for a protein selected from said group.

65. A method of promoting enterocyte-M cell conversion, said method comprising orally administering an antigen, antigenic composition, or antigen-carrying particle to a person and either simultaneously with, or prior to, said administration, also orally administering a bacteria, or pro-biotic yogurts, or bacterial component to said person.
